(19) United States
(12) Patent Application Publication
(10) Pub. No.: US 2020/0268860 A1
(43) Pub. Date: Aug. 27, 2020

(54) METHODS AND SYSTEMS FOR PERFORMING A PATIENT-SPECIFIC IMMUNOTHERAPY PROCEDURE WITH CHAIN-OF-CUSTODY AND CHAIN-OF-IDENTITY BIOLOGICAL SAMPLE TRACKING

(71) Applicant: Kite Pharma, Inc., Santa Monica, CA (US)

(72) Inventors: Debra Susarchick, Summit, NJ (US); John Uhrin, Birmingham, AL (US); Michele Korfin, Glen Gardner, NJ (US)

(21) Appl. No.: 16/646,239

(22) PCT Filed: Sep. 17, 2018
(86) PCT No.: PCT/US2018/051298

§ 371 Date: Mar. 11, 2020

Related U.S. Application Data
(60) Provisional application No. 62/559,330, filed on Sep. 15, 2017.
(60) Provisional application No. 62/566,912, filed on Oct. 2, 2017.

(51) Int. Cl. A61K 39/00; A61K 39/12; G16H 10/60; G16H 10/40; G16H 20/17; G06Q 10/08

(52) U.S. Cl. CPC A61K 39/0011; A61K 39/12; G06Q 10/0833; G16H 10/40; G16H 20/17; G16H 10/60

(57) ABSTRACT

Methods and apparatuses are described for performing a patient-specific immunotherapy procedure. A computing device receives a request to create transfected T cells for a patient. The computing device generates a patient-specific identifier associated with the cell order request. The computing device initiates a process to create transfected T cells for infusion into the patient's bloodstream, comprising: performing a leukapheresis procedure on a sample of the patient's blood to collect T cells from the sample, transferring the collected T cells to a container, labeling the container with the patient-specific identifier, transmitting the collected T cells to a manufacturing facility, creating transfected T cells from the collected T cells using a cell modification technique, receiving the transfected T cells from the manufacturing facility, and infusing the transfected T cells into the patient's bloodstream. The computing device records a tracking event for each step, including the patient-specific identifier, to generate a chain of custody of the patient's T cells.

### RELATED APPLICATIONS

This application claims priority to U.S. Provisional Patent Application No. 62/559,330, filed Sep. 15, 2017, and to U.S. Provisional Patent Application No. 62/566,912, filed Oct. 2, 2017, each of which is incorporated by reference in its entirety.

### TECHNICAL FIELD

This application relates generally to methods and apparatuses, including computer program products, for performing a patient-specific immunotherapy procedure with chain-of-custody and chain-of-identity biological sample tracking

### BACKGROUND

In recent years, advances in medical technology have led to the emerging use of immunotherapies to treat different types of illnesses and diseases, including various forms of cancer. Generally, immunotherapy is the treatment of disease by stimulating or suppressing an immune response. Often, modified versions of a patient's own biological material, such as immune cells, are reintroduced into the patient in order to initiate and/or supplement the immune response.

For example, engineered immune cells have been shown to possess desired qualities in therapeutic treatments, particularly in oncology. Two main types of engineered immune cells are those that contain chimeric antigen receptors (termed “CARs” or “CAR-Ts”) and T-cell receptors (“TCRs”). These engineered cells are engineered to endow them with antigen specificity while retaining or enhancing their ability to recognize and kill a target cell. Chimeric antigen receptors may comprise, for example, (i) an antigen-specific component (“antigen binding molecule”), (ii) an extracellular domain, (iii) one or more costimulatory domains, and (iv) one or more activating domains. Each domain may be heterogeneous, that is, comprised of sequences derived from (or corresponding to) different protein chains.

Because many patients that undergo immunotherapy are critically ill, a crucial factor in the efficacy of such immunotherapy procedures is the ability to provide the modified biological material to the patient as quickly as possible, so that the therapeutic benefits may be maximized. Also, because many types of immunotherapy are tailored for the specific patient (i.e., using the patient's own cells), it is important to ensure that the patient's biological material is accurately tracked throughout the immunotherapy process—from extraction, to modification, and then to infusion back into the patient—to avoid delays in manufacturing, mislabeling of material, and misidentification of the patient. However, existing immunotherapy procedures generally lack a technical mechanism to track the patient's biological material automatically and to ensure that the biological material is tied to the specific patient's identity throughout the manufacturing process.

### SUMMARY

Therefore, what is needed are methods and systems for performing a patient-specific immunotherapy procedure with chain-of-custody and chain-of-identity biological sample tracking. The techniques described herein provide the specific technical advantage over existing systems of providing a continuous and automatic chain of custody and chain of identity for a patient-specific biological sample during an immunotherapy procedure, to create a computerized information portal that interested parties—such as the patient, physician, manufacturer, and other medical personnel—may use to quickly understand and track the current phase of the immunotherapy procedure and the status of the patient's biological sample during the procedure. Such advanced tracking is an improvement over existing systems that do not have technological solutions for maintaining a chain of custody and chain of identity—resulting in delays during the manufacturing process which, for a patient dealing with a life-threatening illness, may be immeasurably severe.

The invention, in one aspect, features a method of performing a patient-specific immunotherapy procedure. A computing device receives a cell order request to create transfected T cells for a patient. The computing device generates a patient-specific identifier associated with the cell order request, the patient-specific identifier comprising a patient identity element, a sales order identifier, and a cell order lot number. The computing device initiates a process to create transfected T cells for infusion into the patient's bloodstream, the process comprising: performing a leukapheresis procedure on a sample of the patient's blood to collect T cells from the sample, transferring the collected T cells to a container, labeling the container with the patient-specific identifier, transmitting the collected T cells to a manufacturing facility, creating transfected T cells from the collected T cells using a cell modification technique, receiving the transfected T cells from the manufacturing facility, and infusing the transfected T cells into the patient's bloodstream. The computing device records a tracking event for each step in the process, each tracking event including the patient-specific identifier. The tracking events comprise a chain of custody of the patient's T cells during the process.

The invention, in another aspect, features a method of tracking a cell order during an immunotherapy procedure. A computing device receives a cell order request for creating transfected T cells for a patient. The computing device generates a patient-specific identifier associated with the cell order request, the patient-specific identifier comprising a patient identity element, a sales order identifier, and a cell order lot number. The computing device monitors a process to create transfected T cells for infusion into the patient's bloodstream, the process comprising: receiving indicia that a leukapheresis procedure has been performed on a sample of the patient's blood to collect T cells from the sample, receiving indicia that the collected T cells have been transferred to a container, receiving indicia that the container has been labeled with the patient-specific identifier, receiving indicia that the collected T cells have been transmitted to a manufacturing facility, receiving indicia that transfected T cells have been created from the collected T cells using a cell modification technique, receiving indicia that the transfected T cells have been received from the manufacturing facility, and receiving indicia that the transfected T cells have been infused into the patient's bloodstream. The computing device records a tracking event when an indicia is received, each tracking event including the patient-specific identifier. The computing device maintains a chain of custody of the patient's T cells by storing the tracking events during the process.

The invention, in another aspect, features a method of performing a patient-specific immunotherapy procedure. A cell order request to create transfected T cells for a patient is received. An event tracking module executed on a processor generates a patient-specific identifier associated with the cell order request. A process to create transfected T cells for infusion into the patient's bloodstream is initiated, comprising: performing a leukapheresis procedure on a sample of the patient's blood to collect T cells from the sample, transferring the collected T cells to a container, labeling the container with the patient-specific identifier, transmitting the collected T cells to a manufacturing facility, creating transfected T cells from the collected T cells using a cell modification technique, receiving the transfected T cells from the manufacturing facility, and infusing the transfected T cells into the patient's bloodstream. The event tracking module receives, from a first client device located at the point of the leukapheresis procedure, a first tracking event that confirms the leukapheresis procedure and contains the patient-specific identifier. The event tracking module integrates the first tracking event in a data structure pertaining to the patient-specific identifier, where the data structure is stored in a database and the integrating step records a first timestamp with the first tracking event. The event tracking module receives, from a second client device located at the manufacturing facility, a second tracking event that confirms the receipt of the collected T cells at the manufacturing facility and contains the patient-specific identifier. The event tracking module integrates the second tracking event in the data structure pertaining to the patient-specific identifier, where the integrating step records a second timestamp with the second tracking event.

The invention, in another aspect, features a method of performing a patient-specific immunotherapy procedure. A tracking module executed on a processor receives a cell order request to create transfected T cells for a patient. The tracking module generates a patient-specific identifier associated with the cell order request, the patient-specific identifier identifying a patient, and a cell order lot. A database generates a data record for tracking the cell order, the data record identified in the database according to the patient-specific identifier. The tracking module receives a first tracking event indicating that the collected T cells are ready for shipment to a manufacturing facility. The data record corresponding to the patient-specific identifier is updated in accordance with the first tracking event. The tracking module receives, based on the container having been received by the manufacturing facility, a second tracking event indicating that the collected T cells have been received by a manufacturing facility. The data record corresponding to the patient-specific identifier is updated in accordance with the second tracking event. The tracking module receives, based on the manufacturing facility having created transfected T cells from the collected T cells using a cell modification technique, a third tracking event indicating that the transfected T cells have been created. The data record corresponding to the patient-specific identifier is updated in accordance with the third tracking event. The tracking module receives, based on the transfected T cells having been received from the manufacturing facility, a fourth tracking event indicating that the transfected T cells have been received. The data record corresponding to the patient-specific identifier is updated in accordance with the fourth tracking event. The tracking module receives, based on the transfected T cells having been infused into the patient's bloodstream, a fifth tracking event indicating that the transfected T cells have been infused into the patient's bloodstream. The data record corresponding to the patient-specific identifier is updated in accordance with the fifth tracking event, where each of the first, second, third, fourth, and fifth tracking events contains the patient-specific identifier, a timestamp, and an event identifier, and where the data record corresponding to the patient-specific identifier stores, in an ordered sequence, the first, second, third, fourth, and fifth tracking events when the data record is updated in accordance with the respective events.

Any of the above aspects may include one or more of the following features:

In some embodiments, the transfected T cells are created by transfecting the collected T cells with a polynucleotide encoding a chimeric antigen receptor (CAR), the CAR comprising an antigen binding molecule that specifically binds to a target molecule. Preferably, the antigen binding molecule is a single chain variable fragment (scFv).

It is envisioned that in some embodiments, the target molecule is a blood borne cancer-associated antigen. The blood borne cancer-associated antigens of the present invention are associated with one or more cancers selected from the group consisting of: acute myeloid leukemia (AML), chronic myelogenous leukemia (CML), chronic myelomonocytic leukemia (CMML), juvenile myelomonocytic leukemia, atypical chronic myeloid leukemia, acute promyelocytic leukemia (APL), acute monoblastic leukemia, acute erythroid leukemia, acute megakaryoblastic leukemia, lymphoblastic leukemia, B-lineage acute lymphoblastic leukemia, B-cell chronic lymphocytic leukemia, B-cell non-Hodgkin's lymphoma, myelodysplastic syndrome (MDS), myeloproliferative disorder, myeloid neoplasm, myeloid sarcoma), and Blastic Plasmacytoid Dendritic Cell Neoplasm (BPDCN).

In some embodiments, the target molecule is a viral infection-associated antigen. The viral infections of the present invention may be caused by any virus, including, for example, HIV.

The chimeric antigen receptors (CARs) of the present invention may further comprise at least one costimulatory domain. The costimulatory domains of the present invention include, but are not limited to, a signaling region, or other suitable portion, of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD11a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

Preferably, the costimulatory domain comprises CD28. CD28 costimulatory domains of the present invention comprise, for example, a sequence selected from the group consisting of SEQ ID NO. 2, SEQ ID NO. 4, SEQ ID NO. 6, and SEQ ID NO. 8.

In other embodiments, the costimulatory domain comprises CD8. CD8 costimulatory domains of the present invention comprise, for example, SEQ ID NO. 14.

The chimeric antigen receptors (CARs) of the present invention may further comprise at least one activating domain. The activating domains of the present invention comprise, for example, CD3. Preferably, the CD3 activating domains comprise CD3 zeta. CD3 zeta activating domains of the present invention comprise, for example, SEQ ID NO. 10.

In a preferred embodiment, the chimeric antigen receptor (CAR) comprises all or part of: an anti-CD19 scFv, CD28, and CD3 zeta.

In additional preferred embodiments, the chimeric antigen receptor (CAR) comprises:
all or part of: an anti-CD19 scFv, CD8, and 4-1BB;
all or part of: an anti-BCMA scFv and CD8;
all or part of: an anti-CD19 scFv, CD28, and 4-1BB;
all or part of: an anti-CD22 scFv and CD8;
all or part of: an anti-CD19 scFv, CD28, and EGFRt/19-28z/4-1BBL;
all or part of: an anti-MUC16 scFv, and CD28;
all or part of: an anti-CD171;
all or part of: an anti-CD123, and CD28;
all or part of: an anti-BCMA, CD8, and 4-1BB;
all or part of: an anti-CD19 and CD28;
all or part of: an anti-CD19 and CD8; or,
all or part of: CD28.

In a further preferred embodiment, the CAR comprises a leader sequence (CSF2RA), an anti-CD19 scFv, a Whitlow linker, a CD28 spacer, a CD28 costimulatory domain, and CD3 zeta. One example of such a CAR is encoded by the nucleotide sequence of SEQ ID NO. 146 and the amino acid sequence of SEQ ID NO. 147.

In an additional preferred embodiment, the CAR comprises a leader sequence (CD8), an anti-CD19 scFv, a Whitlow linker, a CD28T spacer, a CD28 costimulatory domain, and CD3 zeta. One example of such a CAR is encoded by the nucleotide sequence of SEQ ID NO. 148 and the amino acid sequence of SEQ ID NO. 149.

In another preferred embodiment, the CAR comprises a leader sequence (CD8a), an anti-CD19 scFv, a Whitlow linker, a CD8a spacer and transmembrane domain, a CD28 costimulatory domain, and CD3 zeta. One example of such a CAR is encoded by the nucleotide sequence of SEQ ID NO. 150 and the amino acid sequence of SEQ ID NO. 151.

In a further preferred embodiment, the CAR comprises a leader sequence (CD8), an anti-CLL-1 scFv, a G45 linker, a Minispacer, a CD28T (extracellular/transmembrane region of CD28), CD28 (an intracellular costimulatory region of CD28), and CD3 zeta. One example of such a CAR is encoded by the nucleotide sequence of SEQ ID NO. 154 and the amino acid sequence of SEQ ID NO. 155.

In an additional preferred embodiment, the CAR comprises a leader sequence (CD8a), an anti-BCMA scFv, a Whitlow linker, a CD28T spacer, a CD28 costimulatory domain, and CD3 zeta. One example of such a CAR is encoded by the nucleotide sequence of SEQ ID NO. 156 and the amino acid sequence of SEQ ID NO. 157.

In some embodiments, the transfected T cells are created by transfecting the collected T cells with a polynucleotide encoding a T cell receptor (TCR). The TCRs of the present invention may bind to a tumor-associated antigen. Tumor-associated antigens are associated with one or more cancers selected from the group consisting of: adrenocortical carcinoma, anal cancer, bladder cancer, bone cancer, brain cancer, breast cancer, carcinoid cancer, carcinoma, cervical cancer, colon cancer, endometrial cancer, esophageal cancer, extrahepatic bile duct cancer, extracranial germ cell cancer, eye cancer, gallbladder cancer, gastric cancer, germ cell tumor, gestational trophoblastic tumor, head and neck cancer, hypopharyngeal cancer, islet cell carcinoma, kidney cancer, large intestine cancer, laryngeal cancer, leukemia, lip and oral cavity cancer, liver cancer, lung cancer, lymphoma, malignant mesothelioma, Merkel cell carcinoma, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell cancer, pancreatic cancer, paranasal sinus and nasal cavity cancer, parathyroid cancer, penile cancer, pituitary cancer, plasma cell neoplasm, prostate cancer, rhabdomyosarcoma, rectal cancer, renal cell cancer, transitional cell cancer of the renal pelvis and ureter, salivary gland cancer, Sezary syndrome, skin cancers, small intestine cancer, soft tissue sarcoma, stomach cancer, testicular cancer, thymoma, thyroid cancer, urethral cancer, uterine cancer, vaginal cancer, vulvar cancer, and Wilms' tumor.

In certain aspects, the present application may be suitable for target molecule to hematologic cancer. In some embodiments, the cancer is of the white blood cells. In other embodiments, the cancer is of the plasma cells. In some embodiments, the cancer is leukemia, lymphoma, or myeloma. In certain embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBCL), small cell- or a large cell-follicular lymphoma, splenic marginal zone lymphoma (SMZL), systemic amyloid light chain amyloidosis, T-cell acute lymphoid leukemia (TALL), T-cell lymphoma, transformed follicular lymphoma, Waldenstrom macroglobulinemia, or a combination thereof.

In additional preferred embodiments, the TCR comprises an AV38-2 variable alpha chain, a BV7-2 variable beta chain, a murine constant alpha domain, a murine constant beta domain, TRAJ40, Furin-SG SG-P2A, and TRBJ1-3. One example of such a TCR is encoded by the nucleotide sequence of SEQ ID NO. 152 and the amino acid sequence of SEQ ID NO. 153.

In further preferred embodiments, the TCR comprises a TCR beta chain variable region, a TCR beta chain constant region, P2A peptide (with Furin cleavage site and linker), a TCR alpha chain variable region, and a TCR alpha chain constant region. One example of such a TCR is encoded by the nucleotide sequence of SEQ ID NO. 158 and the amino acid sequence of SEQ ID NO. 159. Another example of such a TCR is encoded by the nucleotide sequence of SEQ ID NO. 160 and the amino acid sequence of SEQ ID NO. 161.

In other embodiments, the TCRs of the present invention bind to a viral infection-associated antigen. Viral infections of the present invention may be caused by any virus, including, for example, HIV.

Polynucleotides of the present invention may be a component of a vector. Suitable vectors include, but are not limited to, retroviral vectors, DNA vectors, plasmids, RNA vectors, adenoviral vectors, adenovirus associated vectors, lentiviral vectors, or any combination thereof. Preferably, the vector is a lentiviral vector. The lentiviral vectors of the present invention include, but are not limited to, pGAR and derivatives thereof.

In some embodiments, initiating a process to create transfected T cells further comprises requesting a timeslot for performing the leukapheresis procedure from a remote computing device, receiving indicia responsive to the request for a timeslot that a timeslot is available to perform the leukapheresis procedure, and confirming acceptance of the available timeslot with the remote computing device.

In some embodiments, initiating a process to create transfected T cells further comprises receiving indicia that the transfected T cells have been shipped from a first site after being created and receiving indicia that the transfected T cells have been received at a second site before being infused, where the computing device records a tracking event upon receiving the indicia that the transfected T cells have been shipped, and the computing device records a tracking event upon receiving the indicia that the transfected T cells have been received at a second site.

In some embodiments, the patient identity element comprises a first patient ID associated with the immunotherapy procedure and a second patient ID associated with a facility that administers one or more of: the leukapheresis procedure or the infusion of the transfected T cells. In some embodiments, the computing device stores the tracking events in an ordered sequence. In some embodiments, the ordered sequence is arranged in chronological order.

Other aspects and advantages of the invention will become apparent from the following detailed description, taken in conjunction with the accompanying drawings, illustrating the principles of the invention by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1A is a block diagram of a system for performing a patient-specific immunotherapy procedure with chain-of-custody and chain-of-identity biological sample tracking.

FIG. 1B is a detailed block diagram of a system for performing a patient-specific immunotherapy procedure with chain-of-custody and chain-of-identity biological sample tracking.

FIG. 2 is a flow diagram of a computerized method of performing a patient-specific immunotherapy procedure with chain-of-custody and chain-of-identity biological sample tracking.

FIGS. 3A and 3B are exemplary screenshots generated by a user interface module to receive patient-specific information during a patient-specific immunotherapy procedure.

FIGS. 4A to 4D are exemplary screenshots generated by a user interface module to receive confirmation of extraction and infusion sites, and to schedule an appointment, during a patient-specific immunotherapy procedure.

FIGS. 5A and 5B are exemplary screenshots generated by a user interface module to display a chain of custody for biological material during a patient-specific immunotherapy procedure.

### DETAILED DESCRIPTION

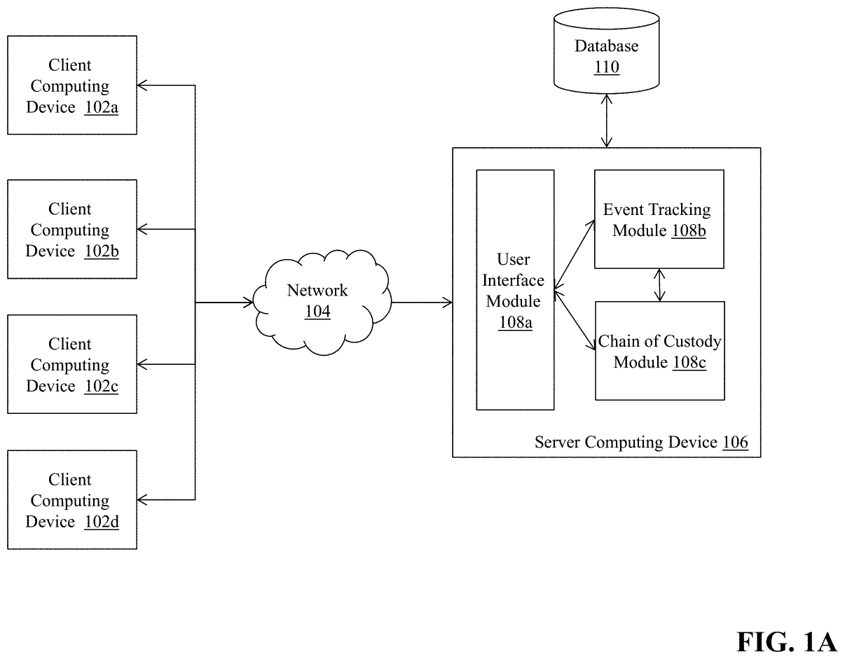

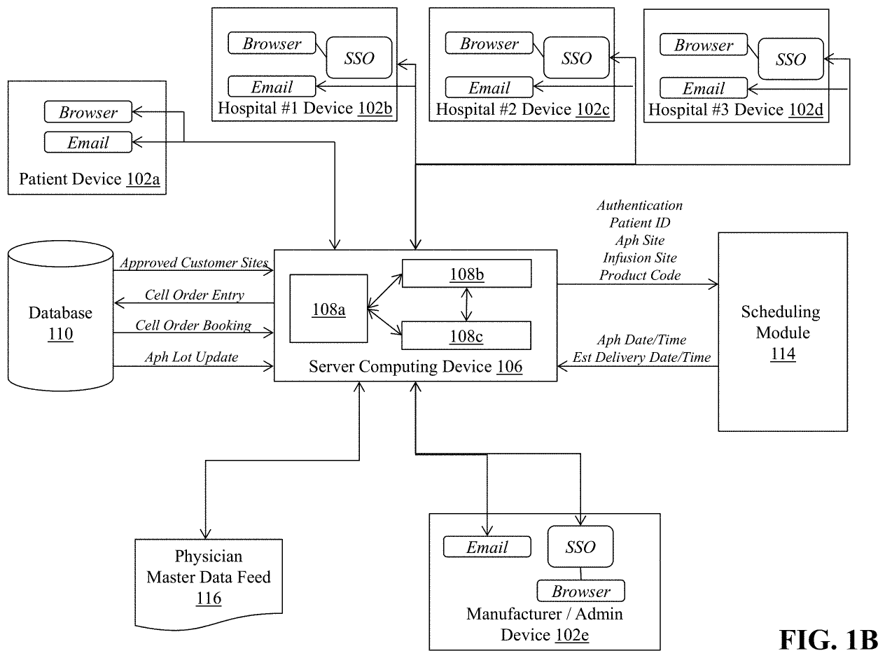

FIG. 1A is a block diagram of a system 100 for performing a patient-specific immunotherapy procedure with chain-of-custody and chain-of-identity biological sample tracking. The system of FIG. 1A includes a plurality of client computing devices 102a-102d, a communications network 104, a server computing device 106 with a user interface module 108a, an event tracking module 108b, and a chain of custody module 108c, and a database 110.

The client computing devices 102a-102d are connected to the communications network 104 in order to communicate with the server computing device 106 to provide input and receive output relating to the process of performing a patient-specific immunotherapy procedure with chain-of-custody and chain-of-identity biological sample tracking as described herein. In some embodiments, each client computing device 102a-102d may be coupled to a respective display device for, e.g., providing a detailed graphical user interface (GUI) that receives input for and presents output resulting from the methods and systems described herein. For example, the client computing device 102a-102d may connect to the user interface module 108a of server computing device 106, which provides, e.g., a web-based portal for users of the client computing devices 102a-102c to access functionality associated with the methods described herein.

Exemplary client devices 102a-102d include but are not limited to desktop computers, laptop computers, tablets, mobile devices, smartphones, and internet appliances. It should be appreciated that other types of computing devices that are capable of connecting to the components of the system of FIG. 1A may be used without departing from the scope of invention. It also should be appreciated that each of the client computing devices 102a-102d may be associated with a different user type—for example, client computing device 102a may be associated with a patient accessing the system of FIG. 1A to generate a user profile and receive updates on a patient-specific immunotherapy procedure; client computing device 102b may be associated with a physician who is treating the patient and who accesses the system of FIG. 1A to initiate an immunotherapy procedure for the patient; client computing device 102c may be associated with a hospital or other facility that is administering the immunotherapy procedure to the patient; and client computing device 102d may be associated with a manufacturing facility that is creating patient-specific immunotherapy product (as will be described herein) for use in the immunotherapy procedure.

The communications network 104 enables the other components of the system 100 to communicate with each other in order to conduct the process of performing a patient-specific immunotherapy procedure with chain-of-custody and chain-of-identity biological sample tracking as described herein. The network 104 may be a local network, such as a LAN, or a wide area network, such as the Internet and/or a cellular network. In some embodiments, the network 104 is comprised of several discrete networks and/or sub-networks (e.g., cellular to Internet) that enable the components of the system of FIG. 1A to communicate with each other.

The server computing device 106 is a combination of hardware and software modules that includes specialized hardware and/or software modules that execute on a processor and interact with memory modules of the server computing device 106 to perform functions for performing a patient-specific immunotherapy procedure with chain-of-custody and chain-of-identity biological sample tracking as described herein. The server computing device 106 includes a user interface module 108a, an event tracking module 108b, and a chain of custody module 108c (as mentioned above) that execute on and/or interact with the processor of the server computing device 106.

In some embodiments, the user interface module 108a, the event tracking module 108b, and the chain of custody module 108c are specialized sets of computer software instructions programmed onto one or more dedicated processors in the server computing device 106 and may include specifically-designated memory locations and/or registers for executing the specialized computer software instructions. Although the modules 108a-108c are shown in FIG. 1A as executing within the same server computing device 106, in some embodiments the functionality of the modules 108a-108c may be distributed among a plurality of server computing devices. As shown in FIG. 1A, the server computing device 106 enables the modules 108a-108c to communicate with each other in order to exchange data for the purposes of performing the described functions. It should be appreciated that any number of computing devices, arranged in a variety of architectures, resources, and configurations (e.g., cluster computing, virtual computing, cloud computing) may be used without departing from the scope of the invention. The exemplary functionality of the modules 108a-108c is described in detail below.

The database 110 is a computing device (or in some embodiments, a set of computing devices) coupled to the server computing device 106 and is configured to receive, generate, and store specific segments of data relating to the process of performing a patient-specific immunotherapy procedure with chain-of-custody and chain-of-identity biological sample tracking as described herein. In some embodiments, all or a portion of the database 110 may be integrated with the server computing device 106 or be located on a separate computing device or devices. The database 110 may comprise one or more databases configured to store portions of data used by the other components of the system of FIG. 1A, as will be described in greater detail below. In some embodiments, the database 110 comprises an enterprise business suite, such as Oracle E-Business Suite (EBS), that includes various modules that enable a spectrum of functionality to support the methods and systems described herein—including logistics, supply chain, transportation, CRM, and other types of modules.

FIG. 1B is a detailed block diagram of the system of FIG. 1A for performing a patient-specific immunotherapy procedure with chain-of-custody and chain-of-identity biological sample tracking. As shown in FIG. 1B, the server computing device 106 is the central component in the overall hardware architecture, interfacing with client computing devices 102a-102e and database 110, and also interfacing with a scheduling module 114 and a physician master data feed 116. In some embodiments, the server computing device 106 and the corresponding modules 108a-108c leverage the Salesforce platform, available from Salesforce.com, Inc. of San Francisco, Calif., to integrate certain of the functions described herein. The client computing devices 102a-102e communicate with the server computing device 106 to perform patient enrollment in the immunotherapy procedure and to monitor the chain-of-custody and chain-of-identity tracking (e.g., via browser-based user interfaces) as described herein.

For example, client computing device 102a may be associated with the patient undergoing the immunotherapy procedure and may include browser software and email software to enable the patient to both monitor the tracking and to electronically sign documents required to participate in the immunotherapy procedure (e.g., via DocuSign or other similar technology). Similarly, the client computing devices 102b-102d may be located at different hospitals where a treating physician may enroll a patient in the immunotherapy procedure, place a cell order with the system, and monitor the chain-of-custody and chain-of identity tracking using the browser software. The client computing devices 102b-102d also include a single-sign-on (SSO) module that enables the devices to authenticate to the server computing device 106 (e.g., using SAML 2.0 supported SSO or a specific username/password for the server). The client computing device 102e may be located at an administration or manufacturing site to enable an administrator of the server computing device 106 to communicate with the server, receive communications such as emails from other participants in the system, and monitor the chain-of-custody and chain-of identity tracking using the browser software.

As described above, the database 110 may comprise an enterprise business suite that manages the data for the server computing device 106 and includes modules to enable chain-of-custody and chain-of-identity tracking and logistics for the biological sample. For example, the database 110 may transmit approved customer sites to the server 106 upon request, receive cell order entry data from the server 106, and provide cell order booking and apheresis lot update information to the server 106.

The scheduling module 114 may be integrated into the server computing device 106 or reside on a separate computing device. The scheduling module 114 may authenticate to and communicate with the server computing device 106 to receive certain information about the cell order and immunotherapy procedure (e.g., patient ID, apheresis site, infusion site, and product code) and provide calendaring and scheduling functionality to the server 106 (e.g., enabling a treating physician to select an apheresis date/time and provide an estimated delivery date/time for the biological sample once it has gone through the manufacturing process). Also, the server computing device 106 may communicate with a physician master data feed 116 (e.g., provided using the Veeva™ CRM platform integrated with the Heroku™ application) to receive certain information about treating physicians.

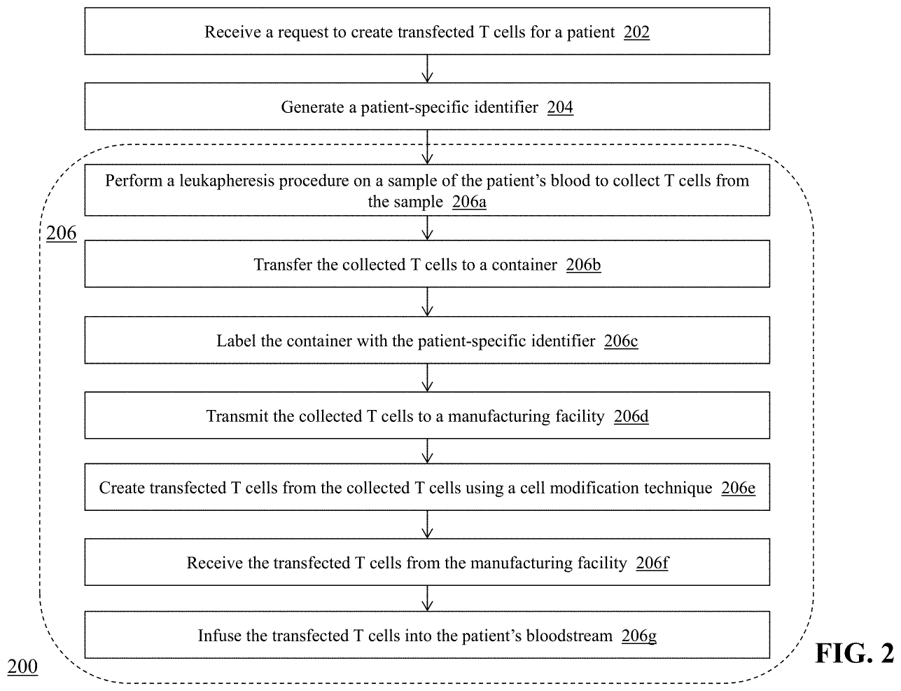

FIG. 2 is a flow diagram of a computerized method 200 of performing a patient-specific immunotherapy procedure with chain-of-custody and chain-of-identity biological sample tracking, using the system of FIG. 1A and/or the system of FIG. 1B. As will be appreciated, the methods and systems described herein are presented in the context of performing a patient-specific immunotherapy procedure. As used herein, “patient-specific immunotherapy procedure” means any procedure that uses molecular or cellular components of the immune system to target and/or destroy cancer, pathogenic, or other disease-causing cells. An immunotherapy procedure is “patient-specific” if it utilizes components of a patient's immune system to treat that patient's own cancer, pathology, or other disease.

As used herein, the terms “patient” and “subject” are used interchangeably and include human and non-human animals, as well as those with formally diagnosed disorders, those without formally recognized disorders, those receiving medical attention, those at risk of developing disorders, etc. In addition to humans, categories of animals within the scope of the present invention include, for example, agricultural animals, domestic animals, laboratory animals, etc. Some examples of agricultural animals include cows, pigs, horses, goats, etc. Some examples of domestic animals include dogs, cats, etc. Some examples of laboratory animals include rats, mice, rabbits, guinea pigs, etc.

The term “leukapheresis” refers to a specific form of apheresis which involves the selective separation and removal of leukocytes from a blood sample. During leukapheresis, the removed blood is passed through a cell separation device which separates nucleated white blood cells, including T cells, from red blood cells and plasma. The separated T cells may then be collected to be used in the cell modification techniques of the present invention. In certain embodiments, the red blood cells and plasma are returned to the individual as part of the separation process. In additional embodiments, the red blood cells and plasma are discarded or stored for further analysis.

As used herein, the terms “T cell” and “T lymphocyte” are interchangeable. T cells are a subset of lymphocytes defined by their development in the thymus and by heterodimeric receptors associated with the proteins of the CD3 complex. T cells of the present invention include, but are not limited to, naïve T cells, cytotoxic T cells, helper T cells, suppressor T cells, regulatory T cells, memory T cells, NKT cells, γδ cells, CD8αα cells, lymphokine activated cells, TCR-expressing cells, subtypes thereof, and any other cell type which may express chimeric receptor chain.

T cells may be engineered to possess specificity to one or more desired targets. For example, T cells may be transduced with DNA or other genetic material encoding an antigen binding molecule, such as one or more single chain variable fragment (“scFv”) of an antibody, in conjunction with one or more signaling molecules, and/or one or more activating domains, such as CD3 zeta. In addition to the CAR-T cells' ability to recognize and destroy the targeted cells, successful T cell therapy benefits from the CAR-T cells' ability to persist and maintain the ability to proliferate in response to antigen.

As used herein, the term “cell modification technique” includes, but is not limited to, transfection and transduction. The term “transfection” and grammatical variations thereof, refer to the introduction of foreign or exogenous DNA into a cell. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Transfection techniques include, but are not limited to, calcium phosphate-DNA co-precipitation, DEAE-dextran-mediated transfection, cationic lipid-mediated delivery, polybrene-mediated transfection, electroporation, sonoporation, microinjection, liposome fusion, lipofection (lipid transfection), polymer transfection, nanoparticles, polyplexes, receptor-mediated gene delivery, delivery mediated by polylysine, histone, chitosan, and peptides, protoplast fusion, retroviral infection, and biolistics (e.g. Gene Gun). The term “transduction” and grammatical variations thereof refer to the process whereby foreign DNA is introduced into a cell via viral vector. See Jones et al., (1998). Genetics: principles and analysis. Boston: Jones & Bartlett Publ.

As used herein, the term “infuse” and grammatical variations thereof mean to introduce a solution into a body through a blood vessel. An infusion of the present invention includes, but is not limited to, therapeutic introduction of a fluid other than whole blood into a blood vessel. For example, transfected T cells of the present invention may be infused into a patient's bloodstream, for example, intramuscularly, intravenously, intraarterially, intraperitoneally, or subcutaneously.

It will be appreciated that chimeric antigen receptors (CARs or CAR-Ts) are, and T cell receptors (TCRs) may, be genetically engineered receptors. These engineered receptors may be readily inserted into and expressed by immune cells, including T cells in accordance with techniques known in the art. With a CAR, a single receptor may be programmed to both recognize a specific antigen and, when bound to that antigen, activate the immune cell to attack and destroy the cell bearing that antigen. When these antigens exist on tumor cells, an immune cell that expresses the CAR may target and kill the tumor cell.

CARs may be engineered to bind to an antigen (such as a cell-surface antigen) by incorporating an antigen binding molecule that interacts with that targeted antigen. An “antigen binding molecule” as used herein means any protein that binds a specified target molecule. Antigen binding molecules include, but are not limited to antibodies and binding parts thereof, such as immunologically functional fragments. Peptibodies (i.e., Fc fusion molecules comprising peptide binding domains) are another example of suitable antigen binding molecules.

The term “immunologically functional fragment” (or “fragment”) of an antigen binding molecule is a species of antigen binding molecule comprising a portion (regardless of how that portion is obtained or synthesized) of an antibody that lacks at least some of the amino acids present in a full-length chain but which is still capable of specifically binding to an antigen. Such fragments are biologically active in that they bind to the target antigen and may compete with other antigen binding molecules, including intact antibodies, for binding to a given epitope. In some embodiments, the fragments are neutralizing fragments. In one aspect, such a fragment will retain at least one CDR present in the full-length light or heavy chain, and in some embodiments will comprise a single heavy chain and/or light chain or portion thereof. These fragments may be produced by recombinant DNA techniques, or may be produced by enzymatic or chemical cleavage of antigen binding molecules, including intact antibodies.

Immunologically functional immunoglobulin fragments include, but are not limited to, scFv fragments, Fab fragments (Fab′, F(ab′)₂, and the like), one or more CDR, a diabody (heavy chain variable domain on the same polypeptide as a light chain variable domain, connected via a short peptide linker that is too short to permit pairing between the two domains on the same chain), domain antibodies, and single-chain antibodies. These fragments may be derived from any mammalian source, including but not limited to human, mouse, rat, camelid or rabbit. As will be appreciated by one of skill in the art, an antigen binding molecule may include non-protein components.

In some embodiments, the antigen binding molecule binds to an antigen on a tumor cell. In some embodiments, the antigen binding molecule binds to an antigen on a cell involved in a hyperproliferative disease or to a viral or bacterial antigen. In further embodiments, the antigen binding molecule is an antibody or fragment thereof, including one or more of the complementarity determining regions (CDRs) thereof. In further embodiments, the antigen binding molecule is a single chain variable fragment (scFv).

Preferably, the antigen binding molecule is an antibody fragment thereof. More preferably, the antigen binding molecule comprises one or more single chain variable fragment (“scFv”). An scFv is a single chain antibody fragment having the variable regions of the heavy and light chains of an antibody linked together. See U.S. Pat. Nos. 7,741,465, 6,319,494, PCT application WO88/01649, and U.S. Pat. Nos. 4,946,778 and 5,260,203, the disclosures of which are incorporated by reference in their entirety, as well as Eshhar et al., Cancer Immunol Immunotherapy (1997) 45: 131-136. An scFv retains the parent antibody's ability to specifically interact with a target antigen. scFvs are preferred for use in chimeric antigen receptors because they may be engineered to be expressed as part of a single chain along with the other CAR components. Id. See also Krause et al., J. Exp. Med., Volume 188, No. 4, 1998 (619-626); Finney et al., Journal of Immunology, 1998, 161: 2791-2797.

It will be appreciated that the antigen binding molecule is typically contained within the extracellular portion of the CAR such that it is capable of recognizing and binding to the antigen of interest. Bispecific and multispecific CARs are contemplated within the scope of the invention, with specificity to more than one target of interest.

In certain embodiments, the polypeptide structure of the antigen binding molecules is based on antibodies, including, but not limited to, monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”), and fragments thereof, respectively. In some embodiments, the antigen binding molecule comprises or consists of avimers.

An antigen binding molecule is said to “specifically bind” its target antigen when the dissociation constant (K_{d}) is ˜1×10⁻⁷ M. The antigen binding molecule specifically binds antigen with “high affinity” when the K_{d} is 1-5×10⁻⁹ M, and with “very high affinity” when the K_{d} is 1-5×10⁻¹⁰ M. In one embodiment, the antigen binding molecule has a K_{d} of 10⁻⁹ M. In one embodiment, the off-rate is <1×10⁻⁵.

Those of ordinary skill in the art will understand that the target molecules of the present invention may be any molecule, the specificity for which is desired to be transferred to transfected T cells. The terms “target molecule” or “antigen” refers to a molecule or a portion of a molecule capable of being bound by an antigen binding molecule. In certain embodiments, a target may have one or more epitopes.

Preferably, target molecules of the present invention include, but are not limited to, blood borne cancer-associated antigens. Non-limiting examples of blood borne cancer-associated antigens include antigens associated with one or more cancers selected from the group consisting of acute myeloid leukemia (AML), chronic myelogenous leukemia (CML), chronic myelomonocytic leukemia (CMML), juvenile myelomonocytic leukemia, atypical chronic myeloid leukemia, acute promyelocytic leukemia (APL), acute monoblastic leukemia, acute erythroid leukemia, acute megakaryoblastic leukemia, lymphoblastic leukemia, B-lineage acute lymphoblastic leukemia, B-cell chronic lymphocytic leukemia, B-cell non-Hodgkin's lymphoma, myelodysplastic syndrome (MDS), myeloproliferative disorder, myeloid neoplasm, myeloid sarcoma), and Blastic Plasmacytoid Dendritic Cell Neoplasm (BPDCN).

In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CSPG4, CTLA-4, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGFI)-1, intestinal carboxyl esterase, kappa chain, LAGA-1a, lambda chain, Lassa Virus-specific antigen, lectin-reactive AFP, lineage-specific or tissue specific antigen such as CD3, MAGE, MAGE-Al, major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp72, mutated p53, mutated p53, mutated ras, neutrophil elastase, NKG2D, Nkp30, NY-ESO-1, p53, PAP, prostase, prostase specific antigen (PSA), prostate carcinoma tumor antigen-1 (PCTA-1), prostate-specific antigen, prostein, PSMA, RAGE-1, ROR1, RUL RU2 (AS), surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gp120), as well as any derivate or variant of these surface markers.

In some embodiments, target molecules of the present invention include viral infection-associated antigens. Viral infections of the present invention may be caused by any virus, including, for example, HIV.

This list of possible target molecules is not intended to be exclusive and those of ordinary skill in the art will be aware of many additional molecules that would be useful to target with the chimeric antigen receptors of the present invention.

Chimeric antigen receptors may incorporate costimulatory (signaling) domains to increase their potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). For example, CD28 is a costimulatory protein found naturally on T-cells. A variety of costimulatory molecules are set forth herein, but it will be appreciated that additional costimulatory molecules are also included within the scope of this invention.

The complete native amino acid sequence of CD28 is described in NCBI Reference Sequence: NP_006130.1. The complete native CD28 nucleic acid sequence is described in NCBI Reference Sequence: NM_006139.1.

Certain CD28 domains have been used in chimeric antigen receptors. In accordance with the present invention, it has now been found that a novel CD28 extracellular (hinge) construct, termed “CD28T”, unexpectedly provides certain benefits when utilized in a CAR construct. This construct demonstrates the ability to retain (and at times exceed) the properties of CD28-containing CARs, despite truncation (removal) of multiple amino acids from the extracellular CD28 sequence. These benefits include equivalent or superior cytokine production, equivalent or superior cytolytic activity, and/or equivalent or superior CAR expression levels.

The nucleotide sequence of the CD28T molecule, including the extracellular domain, and the CD28 transmembrane and intracellular domains is set forth in SEQ ID NO. 1:

CTTGATAATGAAAAGTCAAACGGAACAATCATTCACGTGAAGGGCAAGCA
CCTCTGTCCGTCACCCTTGTTCCCTGGTCCATCCAAGCCATTCTGGGTGT
TGGTCGTAGTGGGTGGAGTCCTCGCTTGTTACTCTCTGCTCGTCACCGTG
GCTTTTATAATCTTCTGGGTTAGATCCAAAAGAAGCCGCCTGCTCCATAG
CGATTACATGAATATGACTCCACGCCGCCCTGGCCCCACAAGGAAACACT
ACCAGCCTTACGCACCACCTAGAGATTTCGCTGCCTATCGGAGC

The corresponding amino acid sequence is set forth in SEQ ID NO. 2:

LDNEKSNGTIIHVKGKHLCPSPLFPGPSKPFWVLVVVGGVLACYSLLVTV
AFIIFWVRSK RSRLLHSDYM NMTPRRPGPT RKHYQPYAPP
RDFAAYRS

The nucleotide sequence of the extracellular portion of CD28T is set forth in SEQ ID NO. 3:

CTTGATAATGAAAAGTCAAACGGAACAATCATTCACGTGAAGGGCAAGCA
CCTCTGTCCGTCACCCTTGTTCCCTGGTCCATCCAAGCCA

The corresponding amino acid sequence of the CD28T extracellular domain is set forth in SEQ ID NO. 4:

LDNEKSNGTI IHVKGKHLCP SPLFPGPSKP

The nucleotide sequence of the CD28 transmembrane domain is set forth in SEQ ID NO. 5:

TTCTGGGTGTTGGTCGTAGTGGGTGGAGTCCTCGCTTGTTACTCTCTGCT
CGTCACCGTGGCTTTTATAATCTTCTGGGTT

The amino acid sequence of the CD28 transmembrane domain is set forth in SEQ ID NO. 6:

FWVLVVVGGV LACYSLLVTV AFIIFWV

The nucleotide sequence of the CD28 intracellular signaling domain is set forth in SEQ ID NO. 7:

AGATCCAAAAGAAGCCGCCTGCTCCATAGCGATTACATGAATATGACTCC
ACGCCGCCCTGGCCCCACAAGGAAACACTACCAGCCTTACGCACCACCTA
GAGATTTCGCTGCCTATCGGAGC

The amino acid sequence of the CD28 intracellular signaling domain is set forth in SEQ ID NO. 8:

RSKRSRLLHSDYMNMTPRRPGPTRKHYQPYAPPRDFAAYRS

Additional CD28 sequences suitable for use in the invention include the CD28 nucleotide sequence set forth in SEQ ID NO. 11:

ATTGAGGTGATGTATCCACCGCCTTACCTGGATAACGAAAAGAGTAACGG
TACCATCATTCACGTGAAAGGTAAACACCTGTGTCCTTCTCCCCTCTTCC
CCGGGCCATCAAAGCCC

The corresponding amino acid sequence is set forth in SEQ ID NO. 12: IEVMYPPPYLDNEKSNGTIIHVKGKHLCPSPLFPGPSKP

It will be appreciated that the invention relates to antigen binding molecules, CARs, TCRs, and the like comprising at least one isolated nucleic acid sequence of SEQ ID NO. 1 or SEQ ID NO. 3. It will further be appreciated that the invention relates to antigen binding molecules, CARs, TCRs, and the like wherein the extracellular portion consists of at least one isolated nucleic acid sequence of SEQ ID NO. 1 or SEQ ID NO. 3. Additionally, it will be appreciated that the invention relates to antigen binding molecules, CARs, TCRs, and the like wherein the extracellular portion consists essentially of at least one isolated nucleic acid sequence of SEQ ID NO. 1 or SEQ ID NO. 3.

It will be appreciated that the invention relates to antigen binding molecules, CARs, TCRs, and the like comprising at least one amino acid sequence of SEQ ID NO. 2 or SEQ ID NO. 4. It will further be appreciated that the invention relates to antigen binding molecules, CARs, TCRs, and the like wherein the extracellular portion consists of at least one amino acid sequence of SEQ ID NO. 2 or SEQ ID NO. 4. It will also be appreciated that the invention relates to antigen binding molecules, CARs, TCRs, and the like wherein the extracellular portion consists essentially of at least one amino acid sequence of SEQ ID NO. 2 or SEQ ID NO. 4.

Another suitable source of extracellular and/or transmembrane domains may be derived from (or correspond to) some or all of CD8.

The nucleotide sequence of a suitable CD8 extracellular and transmembrane domain is set forth in SEQ ID NO. 13:

GCTGCAGCATTGAGCAACTCAATAATGTATTTTAGTCACTTTGTACCAGT
GTTCTTGCCGGCTAAGCCTACTACCACACCCGCTCCACGGCCACCTACCC
CAGCTCCTACCATCGCTTCACAGCCTCTGTCCCTGCGCCCAGAGGCTTGC
CGACCGGCCGCAGGGGGCGCTGTTCATACCAGAGGACTGGATTTCGCCTG
CGATATCTATATCTGGGCACCCCTGGCCGGAACCTGCGGCGTACTCCTGC
TGTCCCTGGTCATCACGCTCTATTGTAATCACAGGAAC

The corresponding amino acid sequence is set forth in SEQ ID NO. 14:

AAALSNSIMYFSHFVPVFLPAKPTTTPAPRPPTPAPTIASQPLSLRPEAC
RPAAGGAVHTRGLDFACDIYIWAPLAGTCGVLLLSLVITLYCNHRN

Structurally, it will be appreciated that the domains described herein correspond to locations relative to an immune or other cell. These domains thus may be part of the (i) “hinge” or extracellular (EC) domain, (ii) the transmembrane (TM) domain, and/or (iii) the intracellular/cytoplasmic domain (IC). The intracellular component frequently comprises, in part, an activating domain such as a portion of a member of the CD3 family, preferably CD3 zeta. This domain is capable of activating the T cell upon binding of the antigen binding molecule to its target. It will be appreciated that the intracellular domain typically further comprises one or more costimulatory molecules as described herein.

A “costimulatory molecule” as used herein refers to a molecule that provides a signal which mediates a T cell response, including, but not limited to, proliferation, activation, differentiation, and the like. Costimulatory molecules may provide a signal in addition to the primary signal provided by an activating molecule as described herein.

It will be appreciated that suitable costimulatory domains within the scope of the invention may be derived from (or correspond to) costimulatory molecules, such as, for example, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CD11a/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNF, TNFr, integrin, signaling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD1-1d, ITGAE, CD103, ITGAL, CD1-la, LFA-1, ITGAM, CD1-1b, ITGAX, CD1-1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD19a, CD83 ligand, or fragments or combinations thereof. It will be appreciated that additional costimulatory molecules, or fragments thereof, not listed above are within the scope of the invention.

In some embodiments, the costimulatory domain may comprise all or a portion of the 4-1BB nucleic acid sequence set forth in SEQ ID NO. 140, and the corresponding amino acid sequence as set forth in SEQ ID NO. 141. In other embodiments, the costimulatory domain may comprise all or a portion of the amino acid sequence of OX40 as set forth in SEQ ID NO. 142. See also Hombach et al., Oncoimmunology. 2012 Jul. 1; 1(4): 458-466. In still other embodiments, the costimulatory domain may comprise all or a portion of the ICOS molecule as described in Guedan et al., Aug. 14, 2014; Blood: 124 (7) and Shen et al., Journal of Hematology & Oncology (2013) 6:33. In still other embodiments, the costimulatory domain may comprise all or a portion of CD27 as described in Song et al., Oncoimmunology. 2012 Jul. 1; 1(4): 547-549.

The engineered T cells of the invention comprise an antigen binding molecule (such as an scFv), an extracellular domain (which may comprise a “hinge” domain), a transmembrane domain, and an intracellular domain. The intracellular domain may comprise at least in part an activating domain, preferably comprised of a CD3 family member such as CD3 zeta, CD3 epsilon, CD3 gamma, or portions thereof.

It will further be appreciated that the antigen binding molecule (e.g., one or more scFvs) is engineered such that it is located in the extracellular portion of the molecule/construct, such that it is capable of recognizing and binding to its target or targets.

It will be appreciated that the hinge region may contain some or all of a member of the immunoglobulin family such as IgG1, IgG2, IgG3, IgG4, IgA, IgD, IgE, IgM, or fragment thereof.

In some embodiments, the extracellular domain is positioned between the antigen binding molecule and the transmembrane domain.

Extracellular domains of particular use in this invention may be derived from (i.e., comprise) all or some of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof. The extracellular domain may be derived either from a natural or from a synthetic source.

Extracellular domains often comprise the hinge portion, sometimes referred to as the “spacer” region. A variety of hinges may be employed in accordance with the invention, including portions or derivatives of the molecules described herein.

The CAR may be designed with a transmembrane domain that is fused to the extracellular domain of the CAR. It may similarly be fused to the intracellular domain of the CAR. In some instances, the transmembrane domain may be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (comprise, or correspond to) CD28, CD28T, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

Optionally, short linkers may form linkages between any or some of the extracellular, transmembrane, and intracellular domains of the CAR.

In other embodiments, the transmembrane domain in the CAR of the invention is a CD8 transmembrane domain. In one embodiment, the CD8 transmembrane domain comprises the transmembrane portion of the nucleic acid sequence of SEQ ID NO: 13. In another embodiment, the CD8 transmembrane domain comprises the nucleic acid sequence that encodes the transmembrane amino acid sequence contained within SEQ ID NO: 14.

In certain embodiments, the transmembrane domain in the CAR of the invention is the CD28 transmembrane domain. In one embodiment, the CD28 transmembrane domain comprises the nucleic acid sequence of SEQ ID NO: 5. In one embodiment, the CD28 transmembrane domain comprises the nucleic acid sequence that encodes the amino acid sequence of SEQ ID NO: 6. In another embodiment, the CD28 transmembrane domain comprises the amino acid sequence of SEQ ID NO: 6.

The intracellular (cytoplasmic) domain of the engineered T cells of the invention may provide activation of at least one of the normal effector functions of the immune cell. Effector function of a T cell, for example, may refer to cytolytic activity or helper activity, including the secretion of cytokines.

It will be appreciated that suitable intracellular molecules include (i.e., comprise), but are not limited to signaling domains derived from (or corresponding to) CD28, CD28T, OX-40, 4-1BB/CD137, CD2, CD7, CD27, CD30, CD40, programmed death-1 (PD-1), inducible T cell costimulator (ICOS), lymphocyte function-associated antigen-1 (LFA-1, CD1-1a/CD18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD1 lb, ITGAX, CD11c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, or any combination thereof.

In a preferred embodiment, the intracellular/cytoplasmic domain of the CAR may be designed to comprise the CD3 zeta domain by itself or combined with any other desired intracellular domain(s) useful in the context of the CAR of the invention. For example, the intracellular domain of the CAR may comprise a CD3 zeta chain portion and a portion of a costimulatory signaling molecule. The intracellular signaling sequences within the intracellular signaling portion of the CAR of the invention may be linked to each other in a random or specified order.

In another preferred embodiment, the intracellular domain is designed to comprise the activating domain of CD3 zeta and a signaling domain of CD28. In another embodiment, the intracellular domain is designed to comprise the activating domain of CD3 zeta and a signaling domain of 4-1BB. In another embodiment, the intracellular domain in the CAR is designed to comprise a portion of CD28 and CD3 zeta, wherein the intracellular CD28 comprises the nucleic acid sequence set forth in SEQ ID NO: 7 and the amino acid sequence set forth in SEQ ID NO. 8. The CD3 zeta nucleic acid sequence is set forth in SEQ ID NO: 9, and the amino acid sequence is set forth in SEQ ID NO. 8.

“Activation” or “stimulation” as used herein, refers to a primary response induced by binding of an activating molecule with its cognate ligand, wherein the binding mediates a signal transduction event.

An “activating molecule” or “stimulating molecule” refers to a molecule on a T cell, e.g., the TCR/CD3 complex that specifically binds with a cognate stimulatory ligand present on an antigen present cell. Suitable activating molecules are described herein.

It will be appreciated that suitable activation domains within the scope of the invention may be derived from (or correspond to) activating/stimulating molecules, such as, for example, CD3 or CD3 zeta. CD3 is an element of the T cell receptor on native T cells, and has been shown to be an important intracellular activating element in CARs.

In a preferred embodiment, the CD3 is CD3 zeta, the nucleotide sequence of which is set forth in SEQ ID NO. 9:

AGGGTGAAGTTTTCCAGATCTGCAGATGCACCAGCGTATCAGCAGGGCCA
GAACCAACTGTATAACGAGCTCAACCTGGGACGCAGGGAAGAGTATGACG
TTTTGGACAAGCGCAGAGGACGGGACCCTGAGATGGGTGGCAAACCAAGA
CGAAAAAACCCCCAGGAGGGTCTCTATAATGAGCTGCAGAAGGATAAGAT
GGCTGAAGCCTATTCTGAAATAGGCATGAAAGGAGAGCGGAGAAGGGGAA
AAGGGCACGACGGTTTGTACCAGGGACTCAGCACTGCTACGAAGGATACT
TATGACGCTCTCCACATGCAAGCCCTGCCACCTAGG

The corresponding amino acid of intracellular CD3 zeta is set forth in SEQ ID NO. 10:

RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

It will be appreciated that one preferred orientation of the CARs in accordance with the invention comprises an antigen binding molecule (such as scFv) in tandem with an extracellular and/or hinge domain, a costimulatory domain, and an activating domain. It will be further appreciated that multiple domains may be utilized in tandem.

Exemplary CAR constructs in accordance with the invention are set forth in Table 1:

TABLE 1

| Construct Name | scFv | Hinge Domain | Activating Domain |
|---|---|---|---|
| 24C1 CD28T | 24C1 | CD28T | CD3 zeta |
| 24C1 CD28 | 24C1 | CD28 | CD3 zeta |
| 24C1 CD8 | 24C1 | CD8 | CD3 zeta |
| 24C8 CD28T | 24C8 | CD28T | CD3 zeta |
| 24C8 CD28 | 24C8 | CD28 | CD3 zeta |
| 24C8 CD8 | 24C8 | CD8 | CD3 zeta |
| 2005.1 CD28T | 2005.1 | CD28T | CD3 zeta |
| 2005.1 CD28 | 2005.1 | CD28 | CD3 zeta |
| 2005.1 CD8 | 2005.1 | CD8 | CD3 zeta |
| 2005.2 CD28T | 2005.2 | CD28T | CD3 zeta |
| 2005.2 CD28 | 2005.2 | CD28 | CD3 zeta |
| 2005.2 CD8 | 2005.2 | CD8 | CD3 zeta |

The term “vector” means any molecule or entity (e.g., nucleic acid, plasmid, bacteriophage or virus) used to transfer protein coding information into a host cell. The term “expression vector” or “expression construct” refers to a vector that is suitable for transformation of a host cell and contains nucleic acid sequences that direct and/or control (in conjunction with the host cell) expression of one or more heterologous coding regions operatively linked thereto. An expression construct may include, but is not limited to, sequences that affect or control transcription, translation, and, if introns are present, affect RNA splicing of a coding region operably linked thereto.

The term “host cell” refers to a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA may recombine with that of the cell by physically integrating into a chromosome of the cell, or may be maintained transiently as an episomal element without being replicated, or may replicate independently as a plasmid. A cell is considered to have been “stably transformed” when the transforming DNA is replicated with the division of the cell.

The invention further relates to isolated polynucleotides encoding the chimeric antigen receptors (CARs) and T cell receptors (TCRs) of the present invention, as well as vectors comprising the polynucleotides. Any vector known in the art may be suitable for the present invention. In some embodiments, the vector is a viral vector. In some embodiments, the vector is a retroviral vector (such as pMSVG1), a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector (such as pGAR), or any derivative or combination thereof.

The pGAR sequence is as follows:

CTGACGCGCCCTGTAGCGGCGCATTAAGCGCGGCGGGTGTGGTGGTTACGCGCA
GCGTGACCGCTACACTTGCCAGCGCCCTAGCGCCCGCTCCTTTCGCTTTCTTCCCT
TCCTTTCTCGCCACGTTCGCCGGCTTTCCCCGTCAAGCTCTAAATCGGGGGCTCCC
TTTAGGGTTCCGATTTAGTGCTTTACGGCACCTCGACCCCAAAAAACTTGATTAG
GGTGATGGTTCACGTAGTGGGCCATCGCCCTGATAGACGGTTTTTCGCCCTTTGA
CGTTGGAGTCCACGTTCTTTAATAGTGGACTCTTGTTCCAAACTGGAACAACACT
CAACCCTATCTCGGTCTATTCTTTTGATTTATAAGGGATTTTGCCGATTTCGGCCT
ATTGGTTAAAAAATGAGCTGATTTAACAAAAATTTAACGCGAATTTTAACAAAAT
ATTAACGCTTACAATTTGCCATTCGCCATTCAGGCTGCGCAACTGTTGGGAAGGG
CGATCGGTGCGGGCCTCTTCGCTATTACGCCAGCTGGCGAAAGGGGGATGTGCT
GCAAGGCGATTAAGTTGGGTAACGCCAGGGTTTTCCCAGTCACGACGTTGTAAA
ACGACGGCCAGTGAATTGTAATACGACTCACTATAGGGCGACCCGGGGATGGCG
CGCCAGTAATCAATTACGGGGTCATTAGTTCATAGCCCATATATGGAGTTCCGCG
TTACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAG
TGTATCATATGCCAAGTACGCCCCCTATTGACGTCAATGACGGTAAATGGCCCGC
CTGGCATTATGCCCAGTACATGACCTTATGGGACTTTCCTACTTGGCAGTACATC
TACGTATTAGTCATCGCTATTACCATGCTGATGCGGTTTTGGCAGTACATCAATG
GGCGTGGATAGCGGTTTGACTCACGGGGATTTCCAAGTCTCCACCCCATTGACGT
CAATGGGAGTTTGTTTTGGCACCAAAATCAACGGGACTTTCCAAAATGTCGTAAC
AACTCCGCCCCATTGACGCAAATGGGCGGTAGGCGTGTACGGTGGGAGGTCTAT
ATAAGCAGAGCTGGTTTAGTGAACCGGGGTCTCTCTGGTTAGACCAGATCTGAGC
CTGGGAGCTCTCTGGCTAACTAGGGAACCCACTGCTTAAGCCTCAATAAAGCTTG
CCTTGAGTGCTTCAAGTAGTGTGTGCCCGTCTGTTGTGTGACTCTGGTAACTAGA
GATCCCTCAGACCCTTTTAGTCAGTGTGGAAAATCTCTAGCAGTGGCGCCCGAAC
AGGGACTTGAAAGCGAAAGGGAAACCAGAGGAGCTCTCTCGACGCAGGACTCG
GCTTGCTGAAGCGCGCACGGCAAGAGGCGAGGGGCGGCGACTGGTGAGTACGCC
AAAAATTTTGACTAGCGGAGGCTAGAAGGAGAGAGATGGGTGCGAGAGCGTCA
GTATTAAGCGGGGGAGAATTAGATCGCGATGGGAAAAAATTCGGTTAAGGCCAG
GGGGAAAGAAAAAATATAAATTAAAACATATAGTATGGGCAAGCAGGGAGCTA
GAACGATTCGCAGTTAATCCTGGCCTGTTAGAAACATCAGAAGGCTGTAGACAA
ATACTGGGACAGCTACAACCATCCCTTCAGACAGGATCAGAAGAACTTAGATCA
TTATATAATACAGTAGCAACCCTCTATTGTGTGCATCAAAGGATAGAGATAAAAG
ACACCAAGGAAGCTTTAGACAAGATAGAGGAAGAGCAAAACAAAAGTAAGACC
ACCGCACAGCAAGCCGCCGCTGATCTTCAGACCTGGAGGAGGAGATATGAGGGA
CAATTGGAGAAGTGAATTATATAAATATAAAGTAGTAAAAATTGAACCATTAGG
AGTAGCACCCACCAAGGCAAAGAGAAGAGTGGTGCAGAGAGAAAAAAGAGCAG
TGGGAATAGGAGCTTTGTTCCTTGGGTTCTTGGGAGCAGCAGGAAGCACTATGGG
CGCAGCGTCAATGACGCTGACGGTACAGGCCAGACAATTATTGTCTGGTATAGT
GCAGCAGCAGAACAATTTGCTGAGGGCTATTGAGGCGCAACAGCATCTGTTGCA
ACTCACAGTCTGGGGCATCAAGCAGCTCCAGGCAAGAATCCTGGCTGTGGAAAG
ATACCTAAAGGATCAACAGCTCCTGGGGATTTGGGGTTGCTCTGGAAAACTCATT
TGCACCACTGCTGTGCCTTGGAATGCTAGTTGGAGTAATAAATCTCTGGAACAGA
TTTGGAATCACACGACCTGGATGGAGTGGGACAGAGAAATTAACAATTACACAA
GCTTAATACACTCCTTAATTGAAGAATCGCAAAACCAGCAAGAAAAGAATGAAC
AAGAATTATTGGAATTAGATAAATGGGCAAGTTTGTGGAATTGGTTTAACATAAC
AAATTGGCTGTGGTATATAAAATTATTCATAATGATAGTAGGAGGCTTGGTAGGT
TTAAGAATAGTTTTTGCTGTACTTTCTATAGTGAATAGAGTTAGGCAGGGATATT
CACCATTATCGTTTCAGACCCACCTCCCAACCCCGAGGGGACCCGACAGGCCCG
AAGGAATAGAAGAAGAAGGTGGAGAGAGAGACAGAGACAGATCCATTCGATTA
GTGAACGGATCTCGACGGTATCGGTTAACTTTTAAAAGAAAAGGGGGGATTGGG
GGGTACAGTGCAGGGGAAAGAATAGTAGACATAATAGCAACAGACATACAAAC
TAAAGAATTACAAAAACAAATTACAAAATTCAAAATTTTATCGCGATCGCGGAA
TGAAAGACCCCACCTGTAGGTTTGGCAAGCTAGCTTAAGTAACGCCATTTTGCAA
GGCATGGAAAATACATAACTGAGAATAGAGAAGTTCAGATCAAGGTTAGGAACA
GAGAGACAGCAGAATATGGGCCAAACAGGATATCTGTGGTAAGCAGTTCCTGCC
CCGGCTCAGGGCCAAGAACAGATGGTCCCCAGATGCGGTCCCGCCCTCAGCAGT
TTCTAGAGAACCATCAGATGTTTCCAGGGTGCCCCAAGGACCTGAAAATGACCCT
GTGCCTTATTTGAACTAACCAATCAGTTCGCTTCTCGCTTCTGTTCGCGCGCTTCT
GCTCCCCGAGCTCAATAAAAGAGCCCACAACCCCTCACTCGGCGCGCCAGTCCTT
CGAAGTAGATCTTTGTCGATCCTACCATCCACTCGACACACCCGCCAGCGGCCGC
TGCCAAGCTTCCGAGCTCTCGAATTAATTCACGGTACCCACCATGGCCTAGGGAG
ACTAGTCGAATCGATATCAACCTCTGGATTACAAAATTTGTGAAAGATTGACTGG
TATTCTTAACTATGTTGCTCCTTTTACGCTATGTGGATACGCTGCTTTAATGCCTTT
GTATCATGCTATTGCTTCCCGTATGGCTTTCATTTTCTCCTCCTTGTATAAATCCTG
GTTGCTGTCTCTTTATGAGGAGTTGTGGCCCGTTGTCAGGCAACGTGGCGTGGTG
TGCACTGTGTTTGCTGACGCAACCCCCACTGGTTGGGGCATTGCCACCACCTGTC
AGCTCCTTTCCGGGACTTTCGCTTTCCCCCTCCCTATTGCCACGGCGGAACTCATC
GCCGCCTGCCTTGCCCGCTGCTGGACAGGGGCTCGGCTGTTGGGCACTGACAATT
CCGTGGTGTTGTCGGGGAAGCTGACGTCCTTTTCATGGCTGCTCGCCTGTGTTGC
CACCTGGATTCTGCGCGGGACGTCCTTCTGCTACGTCCCTTCGGCCCTCAATCCA
GCGGACCTTCCTTCCCGCGGCCTGCTGCCGGCTCTGCGGCCTCTTCCGCGTCTTCG
CCTTCGCCCTCAGACGAGTCGGATCTCCCTTTGGGCCGCCTCCCCGCCTGGTTAA
TTAAAGTACCTTTAAGACCAATGACTTACAAGGCAGCTGTAGATCTTAGCCACTT
TTTAAAAGAAAAGGGGGGACTGGAAGGGCGAATTCACTCCCAACGAAGACAAG
ATCTGCTTTTTGCTTGTACTGGGTCTCTCTGGTTAGACCAGATCTGAGCCTGGGAG
CTCTCTGGCTAACTAGGGAACCCACTGCTTAAGCCTCAATAAAGCTTGCCTTGAG
TGCTTCAAGTAGTGTGTGCCCGTCTGTTGTGTGACTCTGGTAACTAGAGATCCCT
CAGACCCTTTTAGTCAGTGTGGAAAATCTCTAGCAGGCATGCCAGACATGATAA
GATACATTGATGAGTTTGGACAAACCACAACTAGAATGCAGTGAAAAAAATGCT
TTATTTGTGAAATTTGTGATGCTATTGCTTTATTTGTAACCATTATAAGCTGCAAT
AAACAAGTTAACAACAACAATTGCATTCATTTTATGTTTCAGGTTCAGGGGGAGG
TGTGGGAGGTTTTTTGGCGCGCCATCGTCGAGGTTCCCTTTAGTGAGGGTTAATT
GCGAGCTTGGCGTAATCATGGTCATAGCTGTTTCCTGTGTGAAATTGTTATCCGCT
CACAATTCCACACAACATACGAGCCGGAAGCATAAAGTGTAAAGCCTGGGGTGC
CTAATGAGTGAGCTAACTCACATTAATTGCGTTGCGCTCACTGCCCGCTTTCCAG
TCGGGAAACCTGTCGTGCCAGCTGCATTAATGAATCGGCCAACGCGCGGGGAGA
GGCGGTTTGCGTATTGGGCGCTCTTCCGCTTCCTCGCTCACTGACTCGCTGCGCTC
GGTCGTTCGGCTGCGGCGAGCGGTATCAGCTCACTCAAAGGCGGTAATACGGTT
ATCCACAGAATCAGGGGATAACGCAGGAAAGAACATGTGAGCAAAAGGCCAGC
AAAAGGCCAGGAACCGTAAAAAGGCCGCGTTGCTGGCGTTTTTCCATAGGCTCC
GCCCCCCTGACGAGCATCACAAAAATCGACGCTCAAGTCAGAGGTGGCGAAACC
CGACAGGACTATAAAGATACCAGGCGTTTCCCCCTGGAAGCTCCCTCGTGCGCTC
TCCTGTTCCGACCCTGCCGCTTACCGGATACCTGTCCGCCTTTCTCCCTTCGGGAA
GCGTGGCGCTTTCTCATAGCTCACGCTGTAGGTATCTCAGTTCGGTGTAGGTCGT
TCGCTCCAAGCTGGGCTGTGTGCACGAACCCCCCGTTCAGCCCGACCGCTGCGCC
TTATCCGGTAACTATCGTCTTGAGTCCAACCCGGTAAGACACGACTTATCGCCAC
TGGCAGCAGCCACTGGTAACAGGATTAGCAGAGCGAGGTATGTAGGCGGTGCTA
CAGAGTTCTTGAAGTGGTGGCCTAACTACGGCTACACTAGAAGAACAGTATTTG
GTATCTGCGCTCTGCTGAAGCCAGTTACCTTCGGAAAAAGAGTTGGTAGCTCTTG
ATCCGGCAAACAAACCACCGCTGGTAGCGGTGGTTTTTTTGTTTGCAAGCAGCAG
ATTACGCGCAGAAAAAAAGGATCTCAAGAAGATCCTTTGATCTTTTCTACGGGGT
CTGACGCTCAGTGGAACGAAAACTCACGTTAAGGGATTTTGGTCATGAGATTATC
AAAAAGGATCTTCACCTAGATCCTTTTAAATTAAAAATGAAGTTTTAAATCAATC
TAAAGTATATATGAGTAAACTTGGTCTGACAGTTACCAATGCTTAATCAGTGAGG
CACCTATCTCAGCGATCTGTCTATTTCGTTCATCCATAGTTGCCTGACTCCCCGTC
GTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCA
GCCGGAAGGGCCGAGCGCAGAAGTGGTCCTGCAACTTTATCCGCCTCCATCCAG
TCTATTAATTGTTGCCGGGAAGCTAGAGTAAGTAGTTCGCCAGTTAATAGTTTGC
GCAACGTTGTTGCCATTGCTACAGGCATCGTGGTGTCACGCTCGTCGTTTGGTAT
GGCTTCATTCAGCTCCGGTTCCCAACGATCAAGGCGAGTTACATGATCCCCCATG
TTGTGCAAAAAAGCGGTTAGCTCCTTCGGTCCTCCGATCGTTGTCAGAAGTAAGT
TGGCCGCAGTGTTATCACTCATGGTTATGGCAGCACTGCATAATTCTCTTACTGTC
ATGCCATCCGTAAGATGCTTTTCTGTGACTGGTGAGTACTCAACCAAGTCATTCT
GAGAATAGTGTATGCGGCGACCGAGTTGCTCTTGCCCGGCGTCAATACGGGATA
ATACCGCGCCACATAGCAGAACTTTAAAAGTGCTCATCATTGGAAAACGTTCTTC
GGGGCGAAAACTCTCAAGGATCTTACCGCTGTTGAGATCCAGTTCGATGTAACCC
ACTCGTGCACCCAACTGATCTTCAGCATCTTTTACTTTCACCAGCGTTTCTGGGTG
AGCAAAAACAGGAAGGCAAAATGCCGCAAAAAAGGGAATAAGGGCGACACGGA
AATGTTGAATACTCATACTCTTCCTTTTTCAATATTATTGAAGCATTTATCAGGGT
TATTGTCTCATGAGCGGATACATATTTGAATGTATTTAGAAAAATAAACAAATAG
GGGTTCCGCGCACATTTCCCCGAAAAGTGCCAC

Suitable additional exemplary vectors include e.g., pBABE-puro, pBABE-neo largeTcDNA, pBABE-hygro-hTERT, pMKO.1 GFP, MSCV-IRES-GFP, pMSCV PIG (Puro IRES GFP empty plasmid), pMSCV-loxp-dsRed-loxp-eGFP-Puro-WPRE, MSCV IRES Luciferase, pMIG, MDH1-PGK-GFP 2.0, TtRMPVIR, pMSCV-IRES-mCherry FP, pRetroX GFP T2A Cre, pRXTN, pLncEXP, and pLXIN-Luc.

In a further embodiment, a mixture of different expression vectors may be used in genetically modifying a donor population of immune effector cells wherein each vector encodes a different CAR as disclosed herein. The resulting transduced immune effector cells form a mixed population of engineered cells, with a proportion of the engineered cells expressing more than one different CARs.

In a preferred embodiment of the present invention, the CAR comprises all or part of an anti-CD19 scFv, CD28, and CD3 zeta.

Transfected T cells of the present invention may also be created by transfecting the collected T cells with a polynucleotide encoding a T cell receptor (TCR). T cell receptors (TCRs) are molecules found on the surface of T cells that are responsible for recognizing antigen fragments as peptides bound to major histocompatibility complex (MHC) molecules. The TCR is comprised of two different protein chains—in approximately 95% of human TCRs, the TCR consists of an alpha (α) and beta (β) chain. In approximately 5% of human T cells the TCR consists of gamma and delta (γ/δ) chains. Each chain is composed of two extracellular domains: a variable (V) region and a constant (C) region, both of the immunoglobulin superfamily. As in other immunoglobulins, the variable domains of the TCR α-chain and β-chain (or gamma and delta (γ/δ) chains) each have three hypervariable or complementarity determining regions (CDRs). When the TCR engages with antigenic peptide and MHC (peptide/MHC), the T cell becomes activated, enabling it to attack and destroy the target cell.

The TCRs of the present invention may bind to, for example, a tumor-associated antigen. As used herein, “tumor-associated antigen” refers to any antigen that is associated with one or more cancers selected from the group consisting of: adrenocortical carcinoma, anal cancer, bladder cancer, bone cancer, brain cancer, breast cancer, carcinoid cancer, carcinoma, cervical cancer, colon cancer, endometrial cancer, esophageal cancer, extrahepatic bile duct cancer, extracranial germ cell cancer, eye cancer, gallbladder cancer, gastric cancer, germ cell tumor, gestational trophoblastic tumor, head and neck cancer, hypopharyngeal cancer, islet cell carcinoma, kidney cancer, large intestine cancer, laryngeal cancer, leukemia, lip and oral cavity cancer, liver cancer, lung cancer, lymphoma, malignant mesothelioma, Merkel cell carcinoma, mycosis fungoides, myelodysplastic syndrome, myeloproliferative disorders, nasopharyngeal cancer, neuroblastoma, oral cancer, oropharyngeal cancer, osteosarcoma, ovarian epithelial cancer, ovarian germ cell cancer, pancreatic cancer, paranasal sinus and nasal cavity cancer, parathyroid cancer, penile cancer, pituitary cancer, plasma cell neoplasm, prostate cancer, rhabdomyosarcoma, rectal cancer, renal cell cancer, transitional cell cancer of the renal pelvis and ureter, salivary gland cancer, Sezary syndrome, skin cancers, small intestine cancer, soft tissue sarcoma, stomach cancer, testicular cancer, thymoma, thyroid cancer, urethral cancer, uterine cancer, vaginal cancer, vulvar cancer, and Wilms' tumor.

In certain aspects, the present application may be suitable for target molecule to hematologic cancer. In some embodiments, the cancer is of the white blood cells. In other embodiments, the cancer is of the plasma cells. In some embodiments, the cancer is leukemia, lymphoma, or myeloma. In certain embodiments, the cancer is acute lymphoblastic leukemia (ALL) (including non T cell ALL), acute lymphoid leukemia (ALL), and hemophagocytic lymphohistocytosis (HLH)), B cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, diffuse large B cell lymphoma, diffuse large B cell lymphoma (DLBCL), follicular lymphoma, follicular lymphoma (FL), hairy cell leukemia, hemophagocytic syndrome (Macrophage Activating Syndrome (MAS), Hodgkin's Disease, large cell granuloma, leukocyte adhesion deficiency, malignant lymphoproliferative conditions, MALT lymphoma, mantle cell lymphoma, Marginal zone lymphoma, monoclonal gammapathy of undetermined significance (MGUS), multiple myeloma, myelodysplasia and myelodysplastic syndrome (MDS), myeloid diseases including but not limited to acute myeloid leukemia (AML), non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), primary mediastinal large B cell lymphoma (PMBCL), small cell- or a large cell-follicular lymphoma, splenic marginal zone lymphoma (SMZL), systemic amyloid light chain amyloidosis, T-cell acute lymphoid leukemia (TALL), T-cell lymphoma, transformed follicular lymphoma, Waldenstrom macroglobulinemia, or a combination thereof.

In some embodiments, the antigen is selected from a tumor-associated surface antigen, such as 5T4, alphafetoprotein (AFP), B7-1 (CD80), B7-2 (CD86), BCMA, B-human chorionic gonadotropin, CA-125, carcinoembryonic antigen (CEA), carcinoembryonic antigen (CEA), CD123, CD133, CD138, CD19, CD20, CD22, CD23, CD24, CD25, CD30, CD33, CD34, CD4, CD40, CD44, CD56, CD8, CLL-1, c-Met, CMV-specific antigen, CSPG4, CTLA-4, disialoganglioside GD2, ductal-epithelial mucine, EBV-specific antigen, EGFR variant III (EGFRvIII), ELF2M, endoglin, ephrin B2, epidermal growth factor receptor (EGFR), epithelial cell adhesion molecule (EpCAM), epithelial tumor antigen, ErbB2 (HER2/neu), fibroblast associated protein (fap), FLT3, folate binding protein, GD2, GD3, glioma-associated antigen, glycosphingolipids, gp36, HBV-specific antigen, HCV-specific antigen, HER1-HER2, HER2-HER3 in combination, HERV-K, high molecular weight-melanoma associated antigen (HMW-MAA), HIV-1 envelope glycoprotein gp41, HPV-specific antigen, human telomerase reverse transcriptase, IGFI receptor, IGF-II, IL-11Ralpha, IL-13R-a2, Influenza Virus-specific antigen; CD38, insulin growth factor (IGF1)-1, intestinal carboxyl esterase, kappa chain, LAGA-la, lambda chain, Lassa Virus-specific antigen, lectin-reactive AFP, lineage-specific or tissue specific antigen such as CD3, MAGE, MAGE-AL major histocompatibility complex (MHC) molecule, major histocompatibility complex (MHC) molecule presenting a tumor-specific peptide epitope, M-CSF, melanoma-associated antigen, mesothelin, mesothelin, MN-CA IX, MUC-1, mut hsp72, mutated p53, mutated p53, mutated ras, neutrophil elastase, NKG2D, Nkp30, NY-ESO-1, p53, PAP, prostase, prostase specific antigen (PSA), prostate carcinoma tumor antigen-1 (PCTA-1), prostate-specific antigen, prostein, PSMA, RAGE-1, ROR1, RUL RU2 (AS), surface adhesion molecule, surviving and telomerase, TAG-72, the extra domain A (EDA) and extra domain B (EDB) of fibronectin and the Al domain of tenascin-C (TnC Al), thyroglobulin, tumor stromal antigens, vascular endothelial growth factor receptor-2 (VEGFR2), virus-specific surface antigen such as an HIV-specific antigen (such as HIV gp120), as well as any derivate or variant of these surface markers.

The TCRs of the present invention may also bind to a viral infection-associated antigen. Viral infection-associated antigens include antigens associated with any viral infection, including, for example, viral infection caused by HIV.

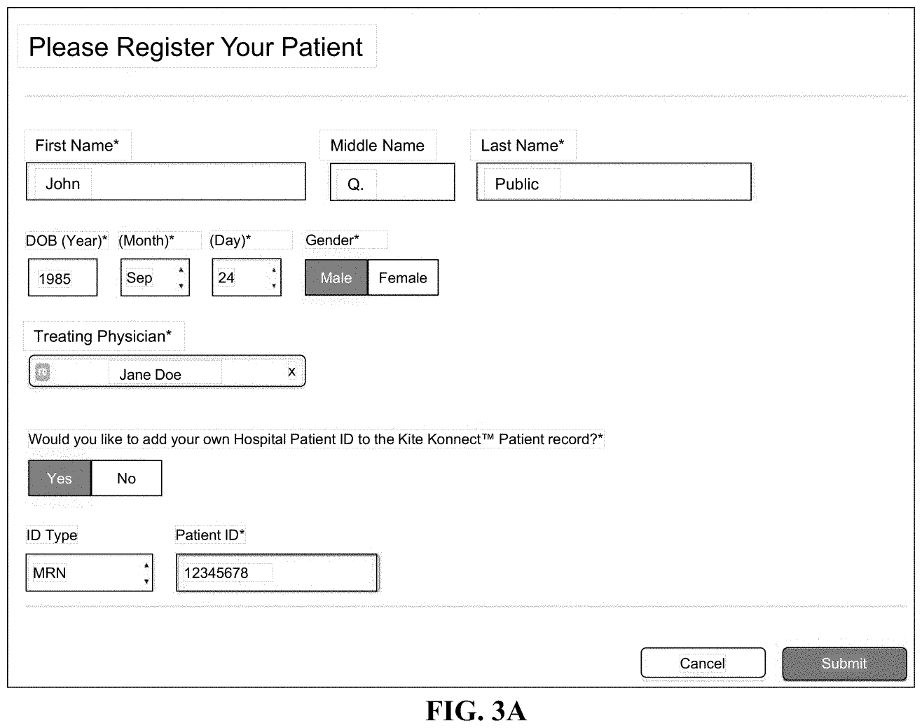

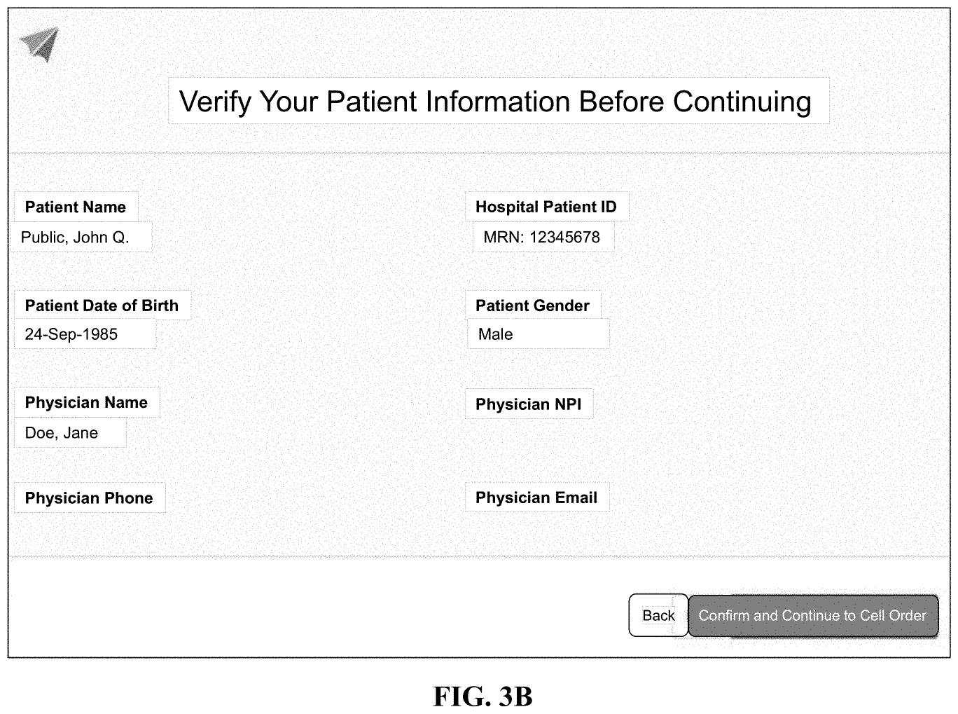

To initiate the patient-specific immunotherapy procedure described herein, a physician or other medical personnel at client computing device 102a accesses the user interface module 108a of server computing device 106 (e.g., via a web portal, web site, or other similar platform). The user interface module 108a generates user interface screens and/or elements for presentation to the physician on the client computing device 102a, in order for the physician to enroll the patient and initiate the patient-specific immunotherapy procedure. The user interface module 108a may generate UI screens to enable the physician to enter the patient's identifying information (e.g., full name, date of birth), demographics (e.g., gender), and healthcare provider information (e.g., physician name, hospital name). The user interface module 108a may also provide a UI element for entry of a healthcare-provider-specific or hospital-specific user identifier (e.g., medical record number, hospital patient ID). FIGS. 3A and 3B are exemplary screenshots generated by the user interface module 108a that enable enrollment of new patients into the system; FIG. 3A depicts the patient enrollment data entry screen, and FIG. 3B depicts a patent information review and confirmation screen.

Turning back to FIG. 2, the client computing device 102a generates a request to create transfected T cells for a patient, and the server computing device 106 receives (202) the request. As described above, the physician at client computing device 102a interacts with the user interface module 108a to enroll the patient by providing the necessary patent information. Once the user interface module 108a receives confirmation from the client computing device 102a that the patient information has been fully entered and is accurate, the user interface module 108a stores the data in database 110. The user interface module 108a also generates (204) a patient-specific identifier that will be used as part of the sample tracking and chain-of-custody/chain-of-identity process described below. In one embodiment, the patient-specific identifier comprises a patient identity element (e.g., a patient ID number), a sales order identifier, and a cell order lot number. For example, the user interface module 108a may generate the patient-specific identifier by mapping the patient identity element, sales order number, and cell order lot number into a database table that is indexed with an identifier (e.g., a nine-digit numeric code) that uniquely identifies the patient, sales order, and cell lot combination.

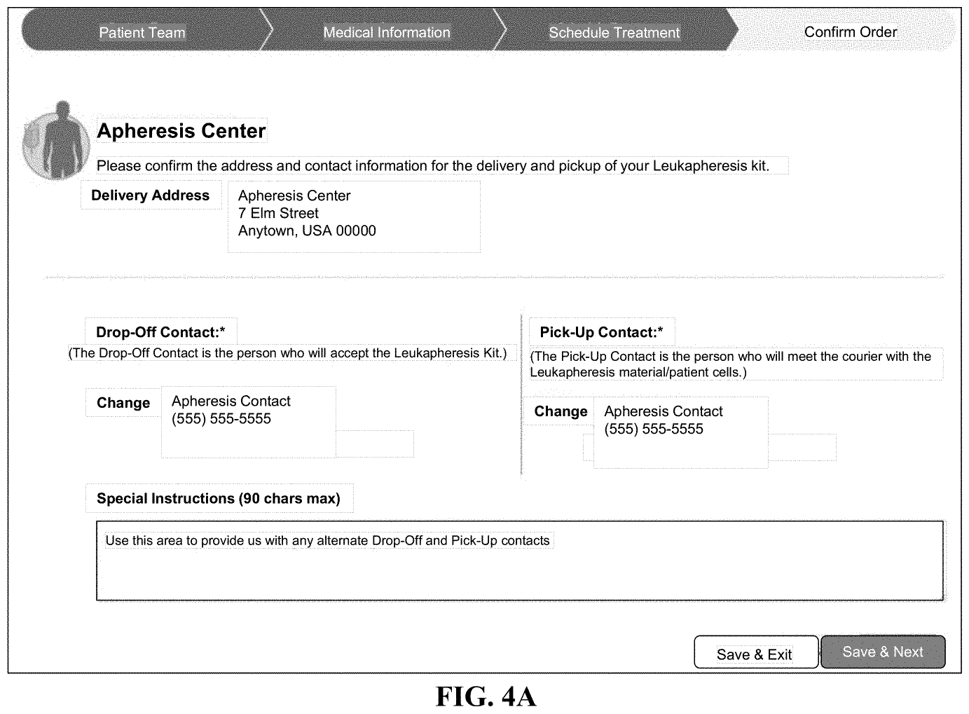

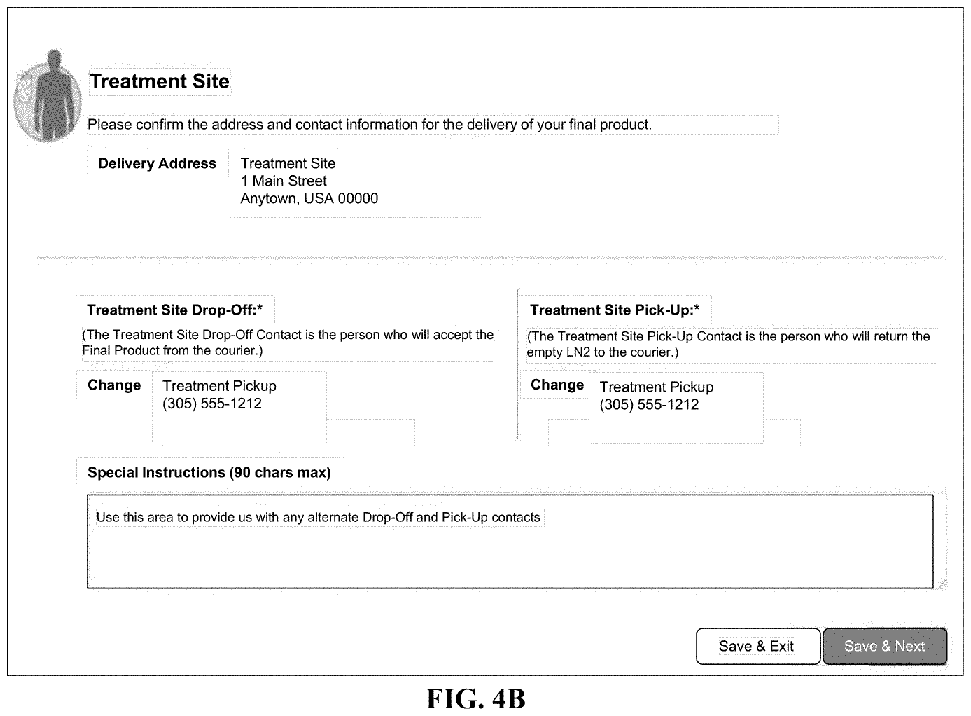

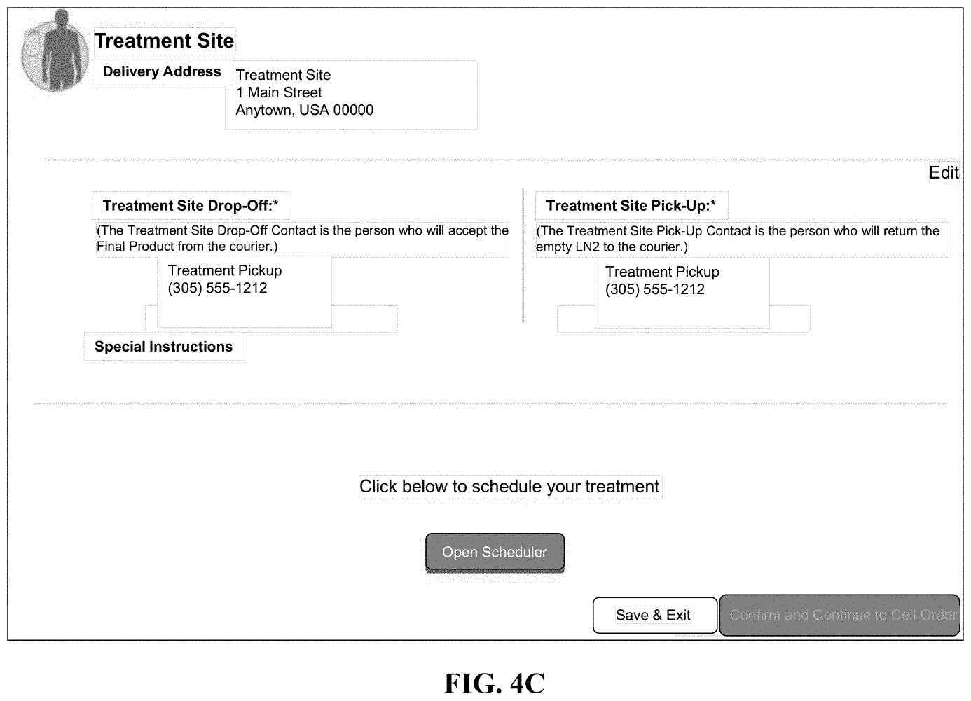

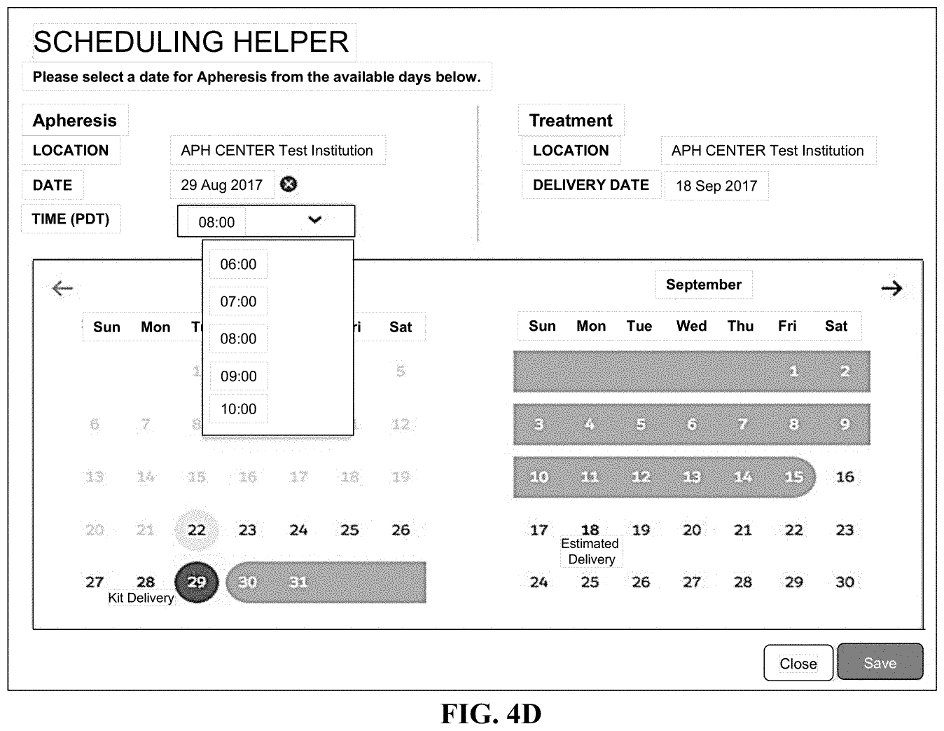

Next, the physician at client computing device 102a interacts with the user interface module 108a to schedule an appointment to obtain the biological material from the patient and, due to the time sensitivity of providing the altered biological material back to the patient quickly, confirming that the manufacturing facility has availability to process the biological material shortly after the material is obtained. The user interface module 108a requests confirmation of the material extraction site (e.g., site name, address, contact information) for drop-off of an extraction kit (e.g., leukapheresis kit) and confirmation of the altered material delivery and treatment site (e.g., site name, address, contact information) for delivery of the material (e.g., transfected T cells) from the manufacturing facility. FIGS. 4A-4D are exemplary screenshots generated by the user interface module 108a that enable confirmation of these sites and scheduling of the appointment; FIG. 4A depicts the drop-off site confirmation screen, FIG. 4B depicts the material delivery site confirmation screen; FIG. 4C depicts the screen to open the appointment scheduler; and FIG. 4D depicts the appointment scheduler. In some embodiments, the user interface module 108a communicates with a remote computing device of the manufacturing facility, in conjunction with the database 110, to coordinate scheduling of the biological material modification to ensure the most efficient processing schedule so that the modified material is returned quickly back to the patient.

Turning back to FIG. 2, once the cell order process is complete as described above, a process 206 is initiated to perform the biological material extraction procedure at the extraction site, ship the extracted material to the manufacturing facility for modification, and send the modified material back to a delivery site for infusion back into the patient's bloodstream. First, the patient arrives at the material extraction site and a procedure (e.g., a leukapheresis procedure) is performed (206a) on a sample of the patient's blood to collect T cells from the sample. When the procedure is performed, a client computing device (e.g., device 102b) at the extraction site communicates with the event tracking module 108b of server computing device 106 to transmit a tracking event to the module 108b that corresponds to performance of the procedure. For example, a clinician at client computing device 102b may submit the tracking event by entering information into a user interface. In another example, the client computing device 102b may automatically transmit the tracking event to the module 108b (e.g., via API) when information about the procedure is captured by the client computing device 102b (e.g., scanning a barcode).

The tracking event may comprise the patient-specific identifier, a timestamp, an event ID (e.g., that indicates a material extraction procedure was performed), and other information relevant to the process (e.g., cell order lot number, sales order number, site location, etc.). The event tracking module 108b stores the tracking event in database 110 based upon the information received from the client computing device 102b. Because this is the first step in the biological material extraction and modification process, the event tracking module 108b notifies the chain of custody module 108c of receipt of the tracking event. The chain of custody module 108c generates a chain of custody data structure (e.g., in database 110) that incorporates the tracking event (and each subsequent tracking event described herein) in an ordered sequence that enables the patient, the physician, the manufacturer, and other parties to understand the precise status of the biological material and to ensure that the biological material is accounted for at all times in avoidance of loss or mishandling. In an example, the chain of custody data structure may be a linked list that connects each of the tracking events together in a sequential manner according to, e.g., timestamp of the tracking event.

Next, the collected T cells are transferred (206b) to a container (e.g., a tube, vial, or other type of biological material carrier) and another tracking event is captured and transmitted to the event tracking module 108b for integration into the chain of custody data structure described above. Then, the container is labeled (206c) with the patient-specific identifier, and another tracking event is captured and transmitted to the event tracking module 108b for communication with the chain of custody module 108c to integrate into the chain of custody data structure. For example, the container that houses the collected T cells is labeled with a barcode comprising the patient-specific identifier, which is then scanned at the extraction site—indicating that the collected T cells are ready for shipment to the manufacturing facility. Upon scanning the barcode, the client computing device 102b generates the tracking event and transmits the event to the event tracking module 108b.

Then, the extraction site transmits (206d) the collected T cells to the manufacturing facility, which performs the procedure to generate the transfected T cells. Both when the collected T cells are shipped to the manufacturing facility and when the collected T cells are received at the manufacturing facility, one or more of the devices used to record the shipment and receipt of the T cells communicate with the event tracking module 108b to transmit a tracking event associated with the particular activity for communication with the chain of custody module 108c to integrate into the chain of custody. In this way, the chain of custody module 108c automatically and continuously updates the chain of custody data structure with the latest information, and that information is reflected in one or more screens generated by the user interface module 108a.

The manufacturing facility then creates (206e) transfected T cells from the collected T cells using a cell modification technique, and a client computing device (e.g., device 102c) generates one or more tracking events based upon the particular cell modification technique being used. For example, a cell modification technique may comprise several phases—such as (i) quality assurance of the collected T cells prior to modification, (ii) modification of the T cells; (ii) release testing of the transfected T cells, and (iv) finalization of the transfected T cells for shipment back to the infusion site. For each of these phases, the client computing device 102c captures a tracking event and transmits the tracking event to the event tracking module 108b for integration by the chain of custody module 108c into the chain of custody data structure.

Once the transfected T cells are shipped, the infusion site receives (2060 the transfected T cells and a client computing device (e.g., device 102d) generates a tracking event for transmission to the event tracking module 108b for integration by the chain of custody module 108c into the chain of custody data structure. For example, the client computing device 102d may scan a barcode associated with the shipment and/or the transfected T cells to automatically generate the tracking event and transmit the event to the server computing device 106.

After receipt, the transfected T cells are infused (206g) into the patient's bloodstream, thereby completing the process. At the same time, the client computing device 102d generates a tracking event and transmits the event to the event tracking module 108b for integration by the chain of custody module 108c into the chain of custody data structure.

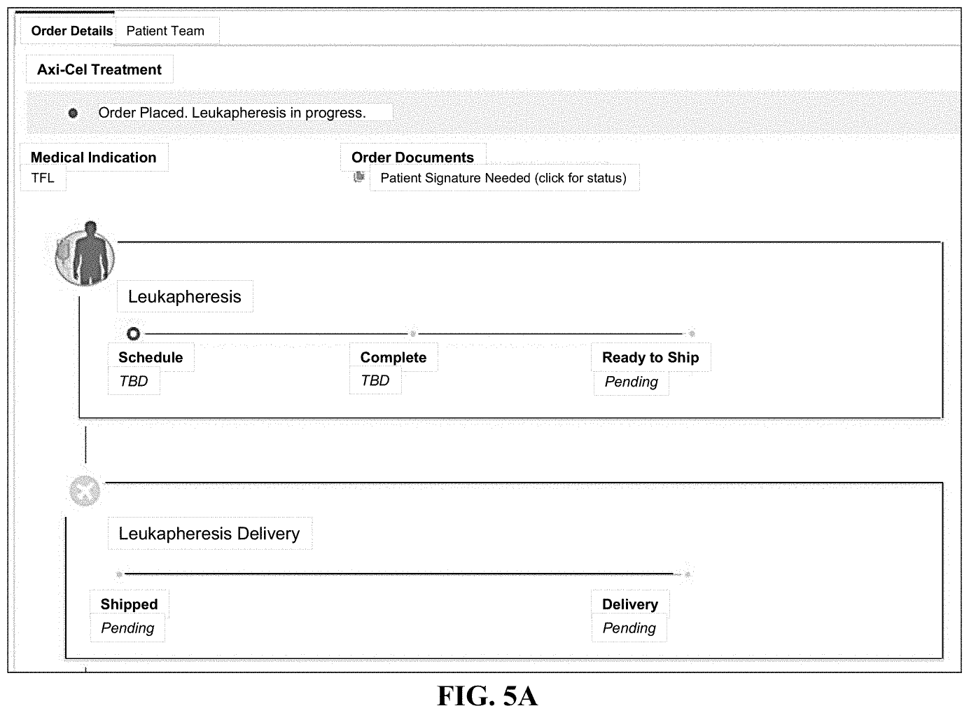

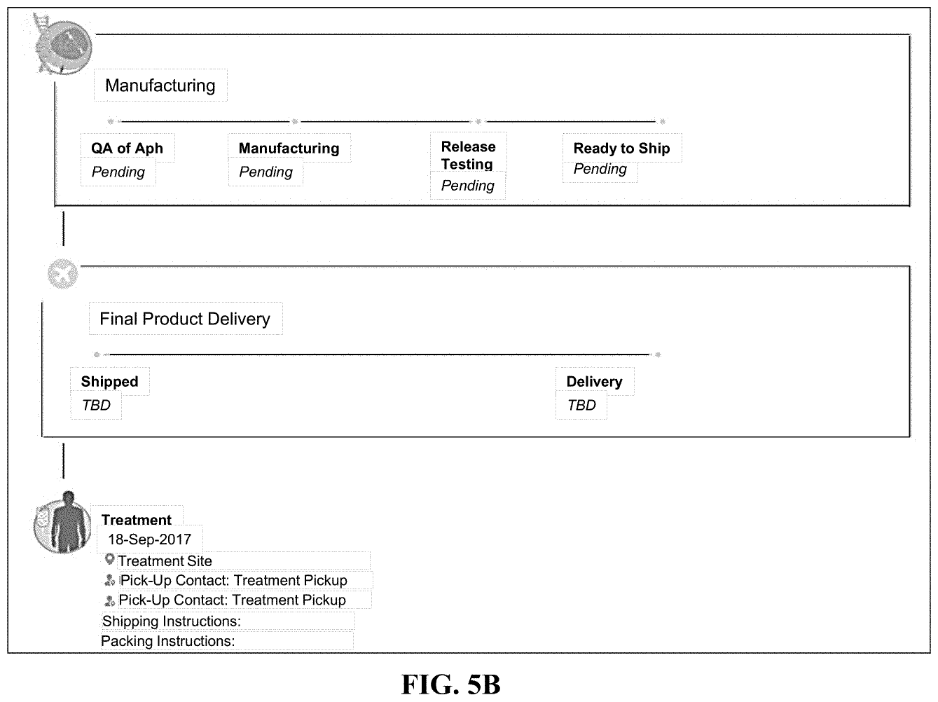

FIGS. 5A and 5B are exemplary screenshots generated by the user interface module 108a to enable the client computing devices 102a-102d to view the chain of custody associated with a particular patient, biological material, and cell modification process. As shown in FIG. 5A, the chain of custody of the biological material during the leukapheresis process (including the steps of scheduling the procedure, completing the procedure, and having the extracted T cells ready for shipment) is captured in a timeline at the top of the screen, where each step of the leukapheresis process is associated with a point on the timeline, and the chain of custody of the biological material during the delivery process (e.g., T cells shipped from extraction site, T cells delivered to manufacturing facility) is captured in a timeline at the bottom of the screen. When the event tracking module 108b and chain of custody module 108c record a tracking event as described above, the user interface module 108a traverses the chain of custody data structure to graphically represent the current status of the chain of custody on screen.

As shown in FIG. 5B, the chain of custody of the biological material during the manufacturing process (including QA, manufacturing, release testing, and finalizing for shipment) is shown in a timeline at the top of the screen, and the chain of custody of the biological material during the final product delivery process (including shipment and delivery to the infusion site) is shown in the middle of the screen. In addition, the treatment details, including the treatment date, are displayed at the bottom of the screen. Also, the chain of custody is constantly associated with the specific patient—thereby ensuring a complete chain of identity between the patient and the biological material during all phases of manufacturing.

The above-described techniques may be implemented in digital and/or analog electronic circuitry, or in computer hardware, firmware, software, or in combinations of them. The implementation may be as a computer program product, i.e., a computer program tangibly embodied in a machine-readable storage device, for execution by, or to control the operation of, a data processing apparatus, e.g., a programmable processor, a computer, and/or multiple computers. A computer program may be written in any form of computer or programming language, including source code, compiled code, interpreted code and/or machine code, and the computer program may be deployed in any form, including as a stand-alone program or as a subroutine, element, or other unit suitable for use in a computing environment. A computer program may be deployed to be executed on one computer or on multiple computers at one or more sites. The computer program may be deployed in a cloud computing environment (e.g., Amazon® AWS, Microsoft® Azure, IBM®).

Method steps may be performed by one or more processors executing a computer program to perform functions of the invention by operating on input data and/or generating output data. Method steps may also be performed by, and an apparatus may be implemented as, special purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), or an ASIC (application-specific integrated circuit), or the like. Subroutines may refer to portions of the stored computer program and/or the processor, and/or the special circuitry that implement one or more functions.

Processors suitable for the execution of a computer program include, by way of example, special purpose microprocessors specifically programmed with instructions executable to perform the methods described herein. Generally, a processor receives instructions and data from a read-only memory or a random access memory or both. The essential elements of a computer are a processor for executing instructions and one or more memory devices for storing instructions and/or data. Memory devices, such as a cache, may be used to temporarily store data. Memory devices may also be used for long-term data storage. Generally, a computer also includes, or is operatively coupled to receive data from or transfer data to, or both, one or more mass storage devices for storing data, e.g., magnetic, magneto-optical disks, or optical disks. A computer may also be operatively coupled to a communications network in order to receive instructions and/or data from the network and/or to transfer instructions and/or data to the network. Computer-readable storage mediums suitable for embodying computer program instructions and data include all forms of volatile and non-volatile memory, including by way of example semiconductor memory devices, e.g., DRAM, SRAM, EPROM, EEPROM, and flash memory devices; magnetic disks, e.g., internal hard disks or removable disks; magneto-optical disks; and optical disks, e.g., CD, DVD, HD-DVD, and Blu-ray disks. The processor and the memory may be supplemented by and/or incorporated in special purpose logic circuitry.

To provide for interaction with a user, the above described techniques may be implemented on a computing device in communication with a display device, e.g., a CRT (cathode ray tube), plasma, or LCD (liquid crystal display) monitor, a mobile device display or screen, a holographic device and/or projector, for displaying information to the user and a keyboard and a pointing device, e.g., a mouse, a trackball, a touchpad, or a motion sensor, by which the user may provide input to the computer (e.g., interact with a user interface element). Other kinds of devices may be used to provide for interaction with a user as well; for example, feedback provided to the user may be any form of sensory feedback, e.g., visual feedback, auditory feedback, or tactile feedback; and input from the user may be received in any form, including acoustic, speech, and/or tactile input.

The above-described techniques may be implemented in a distributed computing system that includes a back-end component. The back-end component may, for example, be a data server, a middleware component, and/or an application server. The above described techniques may be implemented in a distributed computing system that includes a front-end component. The front-end component may, for example, be a client computer having a graphical user interface, a Web browser through which a user may interact with an example implementation, and/or other graphical user interfaces for a transmitting device. The above described techniques may be implemented in a distributed computing system that includes any combination of such back-end, middleware, or front-end components.

The components of the computing system may be interconnected by transmission medium, which may include any form or medium of digital or analog data communication (e.g., a communication network). Transmission medium may include one or more packet-based networks and/or one or more circuit-based networks in any configuration. Packet-based networks may include, for example, the Internet, a carrier internet protocol (IP) network (e.g., local area network (LAN), wide area network (WAN), campus area network (CAN), metropolitan area network (MAN), home area network (HAN)), a private IP network, an IP private branch exchange (IPBX), a wireless network (e.g., radio access network (RAN), Bluetooth, near field communications (NFC) network, Wi-Fi, WiMAX, general packet radio service (GPRS) network, HiperLAN), and/or other packet-based networks. Circuit-based networks may include, for example, the public switched telephone network (PSTN), a legacy private branch exchange (PBX), a wireless network (e.g., RAN, code-division multiple access (CDMA) network, time division multiple access (TDMA) network, global system for mobile communications (GSM) network), and/or other circuit-based networks.

Information transfer over transmission medium may be based on one or more communication protocols. Communication protocols may include, for example, Ethernet protocol, Internet Protocol (IP), Voice over IP (VOIP), a Peer-to-Peer (P2P) protocol, Hypertext Transfer Protocol (HTTP), Session Initiation Protocol (SIP), H.323, Media Gateway Control Protocol (MGCP), Signaling System #7 (SS7), a Global System for Mobile Communications (GSM) protocol, a Push-to-Talk (PTT) protocol, a PTT over Cellular (POC) protocol, Universal Mobile Telecommunications System (UMTS), 3GPP Long Term Evolution (LTE) and/or other communication protocols.

Devices of the computing system may include, for example, a computer, a computer with a browser device, a telephone, an IP phone, a mobile device (e.g., cellular phone, personal digital assistant (PDA) device, smart phone, tablet, laptop computer, electronic mail device), and/or other communication devices. The browser device includes, for example, a computer (e.g., desktop computer and/or laptop computer) with a World Wide Web browser (e.g., Chrome™ from Google, Inc., Microsoft® Internet Explorer® available from Microsoft Corporation, and/or Mozilla® Firefox available from Mozilla Corporation). Mobile computing device include, for example, a Blackberry® from Research in Motion, an iPhone® from Apple Corporation, and/or an Android™-based device. IP phones include, for example, a Cisco® Unified IP Phone 7985G and/or a Cisco® Unified Wireless Phone 7920 available from Cisco Systems, Inc.

### Additional Definitions

The terms “polypeptide” or “protein” refer to a macromolecule having the amino acid sequence of a protein, including deletions from, additions to, and/or substitutions of one or more amino acids of the native sequence, and preferably no more than 8 amino acid substitutions therein. Preferably, the polypeptides or proteins are isolated as defined herein. The term “polypeptide fragment” refers to an isolated polypeptide that has an amino-terminal deletion, a carboxyl-terminal deletion, and/or an internal deletion as compared with the full-length native protein. Such fragments may also contain modified amino acids as compared with the native protein. Useful polypeptide fragments include immunologically functional fragments of antigen binding molecules. Useful fragments include but are not limited to one or more CDR regions, variable domains of a heavy and/or light chain, a portion of other portions of an antibody chain, and the like.

The term “antibody” refers to an intact immunoglobulin of any isotype, or a fragment thereof that may compete with the intact antibody for specific binding to the target antigen/molecule, and includes, for instance, chimeric, humanized, fully human, and bispecific antibodies. An “antibody” is a species of an antigen binding molecule as defined herein. An intact antibody will generally comprise at least two full-length heavy chains and two full-length light chains, but in some instances may include fewer chains such as antibodies naturally occurring in camelids which may comprise only heavy chains. Antibodies may be derived solely from a single source, or may be chimeric, that is, different portions of the antibody may be derived from two different antibodies as described further below. The antigen binding molecules, antibodies, or binding fragments may be produced in hybridomas, by recombinant DNA techniques, or by enzymatic or chemical cleavage of intact antibodies. Unless otherwise indicated, the term “antibody” includes, in addition to antibodies comprising two full-length heavy chains and two full-length light chains, derivatives, variants, fragments, and muteins thereof, examples of which are described below. Furthermore, unless explicitly excluded, antibodies include monoclonal antibodies, bispecific antibodies, minibodies, domain antibodies, synthetic antibodies (sometimes referred to herein as “antibody mimetics”), chimeric antibodies, humanized antibodies, human antibodies, antibody fusions (sometimes referred to herein as “antibody conjugates”) and fragments thereof, respectively.

The variable regions typically exhibit the same general structure of relatively conserved framework regions (FR) joined by the 3 hypervariable regions (i.e., “CDRs”). The CDRs from the two chains of each pair typically are aligned by the framework regions, which may enable binding to a specific epitope. From N-terminal to C-terminal, both light and heavy chain variable regions typically comprise the domains FR1, CDR1, FR2, CDR2, FR3, CDR3 and FR4. By convention, CDR regions in the heavy chain are typically referred to as HC CDR1, CDR2, and CDR3. The CDR regions in the light chain are typically referred to as LC CDR1, CDR2, and CDR3. The assignment of amino acids to each domain is typically in accordance with the definitions of Kabat, Chothia, or the AbM definition.

The term “Kabat numbering” and like terms are recognized in the art and refer to a system of numbering amino acid residues in the heavy and light chain variable regions of an antibody, or an antigen-binding portion thereof. In certain aspects, the CDRs of an antibody may be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally may include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

In certain aspects, the CDRs of an antibody may be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-HI loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In a specific embodiment, the CDRs of the antibodies described herein have been determined according to the Chothia numbering scheme.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

TABLE 2
CDR Numbering

| Loop | Kabat | AbM | Chothia | Contact |
|---|---|---|---|---|
| L1 | L24-L34 | L24-L34 | L24-L34 | L30-L36 |
| L2 | L50-L56 | L50-L56 | L50-L56 | L46-L55 |
| L3 | L89-L97 | L89-L97 | L89-L97 | L89-L96 |
| H1 | H31-H35B | H26-H35B | H26-H32..34 | H30-H35B |
| | (Kabat | | | |
| | Numbering) | | | |
| H1 | H31-H35 | H26-H35 | H26-H32 | H30-H35 |
| | (Chothia | | | |
| | Numbering) | | | |
| H2 | H50-H65 | H50-H58 | H52-H56 | H47-H58 |
| H3 | H95-H102 | H95-H102 | H95-H102 | H93-H101 |

As used herein, the term “heavy chain” when used in reference to an antibody may refer to any distinct type, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG₁, IgG₂, IgG₃ and IgG₄.

As used herein, the term “light chain” when used in reference to an antibody may refer to any distinct type, e.g., kappa (κ) or lambda (λ) based on the amino acid sequence of the constant domains. Light chain amino acid sequences are well known in the art. In specific embodiments, the light chain is a human light chain.

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. The variable region of an antibody typically determines specificity of a particular antibody for its target.

Variability is not evenly distributed throughout the variable domains of antibodies or antigen binding molecules; it is concentrated in sub-domains of each of the heavy and light chain variable regions. These subdomains are called “hypervariable regions” or “complementarity determining regions” (CDRs) as further described herein. The more conserved (i.e., non-hypervariable) portions of the variable domains are called the “framework” regions (FRM or FR) and provide a scaffold for the six CDRs in three dimensional space to form an antigen-binding surface. The variable domains of naturally occurring heavy and light chains each comprise four FRM regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding site (see Kabat et al., described further herein.

Typically, CDRs form a loop structure that may be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure may be characterized by the torsion angles of the polypeptide backbone. Correspondent loops between antibodies may, therefore, have very similar three dimensional structures, despite high amino acid sequence variability in most parts of the loops (Chothia and Lesk, J. MoI. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class may therefore be made based on the presence of these key amino acid residues.

The term “canonical structure” may also include considerations as to the linear sequence of the antibody, for example, as catalogued by Kabat (Kabat et al., herein). The Kabat numbering scheme (system) is a widely adopted standard for numbering the amino acid residues of an antibody variable domain in a consistent manner and is the preferred scheme applied in the present invention as also mentioned elsewhere herein. Additional structural considerations may also be used to determine the canonical structure of an antibody. For example, those differences not fully reflected by Kabat numbering may be described by the numbering system of Chothia et al. and/or revealed by other techniques, for example, crystallography and two- or three-dimensional computational modeling. Accordingly, a given antibody sequence may be placed into a canonical class which allows for, among other things, identifying appropriate chassis sequences (e.g., based on a desire to include a variety of canonical structures in a library). Kabat numbering of antibody amino acid sequences and structural considerations as described by Chothia et al. (herein) and their implications for construing canonical aspects of antibody structure, are described in the literature. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988.

The CDR3 of the light chain and, particularly, the CDR3 of the heavy chain may constitute the most important determinants in antigen binding within the light and heavy chain variable regions. In some antibody constructs, the heavy chain CDR3 appears to constitute the major area of contact between the antigen and the antibody. In vitro selection schemes in which CDR3 alone is varied may be used to vary the binding properties of an antibody or determine which residues contribute to the binding of an antigen. Hence, CDR3 is typically the greatest source of molecular diversity within the antibody-binding site. H3, for example, may be as short as two amino acid residues or greater than 26 amino acids.

As used herein, the terms “constant region” and “constant domain” are interchangeable and have a meaning common in the art. The constant region is an antibody portion, e.g., a carboxyl terminal portion of a light and/or heavy chain which is not directly involved in binding of an antibody to antigen but which may exhibit various effector functions, such as interaction with the Fc receptor. The constant region of an immunoglobulin molecule generally has a more conserved amino acid sequence relative to an immunoglobulin variable domain.

An “Fc” region comprises two heavy chain fragments comprising the CH1 and CH2 domains of an antibody. The two heavy chain fragments are held together by two or more disulfide bonds and by hydrophobic interactions of the CH3 domains.

A “Fab fragment” comprises one light chain and the CH1 and variable regions of one heavy chain. The heavy chain of a Fab molecule cannot form a disulfide bond with another heavy chain molecule. A “Fab′ fragment” comprises one light chain and a portion of one heavy chain that contains the VH domain and the CH1 domain and also the region between the CH1 and CH2 domains, such that an interchain disulfide bond may be formed between the two heavy chains of two Fab′ fragments to form an F(ab′)₂ molecule. An “F(ab′)₂ fragment” contains two light chains and two heavy chains containing a portion of the constant region between the CH1 and CH2 domains, such that an interchain disulfide bond is formed between the two heavy chains. An F(ab′)₂ fragment thus is composed of two Fab′ fragments that are held together by a disulfide bond between the two heavy chains.

The “Fv region” comprises the variable regions from both the heavy and light chains, but lacks the constant regions.

A “bivalent antigen binding molecule” comprises two antigen binding sites. In some instances, the two binding sites have the same antigen specificities. Bivalent antigen binding molecules may be bispecific. A “multispecific antigen binding molecule” is one that targets more than one antigen or epitope. A “bispecific,” “dual-specific” or “bifunctional” antigen binding molecule is a hybrid antigen binding molecule or antibody, respectively, having two different antigen binding sites. The two binding sites of a bispecific antigen binding molecule will bind to two different epitopes, which may reside on the same or different protein targets.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y may generally be represented by the dissociation constant (K_{D}). Affinity may be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} may be determined by techniques known to one of ordinary skill in the art, such as BIAcore® or KinExA.

The term “neutralizing” refers to an antigen binding molecule, scFv, or antibody, respectively, that binds to a ligand and prevents or reduces the biological effect of that ligand. This may be done, for example, by directly blocking a binding site on the ligand or by binding to the ligand and altering the ligand's ability to bind through indirect means (such as structural or energetic alterations in the ligand). In some embodiments, the term may also denote an antigen binding molecule that prevents the protein to which it is bound from performing a biological function.

The term “compete” when used in the context of antigen binding molecules that compete for the same epitope means competition between antigen binding molecules as determined by an assay in which the antigen binding molecule (e.g., antibody or immunologically functional fragment thereof) being tested prevents or inhibits (e.g., reduces) specific binding of a reference antigen binding molecule to an antigen. Numerous types of competitive binding assays may be used to determine if one antigen binding molecule competes with another, for example: solid phase direct or indirect radioimmunoassay (RIA), solid phase direct or indirect enzyme immunoassay (EIA), sandwich competition assay (Stahli et al., 1983, Methods in Enzymology 9:242-253); solid phase direct biotin-avidin EIA (Kirkland et al., 1986, J. Immunol. 137:3614-3619), solid phase direct labeled assay, solid phase direct labeled sandwich assay (Harlow and Lane, 1988, Antibodies, A Laboratory Manual, Cold Spring Harbor Press); solid phase direct label RIA using 1-125 label (Morel et al., 1988, Molec. Immunol. 25:7-15); solid phase direct biotin-avidin EIA (Cheung, et al., 1990, Virology 176:546-552); and direct labeled RIA (Moldenhauer et al., 1990, Scand. J. Immunol. 32:77-82).

As used herein, the term “epitope” refers to a localized region of an antigen to which an antibody may specifically bind. An epitope may be, for example, contiguous amino acids of a polypeptide (linear or contiguous epitope) or an epitope may, for example, come together from two or more non-contiguous regions of a polypeptide or polypeptides (conformational, non-linear, discontinuous, or non-contiguous epitope). In certain embodiments, the epitope to which an antibody binds may be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giege R. et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody:antigen crystals may be studied using well known X-ray diffraction techniques and may be refined using computer software such as X-PLOR (Yale University, 1992, distributed by Molecular Simulations, Inc.; see e.g. Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies may be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

The term “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as, e.g., a chimeric antigen receptor (CAR) or a T cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T cell therapies. T cell therapy may include adoptive T cell therapy, tumor-infiltrating lymphocyte (TIL) immunotherapy, autologous cell therapy, engineered autologous cell therapy (eACT), and allogeneic T cell transplantation. However, one of skill in the art would recognize that the conditioning methods disclosed herein would enhance the effectiveness of any transplanted T cell therapy. Examples of T cell therapies are described in U.S. Patent Publication Nos. 2014/0154228 and 2002/0006409, U.S. Pat. No. 5,728,388, and International Publication No. WO 2008/081035.

The T cells of the immunotherapy may come from any source known in the art. For example, T cells may be differentiated in vitro from a hematopoietic stem cell population, or T cells may be obtained from a subject. T cells may be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells may be derived from one or more T cell lines available in the art. T cells may also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

The term “engineered Autologous Cell Therapy,” which may be abbreviated as “eACT™,” also known as adoptive cell transfer, is a process by which a patient's own T cells are collected and subsequently genetically altered to recognize and target one or more antigens expressed on the cell surface of one or more specific tumor cells or malignancies. T cells may be engineered to express, for example, chimeric antigen receptors (CAR) or T cell receptor (TCR). CAR positive (+) T cells are engineered to express an extracellular single chain variable fragment (scFv) with specificity for a particular tumor antigen linked to an intracellular signaling part comprising at least one costimulatory domain and at least one activating domain. The costimulatory domain may be derived from (or correspond to), e.g., CD28, and the activating domain may be derived from (or correspond to) e.g., CD3-zeta. In certain embodiments, the CAR is designed to have two, three, four, or more costimulatory domains.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T cell transplantation.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

Comprise, include, and/or plural forms of each are open ended and include the listed parts and may include additional parts that are not listed. And/or is open ended and includes one or more of the listed parts and combinations of the listed parts.

It will be understood that descriptions herein are exemplary and explanatory only and are not restrictive of the invention as claimed. In this application, the use of the singular includes the plural unless specifically stated otherwise.

All documents, or portions of documents, cited in this application, including but not limited to patents, patent applications, articles, books, and treatises, are hereby expressly incorporated by reference in their entirety for any purpose. As utilized in accordance with the present disclosure, the following terms, unless otherwise indicated, shall be understood to have the following meanings:

In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

The citation of a reference herein should not be construed as an acknowledgement that such reference is prior art to the present invention. To the extent that any of the definitions or terms provided in the references incorporated by reference differ from the terms and discussion provided herein, the present terms and definitions control.

One skilled in the art will realize the subject matter may be embodied in other specific forms without departing from the spirit or essential characteristics thereof. The foregoing embodiments are therefore to be considered in all respects illustrative rather than limiting of the subject matter described herein.

The following sequences will further exemplify the invention:

CD28T DNA Extracellular, transmembrane, intracellular
(SEQ ID NO. 1)
CTTGATAATGAAAAGTCAAACGGAACAATCATTCACGTGAAGGGC
AAGCACCTCTGTCCGTCACCCTTGTTCCCTGGTCCATCCAAGCCATT
CTGGGTGTTGGTCGTAGTGGGTGGAGTCCTCGCTTGTTACTCTCTGC
TCGTCACCGTGGCTTTTATAATCTTCTGGGTTAGATCCAAAAGAAG
CCGCCTGCTCCATAGCGATTACATGAATATGACTCCACGCCGCCCT
GGCCCCACAAGGAAACACTACCAGCCTTACGCACCACCTAGAGAT
TTCGCTGCCTATCGGAGC
CD28T Extracellular, transmembrane, intracellular AA
(SEQ ID NO. 2)
LDNEKSNGTIIHVKGKHLCPSPLFPGPSKPFWVLVVVGGVLACYSLLV
TVAFIIFWVRSKRSRLLHSDYMNMTPRRPGPTRKHYQPYAPPRDFAAY
RS
CD28T DNA - Extracellular
(SEQ ID NO. 3)
CTTGATAATGAAAAGTCAAACGGAACAATCATTCACGTGAAGGGC
AAGCACCTCTGTCCGTCACCCTTGTTCCCTGGTCCATCCAAGCCA
CD28T AA - Extracellular
(SEQ ID NO. 4)
LDNEKSNGTI IHVKGKHLCP SPLFPGPSKP
CD28 DNA Transmembrane Domain
(SEQ ID NO. 5)
TTCTGGGTGTTGGTCGTAGTGGGTGGAGTCCTCGCTTGTTACTCTCT
GCTCGTCACCGTGGCTTTTATAATCTTCTGGGTT
CD28 AA Transmembrane Domain
(SEQ ID NO. 6)
FWVLVVVGGV LACYSLLVTV AFIIFWV
CD28 DNA Intracellular Domain
(SEQ ID NO. 7)
AGATCCAAAAGAAGCCGCCTGCTCCATAGCGATTACATGAATATG
ACTCCACGCCGCCCTGGCCCCACAAGGAAACACTACCAGCCTTACG
CACCACCTAGAGATTTCGCTGCCTATCGGAGC
CD28 AA Intracellular Domain
(SEQ ID NO. 8)
RSKRSRLLHSDYMNMTPRRPGPTRKHYQPYAPPRDFAAYRS
CD3 zeta DNA
(SEQ ID NO. 9)
AGGGTGAAGTTTTCCAGATCTGCAGATGCACCAGCGTATCAGCAGG
GCCAGAACCAACTGTATAACGAGCTCAACCTGGGACGCAGGGAAG
AGTATGACGTTTTGGACAAGCGCAGAGGACGGGACCCTGAGATGG
GTGGCAAACCAAGACGAAAAAACCCCCAGGAGGGTCTCTATAATG
AGCTGCAGAAGGATAAGATGGCTGAAGCCTATTCTGAAATAGGCA
TGAAAGGAGAGCGGAGAAGGGGAAAAGGGCACGACGGTTTGTAC
CAGGGACTCAGCACTGCTACGAAGGATACTTATGACGCTCTCCACA
TGCAAGCCCTGCCACCTAGG
CD3 zeta AA
(SEQ ID NO. 10)
RVKFSRSADAPAYQQGQNQLYNELNLGRREEYDVLDKRRGRDPEMG
GKPRRKNPQEGLYNELQKDKMAEAYSEIGMKGERRRGKGHDGLYQG
LSTATKDTYDALHMQALPPR
CD28 DNA
(SEQ ID NO. 11)
ATTGAGGTGATGTATCCACCGCCTTACCTGGATAACGAAAAGAGTA
ACGGTACCATCATTCACGTGAAAGGTAAACACCTGTGTCCTTCTCC
CCTCTTCCCCGGGCCATCAAAGCCC
CD28 AA
(SEQ ID NO. 12)
IEVMYPPPYLDNEKSNGTIIHVKGKHLCPSPLFPGPSKP
CD8 DNA extracellular & transmembrane domain
(SEQ ID NO. 13)
GCTGCAGCATTGAGCAACTCAATAATGTATTTTAGTCACTTTGTAC
CAGTGTTCTTGCCGGCTAAGCCTACTACCACACCCGCTCCACGGCC
ACCTACCCCAGCTCCTACCATCGCTTCACAGCCTCTGTCCCTGCGCC
CAGAGGCTTGCCGACCGGCCGCAGGGGGCGCTGTTCATACCAGAG
GACTGGATTTCGCCTGCGATATCTATATCTGGGCACCCCTGGCCGG
AACCTGCGGCGTACTCCTGCTGTCCCTGGTCATCACGCTCTATTGTA
ATCACAGGAAC
CD8 AA extracellular & transmembrane Domain
(SEQ ID NO. 14)
AAALSNSIMYFSHFVPVFLPAKPTTTPAPRPPTPAPTIASQPLSLRPEAC
RPAAGGAVHTRGLDFACDIYIWAPLAGTCGVLLLSLVITLYCNHRN
Clone 24C1 HC DNA
(SEQ ID NO. 15)
CAGGTGCAGCTGCAGGAATCCGGACCGGGGCTGGTGAAGCCCAGC
GAGACTCTGAGTCTCACGTGTACAGTTTCTGGAGGTAGCATTAGCT
CCTACTATTGGTCATGGATAAGGCAGCCCCCCGGGAAGGGATTGG
AATGGATCGGCTATATTTACTACAGTGGGAGCACCAATTACAACCC
CTCACTGAAGTCTAGAGTTACAATCAGCGTTGACACCTCAAAGAAT
CAGTTCAGTTTGAAATTGTCTAGCGTCACAGCAGCTGATACAGCCG
TCTATTATTGTGTTTCTCTGGTCTATTGCGGTGGGGATTGTTACAGT
GGCTTTGACTATTGGGGGCAGGGTACTCTGGTTACAGTTTCTTCC
Clone 24C1 HC AA (CDRs Underlined)
(SEQ ID NO. 16)
QVQLQESGPGLVKPSETLSLTCTVSGGSISSYYWSWIRQPPGKGLEWIG
Y
IYYSGSTNYNPSLKSRVTISVDTSKNQFSLKLSSVTAADTAVYYCVSLV
YCGGDCYSGFDYWGQGTLVTVSS
(SEQ ID NO. 17)
Clone 24C1 HC AA CDR1: GGSISSY
(SEQ ID NO. 18)
Clone 24C1 HC AA CDR2: YYSGS
(SEQ ID NO. 19)
Clone 24C1 HC AA CDR3: LVYCGGDCYSGFDY
Clone 24C1 LC DNA
(SEQ ID NO. 20)
GACATCCAGTTGACACAGAGCCCGAGTTCCTTGTCCGCCTCCGTCG
GGGATAGAGTGTCATTTACCTGTCAGGCCTCTCAGGATATTAATAA
CTTTCTGAATTGGTATCAGCAAAAGCCCGGAAAGGCACCCAAGCTG
TTGATTTACGACGCCAGTAACCTGGAGACAGGCGTGCCCTCCCGGT
TTAGTGGTAGCGGAAGCGGTACGGATTTTACCTTTACTATCAGCTC
TCTCCAACCCGAAGACATTGCAACCTACTATTGTCAACAATATGGA
AACCTGCCTTTTACATTTGGCGGCGGCACCAAGGTGGAGATTAAGC
GG
Clone 24C1 LC AA (CDRs Underlined)
(SEQ ID NO. 21)
DIQLTQSPSSLSASVGDRVSFTCQASQDINNFLNWYQQKPGKAPKLLIY
DASNLETGVPSRFSGSGSGTDFTFTISSLQPEDIATYYCQQYGNLPFTFG
GGTKVEIKR
(SEQ ID NO. 22)
Clone 24C1 LC CDR1 AA: QASQDINNFLN
(SEQ ID NO. 23)
Clone 24C1 LC CDR2 AA: DASNLET
(SEQ ID NO. 24)
Clone 24C1 LC CDR3 AA: QQYGNLPFT
Clone 24C1 CD28T CD3 zeta CAR DNA Heavy & Light Chains
(SEQ ID NO. 25)
ATGGCACTCCCCGTAACTGCTCTGCTGCTGCCGTTGGCATTGCTCCT
GCACGCCGCACGCCCGCAGGTCCAACTGCAAGAAAGCGGACCCGG
ACTGGTGAAGCCTTCTGAGACACTTAGTCTGACGTGCACGGTCAGT
GGCGGCTCCATCTCCTCCTATTATTGGTCATGGATACGACAACCCC
CAGGTAAGGGCCTGGAATGGATTGGCTATATCTACTATTCAGGAAG
CACGAACTACAATCCCAGCCTGAAGTCCCGAGTGACAATTTCAGTA
GATACCAGTAAAAACCAGTTCAGTCTTAAACTGTCAAGCGTGACAG
CTGCCGACACCGCTGTGTATTACTGCGTCTCACTGGTGTATTGTGG
AGGGGATTGTTATAGCGGGTTCGATTATTGGGGACAGGGAACCCTG
GTGACTGTATCTTCCGGCGGCGGCGGCTCAGGGGGTGGCGGTAGTG
GCGGTGGGGGTTCCGATATTCAACTGACACAATCCCCCAGCTCACT
CAGCGCCAGCGTGGGGGACAGGGTTAGCTTTACCTGTCAAGCCTCT
CAGGATATAAATAACTTTCTGAACTGGTATCAACAGAAGCCTGGGA
AGGCGCCCAAACTCCTGATCTATGATGCGTCCAACCTGGAAACTGG
CGTGCCTTCACGCTTTAGCGGCTCTGGCAGTGGTACAGACTTCACT
TTTACCATCTCTTCACTTCAGCCGGAGGACATCGCCACATATTACTG
TCAACAGTACGGAAACTTGCCCTTTACTTTTGGAGGCGGCACCAAA
GTTGAAATCAAAAGGGCCGCTGCCCTGGATAACGAAAAGAGCAAT
GGGACTATAATACATGTTAAAGGAAAACACCTGTGTCCATCTCCCC
TGTTCCCTGGACCGTCAAAGCCATTTTGGGTGCTCGTGGTTGTCGGT
GGCGTTCTCGCCTGTTATAGCTTGCTGGTGACAGTAGCCTTCATTAT
CTTTTGGGTGAGATCCAAAAGAAGCCGCCTGCTCCATAGCGATTAC
ATGAATATGACTCCACGCCGCCCTGGCCCCACAAGGAAACACTACC
AGCCTTACGCACCACCTAGAGATTTCGCTGCCTATCGGAGCAGGGT
GAAGTTTTCCAGATCTGCAGATGCACCAGCGTATCAGCAGGGCCAG
AACCAACTGTATAACGAGCTCAACCTGGGACGCAGGGAAGAGTAT
GACGTTTTGGACAAGCGCAGAGGACGGGACCCTGAGATGGGTGGC
AAACCAAGACGAAAAAACCCCCAGGAGGGTCTCTATAATGAGCTG
CAGAAGGATAAGATGGCTGAAGCCTATTCTGAAATAGGCATGAAA
GGAGAGCGGAGAAGGGGAAAAGGGCACGACGGTTTGTACCAGGG
ACTCAGCACTGCTACGAAGGATACTTATGACGCTCTCCACATGCAA
GCCCTGCCACCTAGGTAA
Clone 24C1 CD28T CD3 zeta CAR AA Heavy & Light Chains
(Signal Peptide in bold)
(SEQ ID NO. 26)
MALPVTALLLPLALLLHAARPQVQLQESGPGLVKPSETLSLTCTVSG
GSISSYYWSWIRQPPGKGLEWIGYIYYSGSTNYNPSLKSRVTISVDTSK
NQFSLKLSSVTAADTAVYYCVSLVYCGGDCYSGFDYWGQGTLVTVS
SGGGGSGGGGSGGGGSDIQLTQSPSSLSASVGDRVSFTCQASQDINNF
LNWYQQKPGKAPKLLIYDASNLETGVPSRFSGSGSGTDFTFTISSLQPE
DIATYYCQQYGNLPFTFGGGTKVEIKRAAALDNEKSNGTIIHVKGKHL
CPSPLFPGPSKPFWVLVVVGGVLACYSLLVTVAFIIFWVRSKRSRLLHS
DYMNMTPRRPGPTRKHYQPYAPPRDFAAYRSRVKFSRSADAPAYQQ
GQNQLYNELNLGRREEYDVLDKRRGRDPEMGGKPRRKNPQEGLYNE
LQKDKMAEAYSEIGMKGERRRGKGHDGLYQGLSTATKDTYDA
LHMQALPPR
Clone 24C1 CD28T CD3 zeta CAR DNA Heavy & Light Chains
(SEQ ID NO. 27)
CAGGTCCAACTGCAAGAAAGCGGACCCGGACTGGTGAAGCCTTCT
GAGACACTTAGTCTGACGTGCACGGTCAGTGGCGGCTCCATCTCCT
CCTATTATTGGTCATGGATACGACAACCCCCAGGTAAGGGCCTGGA
ATGGATTGGCTATATCTACTATTCAGGAAGCACGAACTACAATCCC
AGCCTGAAGTCCCGAGTGACAATTTCAGTAGATACCAGTAAAAAC
CAGTTCAGTCTTAAACTGTCAAGCGTGACAGCTGCCGACACCGCTG
TGTATTACTGCGTCTCACTGGTGTATTGTGGAGGGGATTGTTATAGC
GGGTTCGATTATTGGGGACAGGGAACCCTGGTGACTGTATCTTCCG
GCGGCGGCGGCTCAGGGGGTGGCGGTAGTGGCGGTGGGGGTTCCG
ATATTCAACTGACACAATCCCCCAGCTCACTCAGCGCCAGCGTGGG
GGACAGGGTTAGCTTTACCTGTCAAGCCTCTCAGGATATAAATAAC
TTTCTGAACTGGTATCAACAGAAGCCTGGGAAGGCGCCCAAACTCC
TGATCTATGATGCGTCCAACCTGGAAACTGGCGTGCCTTCACGCTT
TAGCGGCTCTGGCAGTGGTACAGACTTCACTTTTACCATCTCTTCAC
TTCAGCCGGAGGACATCGCCACATATTACTGTCAACAGTACGGAAA
CTTGCCCTTTACTTTTGGAGGCGGCACCAAAGTTGAAATCAAAAGG
GCCGCTGCCCTGGATAACGAAAAGAGCAATGGGACTATAATACAT
GTTAAAGGAAAACACCTGTGTCCATCTCCCCTGTTCCCTGGACCGT
CAAAGCCATTTTGGGTGCTCGTGGTTGTCGGTGGCGTTCTCGCCTGT
TATAGCTTGCTGGTGACAGTAGCCTTCATTATCTTTTGGGTGAGATC
CAAAAGAAGCCGCCTGCTCCATAGCGATTACATGAATATGACTCCA
CGCCGCCCTGGCCCCACAAGGAAACACTACCAGCCTTACGCACCAC
CTAGAGATTTCGCTGCCTATCGGAGCAGGGTGAAGTTTTCCAGATC
TGCAGATGCACCAGCGTATCAGCAGGGCCAGAACCAACTGTATAA
CGAGCTCAACCTGGGACGCAGGGAAGAGTATGACGTTTTGGACAA
GCGCAGAGGACGGGACCCTGAGATGGGTGGCAAACCAAGACGAA
AAAACCCCCAGGAGGGTCTCTATAATGAGCTGCAGAAGGATAAGA
TGGCTGAAGCCTATTCTGAAATAGGCATGAAAGGAGAGCGGAGAA
GGGGAAAAGGGCACGACGGTTTGTACCAGGGACTCAGCACTGCTA
CGAAGGATACTTATGACGCTCTCCACATGCAAGCCCTGCCACCTAG
G
Clone 24C1 CD28T CD3 zeta CAR AA Heavy & Light Chains
(SEQ ID NO. 28)
QVQLQESGPGLVKPSETLSLTCTVSGGSISSYYWSWIRQPPGKGLEWIG
YIYYSGSTNYNPSLKSRVTISVDTSKNQFSLKLSSVTAADTAVYYCVSL
VYCGGDCYSGFDYWGQGTLVTVSSGGGGSGGGGSGGGGSDIQLTQS
PSSLSASVGDRVSFTCQASQDINNFLNWYQQKPGKAPKLLIYDASNLE
TGVPSRFSGSGSGTDFTFTISSLQPEDIATYYCQQYGNLPFTFGGGTKV
EIKRAAALDNEKSNGTIIHVKGKHLCPSPLFPGPSKPFWVLVVVGGVL
ACYSLLVTVAFIIFWVRSKRSRLLHSDYMNMTPRRPGPTRKHYQPYAP
PRDFAAYRSRVKFSRSADAPAYQQGQNQLYNELNLGRREEYDVLDK
RRGRDPEMGGKPRRKNPQEGLYNELQKDKMAEAYSEIGMKGERRRG
KGHDGLYQGLSTATKDTYDALHMQALPPR
Clone 24C1 CD28 CD3 zeta CAR DNA Heavy & Light Chains
(SEQ ID NO. 29)
ATGGCACTCCCCGTAACTGCTCTGCTGCTGCCGTTGGCATTGCTCCT
GCACGCCGCACGCCCGCAGGTGCAGCTGCAGGAATCCGGACCGGG
GCTGGTGAAGCCCAGCGAGACTCTGAGTCTCACGTGTACAGTTTCT
GGAGGTAGCATTAGCTCCTACTATTGGTCATGGATAAGGCAGCCCC
CCGGGAAGGGATTGGAATGGATCGGCTATATTTACTACAGTGGGA
GCACCAATTACAACCCCTCACTGAAGTCTAGAGTTACAATCAGCGT
TGACACCTCAAAGAATCAGTTCAGTTTGAAATTGTCTAGCGTCACA
GCAGCTGATACAGCCGTCTATTATTGTGTTTCTCTGGTCTATTGCGG
TGGGGATTGTTACAGTGGCTTTGACTATTGGGGGCAGGGTACTCTG
GTTACAGTTTCTTCCGGGGGGGGAGGCTCTGGGGGCGGAGGCTCA
GGTGGTGGAGGCAGCGACATCCAGTTGACACAGAGCCCGAGTTCC
TTGTCCGCCTCCGTCGGGGATAGAGTGTCATTTACCTGTCAGGCCT
CTCAGGATATTAATAACTTTCTGAATTGGTATCAGCAAAAGCCCGG
AAAGGCACCCAAGCTGTTGATTTACGACGCCAGTAACCTGGAGAC
AGGCGTGCCCTCCCGGTTTAGTGGTAGCGGAAGCGGTACGGATTTT
ACCTTTACTATCAGCTCTCTCCAACCCGAAGACATTGCAACCTACT
ATTGTCAACAATATGGAAACCTGCCTTTTACATTTGGCGGCGGCAC
CAAGGTGGAGATTAAGCGGGCGGCAGCTATTGAGGTGATGTATCC
ACCGCCTTACCTGGATAACGAAAAGAGTAACGGTACCATCATTCAC
GTGAAAGGTAAACACCTGTGTCCTTCTCCCCTCTTCCCCGGGCCAT
CAAAGCCCTTCTGGGTTCTTGTGGTCGTGGGAGGCGTGCTTGCTTG
TTATTCTCTGCTCGTTACCGTGGCGTTTATCATTTTTTGGGTTAGATC
CAAAAGAAGCCGCCTGCTCCATAGCGATTACATGAATATGACTCCA
CGCCGCCCTGGCCCCACAAGGAAACACTACCAGCCTTACGCACCAC
CTAGAGATTTCGCTGCCTATCGGAGCAGGGTGAAGTTTTCCAGATC
TGCAGATGCACCAGCGTATCAGCAGGGCCAGAACCAACTGTATAA
CGAGCTCAACCTGGGACGCAGGGAAGAGTATGACGTTTTGGACAA
GCGCAGAGGACGGGACCCTGAGATGGGTGGCAAACCAAGACGAA
AAAACCCCCAGGAGGGTCTCTATAATGAGCTGCAGAAGGATAAGA
TGGCTGAAGCCTATTCTGAAATAGGCATGAAAGGAGAGCGGAGAA
GGGGAAAAGGGCACGACGGTTTGTACCAGGGACTCAGCACTGCTA
CGAAGGATACTTATGACGCTCTCCACATGCAAGCCCTGCCACCTAG
GTAA
Clone 24C1 CD28 CD3 zeta CAR AA Heavy & Light Chains
(Signal Peptide in Bold)
(SEQ ID NO. 30)
MALPVTALLLPLALLLHAARPQVQLQESGPGLVKPSETLSLTCTVSG
GSISSYYWSWIRQPPGKGLEWIGYIYYSGSTNYNPSLKSRVTISVDTSK
NQFSLKLSSVTAADTAVYYCVSLVYCGGDCYSGFDYWGQGTLVTVS
SGGGGSGGGGSGGGGSDIQLTQSPSSLSASVGDRVSFTCQASQDINNF
LNWYQQKPGKAPKLLIYDASNLETGVPSRFSGSGSGTDFTFTISSLQPE
DIATYYCQQYGNLPFTFGGGTKVEIKRAAAIEVMYPPPYLDNEKSNGT
IIHVKGKHLCPSPLFPGPSKPFWVLVVVGGVLACYSLLVTVAFIIFWVR
SKRSRLLHSDYMNMTPRRPGPTRKHYQPYAPPRDFAAYRSRVKFSRS
ADAPAYQQGQNQLYNELNLGRREEYDVLDKRRGRDPEMGGKPRRK
NPQEGLYNELQKDKMAEAYSEIGMKGERRRGKGHDGLYQGLSTATK
DTYDALHMQALPPR
Clone 24C1 CD28 CD3 zeta CAR DNA Heavy & Light Chains
(SEQ ID NO. 31)
CAGGTGCAGCTGCAGGAATCCGGACCGGGGCTGGTGAAGCCCAGC
GAGACTCTGAGTCTCACGTGTACAGTTTCTGGAGGTAGCATTAGCT
CCTACTATTGGTCATGGATAAGGCAGCCCCCCGGGAAGGGATTGG
AATGGATCGGCTATATTTACTACAGTGGGAGCACCAATTACAACCC
CTCACTGAAGTCTAGAGTTACAATCAGCGTTGACACCTCAAAGAAT
CAGTTCAGTTTGAAATTGTCTAGCGTCACAGCAGCTGATACAGCCG
TCTATTATTGTGTTTCTCTGGTCTATTGCGGTGGGGATTGTTACAGT
GGCTTTGACTATTGGGGGCAGGGTACTCTGGTTACAGTTTCTTCCG
GGGGGGGAGGCTCTGGGGGCGGAGGCTCAGGTGGTGGAGGCAGCG
ACATCCAGTTGACACAGAGCCCGAGTTCCTTGTCCGCCTCCGTCGG
GGATAGAGTGTCATTTACCTGTCAGGCCTCTCAGGATATTAATAAC
TTTCTGAATTGGTATCAGCAAAAGCCCGGAAAGGCACCCAAGCTGT
TGATTTACGACGCCAGTAACCTGGAGACAGGCGTGCCCTCCCGGTT
TAGTGGTAGCGGAAGCGGTACGGATTTTACCTTTACTATCAGCTCT
CTCCAACCCGAAGACATTGCAACCTACTATTGTCAACAATATGGAA
ACCTGCCTTTTACATTTGGCGGCGGCACCAAGGTGGAGATTAAGCG
GGCGGCAGCTATTGAGGTGATGTATCCACCGCCTTACCTGGATAAC
GAAAAGAGTAACGGTACCATCATTCACGTGAAAGGTAAACACCTG
TGTCCTTCTCCCCTCTTCCCCGGGCCATCAAAGCCCTTCTGGGTTCT
TGTGGTCGTGGGAGGCGTGCTTGCTTGTTATTCTCTGCTCGTTACCG
TGGCGTTTATCATTTTTTGGGTTAGATCCAAAAGAAGCCGCCTGCT
CCATAGCGATTACATGAATATGACTCCACGCCGCCCTGGCCCCACA
AGGAAACACTACCAGCCTTACGCACCACCTAGAGATTTCGCTGCCT
ATCGGAGCAGGGTGAAGTTTTCCAGATCTGCAGATGCACCAGCGTA
TCAGCAGGGCCAGAACCAACTGTATAACGAGCTCAACCTGGGACG
CAGGGAAGAGTATGACGTTTTGGACAAGCGCAGAGGACGGGACCC
TGAGATGGGTGGCAAACCAAGACGAAAAAACCCCCAGGAGGGTCT
CTATAATGAGCTGCAGAAGGATAAGATGGCTGAAGCCTATTCTGA
AATAGGCATGAAAGGAGAGCGGAGAAGGGGAAAAGGGCACGACG
GTTTGTACCAGGGACTCAGCACTGCTACGAAGGATACTTATGACGC
TCTCCACATGCAAGCCCTGCCACCTAGG
Clone 24C1 CD28 CD3 zeta CAR AA Heavy & Light Chains
(SEQ ID NO. 32)
QVQLQESGPGLVKPSETLSLTCTVSGGSISSYYWSWIRQPPGKGLEWIG
YIYYSGSTNYNPSLKSRVTISVDTSKNQFSLKLSSVTAADTAVYYCVSL
VYCGGDCYSGFDYWGQGTLVTVSSGGGGSGGGGSGGGGSDIQLTQS
PSSLSASVGDRVSFTCQASQDINNFLNWYQQKPGKAPKLLIYDASNLE
TGVPSRFSGSGSGTDFTFTISSLQPEDIATYYCQQYGNLPFTFGGGTKV
EIKRAAAIEVMYPPPYLDNEKSNGTIIHVKGKHLCPSPLFPGPSKPFWV
LVVVGGVLACYSLLVTVAFIIFWVRSKRSRLLHSDYMNMTPRRPGPTR
KHYQPYAPPRDFAAYRSRVKFSRSADAPAYQQGQNQLYNELNLGRRE
EYDVLDKRRGRDPEMGGKPRRKNPQEGLYNELQKDKMAEAYSEIGM
KGERRRGKGHDGLYQGLST ATKDTYDALHMQALPPR
Clone 24C1 CD8 CD3 zeta CAR DNA Heavy & Light Chains
(SEQ ID NO. 33)
ATGGCACTCCCCGTAACTGCTCTGCTGCTGCCGTTGGCATTGCTCCT
GCACGCCGCACGCCCGCAGGTGCAATTGCAAGAGTCCGGCCCCGG
ACTCGTTAAACCCAGTGAGACGCTTAGCCTGACCTGTACCGTCTCA
GGGGGCAGCATCTCCTCTTATTACTGGAGCTGGATCAGGCAGCCTC
CAGGAAAAGGCCTTGAATGGATTGGGTACATCTACTACTCTGGCTC
AACAAATTATAATCCATCCCTGAAGTCCCGCGTGACTATCTCTGTG
GACACCAGCAAGAATCAGTTTTCACTGAAGTTGTCTAGTGTTACCG
CGGCCGACACCGCCGTATACTACTGTGTGTCTCTTGTGTACTGTGG
CGGCGACTGCTATTCCGGGTTCGACTACTGGGGCCAAGGGACTCTG
GTAACCGTGTCCTCAGGCGGCGGCGGGTCAGGAGGAGGCGGCAGT
GGAGGTGGCGGCTCCGACATCCAGCTGACACAATCACCATCTTCCC
TTTCAGCTTCAGTCGGGGACAGAGTGTCCTTCACATGCCAGGCCAG
CCAGGATATCAATAACTTCCTGAACTGGTACCAACAGAAACCCGG
AAAGGCTCCAAAGCTCCTGATCTATGATGCTTCCAACCTGGAGACC
GGCGTGCCCTCCAGGTTCAGTGGTTCAGGATCAGGCACTGACTTTA
CGTTCACCATATCCAGTCTTCAGCCCGAAGACATTGCAACCTATTA
CTGCCAACAATACGGGAACCTTCCCTTTACATTCGGAGGCGGCACC
AAGGTGGAAATCAAAAGGGCTGCAGCATTGAGCAACTCAATAATG
TATTTTAGTCACTTTGTACCAGTGTTCTTGCCGGCTAAGCCTACTAC
CACACCCGCTCCACGGCCACCTACCCCAGCTCCTACCATCGCTTCA
CAGCCTCTGTCCCTGCGCCCAGAGGCTTGCCGACCGGCCGCAGGGG
GCGCTGTTCATACCAGAGGACTGGATTTCGCCTGCGATATCTATAT
CTGGGCACCCCTGGCCGGAACCTGCGGCGTACTCCTGCTGTCCCTG
GTCATCACGCTCTATTGTAATCACAGGAACAGATCCAAAAGAAGCC
GCCTGCTCCATAGCGATTACATGAATATGACTCCACGCCGCCCTGG
CCCCACAAGGAAACACTACCAGCCTTACGCACCACCTAGAGATTTC
GCTGCCTATCGGAGCAGGGTGAAGTTTTCCAGATCTGCAGATGCAC
CAGCGTATCAGCAGGGCCAGAACCAACTGTATAACGAGCTCAACC
TGGGACGCAGGGAAGAGTATGACGTTTTGGACAAGCGCAGAGGAC
GGGACCCTGAGATGGGTGGCAAACCAAGACGAAAAAACCCCCAGG
AGGGTCTCTATAATGAGCTGCAGAAGGATAAGATGGCTGAAGCCT
ATTCTGAAATAGGCATGAAAGGAGAGCGGAGAAGGGGAAAAGGG
CACGACGGTTTGTACCAGGGACTCAGCACTGCTACGAAGGATACTT
ATGACGCTCTCCACATGCAAGCCCTGCCACCTAGGTAA
Clone 24C1 CD8 CD3 zeta CAR AA Heavy & Light Chains
(Signal peptide in bold)
(SEQ ID NO. 34)
MALPVTALLLPLALLLHAARPQVQLQESGPGLVKPSETLSLTCTVSG
GSISSYYWSWIRQPPGKGLEWIGYIYYSGSTNYNPSLKSRVTISVDTSK
NQFSLKLSSVTAADTAVYYCVSLVYCGGDCYSGFDYWGQGTLVTVS
SGGGGSGGGGSGGGGSDIQLTQSPSSLSASVGDRVSFTCQASQDINNF
LNWYQQKPGKAPKLLIYDASNLETGVPSRFSGSGSGTDFTFTISSLQPE
DIATYYCQQYGNLPFTFGGGTKVEIKRAAALSNSIMYFSHFVPVFLPA
KPTTTPAPRPPTPAPTIASQPLSLRPEACRPAAGGAVHTRGLDFACDIYI
WAPLAGTCGVLLLSLVITLYCNHRNRSKRSRLLHSDYMNMTPRRPGP
TRKHYQPYAPPRDFAAYRSRVKFSRSADAPAYQQGQNQLYNELNLGR
REEYDVLDKRRGRDPEMGGKPRRKNPQEGLYNELQKDKMAEAYSEI
GMKGERRRGKGHDGLYQGLSTATKDTYDALHMQALPPR
Clone 24C1 CD8 CD3 zeta CAR DNA Heavy & Light Chains
(SEQ ID NO. 35)
CAGGTGCAATTGCAAGAGTCCGGCCCCGGACTCGTTAAACCCAGTG
AGACGCTTAGCCTGACCTGTACCGTCTCAGGGGGCAGCATCTCCTC
TTATTACTGGAGCTGGATCAGGCAGCCTCCAGGAAAAGGCCTTGAA
TGGATTGGGTACATCTACTACTCTGGCTCAACAAATTATAATCCAT
CCCTGAAGTCCCGCGTGACTATCTCTGTGGACACCAGCAAGAATCA
GTTTTCACTGAAGTTGTCTAGTGTTACCGCGGCCGACACCGCCGTA
TACTACTGTGTGTCTCTTGTGTACTGTGGCGGCGACTGCTATTCCGG
GTTCGACTACTGGGGCCAAGGGACTCTGGTAACCGTGTCCTCAGGC
GGCGGCGGGTCAGGAGGAGGCGGCAGTGGAGGTGGCGGCTCCGAC
ATCCAGCTGACACAATCACCATCTTCCCTTTCAGCTTCAGTCGGGG
ACAGAGTGTCCTTCACATGCCAGGCCAGCCAGGATATCAATAACTT
CCTGAACTGGTACCAACAGAAACCCGGAAAGGCTCCAAAGCTCCT
GATCTATGATGCTTCCAACCTGGAGACCGGCGTGCCCTCCAGGTTC
AGTGGTTCAGGATCAGGCACTGACTTTACGTTCACCATATCCAGTC
TTCAGCCCGAAGACATTGCAACCTATTACTGCCAACAATACGGGAA
CCTTCCCTTTACATTCGGAGGCGGCACCAAGGTGGAAATCAAAAGG
GCTGCAGCATTGAGCAACTCAATAATGTATTTTAGTCACTTTGTAC
CAGTGTTCTTGCCGGCTAAGCCTACTACCACACCCGCTCCACGGCC
ACCTACCCCAGCTCCTACCATCGCTTCACAGCCTCTGTCCCTGCGCC
CAGAGGCTTGCCGACCGGCCGCAGGGGGCGCTGTTCATACCAGAG
GACTGGATTTCGCCTGCGATATCTATATCTGGGCACCCCTGGCCGG
AACCTGCGGCGTACTCCTGCTGTCCCTGGTCATCACGCTCTATTGTA
ATCACAGGAACAGATCCAAAAGAAGCCGCCTGCTCCATAGCGATT
ACATGAATATGACTCCACGCCGCCCTGGCCCCACAAGGAAACACT
ACCAGCCTTACGCACCACCTAGAGATTTCGCTGCCTATCGGAGCAG
GGTGAAGTTTTCCAGATCTGCAGATGCACCAGCGTATCAGCAGGGC
CAGAACCAACTGTATAACGAGCTCAACCTGGGACGCAGGGAAGAG
TATGACGTTTTGGACAAGCGCAGAGGACGGGACCCTGAGATGGGT
GGCAAACCAAGACGAAAAAACCCCCAGGAGGGTCTCTATAATGAG
CTGCAGAAGGATAAGATGGCTGAAGCCTATTCTGAAATAGGCATG
AAAGGAGAGCGGAGAAGGGGAAAAGGGCACGACGGTTTGTACCA
GGGACTCAGCACTGCTACGAAGGATACTTATGACGCTCTCCACATG
CAAGCCCTGCCACCTAGG
Clone 24C1 CD8 CD3 zeta CAR AA Heavy & Light Chains
(SEQ ID NO. 36)
QVQLQESGPGLVKPSETLSLTCTVSGGSISSYYWSWIRQPPGKGLEWIG
YIYYSGSTNYNPSLKSRVTISVDTSKNQFSLKLSSVTAADTAVYYCVSL
VYCGGDCYSGFDYWGQGTLVTVSSGGGGSGGGGSGGGGSDIQLTQS
PSSLSASVGDRVSFTCQASQDINNFLNWYQQKPGKAPKLLIYDASNLE
TGVPSRFSGSGSGTDFTFTISSLQPEDIATYYCQQYGNLPFTFGGGTKV
EIKRAAALSNSIMYFSHFVPVFLPAKPTTTPAPRPPTPAPTIASQPLSLRP
EACRPAAGGAVHTRGLDFACDIYIWAPLAGTCGVLLLSLVITLYCNHR
NRSKRSRLLHSDYMNMTPRRPGPTRKHYQPYAPPRDFAAYRSRVKFS
RSADAPAYQQGQNQLYNELNLGRREEYDVLDKRRGRDPEMGGKPRR
KNPQEGLYNELQKDKMAEAYSEIGMKGERRRGKGHDGLYQGLSTAT
KDTYDALHMQALPPR
Clone 24C1 CD28T CD3 zeta CAR DNA Heavy & Light Chains
(SEQ ID NO. 37)
ATGGCACTCCCCGTAACTGCTCTGCTGCTGCCGTTGGCATTGCTCCT
GCACGCCGCACGCCCGGATATCCAGCTCACGCAATCCCCCTCAAGC
TTGAGTGCCTCCGTGGGCGACCGGGTGTCCTTCACATGTCAGGCAA
GCCAAGACATAAATAATTTCCTGAATTGGTACCAACAAAAACCCG
GCAAGGCTCCCAAACTCCTGATTTATGATGCCTCCAATCTGGAGAC
CGGGGTCCCTTCTAGATTCAGCGGAAGTGGCAGCGGCACAGACTTT
ACATTTACTATCTCTTCTCTGCAACCAGAGGACATCGCCACATACT
ATTGCCAGCAATACGGCAATCTGCCCTTCACCTTCGGAGGCGGAAC
CAAGGTAGAAATTAAAAGGGGCGGTGGAGGCTCCGGAGGGGGGG
GCTCTGGCGGAGGGGGCTCCCAAGTACAATTGCAGGAGTCAGGGC
CTGGACTCGTGAAGCCTTCAGAAACTTTGTCACTGACATGTACAGT
GTCCGGCGGAAGCATTTCCAGTTACTATTGGTCCTGGATTAGACAG
CCACCCGGCAAAGGACTGGAATGGATTGGATATATCTACTACTCTG
GATCTACAAACTATAATCCCAGCCTCAAATCCAGGGTCACTATTAG
TGTGGATACATCAAAGAATCAGTTCTCCTTGAAGCTGAGCTCAGTC
ACTGCTGCCGACACCGCAGTGTACTATTGTGTGAGCCTGGTCTACT
GCGGCGGAGATTGCTACAGCGGTTTCGATTACTGGGGCCAGGGCA
CCCTGGTTACCGTTAGTTCCGCGGCTGCTCTTGATAACGAGAAGTC
CAACGGTACGATTATCCACGTTAAGGGTAAGCACCTTTGCCCTAGC
CCGCTGTTCCCAGGCCCCAGTAAGCCCTTTTGGGTCCTCGTTGTGGT
AGGTGGGGTACTCGCCTGCTACTCCCTGCTCGTCACTGTCGCATTC
ATCATCTTCTGGGTCAGATCCAAAAGAAGCCGCCTGCTCCATAGCG
ATTACATGAATATGACTCCACGCCGCCCTGGCCCCACAAGGAAACA
CTACCAGCCTTACGCACCACCTAGAGATTTCGCTGCCTATCGGAGC
AGGGTGAAGTTTTCCAGATCTGCAGATGCACCAGCGTATCAGCAGG
GCCAGAACCAACTGTATAACGAGCTCAACCTGGGACGCAGGGAAG
AGTATGACGTTTTGGACAAGCGCAGAGGACGGGACCCTGAGATGG
GTGGCAAACCAAGACGAAAAAACCCCCAGGAGGGTCTCTATAATG
AGCTGCAGAAGGATAAGATGGCTGAAGCCTATTCTGAAATAGGCA
TGAAAGGAGAGCGGAGAAGGGGAAAAGGGCACGACGGTTTGTAC
CAGGGACTCAGCACTGCTACGAAGGATACTTATGACGCTCTCCACA
TGCAAGCCCTGCCACCTAGGTAA
Clone 24C1 CD28T CD3 zeta CAR AA Heavy & Light Chains
(Signal Peptide in Bold)
(SEQ ID NO. 38)
MALPVTALLLPLALLLHAARPDIQLTQSPSSLSASVGDRVSFTCQAS
QDINNFLNWYQQKPGKAPKLLIYDASNLETGVPSRFSGSGSGTDFTFTI
SSLQPEDIATYYCQQYGNLPFTFGGGTKVEIKRGGGGSGGGGSGGGGS
QVQLQESGPGLVKPSETLSLTCTVSGGSISSYYWSWIRQPPGKGLEWIG
YIYYSGSTNYNPSLKSRVTISVDTSKNQFSLKLSSVTAADTAVYYCVSL
VYCGGDCYSGFDYWGQGTLVTVSSAAALDNEKSNGTIIHVKGKHLCP
SPLFPGPSKPFWVLVVVGGVLACYSLLVTVAFIIFWVRSKRSRLLHSD
YMNMTPRRPGPTRKHYQPYAPPRDFAAYRSRVKFSRSADAPAYQQG
QNQLYNELNLGRREEYDVLDKRRGRDPEMGGKPRRKNPQEGLYNEL
QKDKMAEAYSEIGMKGERRRGKGHDGLYQGLSTATKDTYDA
LHMQALPPR
Clone 24C1 CD28T CD3 zeta CAR DNA Heavy & Light Chains
(SEQ ID NO. 39)
GATATCCAGCTCACGCAATCCCCCTCAAGCTTGAGTGCCTCCGTGG
GCGACCGGGTGTCCTTCACATGTCAGGCAAGCCAAGACATAAATA
ATTTCCTGAATTGGTACCAACAAAAACCCGGCAAGGCTCCCAAACT
CCTGATTTATGATGCCTCCAATCTGGAGACCGGGGTCCCTTCTAGA
TTCAGCGGAAGTGGCAGCGGCACAGACTTTACATTTACTATCTCTT
CTCTGCAACCAGAGGACATCGCCACATACTATTGCCAGCAATACGG
CAATCTGCCCTTCACCTTCGGAGGCGGAACCAAGGTAGAAATTAAA
AGGGGCGGTGGAGGCTCCGGAGGGGGGGGCTCTGGCGGAGGGGG
CTCCCAAGTACAATTGCAGGAGTCAGGGCCTGGACTCGTGAAGCCT
TCAGAAACTTTGTCACTGACATGTACAGTGTCCGGCGGAAGCATTT
CCAGTTACTATTGGTCCTGGATTAGACAGCCACCCGGCAAAGGACT
GGAATGGATTGGATATATCTACTACTCTGGATCTACAAACTATAAT
CCCAGCCTCAAATCCAGGGTCACTATTAGTGTGGATACATCAAAGA
ATCAGTTCTCCTTGAAGCTGAGCTCAGTCACTGCTGCCGACACCGC
AGTGTACTATTGTGTGAGCCTGGTCTACTGCGGCGGAGATTGCTAC
AGCGGTTTCGATTACTGGGGCCAGGGCACCCTGGTTACCGTTAGTT
CCGCGGCTGCTCTTGATAACGAGAAGTCCAACGGTACGATTATCCA
CGTTAAGGGTAAGCACCTTTGCCCTAGCCCGCTGTTCCCAGGCCCC
AGTAAGCCCTTTTGGGTCCTCGTTGTGGTAGGTGGGGTACTCGCCT
GCTACTCCCTGCTCGTCACTGTCGCATTCATCATCTTCTGGGTCAGA
TCCAAAAGAAGCCGCCTGCTCCATAGCGATTACATGAATATGACTC
CACGCCGCCCTGGCCCCACAAGGAAACACTACCAGCCTTACGCACC
ACCTAGAGATTTCGCTGCCTATCGGAGCAGGGTGAAGTTTTCCAGA
TCTGCAGATGCACCAGCGTATCAGCAGGGCCAGAACCAACTGTAT
AACGAGCTCAACCTGGGACGCAGGGAAGAGTATGACGTTTTGGAC
AAGCGCAGAGGACGGGACCCTGAGATGGGTGGCAAACCAAGACG
AAAAAACCCCCAGGAGGGTCTCTATAATGAGCTGCAGAAGGATAA
GATGGCTGAAGCCTATTCTGAAATAGGCATGAAAGGAGAGCGGAG
AAGGGGAAAAGGGCACGACGGTTTGTACCAGGGACTCAGCACTGC
TACGAAGGATACTTATGACGCTCTCCACATGCAAGCCCTGCCACCT
AGG
Clone 24C1 CD28T CD3 zeta CAR AA Heavy & Light Chains
(SEQ ID NO. 40)
DIQLTQSPSSLSASVGDRVSFTCQASQDINNFLNWYQQKPGKAPKLLIY
DASNLETGVPSRFSGSGSGTDFTFTISSLQPEDIATYYCQQYGNLPFTFG
GGTKVEIKRGGGGSGGGGSGGGGSQVQLQESGPGLVKPSETLSLTCT
VSGGSISSYYWSWIRQPPGKGLEWIGYIYYSGSTNYNPSLKSRVTISVD
TSKNQFSLKLSSVTAADTAVYYCVSLVYCGGDCYSGFDYWGQGTLV
TVSSAAALDNEKSNGTIIHVKGKHLCPSPLFPGPSKPFWVLVVVGGVL
ACYSLLVTVAFIIFWVRSKRSRLLHSDYMNMTPRRPGPTRKHYQPYAP
PRDFAAYRSRVKFSRSADAPAYQQGQNQLYNELNLGRREEYDVLDK
RRGRDPEMGGKPRRKNPQEGLYNELQKDKMAEAYSEIGMKGERRRG
KGHDGLYQGLSTATKDTYDALHMQALPPR
Clone 24C1 CD28 CD3 zeta CAR DNA AA Heavy & Light Chains
(SEQ ID NO. 41)
ATGGCACTCCCCGTAACTGCTCTGCTGCTGCCGTTGGCATTGCTCCT
GCACGCCGCACGCCCGGATATCCAGCTGACCCAGTCTCCATCCTCT
TTGAGTGCCTCCGTGGGTGACCGCGTCTCTTTCACTTGCCAAGCCA
GCCAAGACATCAACAACTTTCTGAATTGGTACCAGCAGAAACCAG
GCAAAGCACCAAAGCTCCTCATCTACGACGCCTCCAACCTGGAAAC
CGGGGTGCCCAGCAGGTTTAGCGGGAGCGGTTCTGGCACGGATTTT
ACGTTCACCATCTCCTCTCTGCAGCCCGAGGATATAGCTACTTATTA
CTGTCAGCAGTACGGGAATCTGCCATTTACTTTTGGGGGTGGAACT
AAGGTGGAAATCAAAAGGGGCGGCGGGGGAAGCGGGGGCGGGGG
CTCAGGTGGCGGAGGGAGCCAGGTGCAACTCCAGGAAAGTGGCCC
AGGATTGGTGAAGCCCAGCGAGACCCTTTCCCTTACTTGTACTGTT
AGCGGAGGCAGCATAAGCAGCTACTATTGGTCCTGGATCAGACAG
CCACCAGGGAAAGGGCTTGAATGGATTGGCTACATTTACTATTCCG
GGTCCACCAACTACAACCCATCCCTCAAGTCCCGCGTGACAATTTC
CGTCGACACAAGCAAGAACCAGTTCTCCCTGAAACTTAGTAGCGTC
ACTGCTGCAGATACAGCAGTGTACTATTGTGTCAGCCTTGTCTACT
GTGGCGGCGACTGCTACAGTGGCTTTGATTACTGGGGACAGGGCAC
GCTCGTGACAGTGTCCAGCGCTGCGGCTATCGAGGTAATGTATCCG
CCACCGTATCTGGACAACGAGAAGTCTAATGGGACAATCATTCACG
TGAAGGGGAAGCACCTGTGTCCATCCCCCCTGTTTCCGGGTCCCAG
TAAACCCTTCTGGGTGCTTGTTGTCGTTGGCGGGGTGCTGGCCTGCT
ATTCCCTGCTGGTGACCGTCGCGTTTATTATTTTCTGGGTTAGATCC
AAAAGAAGCCGCCTGCTCCATAGCGATTACATGAATATGACTCCAC
GCCGCCCTGGCCCCACAAGGAAACACTACCAGCCTTACGCACCACC
TAGAGATTTCGCTGCCTATCGGAGCAGGGTGAAGTTTTCCAGATCT
GCAGATGCACCAGCGTATCAGCAGGGCCAGAACCAACTGTATAAC
GAGCTCAACCTGGGACGCAGGGAAGAGTATGACGTTTTGGACAAG
CGCAGAGGACGGGACCCTGAGATGGGTGGCAAACCAAGACGAAA
AAACCCCCAGGAGGGTCTCTATAATGAGCTGCAGAAGGATAAGAT
GGCTGAAGCCTATTCTGAAATAGGCATGAAAGGAGAGCGGAGAAG
GGGAAAAGGGCACGACGGTTTGTACCAGGGACTCAGCACTGCTAC
GAAGGATACTTATGACGCTCTCCACATGCAAGCCCTGCCACCTAGG
TAA
Clone 24C1 CD28 CD3 zeta CAR AA Heavy & Light Chains
(Signal Peptide in Bold)
(SEQ ID NO. 42)
MALPVTALLLPLALLLHAARPDIQLTQSPSSLSASVGDRVSFTCQAS
QDINNFLNWYQQKPGKAPKLLIYDASNLETGVPSRFSGSGSGTDFTFTI
SSLQPEDIATYYCQQYGNLPFTFGGGTKVEIKRGGGGSGGGGSGGGGS
QVQLQESGPGLVKPSETLSLTCTVSGGSISSYYWSWIRQPPGKGLEWIG
YIYYSGSTNYNPSLKSRVTISVDTSKNQFSLKLSSVTAADTAVYYCVSL
VYCGGDCYSGFDYWGQGTLVTVSSAAAIEVMYPPPYLDNEKSNGTII
HVKGKHLCPSPLFPGPSKPFWVLVVVGGVLACYSLLVTVAFIIFWVRS
KRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNP
QEGLYNELQKDKMAEAYSEIGMKGERRRGKGHDGLYQGLSTATKDT
YDALHMQALPPR
Clone 24C1 CD28 CD3 zeta CAR DNA Heavy & Light Chains
(SEQ ID NO. 43)
GATATCCAGCTGACCCAGTCTCCATCCTCTTTGAGTGCCTCCGTGG
GTGACCGCGTCTCTTTCACTTGCCAAGCCAGCCAAGACATCAACAA
CTTTCTGAATTGGTACCAGCAGAAACCAGGCAAAGCACCAAAGCT
CCTCATCTACGACGCCTCCAACCTGGAAACCGGGGTGCCCAGCAGG
TTTAGCGGGAGCGGTTCTGGCACGGATTTTACGTTCACCATCTCCTC
TCTGCAGCCCGAGGATATAGCTACTTATTACTGTCAGCAGTACGGG
AATCTGCCATTTACTTTTGGGGGTGGAACTAAGGTGGAAATCAAAA
GGGGCGGCGGGGGAAGCGGGGGCGGGGGCTCAGGTGGCGGAGGG
AGCCAGGTGCAACTCCAGGAAAGTGGCCCAGGATTGGTGAAGCCC
AGCGAGACCCTTTCCCTTACTTGTACTGTTAGCGGAGGCAGCATAA
GCAGCTACTATTGGTCCTGGATCAGACAGCCACCAGGGAAAGGGC
TTGAATGGATTGGCTACATTTACTATTCCGGGTCCACCAACTACAA
CCCATCCCTCAAGTCCCGCGTGACAATTTCCGTCGACACAAGCAAG
AACCAGTTCTCCCTGAAACTTAGTAGCGTCACTGCTGCAGATACAG
CAGTGTACTATTGTGTCAGCCTTGTCTACTGTGGCGGCGACTGCTA
CAGTGGCTTTGATTACTGGGGACAGGGCACGCTCGTGACAGTGTCC
AGCGCTGCGGCTATCGAGGTAATGTATCCGCCACCGTATCTGGACA
ACGAGAAGTCTAATGGGACAATCATTCACGTGAAGGGGAAGCACC
TGTGTCCATCCCCCCTGTTTCCGGGTCCCAGTAAACCCTTCTGGGTG
CTTGTTGTCGTTGGCGGGGTGCTGGCCTGCTATTCCCTGCTGGTGAC
CGTCGCGTTTATTATTTTCTGGGTTAGATCCAAAAGAAGCCGCCTG
CTCCATAGCGATTACATGAATATGACTCCACGCCGCCCTGGCCCCA
CAAGGAAACACTACCAGCCTTACGCACCACCTAGAGATTTCGCTGC
CTATCGGAGCAGGGTGAAGTTTTCCAGATCTGCAGATGCACCAGCG
TATCAGCAGGGCCAGAACCAACTGTATAACGAGCTCAACCTGGGA
CGCAGGGAAGAGTATGACGTTTTGGACAAGCGCAGAGGACGGGAC
CCTGAGATGGGTGGCAAACCAAGACGAAAAAACCCCCAGGAGGGT
CTCTATAATGAGCTGCAGAAGGATAAGATGGCTGAAGCCTATTCTG
AAATAGGCATGAAAGGAGAGCGGAGAAGGGGAAAAGGGCACGAC
GGTTTGTACCAGGGACTCAGCACTGCTACGAAGGATACTTATGACG
CTCTCCACATGCAAGCCCTGCCACCTAGG
Clone 24C1 CD28 CD3 zeta CAR AA Heavy & Light Chains
(SEQ ID NO. 44)
DIQLTQSPSSLSASVGDRVSFTCQASQDINNFLNWYQQKPGKAPKLLIY
DASNLETGVPSRFSGSGSGTDFTFTISSLQPEDIATYYCQQYGNLPFTFG
GGTKVEIKRGGGGSGGGGSGGGGSQVQLQESGPGLVKPSETLSLTCT
VSGGSISSYYWSWIRQPPGKGLEWIGYIYYSGSTNYNPSLKSRVTISVD
TSKNQFSLKLSSVTAADTAVYYCVSLVYCGGDCYSGFDYWGQGTLV
TVSSAAAIEVMYPPPYLDNEKSNGTIIHVKGKHLCPSPLFPGPSKPFWV
LVVVGGVLACYSLLVTVAFIIFWVRSKRSRLLHSDYMNMTPRRPGPTR
KHYQPYAPPRDFAAYRSRVKFSRSADAPAYQQGQNQLYNELNLGRRE
EYDVLDKRRGRDPEMGGKPRRKNPQEGLYNELQKDKMAEAYSEIGM
KGERRRGKGHDGLYQGLSTATKDTYDALHMQALPPR
Clone 24C1 CD8 CD3 zeta CAR DNA Heavy & Light Chains
(SEQ ID NO. 45)
ATGGCACTCCCCGTAACTGCTCTGCTGCTGCCGTTGGCATTGCTCCT
GCACGCCGCACGCCCGGACATTCAATTGACCCAGTCCCCTAGCAGT
CTCTCAGCAAGTGTGGGAGATAGGGTGTCATTCACCTGTCAGGCTT
CACAGGACATCAACAACTTCCTCAATTGGTATCAGCAGAAGCCAG
GGAAGGCACCAAAGCTGCTCATATATGACGCTTCAAACCTTGAAAC
CGGAGTACCTAGCCGCTTCAGCGGAAGCGGATCAGGGACTGACTT
CACTTTTACCATCTCTTCACTGCAGCCCGAAGACATCGCCACATAC
TACTGCCAGCAGTACGGAAACTTGCCTTTTACATTTGGGGGCGGCA
CCAAAGTGGAGATTAAGCGAGGGGGAGGCGGCTCAGGAGGCGGTG
GCTCCGGAGGCGGGGGTTCCCAGGTCCAGCTCCAGGAATCCGGCC
CAGGTCTGGTTAAGCCCAGTGAAACTTTGTCCCTCACGTGTACTGT
GAGCGGTGGTTCAATCTCCTCATACTATTGGTCTTGGATACGGCAA
CCTCCTGGAAAGGGCCTCGAGTGGATCGGCTATATCTACTATAGTG
GCTCCACTAATTACAACCCTTCCCTCAAGTCCAGAGTCACCATTTCC
GTGGACACATCTAAGAACCAGTTCAGTCTGAAGTTGTCCAGCGTTA
CAGCCGCAGACACAGCCGTTTATTACTGTGTGTCTCTTGTTTACTGC
GGGGGAGACTGTTATAGCGGCTTCGATTACTGGGGCCAGGGCACCT
TGGTCACAGTCTCTTCCGCGGCCGCCCTCTCTAACAGTATTATGTAC
TTTTCTCATTTTGTACCCGTGTTCCTTCCCGCTAAGCCAACTACTAC
CCCGGCCCCACGGCCGCCTACCCCTGCACCCACAATAGCCAGTCAG
CCTTTGAGCCTGAGACCTGAGGCTTGTCGGCCGGCTGCTGGGGGTG
CAGTGCACACACGAGGTCTTGATTTTGCTTGCGACATATACATCTG
GGCCCCTCTGGCCGGGACCTGTGGGGTGCTGCTTCTGAGCTTGGTC
ATCACGCTCTATTGCAACCATCGCAACAGATCCAAAAGAAGCCGCC
TGCTCCATAGCGATTACATGAATATGACTCCACGCCGCCCTGGCCC
CACAAGGAAACACTACCAGCCTTACGCACCACCTAGAGATTTCGCT
GCCTATCGGAGCAGGGTGAAGTTTTCCAGATCTGCAGATGCACCAG
CGTATCAGCAGGGCCAGAACCAACTGTATAACGAGCTCAACCTGG
GACGCAGGGAAGAGTATGACGTTTTGGACAAGCGCAGAGGACGGG
ACCCTGAGATGGGTGGCAAACCAAGACGAAAAAACCCCCAGGAGG
GTCTCTATAATGAGCTGCAGAAGGATAAGATGGCTGAAGCCTATTC
TGAAATAGGCATGAAAGGAGAGCGGAGAAGGGGAAAAGGGCACG
ACGGTTTGTACCAGGGACTCAGCACTGCTACGAAGGATACTTATGA
CGCTCTCCACATGCAAGCCCTGCCACCTAGGTAA
Clone 24C1 CD8 CD3 zeta CAR AA Heavy & Light Chains
(Signal Peptide in Bold)
(SEQ ID NO. 46)
MALPVTALLLPLALLLHAARPDIQLTQSPSSLSASVGDRVSFTCQAS
QDINNFLNWYQQKPGKAPKLLIYDASNLETGVPSRFSGSGSGTDFTFTI
SSLQPEDIATYYCQQYGNLPFTFGGGTKVEIKRGGGGSGGGGSGGGGS
QVQLQESGPGLVKPSETLSLTCTVSGGSISSYYWSWIRQPPGKGLEWIG
YIYYSGSTNYNPSLKSRVTISVDTSKNQFSLKLSSVTAADTAVYYCVSL
VYCGGDCYSGFDYWGQGTLVTVSSAAALSNSIMYFSHFVPVFLPAKP
TTTPAPRPPTPAPTIASQPLSLRPEACRPAAGGAVHTRGLDFACDIYIW
APLAGTCGVLLLSLVITLYCNHRNRSKRSRLLHSDYMNMTPRRPGPTR
KHYQPYAPPRDFAAYRSRVKFSRSADAPAYQQGQNQLYNELNLGRRE
EYDVLDKRRGRDPEMGGKPRRKNPQEGLYNELQKDKMAEAYSEIGM
KGERRRGKGHDGLYQGLSTATKDTYDALHMQALPPR
Clone 24C1 CD8 CD3 zeta CAR DNA Heavy & Light Chains
(SEQ ID NO. 47)
GACATTCAATTGACCCAGTCCCCTAGCAGTCTCTCAGCAAGTGTGG
GAGATAGGGTGTCATTCACCTGTCAGGCTTCACAGGACATCAACAA
CTTCCTCAATTGGTATCAGCAGAAGCCAGGGAAGGCACCAAAGCT
GCTCATATATGACGCTTCAAACCTTGAAACCGGAGTACCTAGCCGC
TTCAGCGGAAGCGGATCAGGGACTGACTTCACTTTTACCATCTCTT
CACTGCAGCCCGAAGACATCGCCACATACTACTGCCAGCAGTACG
GAAACTTGCCTTTTACATTTGGGGGCGGCACCAAAGTGGAGATTAA
GCGAGGGGGAGGCGGCTCAGGAGGCGGTGGCTCCGGAGGCGGGG
GTTCCCAGGTCCAGCTCCAGGAATCCGGCCCAGGTCTGGTTAAGCC
CAGTGAAACTTTGTCCCTCACGTGTACTGTGAGCGGTGGTTCAATC
TCCTCATACTATTGGTCTTGGATACGGCAACCTCCTGGAAAGGGCC
TCGAGTGGATCGGCTATATCTACTATAGTGGCTCCACTAATTACAA
CCCTTCCCTCAAGTCCAGAGTCACCATTTCCGTGGACACATCTAAG
AACCAGTTCAGTCTGAAGTTGTCCAGCGTTACAGCCGCAGACACAG
CCGTTTATTACTGTGTGTCTCTTGTTTACTGCGGGGGAGACTGTTAT
AGCGGCTTCGATTACTGGGGCCAGGGCACCTTGGTCACAGTCTCTT
CCGCGGCCGCCCTCTCTAACAGTATTATGTACTTTTCTCATTTTGTA
CCCGTGTTCCTTCCCGCTAAGCCAACTACTACCCCGGCCCCACGGC
CGCCTACCCCTGCACCCACAATAGCCAGTCAGCCTTTGAGCCTGAG
ACCTGAGGCTTGTCGGCCGGCTGCTGGGGGTGCAGTGCACACACG
AGGTCTTGATTTTGCTTGCGACATATACATCTGGGCCCCTCTGGCCG
GGACCTGTGGGGTGCTGCTTCTGAGCTTGGTCATCACGCTCTATTG
CAACCATCGCAACAGATCCAAAAGAAGCCGCCTGCTCCATAGCGA
TTACATGAATATGACTCCACGCCGCCCTGGCCCCACAAGGAAACAC
TACCAGCCTTACGCACCACCTAGAGATTTCGCTGCCTATCGGAGCA
GGGTGAAGTTTTCCAGATCTGCAGATGCACCAGCGTATCAGCAGGG
CCAGAACCAACTGTATAACGAGCTCAACCTGGGACGCAGGGAAGA
GTATGACGTTTTGGACAAGCGCAGAGGACGGGACCCTGAGATGGG
TGGCAAACCAAGACGAAAAAACCCCCAGGAGGGTCTCTATAATGA
GCTGCAGAAGGATAAGATGGCTGAAGCCTATTCTGAAATAGGCAT
GAAAGGAGAGCGGAGAAGGGGAAAAGGGCACGACGGTTTGTACC
AGGGACTCAGCACTGCTACGAAGGATACTTATGACGCTCTCCACAT
GCAAGCCCTGCCACCTAGG
Clone 24C1 CD8 CD3 zeta CAR AA Heavy & Light Chains
(SEQ ID NO. 48)
DIQLTQSPSSLSASVGDRVSFTCQASQDINNFLNWYQQKPGKAPKLLIY
DASNLETGVPSRFSGSGSGTDFTFTISSLQPEDIATYYCQQYGNLPFTFG
GGTKVEIKRGGGGSGGGGSGGGGSQVQLQESGPGLVKPSETLSLTCT
VSGGSISSYYWSWIRQPPGKGLEWIGYIYYSGSTNYNPSLKSRVTISVD
TSKNQFSLKLSSVTADTAVYYCVSLVYCGGDCYSGFDYWGQGTLVT
VSSAAALSNSIMYFSHFVPVFLPAKPTTTPAPRPPTPAPTIASQPLSLRPE
ACRPAAGGAVHTRGLDFACDIYIWAPLAGTCGVLLLSLVITLYCNHRN
RSKRSRLLHSDYMNMTPRRPGPTRKHYQPYAPPRDFAAYRSRVKFSR
SADAPAYQQGQNQLYNELNLGREEYDVLDKRRGRDPEMGGKPRRKN
PQEGLYNELQKDKMAEAYSEIGMKGERRRGKGHDGLYQGLSTATK
DTYDALHMQA LPPR
Clone 24C8 Heavy Chain (HC) DNA
(SEQ ID NO. 49)
CAGGTACAGCTGCAGGAATCTGGGCCCGGACTTGTCAAGCCAAGT
CAGACACTTTCTCTTACATGTACCGTGAGCGGCGGAAGTATAAGCA
GTGGAGGCTTTTACTGGTCTTGGATACGGCAGCACCCAGGCAAAGG
CTTGGAGTGGATTGGATACATTCATCATTCAGGATCTACACACTAT
AATCCATCCCTTAAGTCCCGGGTCACCATTAGCATTGATACGTCTA
AGAATCTGTTCAGTCTCAGGCTGTCCTCCGTCACTGCTGCCGACAC
AGCCGTGTACTACTGCGCCTCCTTGGTTTACTGCGGAGGCGACTGT
TATAGCGGCTTTGATTATTGGGGGCAGGGGACCCTCGTAACCGTGA
GCTCT
Clone 24C8 AA HC(CDRs in Underline)
(SEQ ID NO. 50)
QVQLQESGPGLVKPSQTLSLTCTVSGGSISSGGFYWSWIRQHPGKGLE
WIGYIHHSGSTHYNPSLKSRVTISIDTSKNLFSLRLSSVTAADTAVYYC
ASLVYCGGDCYSGFDYWGQGTLVTVSS
(SEQ ID NO. 51)
Clone 24C8 HCCDR1 AA: GGSISSGGF
(SEQ ID NO. 52)
Clone 24C8 HCCDR2 AA: HHSGS
(SEQ ID NO. 53)
Clone 24C8 HCCDR3 AA: LVYCGGDCYSGFDY
Clone 24C8 Light Chain (LC) DNA
(SEQ ID NO. 54)
GATATCCAGCTCACTCAAAGCCCCTCTAGTCTCTCTGCCTCAGTGG
GGGATCGGGTCAGTTTTACTTGTCAAGCTTCACAGGATATCAACAA
CTTCCTTAATTGGTATCAGCAGAAGCCAGGAAAAGCACCCAAGCTG
CTCATCTATGATGCCTCAAATTTGGAGACGGGTGTTCCCAGTCGAT
TCTCTGGGTCAGGGTCCGGGACCGACTTTACGTTTACGATCTCCTCT
CTGCAGCCCGAAGACATCGCCACATACTATTGTCAACAGTACGGCA
ACTTGCCTTTCACATTTGGGGGCGGGACTAAGGTTGAAATCAAGAG
G
Clone 24C8 LCAA (CDRs in Underline)
(SEQ ID NO. 55)
DIQLTQSPSSLSASVGDRVSFTCQASQDINNFLNWYQQKPGKAPKLLIY
DASNLETGVPSRFSGSGSGTDFTFTISSLQPEDIATYYCQQYGNLPFTFG
GGTKVEIKR
(SEQ ID NO. 56)
Clone 24C8 LCCDR1 AA: QASQDINNFLN
(SEQ ID NO. 57)
Clone 24C8 LCCDR2 AA: DASNLET
(SEQ ID NO. 58)
Clone 24C8 LCCDR3 AA: QQYGNLPFT
Clone 24C8 CD28T CD3 zeta CAR DNA Heavy & Light Chains
(SEQ ID NO. 59)
ATGGCACTCCCCGTAACTGCTCTGCTGCTGCCGTTGGCATTGCTCCT
GCACGCCGCACGCCCGCAGGTACAGCTGCAGGAATCTGGGCCCGG
ACTTGTCAAGCCAAGTCAGACACTTTCTCTTACATGTACCGTGAGC
GGCGGAAGTATAAGCAGTGGAGGCTTTTACTGGTCTTGGATACGGC
AGCACCCAGGCAAAGGCTTGGAGTGGATTGGATACATTCATCATTC
AGGATCTACACACTATAATCCATCCCTTAAGTCCCGGGTCACCATT
AGCATTGATACGTCTAAGAATCTGTTCAGTCTCAGGCTGTCCTCCG
TCACTGCTGCCGACACAGCCGTGTACTACTGCGCCTCCTTGGTTTAC
TGCGGAGGCGACTGTTATAGCGGCTTTGATTATTGGGGGCAGGGGA
CCCTCGTAACCGTGAGCTCTGGAGGGGGTGGGAGCGGGGGAGGAG
GTTCAGGGGGGGGCGGCTCCGATATCCAGCTCACTCAAAGCCCCTC
TAGTCTCTCTGCCTCAGTGGGGGATCGGGTCAGTTTTACTTGTCAA
GCTTCACAGGATATCAACAACTTCCTTAATTGGTATCAGCAGAAGC
CAGGAAAAGCACCCAAGCTGCTCATCTATGATGCCTCAAATTTGGA
GACGGGTGTTCCCAGTCGATTCTCTGGGTCAGGGTCCGGGACCGAC
TTTACGTTTACGATCTCCTCTCTGCAGCCCGAAGACATCGCCACAT
ACTATTGTCAACAGTACGGCAACTTGCCTTTCACATTTGGGGGCGG
GACTAAGGTTGAAATCAAGAGGGCCGCTGCACTGGACAATGAGAA
GTCCAACGGCACCATCATCCACGTGAAGGGCAAGCACCTGTGCCCT
AGTCCTCTGTTCCCAGGCCCATCCAAACCTTTTTGGGTTCTTGTTGT
GGTCGGGGGGGTGCTGGCCTGCTATTCTCTGCTGGTCACGGTGGCC
TTCATAATTTTCTGGGTTAGATCCAAAAGAAGCCGCCTGCTCCATA
GCGATTACATGAATATGACTCCACGCCGCCCTGGCCCCACAAGGAA
ACACTACCAGCCTTACGCACCACCTAGAGATTTCGCTGCCTATCGG
AGCAGGGTGAAGTTTTCCAGATCTGCAGATGCACCAGCGTATCAGC
AGGGCCAGAACCAACTGTATAACGAGCTCAACCTGGGACGCAGGG
AAGAGTATGACGTTTTGGACAAGCGCAGAGGACGGGACCCTGAGA
TGGGTGGCAAACCAAGACGAAAAAACCCCCAGGAGGGTCTCTATA
ATGAGCTGCAGAAGGATAAGATGGCTGAAGCCTATTCTGAAATAG
GCATGAAAGGAGAGCGGAGAAGGGGAAAAGGGCACGACGGTTTG
TACCAGGGACTCAGCACTGCTACGAAGGATACTTATGACGCTCTCC
ACATGCAAGCCCTGCCACCTAGGTAA
Clone 24C8 CD28T CD3 zeta CAR AA Heavy & Light Chains
(Signal Peptide in Bold)
(SEQ ID NO. 60)
MALPVTALLLPLALLLHAARPQVQLQESGPGLVKPSQTLSLTCTVSG
GSISSGGFYWSWIRQHPGKGLEWIGYIHHSGSTHYNPSLKSRVTISIDTS
KNLFSLRLSSVTAADTAVYYCASLVYCGGDCYSGFDYWGQGTLVTV
SSGGGGSGGGGSGGGGSDIQLTQSPSSLSASVGDRVSFTCQASQDINNF
LNWYQQKPGKAPKLLIYDASNLETGVPSRFSGSGSGTDFTFTISSLQPE
DIATYYCQQYGNLPFTFGGGTKVEIKRAAALDNEKSNGTIIHVKGKHL
CPSPLFPGPSKPFWVLVVVGGVLACYSLLVTVAFIIFWVRSKRSRLLHS
DYMNMTPRRPGPTRKHYQPYAPPRDFAAYRSRVKFSRSADAPAYQQ
GQNQLYNELNLGRREEYDVLDKRRGRDPEMGGKPRRKNPQEGLYNE
LQKDKMAEAYSEIGMKGERRRGKGHDGLYQGLSTATKDTYDALHM
QALPPR
Clone 24C8 CD28T CD3 zeta CAR DNA Heavy & Light Chains
(SEQ ID NO. 61)
CAGGTACAGCTGCAGGAATCTGGGCCCGGACTTGTCAAGCCAAGT
CAGACACTTTCTCTTACATGTACCGTGAGCGGCGGAAGTATAAGCA
GTGGAGGCTTTTACTGGTCTTGGATACGGCAGCACCCAGGCAAAGG
CTTGGAGTGGATTGGATACATTCATCATTCAGGATCTACACACTAT
AATCCATCCCTTAAGTCCCGGGTCACCATTAGCATTGATACGTCTA
AGAATCTGTTCAGTCTCAGGCTGTCCTCCGTCACTGCTGCCGACAC
AGCCGTGTACTACTGCGCCTCCTTGGTTTACTGCGGAGGCGACTGT
TATAGCGGCTTTGATTATTGGGGGCAGGGGACCCTCGTAACCGTGA
GCTCTGGAGGGGGTGGGAGCGGGGGAGGAGGTTCAGGGGGGGGC
GGCTCCGATATCCAGCTCACTCAAAGCCCCTCTAGTCTCTCTGCCTC
AGTGGGGGATCGGGTCAGTTTTACTTGTCAAGCTTCACAGGATATC
AACAACTTCCTTAATTGGTATCAGCAGAAGCCAGGAAAAGCACCC
AAGCTGCTCATCTATGATGCCTCAAATTTGGAGACGGGTGTTCCCA
GTCGATTCTCTGGGTCAGGGTCCGGGACCGACTTTACGTTTACGAT
CTCCTCTCTGCAGCCCGAAGACATCGCCACATACTATTGTCAACAG
TACGGCAACTTGCCTTTCACATTTGGGGGCGGGACTAAGGTTGAAA
TCAAGAGGGCCGCTGCACTGGACAATGAGAAGTCCAACGGCACCA
TCATCCACGTGAAGGGCAAGCACCTGTGCCCTAGTCCTCTGTTCCC
AGGCCCATCCAAACCTTTTTGGGTTCTTGTTGTGGTCGGGGGGGTG
CTGGCCTGCTATTCTCTGCTGGTCACGGTGGCCTTCATAATTTTCTG
GGTTAGATCCAAAAGAAGCCGCCTGCTCCATAGCGATTACATGAAT
ATGACTCCACGCCGCCCTGGCCCCACAAGGAAACACTACCAGCCTT
ACGCACCACCTAGAGATTTCGCTGCCTATCGGAGCAGGGTGAAGTT
TTCCAGATCTGCAGATGCACCAGCGTATCAGCAGGGCCAGAACCA
ACTGTATAACGAGCTCAACCTGGGACGCAGGGAAGAGTATGACGT
TTTGGACAAGCGCAGAGGACGGGACCCTGAGATGGGTGGCAAACC
AAGACGAAAAAACCCCCAGGAGGGTCTCTATAATGAGCTGCAGAA
GGATAAGATGGCTGAAGCCTATTCTGAAATAGGCATGAAAGGAGA
GCGGAGAAGGGGAAAAGGGCACGACGGTTTGTACCAGGGACTCAG
CACTGCTACGAAGGATACTTATGACGCTCTCCACATGCAAGCCCTG
CCACCTAGG
Clone 24C8 CD28T CD3 zeta CAR AA Heavy & Light Chains
(SEQ ID NO. 62)
QVQLQESGPGLVKPSQTLSLTCTVSGGSISSGGFYVVSWIRQHPGKGLE
WIGYIHHSGSTHYNPSLKSRVTISIDTSKNLFSLRLSSVTAADTAVYYC
ASLVYCGGDCYSGFDYWGQGTLVTVSSGGGGSGGGGSGGGGSDIQL
TQSPSSLSASVGDRVSFTCQASQDINNFLNWYQQKPGKAPKLLIYDAS
NLETGVPSRFSGSGSGTDFTFTISSLQPEDIATYYCQQYGNLPFTFGGGT
KVEIKRAAALDNEKSNGTIIHVKGKHLCPSPLFPGPSKPFWVLVVVGG
VLACYSLLVTVAFIIFWVRSKRSRLLHSDYMNMTPRRPGPTRKHYQPY
APPRDFAAYRSRVKFSRSADAPAYQQGQNQLYNELNLGRREEYDVLD
KRRGRDPEMGGKPRRKNPQEGLYNELQKDKMAEAYSEIGMKGERRR
GKGHDGLYQGLSTATKDTYDALHMQALPPR
Clone 24C8 CD28 CD3 zeta CAR DNA Heavy & Light Chains
(SEQ ID NO. 63)
ATGGCACTCCCCGTAACTGCTCTGCTGCTGCCGTTGGCATTGCTCCT
GCACGCCGCACGCCCGCAGGTGCAGCTGCAGGAAAGCGGTCCGGG
ACTTGTCAAGCCGTCCCAAACGCTGAGTCTGACGTGTACTGTCTCT
GGTGGCTCTATTTCTTCCGGGGGCTTTTATTGGTCTTGGATCAGACA
ACACCCTGGCAAAGGGCTGGAGTGGATAGGGTATATTCACCACTCT
GGGTCCACTCACTACAACCCATCATTGAAATCCAGAGTGACTATCT
CAATCGACACATCCAAGAACCTTTTCAGCCTGAGGTTGTCATCAGT
TACCGCCGCTGACACCGCGGTGTATTATTGCGCCTCTCTCGTGTACT
GCGGTGGCGATTGTTATAGTGGCTTTGACTACTGGGGGCAGGGGAC
ATTGGTTACCGTTTCAAGTGGAGGCGGTGGGTCTGGCGGGGGCGGT
AGCGGAGGTGGGGGGAGCGACATACAGCTTACGCAGAGCCCCTCC
AGCCTTTCAGCCTCCGTGGGGGATAGGGTGTCCTTTACCTGCCAGG
CTTCCCAGGACATAAACAACTTCCTCAATTGGTATCAGCAAAAGCC
CGGGAAAGCACCAAAGCTGCTCATCTACGATGCCAGCAACCTGGA
AACCGGAGTGCCGTCTCGCTTCTCTGGAAGTGGCAGTGGGACCGAT
TTCACTTTTACAATCTCAAGTTTGCAGCCAGAAGACATTGCAACAT
ACTACTGTCAACAGTACGGCAATCTCCCCTTTACATTTGGGGGGGG
AACTAAAGTGGAGATTAAGCGCGCTGCAGCCATTGAAGTTATGTAT
CCGCCCCCGTATCTGGATAACGAGAAATCTAATGGTACCATAATAC
ATGTGAAGGGGAAGCACCTCTGTCCATCACCGCTGTTCCCCGGCCC
TTCAAAACCTTTCTGGGTACTCGTTGTCGTGGGTGGAGTTCTGGCCT
GCTATAGTCTGCTGGTGACCGTGGCGTTTATCATCTTCTGGGTAAG
ATCCAAAAGAAGCCGCCTGCTCCATAGCGATTACATGAATATGACT
CCACGCCGCCCTGGCCCCACAAGGAAACACTACCAGCCTTACGCAC
CACCTAGAGATTTCGCTGCCTATCGGAGCAGGGTGAAGTTTTCCAG
ATCTGCAGATGCACCAGCGTATCAGCAGGGCCAGAACCAACTGTA
TAACGAGCTCAACCTGGGACGCAGGGAAGAGTATGACGTTTTGGA
CAAGCGCAGAGGACGGGACCCTGAGATGGGTGGCAAACCAAGACG
AAAAAACCCCCAGGAGGGTCTCTATAATGAGCTGCAGAAGGATAA
GATGGCTGAAGCCTATTCTGAAATAGGCATGAAAGGAGAGCGGAG
AAGGGGAAAAGGGCACGACGGTTTGTACCAGGGACTCAGCACTGC
TACGAAGGATACTTATGACGCTCTCCACATGCAAGCCCTGCCACCT
AGGTAA
Clone 24C8 CD28 CD3 zeta CAR AA Heavy & Light Chains
(Signal Peptide in Bold)
(SEQ ID NO. 64)
MALPVTALLLPLALLLHAARPQVQLQESGPGLVKPSQTLSLTCTVSG
GSISSGGFYWSWIRQHPGKGLEWIGYIHHSGSTHYNPSLKSRVTISIDTS
KNLFSLRLSSVTAADTAVYYCASLVYCGGDCYSGFDYWGQGTLVTV
SSGGGGSGGGGSGGGGSDIQLTQSPSSLSASVGDRVSFTCQASQDINNF
LNWYQQKPGKAPKLLIYDASNLETGVPSRFSGSGSGTDFTFTISSLQPE
DIATYYCQQYGNLPFTFGGGTKVEIKRAAAIEVMYPPPYLDNEKSNGT
IIHVKGKHLCPSPLFPGPSKPFWVLVVVGGVLACYSLLVTVAFIIFWVR
SKRSRLLHSDYMNMTPRRPGPTRKHYQPYAPPRDFAAYRSRVKFSRS
ADAPAYQQGQNQLYNELNLGRREEYDVLDKRRGRDPEMGGKPRRK
NPQEGLYNELQKDKMAEAYSEIGMKGERRRGKGHDGLYQGLSTATK
DTYDALHMQALPPR
Clone 24C8 CD28 CD3 zeta CAR DNA Heavy & Light Chains
(SEQ ID NO. 65)
CAGGTGCAGCTGCAGGAAAGCGGTCCGGGACTTGTCAAGCCGTCC
CAAACGCTGAGTCTGACGTGTACTGTCTCTGGTGGCTCTATTTCTTC
CGGGGGCTTTTATTGGTCTTGGATCAGACAACACCCTGGCAAAGGG
CTGGAGTGGATAGGGTATATTCACCACTCTGGGTCCACTCACTACA
ACCCATCATTGAAATCCAGAGTGACTATCTCAATCGACACATCCAA
GAACCTTTTCAGCCTGAGGTTGTCATCAGTTACCGCCGCTGACACC
GCGGTGTATTATTGCGCCTCTCTCGTGTACTGCGGTGGCGATTGTTA
TAGTGGCTTTGACTACTGGGGGCAGGGGACATTGGTTACCGTTTCA
AGTGGAGGCGGTGGGTCTGGCGGGGGCGGTAGCGGAGGTGGGGGG
AGCGACATACAGCTTACGCAGAGCCCCTCCAGCCTTTCAGCCTCCG
TGGGGGATAGGGTGTCCTTTACCTGCCAGGCTTCCCAGGACATAAA
CAACTTCCTCAATTGGTATCAGCAAAAGCCCGGGAAAGCACCAAA
GCTGCTCATCTACGATGCCAGCAACCTGGAAACCGGAGTGCCGTCT
CGCTTCTCTGGAAGTGGCAGTGGGACCGATTTCACTTTTACAATCT
CAAGTTTGCAGCCAGAAGACATTGCAACATACTACTGTCAACAGTA
CGGCAATCTCCCCTTTACATTTGGGGGGGGAACTAAAGTGGAGATT
AAGCGCGCTGCAGCCATTGAAGTTATGTATCCGCCCCCGTATCTGG
ATAACGAGAAATCTAATGGTACCATAATACATGTGAAGGGGAAGC
ACCTCTGTCCATCACCGCTGTTCCCCGGCCCTTCAAAACCTTTCTGG
GTACTCGTTGTCGTGGGTGGAGTTCTGGCCTGCTATAGTCTGCTGGT
GACCGTGGCGTTTATCATCTTCTGGGTAAGATCCAAAAGAAGCCGC
CTGCTCCATAGCGATTACATGAATATGACTCCACGCCGCCCTGGCC
CCACAAGGAAACACTACCAGCCTTACGCACCACCTAGAGATTTCGC
TGCCTATCGGAGCAGGGTGAAGTTTTCCAGATCTGCAGATGCACCA
GCGTATCAGCAGGGCCAGAACCAACTGTATAACGAGCTCAACCTG
GGACGCAGGGAAGAGTATGACGTTTTGGACAAGCGCAGAGGACGG
GACCCTGAGATGGGTGGCAAACCAAGACGAAAAAACCCCCAGGAG
GGTCTCTATAATGAGCTGCAGAAGGATAAGATGGCTGAAGCCTATT
CTGAAATAGGCATGAAAGGAGAGCGGAGAAGGGGAAAAGGGCAC
GACGGTTTGTACCAGGGACTCAGCACTGCTACGAAGGATACTTATG
ACGCTCTCCACATGCAAGCCCTGCCACCTAGG
Clone 24C8 CD28 CD3 zeta CAR AA Heavy & Light Chains
(SEQ ID NO. 66)
QVQLQESGPGLVKPSQTLSLTCTVSGGSISSGGFYVVSWIRQHPGKGLE
WIGYIHHSGSTHYNPSLKSRVTISIDTSKNLFSLRLSSVTAADTAVYYC
ASLVYCGGDCYSGFDYWGQGTLVTVSSGGGGSGGGGSGGGGSDIQL
TQSPSSLSASVGDRVSFTCQASQDINNFLNWYQQKPGKAPKLLIYDAS
NLETGVPSRFSGSGSGTDFTFTISSLQPEDIATYYCQQYGNLPFTFGGGT
KVEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLG
RREEYDVLDKRRGRDPEMGGKPRRKNPQEGLYNELQKDKMAEAYSE
IGMKGERRRGKGHDGLYQGLSTATKDTYDALHMQALPPR
Clone 24C8 CD8 CD3 zeta CAR DNA Heavy & Light Chains
(SEQ ID NO. 67)
ATGGCACTCCCCGTAACTGCTCTGCTGCTGCCGTTGGCATTGCTCCT
GCACGCCGCACGCCCGCAGGTGCAGTTGCAGGAAAGCGGGCCTGG
CCTTGTGAAACCAAGCCAGACACTGAGCCTGACATGCACTGTGTCC
GGCGGGTCCATATCTTCCGGGGGTTTTTATTGGTCCTGGATACGCC
AGCATCCCGGGAAAGGACTTGAATGGATTGGATATATCCACCATTC
CGGAAGCACCCACTACAATCCAAGCCTTAAATCCCGGGTGACAATC
TCCATCGACACCTCAAAGAATCTTTTTTCCCTGCGGTTGTCTTCAGT
AACTGCCGCCGATACCGCTGTGTACTACTGTGCCAGCCTCGTCTAT
TGCGGCGGAGATTGTTATTCTGGGTTCGATTATTGGGGTCAAGGCA
CACTGGTAACTGTCAGCAGCGGAGGCGGCGGTTCCGGGGGCGGGG
GCAGTGGAGGGGGCGGATCTGACATTCAGCTTACGCAGTCCCCATC
TTCACTTAGCGCCAGCGTTGGCGATCGGGTCAGCTTCACGTGTCAA
GCAAGTCAGGATATCAACAACTTTCTTAACTGGTACCAGCAGAAGC
CAGGCAAGGCACCCAAGTTGCTGATTTACGATGCTTCTAACCTCGA
GACGGGAGTGCCTAGCCGCTTCTCCGGGAGCGGCAGCGGCACAGA
CTTTACCTTTACGATTTCCAGTCTGCAGCCAGAGGATATAGCAACT
TATTACTGTCAGCAGTATGGCAACCTCCCTTTTACCTTCGGTGGTGG
CACAAAGGTCGAGATTAAAAGAGCCGCAGCGTTGTCCAACTCCAT
AATGTATTTTTCTCATTTTGTGCCCGTCTTTCTGCCTGCCAAACCTA
CCACCACCCCCGCCCCACGACCACCTACTCCAGCCCCCACCATCGC
CTCCCAGCCCCTCAGCCTGAGGCCAGAGGCTTGTCGCCCTGCTGCG
GGGGGCGCTGTCCATACCAGAGGACTCGACTTCGCCTGCGATATTT
ATATATGGGCCCCCCTCGCCGGCACCTGCGGAGTCTTGCTCCTGAG
CCTTGTGATCACGCTTTATTGTAACCATCGGAATAGATCCAAAAGA
AGCCGCCTGCTCCATAGCGATTACATGAATATGACTCCACGCCGCC
CTGGCCCCACAAGGAAACACTACCAGCCTTACGCACCACCTAGAG
ATTTCGCTGCCTATCGGAGCAGGGTGAAGTTTTCCAGATCTGCAGA
TGCACCAGCGTATCAGCAGGGCCAGAACCAACTGTATAACGAGCT
CAACCTGGGACGCAGGGAAGAGTATGACGTTTTGGACAAGCGCAG
AGGACGGGACCCTGAGATGGGTGGCAAACCAAGACGAAAAAACCC
CCAGGAGGGTCTCTATAATGAGCTGCAGAAGGATAAGATGGCTGA
AGCCTATTCTGAAATAGGCATGAAAGGAGAGCGGAGAAGGGGAAA
AGGGCACGACGGTTTGTACCAGGGACTCAGCACTGCTACGAAGGA
TACTTATGACGCTCTCCACATGCAAGCCCTGCCACCTAGGTAA
Clone 24C8 CD8 CD3 zeta CAR AA Heavy & Light Chains
(Signal Peptide in Bold)
(SEQ ID NO. 68)
MALPVTALLLPLALLLHAARPQVQLQESGPGLVKPSQTLSLTCTVSG
GSISSGGFYWSWIRQHPGKGLEWIGYIHHSGSTHYNPSLKSRVTISIDTS
KNLFSLRLSSVTAADTAVYYCASLVYCGGDCYSGFDYWGQGTLVTV
SSGGGGSGGGGSGGGGSDIQLTQSPSSLSASVGDRVSFTCQASQDINNF
LNWYQQKPGKAPKLLIYDASNLETGVPRFSGSGSGTDFTFTISSLQPED
IATYYCQYGNLPFTFGGGTKVEIKRAAALSNSIMYFSHFVPVFLPAKPT
TTPAPRPPTPAPTIASQPLSLRPEACRPAAGGAVHTRGLDFACDIYIWA
PLAGTCGVLLLSLVITLYCNHRNRSKRSRLLHSDYMNMTPRRPGPTRK
HYQPYAPPRDFAAYRSRVKFSRSADAPAYQQGQNQLYNELNLGRREE
YDVLDKRRGRDPEMGGKPRRKNPQEGLYNELQKDKMAEAYSEIGMK
GERRRGKGHDGLYQGLSTATKDTYDALHMQALPPR
Clone 24C8 CD8 CD3 zeta CAR DNA Heavy & Light Chains
(SEQ ID NO. 69)
CAGGTGCAGTTGCAGGAAAGCGGGCCTGGCCTTGTGAAACCAAGC
CAGACACTGAGCCTGACATGCACTGTGTCCGGCGGGTCCATATCTT
CCGGGGGTTTTTATTGGTCCTGGATACGCCAGCATCCCGGGAAAGG
ACTTGAATGGATTGGATATATCCACCATTCCGGAAGCACCCACTAC
AATCCAAGCCTTAAATCCCGGGTGACAATCTCCATCGACACCTCAA
AGAATCTTTTTTCCCTGCGGTTGTCTTCAGTAACTGCCGCCGATACC
GCTGTGTACTACTGTGCCAGCCTCGTCTATTGCGGCGGAGATTGTT
ATTCTGGGTTCGATTATTGGGGTCAAGGCACACTGGTAACTGTCAG
CAGCGGAGGCGGCGGTTCCGGGGGCGGGGGCAGTGGAGGGGGCG
GATCTGACATTCAGCTTACGCAGTCCCCATCTTCACTTAGCGCCAG
CGTTGGCGATCGGGTCAGCTTCACGTGTCAAGCAAGTCAGGATATC
AACAACTTTCTTAACTGGTACCAGCAGAAGCCAGGCAAGGCACCC
AAGTTGCTGATTTACGATGCTTCTAACCTCGAGACGGGAGTGCCTA
GCCGCTTCTCCGGGAGCGGCAGCGGCACAGACTTTACCTTTACGAT
TTCCAGTCTGCAGCCAGAGGATATAGCAACTTATTACTGTCAGCAG
TATGGCAACCTCCCTTTTACCTTCGGTGGTGGCACAAAGGTCGAGA
TTAAAAGAGCCGCAGCGTTGTCCAACTCCATAATGTATTTTTCTCAT
TTTGTGCCCGTCTTTCTGCCTGCCAAACCTACCACCACCCCCGCCCC
ACGACCACCTACTCCAGCCCCCACCATCGCCTCCCAGCCCCTCAGC
CTGAGGCCAGAGGCTTGTCGCCCTGCTGCGGGGGGCGCTGTCCATA
CCAGAGGACTCGACTTCGCCTGCGATATTTATATATGGGCCCCCCT
CGCCGGCACCTGCGGAGTCTTGCTCCTGAGCCTTGTGATCACGCTT
TATTGTAACCATCGGAATAGATCCAAAAGAAGCCGCCTGCTCCATA
GCGATTACATGAATATGACTCCACGCCGCCCTGGCCCCACAAGGAA
ACACTACCAGCCTTACGCACCACCTAGAGATTTCGCTGCCTATCGG
AGCAGGGTGAAGTTTTCCAGATCTGCAGATGCACCAGCGTATCAGC
AGGGCCAGAACCAACTGTATAACGAGCTCAACCTGGGACGCAGGG
AAGAGTATGACGTTTTGGACAAGCGCAGAGGACGGGACCCTGAGA
TGGGTGGCAAACCAAGACGAAAAAACCCCCAGGAGGGTCTCTATA
ATGAGCTGCAGAAGGATAAGATGGCTGAAGCCTATTCTGAAATAG
GCATGAAAGGAGAGCGGAGAAGGGGAAAAGGGCACGACGGTTTG
TACCAGGGACTCAGCACTGCTACGAAGGATACTTATGACGCTCTCC
ACATGCAAGCCCTGCCACCTAGG
Clone 24C8 CD8 CD3 zeta CAR AA Heavy & Light Chains
(SEQ ID NO. 70)
QVQLQESGPGLVKPSQTLSLTCTVSGGSISSGGFYVVSWIRQHPGKGLE
WIGYIHHSGSTHYNPSLKSRVTISIDTSKNLFSLRLSSVTAADTAVYYC
ASLVYCGGDCYSGFDYWGQGTLVTVSSGGGGSGGGGSGGGGSDIQL
TQSPSSLSASVGDRVSFTCQASQDINNFLNWYQQKPGKAPKLLIYDAS
NLETGVPSRFSGSGSGTDFTFTISSLQPEDIATYYCQQYGNLPFTFGGGT
KVEIKRAAALSNSIMYFSHFVPVFLPAKPTTTPAPRPPTPAPTIASQPLS
LRPEACRPAAGGAVHTRGLDFACDIYIWAPLAGTCGVLLLSLVITLYC
NHRNRSKRSRLLHSDYMNMTPRRPGPTRKHYQPYAPPRDFAAYRSRV
KFSRSADAPAYQQGQNQLYNELNLGRREEYDVLDKRRGRDPEMGGK
PRRKNPQEGLYNELQKDKMAEAYSEIGMKGERRRGKGHDGLYQGLS
TA TKDTYDALHM QALPPR
Clone 20C5.1 HC DNA
(SEQ ID NO. 71)
CAGGTCCAACTGGTGCAGTCCGGAGCCGAAGTCAAGAAACCAGGT
GCCTCCGTTAAAGTGAGTTGCAAAGTCTCTGGATACACTCTGACCG
AGCTCTCTATGCACTGGGTCCGGCAGGCCCCCGGCAAGGGATTGGA
ATGGATGGGCGGGTTCGATCCTGAGGACGGAGAGACTATCTACGC
TCAAAAATTCCAGGGACGAGTGACTGTGACCGAAGACACTAGTAC
CGACACTGCCTACATGGAACTTTCCTCTCTGCGATCAGAAGATACC
GCAGTGTACTACTGTGCTACTGAATCTAGGGGCATTGGATGGCCCT
ACTTCGATTACTGGGGTCAGGGAACTCTGGTGACTGTCTCCAGC
Clone 20C5.1 AA HC (CDRs in Underline)
(SEQ ID NO. 72)
QVQLVQSGAEVKKPGASVKVSCKVSGYTLTELSMHWVRQAPGKGLE
WMGGFDPEDGETIYAQKFQGRVTVTEDTSTDTAYMELSSLRSEDTAV
YYCATESRGIGWPYFDYWGQGTLVTVSS
(SEQ ID NO. 73)
Clone 20C5.1 HC AA CDR1: GYTLTEL
(SEQ ID NO. 74)
Clone 20C5.1 HC AA CDR2: DPEDGE
(SEQ ID NO. 75)
Clone 20C5.1 HC AA CDR3: ESRGIGWPYFDY
Clone 20C5.1 LC DNA
(SEQ ID NO. 76)
GATATTCAGATGACTCAATCTCCTTCTTCTCTGTCCGCTTCCGTGGG
CGATAGAGTGACCATTACTTGTAGGGCGTCCCAGTCAATCTCCAGT
TATTTGAATTGGTATCAGCAGAAGCCCGGGAAAGCACCTAAGCTGT
TGATCAGCGGGGCTTCTAGCCTGAAGAGTGGGGTACCTTCACGGTT
CAGCGGAAGCGGAAGCGGAACCGATTTCACCCTGACTATCAGCAG
CCTGCCACCTGAGGACTTTGCAACTTACTACTGCCAACAGTCATAC
AGCACTCCGATCACTTTCGGCCAGGGCACCCGGCTCGAAATCAAGC
GC
Clone 20C5.1 AA LC (CDRs in Underline)
(SEQ ID NO. 77)
DIQMTQSPSSLSASVGDRVTITCRASQSISSYLNWYQQKPGKAPKLLIS
GASSLKSGVPSRFSGSGSGTDFTLTISSLPPEDFATYYCQQSYSTPITFG
QGTRLEIKR
(SEQ ID NO. 78)
Clone 20C5.1 AA LC CDR1: RASQSISSYLN
(SEQ ID NO. 79)
Clone 20C5.1 AA LC CDR2: GASSLKS
(SEQ ID NO. 80)
Clone 20C5.1 AA LC CDR3: QQSYSTPIT
Clone 20C5.1 CD28T CD3 zeta CAR DNA Heavy & Light Chains
(SEQ ID NO. 81)
ATGGCACTCCCCGTAACTGCTCTGCTGCTGCCGTTGGCATTGCTCCT
GCACGCCGCACGCCCGCAGGTCCAACTGGTGCAGTCCGGAGCCGA
AGTCAAGAAACCAGGTGCCTCCGTTAAAGTGAGTTGCAAAGTCTCT
GGATACACTCTGACCGAGCTCTCTATGCACTGGGTCCGGCAGGCCC
CCGGCAAGGGATTGGAATGGATGGGCGGGTTCGATCCTGAGGACG
GAGAGACTATCTACGCTCAAAAATTCCAGGGACGAGTGACTGTGA
CCGAAGACACTAGTACCGACACTGCCTACATGGAACTTTCCTCTCT
GCGATCAGAAGATACCGCAGTGTACTACTGTGCTACTGAATCTAGG
GGCATTGGATGGCCCTACTTCGATTACTGGGGTCAGGGAACTCTGG
TGACTGTCTCCAGCGGTGGAGGTGGCAGCGGTGGTGGCGGAAGCG
GGGGGGGCGGCTCTGATATTCAGATGACTCAATCTCCTTCTTCTCT
GTCCGCTTCCGTGGGCGATAGAGTGACCATTACTTGTAGGGCGTCC
CAGTCAATCTCCAGTTATTTGAATTGGTATCAGCAGAAGCCCGGGA
AAGCACCTAAGCTGTTGATCAGCGGGGCTTCTAGCCTGAAGAGTGG
GGTACCTTCACGGTTCAGCGGAAGCGGAAGCGGAACCGATTTCAC
CCTGACTATCAGCAGCCTGCCACCTGAGGACTTTGCAACTTACTAC
TGCCAACAGTCATACAGCACTCCGATCACTTTCGGCCAGGGCACCC
GGCTCGAAATCAAGCGCGCTGCTGCTTTGGACAATGAGAAGTCAA
ACGGCACCATCATACATGTTAAAGGTAAACATCTGTGTCCCTCCCC
GCTGTTCCCCGGCCCTTCCAAACCGTTCTGGGTTCTGGTGGTGGTCG
GAGGCGTACTCGCTTGCTATAGTCTGCTGGTAACTGTCGCCTTCATC
ATCTTTTGGGTGAGATCCAAAAGAAGCCGCCTGCTCCATAGCGATT
ACATGAATATGACTCCACGCCGCCCTGGCCCCACAAGGAAACACT
ACCAGCCTTACGCACCACCTAGAGATTTCGCTGCCTATCGGAGCAG
GGTGAAGTTTTCCAGATCTGCAGATGCACCAGCGTATCAGCAGGGC
CAGAACCAACTGTATAACGAGCTCAACCTGGGACGCAGGGAAGAG
TATGACGTTTTGGACAAGCGCAGAGGACGGGACCCTGAGATGGGT
GGCAAACCAAGACGAAAAAACCCCCAGGAGGGTCTCTATAATGAG
CTGCAGAAGGATAAGATGGCTGAAGCCTATTCTGAAATAGGCATG
AAAGGAGAGCGGAGAAGGGGAAAAGGGCACGACGGTTTGTACCA
GGGACTCAGCACTGCTACGAAGGATACTTATGACGCTCTCCACATG
CAAGCCCTGCCACCTAGGTAA
Clone 20C5.1 CD28T CD3 zeta CAR AA Heavy & Light Chains
(Signal Peptide in Bold)
(SEQ ID NO. 82)
MALPVTALLLPLALLLHAARPQVQLVQSGAEVKKPGASVKVSCKVS
GYTLTELSMHWVRQAPGKGLEWMGGFDPEDGETIYAQKFQGRVTVT
EDTSTDTAYMELSSLRSEDTAVYYCATESRGIGWPYFDYWGQGTLVT
VSSGGGGSGGGGSGGGGSDIQMTQSPSSLSASVGDRVTITCRASQSISS
YLNWYQQKPGKAPKLLISGASSLKSGVPSRFSGSGSGTDFTLTISSLPPE
DFATYYCQQSYSTPITFGQGTRLEIKRAAALDNEKSNGTIIHVKGKHLC
PSPLFPGPSKPFWVLVVVGGVLACYSLLVTVAFIIFWVRSKRSRLLHSD
YMNMTPRRPGPTRKHYQPYAPPRDFAAYRSRVKFSRSADAPAYQQG
QNQLYNELNLGRREEYDVLDKRRGRDPEMGGKPRRKNPQEGLYNEL
QKDKMAEAYSEIGMKGERRRGKGHDGLYQGLSTATKDTYDALHMQ
ALPPR
Clone 20C5.1 CD28T CD3 zeta CAR DNA Heavy & Light Chains
(SEQ ID NO. 83)
CAGGTCCAACTGGTGCAGTCCGGAGCCGAAGTCAAGAAACCAGGT
GCCTCCGTTAAAGTGAGTTGCAAAGTCTCTGGATACACTCTGACCG
AGCTCTCTATGCACTGGGTCCGGCAGGCCCCCGGCAAGGGATTGGA
ATGGATGGGCGGGTTCGATCCTGAGGACGGAGAGACTATCTACGC
TCAAAAATTCCAGGGACGAGTGACTGTGACCGAAGACACTAGTAC
CGACACTGCCTACATGGAACTTTCCTCTCTGCGATCAGAAGATACC
GCAGTGTACTACTGTGCTACTGAATCTAGGGGCATTGGATGGCCCT
ACTTCGATTACTGGGGTCAGGGAACTCTGGTGACTGTCTCCAGCGG
TGGAGGTGGCAGCGGTGGTGGCGGAAGCGGGGGGGGCGGCTCTGA
TATTCAGATGACTCAATCTCCTTCTTCTCTGTCCGCTTCCGTGGGCG
ATAGAGTGACCATTACTTGTAGGGCGTCCCAGTCAATCTCCAGTTA
TTTGAATTGGTATCAGCAGAAGCCCGGGAAAGCACCTAAGCTGTTG
ATCAGCGGGGCTTCTAGCCTGAAGAGTGGGGTACCTTCACGGTTCA
GCGGAAGCGGAAGCGGAACCGATTTCACCCTGACTATCAGCAGCC
TGCCACCTGAGGACTTTGCAACTTACTACTGCCAACAGTCATACAG
CACTCCGATCACTTTCGGCCAGGGCACCCGGCTCGAAATCAAGCGC
GCTGCTGCTTTGGACAATGAGAAGTCAAACGGCACCATCATACATG
TTAAAGGTAAACATCTGTGTCCCTCCCCGCTGTTCCCCGGCCCTTCC
AAACCGTTCTGGGTTCTGGTGGTGGTCGGAGGCGTACTCGCTTGCT
ATAGTCTGCTGGTAACTGTCGCCTTCATCATCTTTTGGGTGAGATCC
AAAAGAAGCCGCCTGCTCCATAGCGATTACATGAATATGACTCCAC
GCCGCCCTGGCCCCACAAGGAAACACTACCAGCCTTACGCACCACC
TAGAGATTTCGCTGCCTATCGGAGCAGGGTGAAGTTTTCCAGATCT
GCAGATGCACCAGCGTATCAGCAGGGCCAGAACCAACTGTATAAC
GAGCTCAACCTGGGACGCAGGGAAGAGTATGACGTTTTGGACAAG
CGCAGAGGACGGGACCCTGAGATGGGTGGCAAACCAAGACGAAA
AAACCCCCAGGAGGGTCTCTATAATGAGCTGCAGAAGGATAAGAT
GGCTGAAGCCTATTCTGAAATAGGCATGAAAGGAGAGCGGAGAAG
GGGAAAAGGGCACGACGGTTTGTACCAGGGACTCAGCACTGCTAC
GAAGGATACTTATGACGCTCTCCACATGCAAGCCCTGCCACCTAGG
Clone 20C5.1 CD28T CD3 zeta CAR AA Heavy & Light Chains
(SEQ ID NO. 84)
QVQLVQSGAEVKKPGASVKVSCKVSGYTLTELSMHWVRQAPGKGLE
WMGGFDPEDGETIYAQKFQGRVTVTEDTSTDTAYMELSSLRSEDTAV
YYCATESRGIGWPYFDYWGQGTLVTVSSGGGGSGGGGSGGGGSDIQ
MTQSPSSLSASVGDRVTITCRASQSISSYLNWYQQKPGKAPKLLISGAS
SLKSGVPSRFSGSGSGTDFTLTISSLPPEDFATYYCQQSYSTPITFGQGT
RLEIKRAAALDNEKSNGTIIHVKGKHLCPSPLFPGPSKPFWVLVVVGG
VLACYSLLVTVAFIIFWVRSKRSRLLHSDYMNMTPRRPGPTRKHYQPY
APPRDFAAYRSRVKFSRSADAPAYQQGQNQLYNELNLGRREEYDVLD
KRRGRDPEMGGKPRRKNPQEGLYNELQKDKMAEAYSEIGMKGERRR
GKGHDGLYQGLSTATKDTYDALHMQALPPR
Clone 20C5.1 CD28 CD3 zeta CAR DNA Heavy & Light Chains
(SEQ ID NO. 85)
ATGGCACTCCCCGTAACTGCTCTGCTGCTGCCGTTGGCATTGCTCCT
GCACGCCGCACGCCCGCAGGTGCAGCTTGTGCAGAGCGGGGCCGA
GGTGAAGAAGCCCGGGGCCAGCGTCAAAGTGTCCTGTAAGGTCAG
CGGTTACACCCTCACCGAGCTGAGCATGCACTGGGTACGGCAGGCT
CCCGGCAAAGGTCTTGAGTGGATGGGTGGATTTGATCCAGAAGAT
GGAGAGACTATCTACGCCCAGAAGTTCCAGGGCCGGGTCACCGTA
ACAGAAGACACCTCAACTGACACCGCTTACATGGAGCTGAGTTCAC
TGCGGTCCGAGGACACGGCCGTGTATTATTGTGCCACCGAGAGCCG
CGGAATCGGATGGCCTTACTTCGACTACTGGGGACAGGGTACACTT
GTTACAGTATCATCCGGGGGTGGCGGCTCTGGTGGGGGCGGCTCCG
GAGGGGGTGGATCAGATATCCAAATGACTCAAAGTCCAAGTTCCCT
GTCTGCCTCAGTCGGAGATAGAGTCACCATAACCTGCAGGGCAAGT
CAGTCCATCTCCTCCTATCTGAACTGGTACCAACAGAAACCTGGAA
AGGCGCCTAAGCTCCTGATCTCCGGAGCCTCATCTTTGAAATCCGG
TGTCCCATCTCGCTTCAGTGGCTCTGGAAGCGGTACAGATTTTACTT
TGACCATTAGCAGCCTCCCACCGGAAGACTTTGCTACATATTACTG
CCAGCAGTCTTACTCAACCCCAATCACCTTCGGGCAAGGCACCAGA
CTCGAAATAAAAAGAGCAGCTGCTATCGAGGTTATGTACCCACCGC
CGTACTTGGATAACGAAAAAAGCAATGGGACCATCATTCATGTGA
AGGGTAAGCACCTTTGCCCTAGCCCACTGTTTCCTGGCCCGAGTAA
ACCCTTTTGGGTACTTGTGGTCGTCGGCGGCGTGCTGGCCTGCTACT
CACTCCTGGTTACCGTCGCATTCATCATCTTTTGGGTGAGATCCAAA
AGAAGCCGCCTGCTCCATAGCGATTACATGAATATGACTCCACGCC
GCCCTGGCCCCACAAGGAAACACTACCAGCCTTACGCACCACCTAG
AGATTTCGCTGCCTATCGGAGCAGGGTGAAGTTTTCCAGATCTGCA
GATGCACCAGCGTATCAGCAGGGCCAGAACCAACTGTATAACGAG
CTCAACCTGGGACGCAGGGAAGAGTATGACGTTTTGGACAAGCGC
AGAGGACGGGACCCTGAGATGGGTGGCAAACCAAGACGAAAAAA
CCCCCAGGAGGGTCTCTATAATGAGCTGCAGAAGGATAAGATGGC
TGAAGCCTATTCTGAAATAGGCATGAAAGGAGAGCGGAGAAGGGG
AAAAGGGCACGACGGTTTGTACCAGGGACTCAGCACTGCTACGAA
GGATACTTATGACGCTCTCCACATGCAAGCCCTGCCACCTAGGTAA
Clone 20C5.1 CD28 CD3 zeta CAR AA Heavy & Light Chains
(Signal Peptide in Bold)
(SEQ ID NO. 86)
MALPVTALLLPLALLLHAARPQVQLVQSGAEVKKPGASVKVSCKVS
GYTLTELSMHWVRQAPGKGLEWMGGFDPEDGETIYAQKFQGRVTVT
EDTSTDTAYMELSSLRSEDTAVYYCATESRGIGWPYFDYWGQGTLVT
VSSGGGGSGGGGSGGGGSDIQMTQSPSSLSASVGDRVTITCRASQSISS
YLNWYQQKPGKAPKLLISGASSLKSGVPSRFSGSGSGTDFTLTISSLPPE
DFATYYCQQSYSTPITFGQGTRLEIKRAAAIEVMYPPPYLDNEKSNGTII
HVKGKHLCPSPLFPGPSKPFWVLVVVGGVLACYSLLVTVAFIIFWVRS
KRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNP
QEGLYNELQKDKMAEAYSEIGMKGERRRGKGHDGLYQGLST
ATKDTYDALH MQALPPR
Clone 20C5.1 CD28 CD3 zeta CAR DNA Heavy & Light Chains
(SEQ ID NO. 87)
CAGGTGCAGCTTGTGCAGAGCGGGGCCGAGGTGAAGAAGCCCGGG
GCCAGCGTCAAAGTGTCCTGTAAGGTCAGCGGTTACACCCTCACCG
AGCTGAGCATGCACTGGGTACGGCAGGCTCCCGGCAAAGGTCTTG
AGTGGATGGGTGGATTTGATCCAGAAGATGGAGAGACTATCTACG
CCCAGAAGTTCCAGGGCCGGGTCACCGTAACAGAAGACACCTCAA
CTGACACCGCTTACATGGAGCTGAGTTCACTGCGGTCCGAGGACAC
GGCCGTGTATTATTGTGCCACCGAGAGCCGCGGAATCGGATGGCCT
TACTTCGACTACTGGGGACAGGGTACACTTGTTACAGTATCATCCG
GGGGTGGCGGCTCTGGTGGGGGCGGCTCCGGAGGGGGTGGATCAG
ATATCCAAATGACTCAAAGTCCAAGTTCCCTGTCTGCCTCAGTCGG
AGATAGAGTCACCATAACCTGCAGGGCAAGTCAGTCCATCTCCTCC
TATCTGAACTGGTACCAACAGAAACCTGGAAAGGCGCCTAAGCTC
CTGATCTCCGGAGCCTCATCTTTGAAATCCGGTGTCCCATCTCGCTT
CAGTGGCTCTGGAAGCGGTACAGATTTTACTTTGACCATTAGCAGC
CTCCCACCGGAAGACTTTGCTACATATTACTGCCAGCAGTCTTACT
CAACCCCAATCACCTTCGGGCAAGGCACCAGACTCGAAATAAAAA
GAGCAGCTGCTATCGAGGTTATGTACCCACCGCCGTACTTGGATAA
CGAAAAAAGCAATGGGACCATCATTCATGTGAAGGGTAAGCACCT
TTGCCCTAGCCCACTGTTTCCTGGCCCGAGTAAACCCTTTTGGGTAC
TTGTGGTCGTCGGCGGCGTGCTGGCCTGCTACTCACTCCTGGTTACC
GTCGCATTCATCATCTTTTGGGTGAGATCCAAAAGAAGCCGCCTGC
TCCATAGCGATTACATGAATATGACTCCACGCCGCCCTGGCCCCAC
AAGGAAACACTACCAGCCTTACGCACCACCTAGAGATTTCGCTGCC
TATCGGAGCAGGGTGAAGTTTTCCAGATCTGCAGATGCACCAGCGT
ATCAGCAGGGCCAGAACCAACTGTATAACGAGCTCAACCTGGGAC
GCAGGGAAGAGTATGACGTTTTGGACAAGCGCAGAGGACGGGACC
CTGAGATGGGTGGCAAACCAAGACGAAAAAACCCCCAGGAGGGTC
TCTATAATGAGCTGCAGAAGGATAAGATGGCTGAAGCCTATTCTGA
AATAGGCATGAAAGGAGAGCGGAGAAGGGGAAAAGGGCACGACG
GTTTGTACCAGGGACTCAGCACTGCTACGAAGGATACTTATGACGC
TCTCCACATGCAAGCCCTGCCACCTAGG
Clone 20C5.1 CD28 CD3 zeta CAR AA Heavy & Light Chains
(SEQ ID NO. 88)
QVQLVQSGAEVKKPGASVKVSCKVSGYTLTELSMHWVRQAPGKGLE
WMGGFDPEDGETIYAQKFQGRVTVTEDTSTDTAYMELSSLRSEDTAV
YYCATESRGIGWPYFDYWGQGTLVTVSSGGGGSGGGGSGGGGSDIQ
MTQSPSSLSASVGDRVTITCRASQSISSYLNWYQQKPGKAPKLLISGAS
SLKSGVPSRFSGSGSGTDFTLTISSLPPEDFATYYCQQSYSTPITFGQGT
RLEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLG
RREEYDVLDKRRGRDPEMGGKPRRKNPQEGLYNELQKDKMAEAYSE
IGMKGERRRGKGHDGLYQGLSTATKDTYDALHM QALPPR
Clone 20C5.1 CD8 CD3 zeta CAR DNA Heavy & Light Chains
(SEQ ID NO. 89)
ATGGCACTCCCCGTAACTGCTCTGCTGCTGCCGTTGGCATTGCTCCT
GCACGCCGCACGCCCGCAGGTGCAGTTGGTGCAAAGCGGCGCAGA
AGTTAAGAAACCTGGGGCGTCAGTTAAGGTGTCTTGCAAAGTATCT
GGCTATACCCTCACTGAGCTGTCCATGCATTGGGTAAGGCAGGCTC
CTGGAAAGGGGCTCGAATGGATGGGAGGATTTGACCCTGAAGACG
GAGAGACCATCTACGCCCAGAAATTCCAGGGTAGAGTAACAGTGA
CTGAGGACACTAGCACTGACACAGCGTACATGGAGCTGAGTTCTCT
GAGAAGTGAGGACACAGCCGTTTACTACTGCGCTACCGAGTCCAG
AGGTATTGGCTGGCCATACTTCGACTATTGGGGTCAGGGCACCCTG
GTTACAGTGAGTTCAGGAGGCGGGGGCTCTGGGGGGGGCGGTTCC
GGAGGGGGGGGCTCAGATATACAGATGACGCAGAGTCCATCAAGT
CTCTCAGCCAGCGTGGGAGATCGCGTGACTATTACTTGCCGCGCCA
GCCAGAGTATTAGCTCCTATCTGAATTGGTACCAGCAAAAGCCCGG
GAAGGCCCCTAAGCTTCTGATTTCTGGCGCCTCCTCTTTGAAGTCA
GGTGTGCCAAGCAGATTTAGCGGGTCTGGAAGTGGCACTGACTTTA
CACTTACTATCTCCAGCCTGCCCCCAGAGGATTTTGCCACATATTAC
TGTCAGCAAAGCTACTCTACTCCAATCACTTTCGGCCAGGGCACAA
GATTGGAGATTAAGAGGGCTGCCGCACTTTCAAATTCCATCATGTA
TTTCAGCCATTTTGTGCCTGTTTTTCTTCCGGCCAAACCTACAACCA
CTCCCGCCCCACGCCCACCTACTCCCGCCCCTACCATTGCCTCCCAG
CCTCTGTCTCTTAGACCTGAGGCTTGTAGACCTGCTGCCGGCGGAG
CCGTGCACACTCGCGGTCTGGACTTCGCCTGCGACATCTATATCTG
GGCCCCTCTGGCCGGCACCTGCGGCGTTCTCCTTCTCTCACTCGTAA
TCACACTCTATTGCAATCACAGGAACAGATCCAAAAGAAGCCGCCT
GCTCCATAGCGATTACATGAATATGACTCCACGCCGCCCTGGCCCC
ACAAGGAAACACTACCAGCCTTACGCACCACCTAGAGATTTCGCTG
CCTATCGGAGCAGGGTGAAGTTTTCCAGATCTGCAGATGCACCAGC
GTATCAGCAGGGCCAGAACCAACTGTATAACGAGCTCAACCTGGG
ACGCAGGGAAGAGTATGACGTTTTGGACAAGCGCAGAGGACGGGA
CCCTGAGATGGGTGGCAAACCAAGACGAAAAAACCCCCAGGAGGG
TCTCTATAATGAGCTGCAGAAGGATAAGATGGCTGAAGCCTATTCT
GAAATAGGCATGAAAGGAGAGCGGAGAAGGGGAAAAGGGCACGA
CGGTTTGTACCAGGGACTCAGCACTGCTACGAAGGATACTTATGAC
GCTCTCCACATGCAAGCCCTGCCACCTAGGTAA
Clone 20C5.1 CD8 CD3 zeta CAR AA Heavy & Light Chains
(Signal Peptide in Bold)
(SEQ ID NO. 90)
MALPVTALLLPLALLLHAARPQVQLVQSGAEVKKPGASVKVSCKVS
GYTLTELSMHWVRQAPGKGLEWMGGFDPEDGETIYAQKFQGRVTVT
EDTSTDTAYMELSSLRSEDTAVYYCATESRGIGWPYFDYWGQGTLVT
VSSGGGGSGGGGSGGGGSDIQMTQSPSSLSASVGDRVTITCRASQSISS
YLNWYQQKPGKAPKLLISGASSLKSGVPSRFSGSGSGTDFTLTISSLPPE
DFATYYCQQSYSTPITFGQGTRLEIKRAAALSNSIMYFSHFVPVFLPAK
PTTTPAPRPPTPAPTIASQPLSLRPEACRPAAGGAVHTRGLDFACDIYIW
APLAGTCGVLLLSLVITLYCNHRNRSKRSRLLHSDYMNMTPRRPGPTR
KHYQPYAPPRDFAAYRSRVKFSRSADAPAYQQGQNQLYNELNLGRRE
EYDVLDKRRGRDPEMGGKPRRKNPQEGLYNELQKDKMAEAYSEIGM
KGERRRGKGHDGLYQGLSTATKDTYDALHMQALPPR
Clone 20C5.1 CD8 CD3 zeta CAR DNA Heavy & Light Chains
(SEQ ID NO. 91)
CAGGTGCAGTTGGTGCAAAGCGGCGCAGAAGTTAAGAAACCTGGG
GCGTCAGTTAAGGTGTCTTGCAAAGTATCTGGCTATACCCTCACTG
AGCTGTCCATGCATTGGGTAAGGCAGGCTCCTGGAAAGGGGCTCG
AATGGATGGGAGGATTTGACCCTGAAGACGGAGAGACCATCTACG
CCCAGAAATTCCAGGGTAGAGTAACAGTGACTGAGGACACTAGCA
CTGACACAGCGTACATGGAGCTGAGTTCTCTGAGAAGTGAGGACA
CAGCCGTTTACTACTGCGCTACCGAGTCCAGAGGTATTGGCTGGCC
ATACTTCGACTATTGGGGTCAGGGCACCCTGGTTACAGTGAGTTCA
GGAGGCGGGGGCTCTGGGGGGGGCGGTTCCGGAGGGGGGGGCTCA
GATATACAGATGACGCAGAGTCCATCAAGTCTCTCAGCCAGCGTGG
GAGATCGCGTGACTATTACTTGCCGCGCCAGCCAGAGTATTAGCTC
CTATCTGAATTGGTACCAGCAAAAGCCCGGGAAGGCCCCTAAGCTT
CTGATTTCTGGCGCCTCCTCTTTGAAGTCAGGTGTGCCAAGCAGAT
TTAGCGGGTCTGGAAGTGGCACTGACTTTACACTTACTATCTCCAG
CCTGCCCCCAGAGGATTTTGCCACATATTACTGTCAGCAAAGCTAC
TCTACTCCAATCACTTTCGGCCAGGGCACAAGATTGGAGATTAAGA
GGGCTGCCGCACTTTCAAATTCCATCATGTATTTCAGCCATTTTGTG
CCTGTTTTTCTTCCGGCCAAACCTACAACCACTCCCGCCCCACGCCC
ACCTACTCCCGCCCCTACCATTGCCTCCCAGCCTCTGTCTCTTAGAC
CTGAGGCTTGTAGACCTGCTGCCGGCGGAGCCGTGCACACTCGCGG
TCTGGACTTCGCCTGCGACATCTATATCTGGGCCCCTCTGGCCGGC
ACCTGCGGCGTTCTCCTTCTCTCACTCGTAATCACACTCTATTGCAA
TCACAGGAACAGATCCAAAAGAAGCCGCCTGCTCCATAGCGATTA
CATGAATATGACTCCACGCCGCCCTGGCCCCACAAGGAAACACTAC
CAGCCTTACGCACCACCTAGAGATTTCGCTGCCTATCGGAGCAGGG
TGAAGTTTTCCAGATCTGCAGATGCACCAGCGTATCAGCAGGGCCA
GAACCAACTGTATAACGAGCTCAACCTGGGACGCAGGGAAGAGTA
TGACGTTTTGGACAAGCGCAGAGGACGGGACCCTGAGATGGGTGG
CAAACCAAGACGAAAAAACCCCCAGGAGGGTCTCTATAATGAGCT
GCAGAAGGATAAGATGGCTGAAGCCTATTCTGAAATAGGCATGAA
AGGAGAGCGGAGAAGGGGAAAAGGGCACGACGGTTTGTACCAGG
GACTCAGCACTGCTACGAAGGATACTTATGACGCTCTCCACATGCA
AGCCCTGCCACCTAGG
Clone 20C5.1 CD8 CD3 zeta CAR AA Heavy & Light Chains
(SEQ ID NO. 92)
QVQLVQSGAEVKKPGASVKVSCKVSGYTLTELSMHWVRQAPGKGLE
WMGGFDPEDGETIYAQKFQGRVTVTEDTSTDTAYMELSSLRSEDTAV
YYCATESRGIGWPYFDYWGQGTLVTVSSGGGGSGGGGSGGGGSDIQ
MTQSPSSLSASVGDRVTITCRASQSISSYLNWYQQKPGKAPKLLISGAS
SLKSGVPSRFSGSGSGTDFTLTISSLPPEDFATYYCQQSYSTPITFGQGT
RLEIKRAAALSNSIMYFSHFVPVFLPAKPTTTPAPRPPTPAPTIASQPLSL
RPEACRPAAGGAVHTRGLDFACDIYIWAPLAGTCGVLLLSLVITLYCN
HRNRSKRSRLLHSDYMNMTPRRPGPTRKHYQPYAPPRDFAAYRSRVK
FSRSADAPAYQQGQNQLYNELNLGRREEYDVLDKRRGRDPEMGGKP
RRKNPQEGLYNELQKDKMAEAYSEIGMKGERRRGKGHDGLYQGLST
ATKDTYDALHMQALPPR
Clone 20C5.2 HC DNA
(SEQ ID NO. 93)
CAGGTCCAGTTGGTCGAAAGTGGCGGTGGTGTAGTGCAGCCGGGC
CGCAGTTTGAGGCTTTCCTGTGCGGCTTCAGGCTTTACTTTTTCCAG
CTATGGAATGCACTGGGTGCGGCAGGCCCCCGGCAAAGGACTTGA
GTGGGTGGCCGTCATTTCTTATGACGGATCAGATAAGTACTACGTG
GACAGCGTCAAGGGCAGATTCACCATCTCTAGGGACAACAGTAAA
AATAGACTCTACCTCCAGATGAATAGCCTCAGAGCTGAAGACACG
GCCGTCTACTATTGTGCTCGGGAGCGGTATAGTGGCAGAGACTACT
GGGGGCAGGGCACACTCGTTACAGTGAGTAGC
Clone 20C5.2 AA HC (CDRs in Underline)
(SEQ ID NO. 94)
QVQLVESGGGVVQPGRSLRLSCAASGFTFSSYGMHWVRQAPGKGLE
WVAVISYDGSDKYYVDSVKGRFTISRDNSKNRLYLQMNSLRAEDTAV
YYCARERYSGRDYWGQGTLVTVSS
(SEQ ID NO. 95)
Clone 20C5.2 HC AA CDR1: GFTFSSY
(SEQ ID NO. 96)
Clone 20C5.2 HC AA CDR2: SYDGSD
(SEQ ID NO. 97)
Clone 20C5.2 HC AA CDR3: ERYSGRDY
Clone 20C5.2 LC DNA
(SEQ ID NO. 98)
GAGATTGTTATGACCCAGAGTCCTGCGACCCTCTCAGTCAGCCCCG
GGGAGCGCGCAACTTTGTCTTGCAGAGCTAGTCAGTCCGTGTCCTC
TCTTCTGACATGGTACCAGCAAAAGCCCGGGCAGGCTCCGCGCCTT
TTGATCTTTGGGGCTTCAACAAGAGCCACTGGGATTCCCGCACGAT
TCTCTGGCTCCGGGAGCGGTACTGGTTTCACCCTGACGATTAGCAG
TCTCCAGAGCGAGGACTTCGCCGTATACTACTGCCAGCAGTACGAT
ACGTGGCCATTCACTTTTGGACCAGGGACTAAAGTGGATTTTAAGC
GC
Clone 20C5.2 AA LC (CDRs in Underline)
(SEQ ID NO. 99)
EIVMTQSPATLSVSPGERATLSCRASQSVSSLLTWYQQKPGQAPRLLIF
GASTRATGIPARFSGSGSGTGFTLTISSLQSEDFAVYYCQQYDTWPFTF
GPGTKVDFKR
(SEQ ID NO. 100)
Clone 20C5.2 AA LC CDR1: RASQSVSSLLT
(SEQ ID NO. 101)
Clone 20C5.2 AA LC CDR2: GASTRAT
(SEQ ID NO. 102)
Clone 20C5.2 AA LC CDR3: QQYDTWPFT
Clone 20C5.2 CD28T CD3 zeta CAR DNA Heavy & Light Chains
(SEQ ID NO. 103)
ATGGCACTCCCCGTAACTGCTCTGCTGCTGCCGTTGGCATTGCTCCT
GCACGCCGCACGCCCGCAGGTCCAGTTGGTCGAAAGTGGCGGTGG
TGTAGTGCAGCCGGGCCGCAGTTTGAGGCTTTCCTGTGCGGCTTCA
GGCTTTACTTTTTCCAGCTATGGAATGCACTGGGTGCGGCAGGCCC
CCGGCAAAGGACTTGAGTGGGTGGCCGTCATTTCTTATGACGGATC
AGATAAGTACTACGTGGACAGCGTCAAGGGCAGATTCACCATCTCT
AGGGACAACAGTAAAAATAGACTCTACCTCCAGATGAATAGCCTC
AGAGCTGAAGACACGGCCGTCTACTATTGTGCTCGGGAGCGGTATA
GTGGCAGAGACTACTGGGGGCAGGGCACACTCGTTACAGTGAGTA
GCGGCGGAGGAGGGAGTGGGGGCGGTGGCTCCGGTGGAGGAGGTT
CTGAGATTGTTATGACCCAGAGTCCTGCGACCCTCTCAGTCAGCCC
CGGGGAGCGCGCAACTTTGTCTTGCAGAGCTAGTCAGTCCGTGTCC
TCTCTTCTGACATGGTACCAGCAAAAGCCCGGGCAGGCTCCGCGCC
TTTTGATCTTTGGGGCTTCAACAAGAGCCACTGGGATTCCCGCACG
ATTCTCTGGCTCCGGGAGCGGTACTGGTTTCACCCTGACGATTAGC
AGTCTCCAGAGCGAGGACTTCGCCGTATACTACTGCCAGCAGTACG
ATACGTGGCCATTCACTTTTGGACCAGGGACTAAAGTGGATTTTAA
GCGCGCCGCCGCTCTCGATAACGAAAAGTCAAATGGCACCATAAT
CCACGTCAAAGGCAAGCACCTGTGCCCTTCCCCGCTCTTCCCCGGA
CCCAGTAAACCATTTTGGGTGCTGGTTGTTGTGGGGGGCGTGCTGG
CCTGCTATAGCCTTTTGGTCACTGTAGCCTTCATTATTTTTTGGGTC
AGATCCAAAAGAAGCCGCCTGCTCCATAGCGATTACATGAATATG
ACTCCACGCCGCCCTGGCCCCACAAGGAAACACTACCAGCCTTACG
CACCACCTAGAGATTTCGCTGCCTATCGGAGCAGGGTGAAGTTTTC
CAGATCTGCAGATGCACCAGCGTATCAGCAGGGCCAGAACCAACT
GTATAACGAGCTCAACCTGGGACGCAGGGAAGAGTATGACGTTTT
GGACAAGCGCAGAGGACGGGACCCTGAGATGGGTGGCAAACCAA
GACGAAAAAACCCCCAGGAGGGTCTCTATAATGAGCTGCAGAAGG
ATAAGATGGCTGAAGCCTATTCTGAAATAGGCATGAAAGGAGAGC
GGAGAAGGGGAAAAGGGCACGACGGTTTGTACCAGGGACTCAGCA
CTGCTACGAAGGATACTTATGACGCTCTCCACATGCAAGCCCTGCC
ACCTAGGTAA
Clone 20C5.2 CD28T CD3 zeta CAR AA Heavy & Light Chains
(Signal Peptide in Bold)
(SEQ ID NO. 104)
MALPVTALLLPLALLLHAARPQVQLVESGGGVVQPGRSLRLSCAAS
GFTFSSYGMHWVRQAPGKGLEWVAVISYDGSDKYYVDSVKGRFTISR
DNSKNRLYLQMNSLRAEDTAVYYCARERYSGRDYWGQGTLVTVSSG
GGGSGGGGSGGGGSEIVMTQSPATLSVSPGERATLSCRASQSVSSLLT
WYQQKPGQAPRLLIFGASTRATGIPARFSGSGSGTGFTLTISSLQSEDFA
VYYCQQYDTWPFTFGPGTKVDFKRAAALDNEKSNGTIIHVKGKHLCP
SPLFPGPSKPFWVLVVVGGVLACYSLLVTVAFIIFWVRSKRSRLLHSD
YMNMTPRRPGPTRKHYQPYAPPRDFAAYRSRVKFSRSADAPAYQQG
QNQLYNELNLGRREEYDVLDKRRGRDPEMGGKPRRKNPQEGLYNEL
QKDKMAEAYSEIGMKGERRRGKGHDGLYQGLSTATK
DTYDALHMQALPPR
Clone 20C5.2 CD28T CD3 zeta CAR DNA Heavy & Light Chains
(SEQ ID NO. 105)
CAGGTCCAGTTGGTCGAAAGTGGCGGTGGTGTAGTGCAGCCGGGC
CGCAGTTTGAGGCTTTCCTGTGCGGCTTCAGGCTTTACTTTTTCCAG
CTATGGAATGCACTGGGTGCGGCAGGCCCCCGGCAAAGGACTTGA
GTGGGTGGCCGTCATTTCTTATGACGGATCAGATAAGTACTACGTG
GACAGCGTCAAGGGCAGATTCACCATCTCTAGGGACAACAGTAAA
AATAGACTCTACCTCCAGATGAATAGCCTCAGAGCTGAAGACACG
GCCGTCTACTATTGTGCTCGGGAGCGGTATAGTGGCAGAGACTACT
GGGGGCAGGGCACACTCGTTACAGTGAGTAGCGGCGGAGGAGGGA
GTGGGGGCGGTGGCTCCGGTGGAGGAGGTTCTGAGATTGTTATGAC
CCAGAGTCCTGCGACCCTCTCAGTCAGCCCCGGGGAGCGCGCAACT
TTGTCTTGCAGAGCTAGTCAGTCCGTGTCCTCTCTTCTGACATGGTA
CCAGCAAAAGCCCGGGCAGGCTCCGCGCCTTTTGATCTTTGGGGCT
TCAACAAGAGCCACTGGGATTCCCGCACGATTCTCTGGCTCCGGGA
GCGGTACTGGTTTCACCCTGACGATTAGCAGTCTCCAGAGCGAGGA
CTTCGCCGTATACTACTGCCAGCAGTACGATACGTGGCCATTCACT
TTTGGACCAGGGACTAAAGTGGATTTTAAGCGCGCCGCCGCTCTCG
ATAACGAAAAGTCAAATGGCACCATAATCCACGTCAAAGGCAAGC
ACCTGTGCCCTTCCCCGCTCTTCCCCGGACCCAGTAAACCATTTTGG
GTGCTGGTTGTTGTGGGGGGCGTGCTGGCCTGCTATAGCCTTTTGGT
CACTGTAGCCTTCATTATTTTTTGGGTCAGATCCAAAAGAAGCCGC
CTGCTCCATAGCGATTACATGAATATGACTCCACGCCGCCCTGGCC
CCACAAGGAAACACTACCAGCCTTACGCACCACCTAGAGATTTCGC
TGCCTATCGGAGCAGGGTGAAGTTTTCCAGATCTGCAGATGCACCA
GCGTATCAGCAGGGCCAGAACCAACTGTATAACGAGCTCAACCTG
GGACGCAGGGAAGAGTATGACGTTTTGGACAAGCGCAGAGGACGG
GACCCTGAGATGGGTGGCAAACCAAGACGAAAAAACCCCCAGGAG
GGTCTCTATAATGAGCTGCAGAAGGATAAGATGGCTGAAGCCTATT
CTGAAATAGGCATGAAAGGAGAGCGGAGAAGGGGAAAAGGGCAC
GACGGTTTGTACCAGGGACTCAGCACTGCTACGAAGGATACTTATG
ACGCTCTCCACATGCAAGCCCTGCCACCTAGG
Clone 20C5.2 CD28T CD3 zeta CAR AA Heavy & Light Chains
(SEQ ID NO. 106)
QVQLVESGGGVVQPGRSLRLSCAASGFTFSSYGMHWVRQAPGKGLE
WVAVISYDGSDKYYVDSVKGRFTISRDNSKNRLYLQMNSLRAEDTAV
YYCARERYSGRDYWGQGTLVTVSSGGGGSGGGGSGGGGSEIVMTQS
PATLSVSPGERATLSCRASQSVSSLLTWYQQKPGQAPRLLIFGASTRAT
GIPARFSGSGSGTGFTLTISSLQSEDFAVYYCQQYDTWPFTFGPGTKVD
FKRAAALDNEKSNGTIIHVKGKHLCPSPLFPGPSKPFWVLVVVGGVLA
CYSLLVTVAFIIFWVRSKRSRLLHSDYMNMTPRRPGPTRKHYQPYAPP
RDFAAYRSRVKFSRSADAPAYQQGQNQLYNELNLGRREEYDVLDKR
RGRDPEMGGKPRRKNPQEGLYNELQKDKMAEAYSEIGMKGERRRGK
GHDGLYQGLSTATKDTYDALHMQALPPR
Clone 20C5.2 CD28 CD3 zeta CAR DNA Heavy & Light Chains
(SEQ ID NO. 107)
ATGGCACTCCCCGTAACTGCTCTGCTGCTGCCGTTGGCATTGCTCCT
GCACGCCGCACGCCCGCAGGTGCAGCTCGTGGAGTCTGGCGGCGG
CGTGGTCCAGCCCGGCCGGTCCCTGCGCCTGTCCTGCGCCGCCAGC
GGGTTTACTTTTTCCTCCTACGGCATGCACTGGGTGCGCCAGGCTCC
CGGCAAGGGCCTCGAGTGGGTCGCCGTGATCTCATACGATGGGTCA
GACAAATACTATGTCGATTCTGTTAAAGGGCGGTTTACCATTTCAA
GAGATAACTCTAAGAATAGGCTGTATTTGCAGATGAACAGCCTGA
GGGCTGAAGATACCGCAGTGTACTATTGCGCTAGGGAGCGGTATA
GTGGCCGCGATTACTGGGGACAGGGTACACTGGTGACCGTGAGCT
CTGGGGGTGGCGGAAGCGGGGGTGGCGGAAGCGGCGGAGGGGGT
AGTGAAATTGTGATGACCCAGTCTCCGGCTACACTTTCAGTCTCCC
CTGGGGAGAGAGCTACACTGTCATGCAGAGCGTCCCAGTCCGTCTC
TTCTCTCCTTACCTGGTATCAGCAGAAGCCCGGCCAGGCTCCTCGA
CTGCTGATCTTCGGTGCCTCCACAAGGGCGACCGGGATTCCAGCCC
GCTTCTCAGGTTCTGGGAGCGGAACTGGTTTCACTTTGACAATCAG
TTCACTGCAGTCAGAGGATTTCGCCGTGTACTACTGCCAGCAATAC
GACACATGGCCATTCACTTTCGGACCCGGTACCAAAGTCGATTTCA
AGAGAGCCGCGGCCATCGAGGTTATGTACCCACCACCATATCTGGA
CAATGAAAAAAGCAATGGAACCATTATCCATGTGAAGGGTAAACA
CCTCTGCCCTAGCCCACTTTTCCCTGGCCCATCAAAGCCCTTCTGGG
TCTTGGTGGTCGTGGGGGGTGTGCTGGCCTGTTACAGCCTTCTGGT
GACGGTTGCTTTCATTATCTTCTGGGTTAGATCCAAAAGAAGCCGC
CTGCTCCATAGCGATTACATGAATATGACTCCACGCCGCCCTGGCC
CCACAAGGAAACACTACCAGCCTTACGCACCACCTAGAGATTTCGC
TGCCTATCGGAGCAGGGTGAAGTTTTCCAGATCTGCAGATGCACCA
GCGTATCAGCAGGGCCAGAACCAACTGTATAACGAGCTCAACCTG
GGACGCAGGGAAGAGTATGACGTTTTGGACAAGCGCAGAGGACGG
GACCCTGAGATGGGTGGCAAACCAAGACGAAAAAACCCCCAGGAG
GGTCTCTATAATGAGCTGCAGAAGGATAAGATGGCTGAAGCCTATT
CTGAAATAGGCATGAAAGGAGAGCGGAGAAGGGGAAAAGGGCAC
GACGGTTTGTACCAGGGACTCAGCACTGCTACGAAGGATACTTATG
ACGCTCTCCACATGCAAGCCCTGCCACCTAGGTAA
Clone 20C5.2 CD28 CD3 zeta CAR AA Heavy & Light Chains
(Signal Peptide in Bold)
(SEQ ID NO. 108)
MALPVTALLLPLALLLHAARPQVQLVESGGGVVQPGRSLRLSCAAS
GFTFSSYGMHWVRQAPGKGLEWVAVISYDGSDKYYVDSVKGRFTISR
DNSKNRLYLQMNSLRAEDTAVYYCARERYSGRDYWGQGTLVTVSSG
GGGSGGGGSGGGGSEIVMTQSPATLSVSPGERATLSCRASQSVSSLLT
WYQQKPGQAPRLLIFGASTRATGIPARFSGSGSGTGFTLTISSLQSEDFA
VYYCQQYDTWPFTFGPGTKVDFKRAAAIEVMYPPPYLDNEKSNGTIIH
VKGKHLCPSPLFPGPSKPFWVLVVVGGVLACYSLLVTVAFIIFWVRSK
RSRLLHSDYMNMTPRRPGPTRKHYQPYAPPRDFAAYRSRVKFSRSAD
APAYQQGQNQLYNELNLGRREEYDVLDKRRGRDPEMGGKPRRKNPQ
EGLYNELQKDKMAEAYSEIGMKGERRRGKGHDGLYQGLSTATKDTY
DALHMQALPPR
Clone 20C5.2 CD28 CD3 zeta CAR DNA Heavy & Light Chains
(SEQ ID NO. 109)
CAGGTGCAGCTCGTGGAGTCTGGCGGCGGCGTGGTCCAGCCCGGC
CGGTCCCTGCGCCTGTCCTGCGCCGCCAGCGGGTTTACTTTTTCCTC
CTACGGCATGCACTGGGTGCGCCAGGCTCCCGGCAAGGGCCTCGA
GTGGGTCGCCGTGATCTCATACGATGGGTCAGACAAATACTATGTC
GATTCTGTTAAAGGGCGGTTTACCATTTCAAGAGATAACTCTAAGA
ATAGGCTGTATTTGCAGATGAACAGCCTGAGGGCTGAAGATACCG
CAGTGTACTATTGCGCTAGGGAGCGGTATAGTGGCCGCGATTACTG
GGGACAGGGTACACTGGTGACCGTGAGCTCTGGGGGTGGCGGAAG
CGGGGGTGGCGGAAGCGGCGGAGGGGGTAGTGAAATTGTGATGAC
CCAGTCTCCGGCTACACTTTCAGTCTCCCCTGGGGAGAGAGCTACA
CTGTCATGCAGAGCGTCCCAGTCCGTCTCTTCTCTCCTTACCTGGTA
TCAGCAGAAGCCCGGCCAGGCTCCTCGACTGCTGATCTTCGGTGCC
TCCACAAGGGCGACCGGGATTCCAGCCCGCTTCTCAGGTTCTGGGA
GCGGAACTGGTTTCACTTTGACAATCAGTTCACTGCAGTCAGAGGA
TTTCGCCGTGTACTACTGCCAGCAATACGACACATGGCCATTCACT
TTCGGACCCGGTACCAAAGTCGATTTCAAGAGAGCCGCGGCCATCG
AGGTTATGTACCCACCACCATATCTGGACAATGAAAAAAGCAATG
GAACCATTATCCATGTGAAGGGTAAACACCTCTGCCCTAGCCCACT
TTTCCCTGGCCCATCAAAGCCCTTCTGGGTCTTGGTGGTCGTGGGG
GGTGTGCTGGCCTGTTACAGCCTTCTGGTGACGGTTGCTTTCATTAT
CTTCTGGGTTAGATCCAAAAGAAGCCGCCTGCTCCATAGCGATTAC
ATGAATATGACTCCACGCCGCCCTGGCCCCACAAGGAAACACTACC
AGCCTTACGCACCACCTAGAGATTTCGCTGCCTATCGGAGCAGGGT
GAAGTTTTCCAGATCTGCAGATGCACCAGCGTATCAGCAGGGCCAG
AACCAACTGTATAACGAGCTCAACCTGGGACGCAGGGAAGAGTAT
GACGTTTTGGACAAGCGCAGAGGACGGGACCCTGAGATGGGTGGC
AAACCAAGACGAAAAAACCCCCAGGAGGGTCTCTATAATGAGCTG
CAGAAGGATAAGATGGCTGAAGCCTATTCTGAAATAGGCATGAAA
GGAGAGCGGAGAAGGGGAAAAGGGCACGACGGTTTGTACCAGGG
ACTCAGCACTGCTACGAAGGATACTTATGACGCTCTCCACATGCAA
GCCCTGCCACCTAGG
Clone 20C5.2 CD28 CD3 zeta CAR AA Heavy & Light Chains
(SEQ ID NO. 110)
QVQLVESGGGVVQPGRSLRLSCAASGFTFSSYGMHWVRQAPGKGLE
WVAVISYDGSDKYYVDSVKGRFTISRDNSKNRLYLQMNSLRAEDTAV
YYCARERYSGRDYWGQGTLVTVSSGGGGSGGGGSGGGGSEIVMTQS
PATLSVSPGERATLSCRASQSVSSLLTWYQQKPGQAPRLLIFGASTRAT
GIPARFSGSGSGTGFTLTISSLQSEDFAVYYCQQYDTWPFTFGPGTKVD
FKRAAAIEVMYPPPYLDNEKSNGTIIHVKGKHLCPSPLFPGPSKPFWVL
VVVGGVLACYSLLVTVAFIIFWVRSKRSRLLHSDYMNMTPRRPGPTR
KHYQPYAPPRDFAAYRSRVKFSRSADAPAYQQGQNQLYNELNLGRRE
EYDVLDKRRGRDPEMGGKPRRKNPQEGLYNELQKDKMAEAYSEIGM
KGERRRGKGHDGLYQGLSTATKDTYDALHMQALPPR
Clone 20C5.2 CD8 CD3 zeta CAR DNA Heavy & Light Chains
(SEQ ID NO. 111)
ATGGCACTCCCCGTAACTGCTCTGCTGCTGCCGTTGGCATTGCTCCT
GCACGCCGCACGCCCGCAGGTGCAGTTGGTTGAATCAGGAGGGGG
TGTGGTGCAACCCGGTCGGTCACTGCGCCTCAGTTGTGCTGCTTCC
GGGTTTACTTTCAGCTCATATGGGATGCACTGGGTACGGCAGGCTC
CAGGTAAAGGCTTGGAATGGGTGGCGGTGATCAGCTATGACGGCT
CTGACAAATATTATGTGGACTCCGTGAAAGGCAGATTCACCATCAG
TCGAGACAACTCAAAGAATAGACTCTACTTGCAGATGAATAGCCTC
CGGGCCGAAGATACTGCAGTCTATTATTGCGCCCGGGAGCGCTACA
GTGGAAGAGACTATTGGGGGCAAGGAACTCTTGTCACAGTCTCATC
TGGCGGCGGCGGCAGCGGTGGGGGCGGATCTGGCGGGGGCGGCAG
CGAAATCGTTATGACTCAGAGTCCTGCCACACTGAGCGTTAGCCCT
GGTGAGAGAGCAACACTTAGCTGCAGAGCTAGTCAGAGTGTTTCC
AGTCTTTTGACATGGTACCAACAGAAGCCCGGTCAAGCTCCACGAC
TGCTCATCTTCGGTGCATCCACCCGCGCAACCGGGATACCCGCCCG
GTTTTCCGGTTCTGGAAGTGGCACAGGATTCACGCTCACCATTTCTT
CTCTGCAGTCTGAAGACTTTGCCGTGTATTACTGCCAGCAGTACGA
TACCTGGCCCTTTACCTTTGGCCCAGGTACTAAAGTGGATTTTAAA
CGAGCTGCTGCACTTTCCAATAGTATTATGTACTTTTCACATTTTGT
GCCCGTGTTCCTGCCTGCGAAGCCTACGACAACCCCAGCCCCTAGG
CCGCCCACACCGGCCCCAACTATTGCCTCCCAGCCATTGTCTCTGA
GACCCGAAGCTTGCAGACCTGCTGCTGGAGGCGCCGTTCACACCCG
AGGATTGGATTTCGCATGTGACATTTACATCTGGGCCCCTTTGGCC
GGAACCTGCGGTGTGCTGCTGCTGTCACTCGTGATTACACTTTACT
GCAACCACCGAAACAGATCCAAAAGAAGCCGCCTGCTCCATAGCG
ATTACATGAATATGACTCCACGCCGCCCTGGCCCCACAAGGAAACA
CTACCAGCCTTACGCACCACCTAGAGATTTCGCTGCCTATCGGAGC
AGGGTGAAGTTTTCCAGATCTGCAGATGCACCAGCGTATCAGCAGG
GCCAGAACCAACTGTATAACGAGCTCAACCTGGGACGCAGGGAAG
AGTATGACGTTTTGGACAAGCGCAGAGGACGGGACCCTGAGATGG
GTGGCAAACCAAGACGAAAAAACCCCCAGGAGGGTCTCTATAATG
AGCTGCAGAAGGATAAGATGGCTGAAGCCTATTCTGAAATAGGCA
TGAAAGGAGAGCGGAGAAGGGGAAAAGGGCACGACGGTTTGTAC
CAGGGACTCAGCACTGCTACGAAGGATACTTATGACGCTCTCCACA
TGCAAGCCCTGCCACCTAGGTAA
Clone 20C5.2 CD8 CD3 zeta CAR AA Heavy & Light Chains
(Signal peptide in Bold)
(SEQ ID NO. 112)
MALPVTALLLPLALLLHAARPQVQLVESGGGVVQPGRSLRLSCAAS
GFTFSSYGMHWVRQAPGKGLEWVAVISYDGSDKYYVDSVKGRFTISR
DNSKNRLYLQMNSLRAEDTAVYYCARERYSGRDYWGGTLVTVSSGG
GGSGGGGSGGGGSEIVMTQSPATLSVSPGERATLSCRASQSVSSLLTW
YQQKPGQAPRLLIFGASTRATGIPARFSGSGSGTGFTLTISSLQSEDFAV
YYCQQYDTWPFTFGPGTKVDFKRAAALSNSIMYFSHFVPVFLPAKPTT
TPAPRPPTPAPTIASQPLSLRPEACRPAAGGAVHTRGLDFACDIYIWAP
LAGTCGVLLLSLVITLYCNHRNRSKRSRLLHSDYMNMTPRRPGPTRK
HYQPYAPPRDFAAYRSRVKFSRSADAPAYQQGQNQLYNELNLGRREE
YDVLDKRRGRDPEMGGKPRRKNPQEGLYNELQKDKMAEAYSEIGMK
GERRRGKGHDGLYQGLSTATKDTYDALHMQALPPR
Clone 20C5.2 CD8 CD3 zeta CAR DNA Heavy & Light Chains
(SEQ ID NO. 113)
CAGGTGCAGTTGGTTGAATCAGGAGGGGGTGTGGTGCAACCCGGT
CGGTCACTGCGCCTCAGTTGTGCTGCTTCCGGGTTTACTTTCAGCTC
ATATGGGATGCACTGGGTACGGCAGGCTCCAGGTAAAGGCTTGGA
ATGGGTGGCGGTGATCAGCTATGACGGCTCTGACAAATATTATGTG
GACTCCGTGAAAGGCAGATTCACCATCAGTCGAGACAACTCAAAG
AATAGACTCTACTTGCAGATGAATAGCCTCCGGGCCGAAGATACTG
CAGTCTATTATTGCGCCCGGGAGCGCTACAGTGGAAGAGACTATTG
GGGGCAAGGAACTCTTGTCACAGTCTCATCTGGCGGCGGCGGCAG
CGGTGGGGGCGGATCTGGCGGGGGCGGCAGCGAAATCGTTATGAC
TCAGAGTCCTGCCACACTGAGCGTTAGCCCTGGTGAGAGAGCAAC
ACTTAGCTGCAGAGCTAGTCAGAGTGTTTCCAGTCTTTTGACATGG
TACCAACAGAAGCCCGGTCAAGCTCCACGACTGCTCATCTTCGGTG
CATCCACCCGCGCAACCGGGATACCCGCCCGGTTTTCCGGTTCTGG
AAGTGGCACAGGATTCACGCTCACCATTTCTTCTCTGCAGTCTGAA
GACTTTGCCGTGTATTACTGCCAGCAGTACGATACCTGGCCCTTTA
CCTTTGGCCCAGGTACTAAAGTGGATTTTAAACGAGCTGCTGCACT
TTCCAATAGTATTATGTACTTTTCACATTTTGTGCCCGTGTTCCTGC
CTGCGAAGCCTACGACAACCCCAGCCCCTAGGCCGCCCACACCGG
CCCCAACTATTGCCTCCCAGCCATTGTCTCTGAGACCCGAAGCTTG
CAGACCTGCTGCTGGAGGCGCCGTTCACACCCGAGGATTGGATTTC
GCATGTGACATTTACATCTGGGCCCCTTTGGCCGGAACCTGCGGTG
TGCTGCTGCTGTCACTCGTGATTACACTTTACTGCAACCACCGAAA
CAGATCCAAAAGAAGCCGCCTGCTCCATAGCGATTACATGAATATG
ACTCCACGCCGCCCTGGCCCCACAAGGAAACACTACCAGCCTTACG
CACCACCTAGAGATTTCGCTGCCTATCGGAGCAGGGTGAAGTTTTC
CAGATCTGCAGATGCACCAGCGTATCAGCAGGGCCAGAACCAACT
GTATAACGAGCTCAACCTGGGACGCAGGGAAGAGTATGACGTTTT
GGACAAGCGCAGAGGACGGGACCCTGAGATGGGTGGCAAACCAA
GACGAAAAAACCCCCAGGAGGGTCTCTATAATGAGCTGCAGAAGG
ATAAGATGGCTGAAGCCTATTCTGAAATAGGCATGAAAGGAGAGC
GGAGAAGGGGAAAAGGGCACGACGGTTTGTACCAGGGACTCAGCA
CTGCTACGAAGGATACTTATGACGCTCTCCACATGCAAGCCCTGCC
ACCTAGG
Clone 20C5.2 CD8 CD3 zeta CAR AA Heavy & Light Chains
(SEQ ID NO. 114)
QVQLVESGGGVVQPGRSLRLSCAASGFTFSSYGMHWVRQAPGKGLE
WVAVISYDGSDKYYVDSVKGRFTISRDNSKNRLYLQMNSLRAEDTAV
YYCARERYSGRDYWGQGTLVTVSSGGGGSGGGGSGGGGSEIVMTQS
PATLSVSPGERATLSCRASQSVSSLLTWYQQKPGQAPRLLIFGASTRAT
GIPARFSGSGSGTGFTLTISSLQSEDFAVYYCQQYDTWPFTFGPGTKVD
FKRAAALSNSIMYFSHFVPVFLPAKPTTTPAPRPPTPAPTIASQPLSLRP
EACRPAAGGAVHTRGLDFACDIYIWAPLAGTCGVLLLSLVITLYCNHR
NRSKRSRLLHSDYMNMTPRRPGPTRKHYQPYAPPRDFAAYRSRVKFS
RSADAPAYQQGQNQLYNELNLGRREEYDVLDKRRGRDPEMGGKPRR
KNPQEGLYNELQKDKMAEAYSEIGMKGERRRGKGHDGLYQGLSTAT
KDTYDALHMQALPPR
Clone 20C5.2 CD28T CD3 zeta CAR DNA Heavy & Light Chains
(SEQ ID NO. 115)
ATGGCACTCCCCGTAACTGCTCTGCTGCTGCCGTTGGCATTGCTCCT
GCACGCCGCACGCCCGGAGATTGTGATGACCCAGTCCCCTGCTACC
CTGTCCGTCAGTCCGGGCGAGAGAGCCACCTTGTCATGCCGGGCCA
GCCAGTCCGTCAGCAGTCTCCTGACTTGGTATCAGCAAAAACCAGG
GCAGGCACCGCGGCTTTTGATTTTTGGTGCAAGCACACGCGCCACT
GGCATTCCAGCTAGGTTTTCTGGAAGTGGATCTGGGACAGGCTTCA
CTCTGACAATCAGTAGCCTGCAGAGTGAGGACTTTGCTGTTTACTA
CTGTCAACAGTACGACACCTGGCCATTCACATTCGGGCCCGGCACC
AAGGTCGACTTCAAGAGGGGCGGTGGAGGTTCAGGTGGTGGCGGG
TCAGGCGGCGGTGGGTCTCAGGTTCAACTGGTGGAATCAGGTGGC
GGCGTTGTCCAACCGGGGCGATCACTTCGACTTTCCTGTGCTGCCT
CAGGCTTTACTTTTTCATCCTATGGGATGCACTGGGTTCGGCAGGCT
CCCGGAAAAGGACTCGAGTGGGTTGCAGTGATCTCTTACGATGGCT
CAGACAAGTATTATGTGGACTCAGTCAAGGGGAGATTCACAATAA
GCCGAGACAACTCCAAAAACCGGCTTTATCTCCAGATGAACAGCCT
TAGAGCGGAAGATACCGCGGTATACTACTGTGCCCGCGAGAGGTA
TTCCGGCAGAGACTACTGGGGACAGGGCACACTGGTCACCGTGAG
TTCTGCCGCAGCGCTCGATAACGAAAAGAGCAACGGAACCATTAT
CCACGTTAAGGGCAAGCACCTGTGCCCCAGTCCCCTCTTCCCAGGA
CCATCTAAACCCTTCTGGGTTCTGGTAGTAGTTGGAGGGGTCCTTG
CATGTTACTCCCTTTTGGTCACCGTCGCCTTCATTATTTTCTGGGTG
AGATCCAAAAGAAGCCGCCTGCTCCATAGCGATTACATGAATATG
ACTCCACGCCGCCCTGGCCCCACAAGGAAACACTACCAGCCTTACG
CACCACCTAGAGATTTCGCTGCCTATCGGAGCAGGGTGAAGTTTTC
CAGATCTGCAGATGCACCAGCGTATCAGCAGGGCCAGAACCAACT
GTATAACGAGCTCAACCTGGGACGCAGGGAAGAGTATGACGTTTT
GGACAAGCGCAGAGGACGGGACCCTGAGATGGGTGGCAAACCAA
GACGAAAAAACCCCCAGGAGGGTCTCTATAATGAGCTGCAGAAGG
ATAAGATGGCTGAAGCCTATTCTGAAATAGGCATGAAAGGAGAGC
GGAGAAGGGGAAAAGGGCACGACGGTTTGTACCAGGGACTCAGCA
CTGCTACGAAGGATACTTATGACGCTCTCCACATGCAAGCCCTGCC
ACCTAGGTAA
Clone 20C5.2 CD28T CD3 zeta CAR AA Heavy & Light Chains
(Signal Peptide in Bold)
(SEQ ID NO. 116)
MALPVTALLLPLALLLHAARPEIVMTQSPATLSVSPGERATLSCRAS
QSVSSLLTWYQQKPGQAPRLLIFGASTRATGIPARFSGSGSGTGFTLTIS
SLQSEDFAVYYCQQYDTWPFTFGPGTKVDFKRGGGGSGGGGSGGGG
SQVQLVESGGGVVQPGRSLRLSCAASGFTFSSYGMHWVRQAPGKGLE
WVAVISYDGSDKYYVDSVKGRFTISRDNSKNRLYLQMNSLRAEDTAV
YYCARERYSGRDYWGQGTLVTVSSAAALDNEKSNGTIIHVKGKHLCP
SPLFPGPSKPFWVLVVVGGVLACYSLLVTVAFIIFWVRSKRSRLLHSD
YMNMTPRRPGPTRKHYQPYAPPRDFAAYRSRVKFSRSADAPAYQQG
QNQLYNELNLGRREEYDVLDKRRGRDPEMGGKPRRKNPQEGLYNEL
QKDKMAEAYSEIGMKGERRRGKGHDGLYQGLSTATKDTYDALHMQ
ALPPR
Clone 20C5.2 CD28T CD3 zeta CAR DNA Heavy & Light Chains
(SEQ ID NO. 117)
GAGATTGTGATGACCCAGTCCCCTGCTACCCTGTCCGTCAGTCCGG
GCGAGAGAGCCACCTTGTCATGCCGGGCCAGCCAGTCCGTCAGCA
GTCTCCTGACTTGGTATCAGCAAAAACCAGGGCAGGCACCGCGGCT
TTTGATTTTTGGTGCAAGCACACGCGCCACTGGCATTCCAGCTAGG
TTTTCTGGAAGTGGATCTGGGACAGGCTTCACTCTGACAATCAGTA
GCCTGCAGAGTGAGGACTTTGCTGTTTACTACTGTCAACAGTACGA
CACCTGGCCATTCACATTCGGGCCCGGCACCAAGGTCGACTTCAAG
AGGGGCGGTGGAGGTTCAGGTGGTGGCGGGTCAGGCGGCGGTGGG
TCTCAGGTTCAACTGGTGGAATCAGGTGGCGGCGTTGTCCAACCGG
GGCGATCACTTCGACTTTCCTGTGCTGCCTCAGGCTTTACTTTTTCA
TCCTATGGGATGCACTGGGTTCGGCAGGCTCCCGGAAAAGGACTCG
AGTGGGTTGCAGTGATCTCTTACGATGGCTCAGACAAGTATTATGT
GGACTCAGTCAAGGGGAGATTCACAATAAGCCGAGACAACTCCAA
AAACCGGCTTTATCTCCAGATGAACAGCCTTAGAGCGGAAGATACC
GCGGTATACTACTGTGCCCGCGAGAGGTATTCCGGCAGAGACTACT
GGGGACAGGGCACACTGGTCACCGTGAGTTCTGCCGCAGCGCTCG
ATAACGAAAAGAGCAACGGAACCATTATCCACGTTAAGGGCAAGC
ACCTGTGCCCCAGTCCCCTCTTCCCAGGACCATCTAAACCCTTCTGG
GTTCTGGTAGTAGTTGGAGGGGTCCTTGCATGTTACTCCCTTTTGGT
CACCGTCGCCTTCATTATTTTCTGGGTGAGATCCAAAAGAAGCCGC
CTGCTCCATAGCGATTACATGAATATGACTCCACGCCGCCCTGGCC
CCACAAGGAAACACTACCAGCCTTACGCACCACCTAGAGATTTCGC
TGCCTATCGGAGCAGGGTGAAGTTTTCCAGATCTGCAGATGCACCA
GCGTATCAGCAGGGCCAGAACCAACTGTATAACGAGCTCAACCTG
GGACGCAGGGAAGAGTATGACGTTTTGGACAAGCGCAGAGGACGG
GACCCTGAGATGGGTGGCAAACCAAGACGAAAAAACCCCCAGGAG
GGTCTCTATAATGAGCTGCAGAAGGATAAGATGGCTGAAGCCTATT
CTGAAATAGGCATGAAAGGAGAGCGGAGAAGGGGAAAAGGGCAC
GACGGTTTGTACCAGGGACTCAGCACTGCTACGAAGGATACTTATG
ACGCTCTCCACATGCAAGCCCTGCCACCTAGG
Clone 20C5.2 CD28T CD3 zeta CAR AA Heavy & Light Chains
(SEQ ID NO. 118)
EIVMTQSPATLSVSPGERATLSCRASQSVSSLLTWYQQKPGQAPRLLIF
GASTRATGIPARFSGSGSGTGFTLTISSLQSEDFAVYYCQQYDTWPFTF
GPGTKVDFKRGGGGSGGGGSGGGGSQVQLVESGGGVVQPGRSLRLS
CAASGFTFSSYGMHWVRQAPGKGLEWVAVISYDGSDKYYVDSVKGR
FTISRDNSKNRLYLQMNSLRAEDTAVYYCARERYSGRDYWGQGTLV
TVSSAAALDNEKSNGTIIHVKGKHLCPSPLFPGPSKPFWVLVVVGGVL
ACYSLLVTVAFIIFWVRSKRSRLLHSDYMNMTPRRPGPTRKHYQPYAP
PRDFAAYRSRVKFSRSADAPAYQQGQNQLYNELNLGRREEYDVLDK
RRGRDPEMGGKPRRKNPQEGLYNELQKDKMAEAYSEIGMKGERRRG
KGHDGLYQGLSTATKDTYDALHMQALPPR
Clone 20C5.2 CD28 CD3 zeta CAR DNA Heavy & Light Chains
(SEQ ID NO. 119)
ATGGCACTCCCCGTAACTGCTCTGCTGCTGCCGTTGGCATTGCTCCT
GCACGCCGCACGCCCGGAGATCGTCATGACACAGAGTCCAGCTAC
CCTGAGCGTGTCCCCTGGAGAGAGAGCCACCCTGTCCTGTAGGGCT
AGTCAGAGTGTGTCCAGCCTCCTCACCTGGTATCAACAGAAGCCTG
GTCAAGCTCCCCGGCTGCTTATCTTCGGGGCCAGCACGCGAGCCAC
AGGCATCCCGGCCAGATTCTCTGGCTCTGGCAGTGGCACCGGGTTC
ACTCTCACGATCTCATCCCTGCAGTCAGAGGATTTCGCTGTGTATTA
CTGTCAGCAGTACGATACATGGCCCTTCACCTTCGGCCCGGGCACA
AAAGTAGATTTCAAGCGCGGCGGCGGGGGTAGTGGGGGCGGGGGA
TCAGGAGGAGGGGGCTCCCAAGTACAGCTGGTTGAGAGCGGCGGC
GGGGTGGTTCAGCCCGGGCGCAGCCTCAGGCTGAGTTGCGCAGCA
TCAGGATTCACATTCAGTTCTTATGGAATGCATTGGGTCAGACAGG
CTCCCGGGAAGGGCCTTGAATGGGTGGCAGTCATTAGCTACGACG
GAAGCGATAAGTACTATGTGGACTCAGTTAAAGGGAGATTTACTAT
CAGCCGCGACAATTCCAAAAACAGATTGTATTTGCAGATGAACTCC
CTCAGGGCGGAGGACACTGCTGTATATTACTGCGCACGAGAGAGA
TACTCCGGCCGAGACTATTGGGGCCAAGGAACATTGGTAACTGTGA
GCTCCGCCGCAGCTATTGAGGTCATGTACCCCCCACCTTATCTCGA
TAATGAGAAGAGTAATGGGACTATAATTCACGTAAAGGGCAAACA
CCTGTGCCCTTCCCCGCTGTTTCCAGGTCCAAGTAAGCCGTTCTGGG
TCCTGGTTGTGGTGGGAGGGGTGCTGGCCTGCTATTCTCTGTTGGTT
ACCGTGGCCTTTATCATTTTCTGGGTGAGATCCAAAAGAAGCCGCC
TGCTCCATAGCGATTACATGAATATGACTCCACGCCGCCCTGGCCC
CACAAGGAAACACTACCAGCCTTACGCACCACCTAGAGATTTCGCT
GCCTATCGGAGCAGGGTGAAGTTTTCCAGATCTGCAGATGCACCAG
CGTATCAGCAGGGCCAGAACCAACTGTATAACGAGCTCAACCTGG
GACGCAGGGAAGAGTATGACGTTTTGGACAAGCGCAGAGGACGGG
ACCCTGAGATGGGTGGCAAACCAAGACGAAAAAACCCCCAGGAGG
GTCTCTATAATGAGCTGCAGAAGGATAAGATGGCTGAAGCCTATTC
TGAAATAGGCATGAAAGGAGAGCGGAGAAGGGGAAAAGGGCACG
ACGGTTTGTACCAGGGACTCAGCACTGCTACGAAGGATACTTATGA
CGCTCTCCACATGCAAGCCCTGCCACCTAGGTAA
Clone 20C5.2 CD28 CD3 zeta CAR AA Heavy & Light Chains
(Signal Peptide in Bold)
(SEQ ID NO. 120)
MALPVTALLLPLALLLHAARPEIVMTQSPATLSVSPGERATLSCRAS
QSVSSLLTWYQQKPGQAPRLLIFGASTRATGIPARFSGSGSGTGFTLTIS
SLQSEDFAVYYCQQYDTWPFTFGPGTKVDFKRGGGGSGGGGSGGGG
SQVQLVESGGGVVQPGRSLRLSCAASGFTFSSYGMHWVRQAPGKGLE
WVAVISYDGSDKYYVDSVKGRFTISRDNSKNRLYLQMNSLRAEDTAV
YYCARERYSGRDYWGQGTLVTVSSAAAIEVMYPPPYLDNEKSNGTII
HVKGKHLCPSPLFPGPSKPFWVLVVVGGVLACYSLLVTVAFIIFWVRS
KRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNP
QEGLYNELQKDKMAEAYSEIGMKGERRRGKGHDGLYQGLSTATKD
TYDALHMQALPPR
Clone 20C5.2 CD28 CD3 zeta CAR DNA Heavy & Light Chains
(SEQ ID NO. 121)
GAGATCGTCATGACACAGAGTCCAGCTACCCTGAGCGTGTCCCCTG
GAGAGAGAGCCACCCTGTCCTGTAGGGCTAGTCAGAGTGTGTCCA
GCCTCCTCACCTGGTATCAACAGAAGCCTGGTCAAGCTCCCCGGCT
GCTTATCTTCGGGGCCAGCACGCGAGCCACAGGCATCCCGGCCAG
ATTCTCTGGCTCTGGCAGTGGCACCGGGTTCACTCTCACGATCTCAT
CCCTGCAGTCAGAGGATTTCGCTGTGTATTACTGTCAGCAGTACGA
TACATGGCCCTTCACCTTCGGCCCGGGCACAAAAGTAGATTTCAAG
CGCGGCGGCGGGGGTAGTGGGGGCGGGGGATCAGGAGGAGGGGG
CTCCCAAGTACAGCTGGTTGAGAGCGGCGGCGGGGTGGTTCAGCC
CGGGCGCAGCCTCAGGCTGAGTTGCGCAGCATCAGGATTCACATTC
AGTTCTTATGGAATGCATTGGGTCAGACAGGCTCCCGGGAAGGGCC
TTGAATGGGTGGCAGTCATTAGCTACGACGGAAGCGATAAGTACT
ATGTGGACTCAGTTAAAGGGAGATTTACTATCAGCCGCGACAATTC
CAAAAACAGATTGTATTTGCAGATGAACTCCCTCAGGGCGGAGGA
CACTGCTGTATATTACTGCGCACGAGAGAGATACTCCGGCCGAGAC
TATTGGGGCCAAGGAACATTGGTAACTGTGAGCTCCGCCGCAGCTA
TTGAGGTCATGTACCCCCCACCTTATCTCGATAATGAGAAGAGTAA
TGGGACTATAATTCACGTAAAGGGCAAACACCTGTGCCCTTCCCCG
CTGTTTCCAGGTCCAAGTAAGCCGTTCTGGGTCCTGGTTGTGGTGG
GAGGGGTGCTGGCCTGCTATTCTCTGTTGGTTACCGTGGCCTTTATC
ATTTTCTGGGTGAGATCCAAAAGAAGCCGCCTGCTCCATAGCGATT
ACATGAATATGACTCCACGCCGCCCTGGCCCCACAAGGAAACACT
ACCAGCCTTACGCACCACCTAGAGATTTCGCTGCCTATCGGAGCAG
GGTGAAGTTTTCCAGATCTGCAGATGCACCAGCGTATCAGCAGGGC
CAGAACCAACTGTATAACGAGCTCAACCTGGGACGCAGGGAAGAG
TATGACGTTTTGGACAAGCGCAGAGGACGGGACCCTGAGATGGGT
GGCAAACCAAGACGAAAAAACCCCCAGGAGGGTCTCTATAATGAG
CTGCAGAAGGATAAGATGGCTGAAGCCTATTCTGAAATAGGCATG
AAAGGAGAGCGGAGAAGGGGAAAAGGGCACGACGGTTTGTACCA
GGGACTCAGCACTGCTACGAAGGATACTTATGACGCTCTCCACATG
CAAGCCCTGCCACCTAGG
Clone 20C5.2 CD28 CD3 zeta CAR AA Heavy & Light Chains
(SEQ ID NO. 122)
EIVMTQSPATLSVSPGERATLSCRASQSVSSLLTWYQQKPGQAPRLLIF
GASTRATGIPARFSGSGSGTGFTLTISSLQSEDFAVYYCQQYDTWPFTF
GPGTKVDFKRGGGGSGGGGSGGGGSQVQLVESGGGVVQPGRSLRLS
CAASGFTFSSYGMHWVRQAPGKGLEWVAVISYDGSDKYYVDSVKGR
FTISRDNSKNRLYLQMNSLRAEDTAVYYCARERYSGRDYWGQGTLV
TVSSAAAIEVMYPPPYLDNEKSNGTIIHVKGKHLCPSPLFPGPSKPFWV
LVVVGGVLACYSLLVTVAFIIFWVRSKRSRLLHSDYMNMTPRRPGPTR
KHYQPYAPPRDFAAYRSRVKFSRSADAPAYQQGQNQLYNELNLGRRE
EYDVLDKRRGRDPEMGGKPRRKNPQEGLYNELQKDKMAEAYSEIGM
KGERRRGKGHDGLYQGLSTATKDTYDALHMQALPPR
Clone 20C5.2 CD8 CD3 zeta CAR DNA Heavy & Light Chains
(SEQ ID NO. 123)
ATGGCACTCCCCGTAACTGCTCTGCTGCTGCCGTTGGCATTGCTCCT
GCACGCCGCACGCCCGGAAATAGTGATGACTCAGTCCCCGGCCAC
CCTCAGCGTGTCCCCCGGGGAGCGAGCGACCCTGTCATGCAGGGCT
TCCCAGAGTGTCAGCTCCCTGCTCACTTGGTATCAGCAAAAGCCGG
GGCAGGCTCCCCGCCTCCTCATCTTCGGGGCATCAACTAGGGCCAC
CGGCATTCCTGCAAGATTTTCCGGGTCTGGCAGCGGCACCGGCTTC
ACCCTTACCATTAGCTCTCTGCAGTCTGAGGACTTCGCCGTTTACTA
TTGTCAGCAGTATGATACTTGGCCCTTTACCTTCGGTCCCGGAACTA
AGGTGGACTTCAAGCGCGGGGGGGGTGGATCTGGAGGTGGTGGCT
CCGGGGGCGGTGGAAGCCAGGTCCAGTTGGTTGAGAGCGGCGGCG
GAGTGGTGCAGCCCGGGAGGTCCTTGCGGCTGAGCTGTGCAGCCTC
CGGTTTTACTTTTTCTAGCTATGGAATGCATTGGGTAAGACAGGCTC
CCGGAAAAGGCCTCGAGTGGGTGGCGGTCATTAGCTATGATGGAT
CTGATAAATACTATGTGGACTCAGTTAAGGGGCGCTTCACAATCTC
AAGAGACAATAGCAAAAATAGACTGTACCTGCAGATGAATAGTCT
GCGCGCCGAGGACACTGCCGTGTACTACTGCGCCCGCGAGAGATA
CAGCGGACGGGATTACTGGGGCCAGGGTACCCTCGTAACGGTGTC
CTCCGCTGCCGCCCTTAGCAACAGCATTATGTACTTTTCTCATTTCG
TGCCAGTCTTTCTCCCAGCAAAGCCCACCACTACCCCGGCCCCCAG
GCCGCCTACTCCTGCCCCCACTATCGCGTCTCAGCCTCTCTCCTTGC
GGCCCGAGGCCTGCCGGCCAGCCGCAGGGGGCGCCGTACATACTC
GGGGTTTGGATTTCGCTTGCGACATATATATTTGGGCCCCCCTCGCC
GGCACATGTGGAGTGCTGCTCCTGAGTCTCGTTATAACCCTCTATT
GCAACCATAGAAACAGATCCAAAAGAAGCCGCCTGCTCCATAGCG
ATTACATGAATATGACTCCACGCCGCCCTGGCCCCACAAGGAAACA
CTACCAGCCTTACGCACCACCTAGAGATTTCGCTGCCTATCGGAGC
AGGGTGAAGTTTTCCAGATCTGCAGATGCACCAGCGTATCAGCAGG
GCCAGAACCAACTGTATAACGAGCTCAACCTGGGACGCAGGGAAG
AGTATGACGTTTTGGACAAGCGCAGAGGACGGGACCCTGAGATGG
GTGGCAAACCAAGACGAAAAAACCCCCAGGAGGGTCTCTATAATG
AGCTGCAGAAGGATAAGATGGCTGAAGCCTATTCTGAAATAGGCA
TGAAAGGAGAGCGGAGAAGGGGAAAAGGGCACGACGGTTTGTAC
CAGGGACTCAGCACTGCTACGAAGGATACTTATGACGCTCTCCACA
TGCAAGCCCTGCCACCTAGGTAA
Clone 20C5.2 CD8 CD3 zeta CAR AA Heavy & Light Chains
(Signal Peptide in Bold)
(SEQ ID NO. 124)
MALPVTALLLPLALLLHAARPEIVMTQSPATLSVSPGERATLSCRAS
QSVSSLLTWYQQKPGQAPRLLIFGASTRATGIPARFSGSGSGTGFTLTIS
SLQSEDFAVYYCQQYDTWPFTFGPGTKVDFKRGGGGSGGGGSGGGG
SQVQLVESGGGVVQPGRSLRLSCAASGFTFSSYGMHWVRQAPGKGLE
WVAVISYDGSDKYYVDSVKGRFTISRDNSKNRLYLQMNSLRAEDTAV
YYCARERYSGRDYWGQGTLVTVSSAAALSNSIMYFSHFVPVFLPAKP
TTTPAPRPPTPAPTIASQPLSLRPEACRPAAGGAVHTRGLDFACDIYIW
APLAGTCGVLLLSLVITLYCNHRNRSKRSRLLHSDYMNMTPRRPGPTR
KHYQPYAPPRDFAAYRSRVKFSRSADAPAYQQGQNQLYNELNLGRRE
EYDVLDKRRGRDPEMGGKPRRKNPQEGLYNELQKDKMAEAYSEIGM
KGERRRGKGHDGLYQGLSTATKDTYDALHMQALPPR
Clone 20C5.2 CD8 CD3 zeta CAR DNA Heavy & Light Chains
(SEQ ID NO. 125)
GAAATAGTGATGACTCAGTCCCCGGCCACCCTCAGCGTGTCCCCCG
GGGAGCGAGCGACCCTGTCATGCAGGGCTTCCCAGAGTGTCAGCTC
CCTGCTCACTTGGTATCAGCAAAAGCCGGGGCAGGCTCCCCGCCTC
CTCATCTTCGGGGCATCAACTAGGGCCACCGGCATTCCTGCAAGAT
TTTCCGGGTCTGGCAGCGGCACCGGCTTCACCCTTACCATTAGCTCT
CTGCAGTCTGAGGACTTCGCCGTTTACTATTGTCAGCAGTATGATA
CTTGGCCCTTTACCTTCGGTCCCGGAACTAAGGTGGACTTCAAGCG
CGGGGGGGGTGGATCTGGAGGTGGTGGCTCCGGGGGCGGTGGAAG
CCAGGTCCAGTTGGTTGAGAGCGGCGGCGGAGTGGTGCAGCCCGG
GAGGTCCTTGCGGCTGAGCTGTGCAGCCTCCGGTTTTACTTTTTCTA
GCTATGGAATGCATTGGGTAAGACAGGCTCCCGGAAAAGGCCTCG
AGTGGGTGGCGGTCATTAGCTATGATGGATCTGATAAATACTATGT
GGACTCAGTTAAGGGGCGCTTCACAATCTCAAGAGACAATAGCAA
AAATAGACTGTACCTGCAGATGAATAGTCTGCGCGCCGAGGACAC
TGCCGTGTACTACTGCGCCCGCGAGAGATACAGCGGACGGGATTA
CTGGGGCCAGGGTACCCTCGTAACGGTGTCCTCCGCTGCCGCCCTT
AGCAACAGCATTATGTACTTTTCTCATTTCGTGCCAGTCTTTCTCCC
AGCAAAGCCCACCACTACCCCGGCCCCCAGGCCGCCTACTCCTGCC
CCCACTATCGCGTCTCAGCCTCTCTCCTTGCGGCCCGAGGCCTGCC
GGCCAGCCGCAGGGGGCGCCGTACATACTCGGGGTTTGGATTTCGC
TTGCGACATATATATTTGGGCCCCCCTCGCCGGCACATGTGGAGTG
CTGCTCCTGAGTCTCGTTATAACCCTCTATTGCAACCATAGAAACA
GATCCAAAAGAAGCCGCCTGCTCCATAGCGATTACATGAATATGAC
TCCACGCCGCCCTGGCCCCACAAGGAAACACTACCAGCCTTACGCA
CCACCTAGAGATTTCGCTGCCTATCGGAGCAGGGTGAAGTTTTCCA
GATCTGCAGATGCACCAGCGTATCAGCAGGGCCAGAACCAACTGT
ATAACGAGCTCAACCTGGGACGCAGGGAAGAGTATGACGTTTTGG
ACAAGCGCAGAGGACGGGACCCTGAGATGGGTGGCAAACCAAGAC
GAAAAAACCCCCAGGAGGGTCTCTATAATGAGCTGCAGAAGGATA
AGATGGCTGAAGCCTATTCTGAAATAGGCATGAAAGGAGAGCGGA
GAAGGGGAAAAGGGCACGACGGTTTGTACCAGGGACTCAGCACTG
CTACGAAGGATACTTATGACGCTCTCCACATGCAAGCCCTGCCACC
TAGG
Clone 20C5.2 CD8 CD3 zeta CAR AA Heavy & Light Chains
(SEQ ID NO. 126)
EIVMTQSPATLSVSPGERATLSCRASQSVSSLLTWYQQKPGQAPRLLIF
GASTRATGIPARFSGSGSGTGFTLTISSLQSEDFAVYYCQQYDTWPFTF
GPGTKVDFKRGGGGSGGGGSGGGGSQVQLVESGGGVVQPGRSLRLS
CAASGFTFSSYGMHWVRQAPGKGLEWVAVISYDGSDKYYVDSVKGR
FTISRDNSKNRLYLQMNSLRAEDTAVYYCARERYSGRDYWGQGTLV
TVSSAAALSNSIMYFSHFVPVFLPAKPTTTPAPRPPTPAPTIASQPLSLRP
EACRPAAGGAVHTRGLDFACDIYIWAPLAGTCGVLLLSLVITLYCNHR
NRSKRSRLLHSDYMNMTPRRPGPTRKHYQPYAPPRDFAAYRSRVKFS
RSADAPAYQQGQNQLYNELNLGRREEYDVLDKRRGRDPEMGGKPRR
KNPQEGLYNELQKDKMAEAYSEIGMKGERRRGKGHDGLYQGLSTAT
KDTYDALHMQALPPR
CAR Signal Peptide DNA
(SEQ ID NO. 127)
ATGGCACTCCCCGTAACTGCTCTGCTGCTGCCGTTGGCATTGCTCCT
GCACGCCGCACGCCCG
(SEQ ID NO. 128)
CAR Signal Peptide: MALPVTALLLPLALLLHAARP
scFv G4S linker DNA
(SEQ ID NO. 129)
GGCGGTGGAGGCTCCGGAGGGGGGGGCTCTGGCGGAGGGGGCTCC
(SEQ ID NO. 130)
scFv G4s linker: GGGGSGGGGSGGGGS
scFv Whitlow linker DNA
(SEQ ID NO. 131)
GGGTCTACATCCGGCTCCGGGAAGCCCGGAAGTGGCGAAGGTAGT
ACAAAGGGG
(SEQ ID NO. 132)
scFv Whitlow linker: GSTSGSGKPGSGEGSTKG
CD28 AA Extracellular Domain
(SEQ ID NO. 133)
MLRLLLALNLFPSIQVTGNKILVKQSPMLVAYDNAVNLSCKYSYNLFS
REFRASLHKGLDSAVEVCVVYGNYSQQLQVYSKTGFNCDGKLGNES
VTFYLQNLYVNQTDIYFCKIEVMYPPPYLDNEKSNGTIIHVKGKHLCPS
PLFPGPSKP
(SEQ ID NO: 134)
GX₂X₃X₄X₅X₆X₇X₈X₉
(SEQ ID NO: 135)
X₁X₂X₃X₄X₅X₆
(SEQ ID NO: 136)
X₁X₂X₃X₄X₅X₆X₇X₈X₉X₁₀X₁₁X₁₂DY
(SEQ ID NO: 137)
X₁ASQX₅X₆X₇X₈X₉LX₁₁
(SEQ ID NO: 138)
X₁ASX₄X₅X₆X₇
(SEQ ID NO: 139)
QQX₃X₄X₅X₆PX₈T
4-1BB Nucleic Acid Sequence (intracellular domain)
(SEQ ID NO. 140)
AAGCGCGGCAGGAAGAAGCTCCTCTACATTTTTAAGCAGCCTTTTA
TGAGGCCCGTACAGACAACACAGGAGGAAGATGGCTGTAGCTGCA
GATTTCCCGAGGAGGAGGAAGGTGGGTGCGAGCTG
4-1BB AA (intracellular domain)
(SEQ ID NO. 141)
KRGRKKLLYIFKQPFMRPVQTTQEEDGCSCRFPEEEEGGCEL
OX40 AA
(SEQ ID NO. 142)
RRDQRLPPDAHKPPGGGSFRTPIQEEQADAHSTLAKI
Leader Sequence AA
(SEQ ID NO: 143)
MALPVTALLLPLALLLHAARP
(SEQ ID NO. 144)
Additional G4S linker: GGGGSGGGGSGGGGSGGGGS
CD3 zeta variant AA
(SEQ ID NO. 145)
RVKFSRSADAPAYKQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
Axi Cel (KTE-C19) DNA
(SEQ ID NO. 146)
Atgcttctcctggtgacaagccttctgctctgtgagttaccacacccagcattcctcctgatcccagacatccagatgacacagactaca
tcctccctgtctgcctctctgggagacagagtcaccatcagttgcagggcaagtcaggacattagtaaatatttaaattggtatcagcaga
aaccagatggaactgttaaactcctgatctaccatacatcaagattacactcaggagtcccatcaaggttcagtggcagtgggtctggaa
cagattattctctcaccattagcaacctggagcaagaagatattgccacttacttttgccaacagggtaatacgcttccgtacacgttcgg
aggggggactaagttggaaataacaggctccacctctggatccggcaagcccggatctggcgagggatccaccaagggcgaggtg
aaactgcaggagtcaggacctggcctggtggcgccctcacagagcctgtccgtcacatgcactgtctcaggggtctcattacccgact
atggtgtaagctggattcgccagcctccacgaaagggtctggagtggctgggagtaatatggggtagtgaaaccacatactataattca
gctctcaaatccagactgaccatcatcaaggacaactccaagagccaagttttcttaaaaatgaacagtctgcaaactgatgacacagc
catttactactgtgccaaacattattactacggtggtagctatgctatggactactggggtcaaggaacctcagtcaccgtctcctcagcg
gccgcaattgaagttatgtatcctcctccttacctagacaatgagaagagcaatggaaccattatccatgtgaaagggaaacacctttgtc
caagtcccctatttcccggaccttctaagcccttttgggtgctggtggtggttgggggagtcctggcttgctatagcttgctagtaacagtg
gcctttattattttctgggtgaggagtaagaggagcaggctcctgcacagtgactacatgaacatgactccccgccgccccgggccca
cccgcaagcattaccagccctatgccccaccacgcgacttcgcagcctatcgctccagagtgaagttcagcaggagcgcagacgcc
cccgcgtaccagcagggccagaaccagctctataacgagctcaatctaggacgaagagaggagtacgatgttttggacaagagacg
tggccgggaccctgagatggggggaaagccgagaaggaagaaccctcaggaaggcctgtacaatgaactgcagaaagataagat
ggcggaggcctacagtgagattgggatgaaaggcgagcgccggaggggcaaggggcacgatggcctttaccagggtctcagtac
agccaccaaggacacctacgacgcccttcacatgcaggccctgccccctcgc
Axi Cel (KTE-C19) AA
(SEQ ID NO. 147)
MLLLVTSLLLCELPHPAFLLIPDIQMTQTTSSLSASLGDRVTISCRASQDISKYLNWYQ
QKPDGTVKLLIYHTSRLHSGVPSRFSGSGSGTDYSLTISNLEQEDIATYFCQQGNTLPY

TFGGGTKLEITGSTSGSGKPGSGEGSTKGEVKLQESGPGLVAPSQSLSVTCTVSGVSL

PDYGVSWIRQPPRKGLEWLGVIWGSETTYYNSALKSRLTIIKDNSKSQVFLKMNSLQ
TDDTAIYYCAKHYYYGGSYAMDYWGQGTSVTVSSAAAIEVMYPPPYLDNEKSNGTI
IHVKGKHLCPSPLFPGPSKPFWVLVVVGGVLACYSLLVTVAFIIFWVRSKRSRLLHSD
YMNMTPRRPGPTRKHYQPYAPPRDFAAYRSRVKFSRSADAPAYQQGQNQLYNELN
LGRREEYDVLDKRRGRDPEMGGKPRRKNPQEGLYNELQKDKMAEAYSEIGMKGER
RRGKGHDGLYQGLSTATKDTYDALHMQALPPR
Humanized anti-CD19 CAR DNA
(SEQ ID NO. 148)
ATGGCACTCCCCGTAACTGCTCTGCTGCTGCCGTTGGCATTGCTCCTGCACGCCG
CACGCCCGGATATTCAAATGACCCAGTCCCCGTCCTCCCTGAGTGCCTCCGTCGG
TGACCGTGTTACGATTACCTGCCGTGCGAGCCAAGACATCTCTAAATACCTGAAC
TGGTATCAGCAAAAACCGGATCAGGCACCGAAACTGCTGATCAAACATACCTCA
CGTCTGCACTCGGGTGTGCCGAGCCGCTTTAGTGGTTCCGGCTCAGGTACCGATT
ACACCCTGACGATCAGCTCTCTGCAGCCGGAAGACTTTGCCACGTATTACTGCCA
GCAAGGTAATACCCTGCCGTATACGTTCGGCCAAGGTACCAAACTGGAAATCAA
AGGCTCGACGAGCGGCTCTGGTAAACCGGGCTCTGGTGAAGGCAGTACCAAAGG
TGAAGTGCAGCTGGTTGAAAGCGGTGGTGGTCTGGTTCAACCGGGTCGTTCCCTG
CGTCTGTCATGTACGGCGAGTGGTGTCTCCCTGCCGGACTATGGCGTGTCCTGGA
TTCGTCAGCCGCCGGGTAAAGGCCTGGAATGGATTGGTGTCATCTGGGGCAGTG
AAACCACGTATTACAACTCGGCCCTGAAAAGCCGTTTCACCATCTCTCGCGATAA
CAGTAAAAATACGCTGTACCTGCAGATGAATAGCCTGCGCGCGGAAGACACCGC
CGTTTACTACTGCGCAAAACATTACTACTACGGTGGCAGCTATGCTATGGATTAC
TGGGGTCAAGGCACGCTGGTCACCGTTTCGTCAGCCGCTGCCCTTGATAATGAAA
AGTCAAACGGAACAATCATTCACGTGAAGGGCAAGCACCTCTGTCCGTCACCCTT
GTTCCCTGGTCCATCCAAGCCATTCTGGGTGTTGGTCGTAGTGGGTGGAGTCCTC
GCTTGTTACTCTCTGCTCGTCACCGTGGCTTTTATAATCTTCTGGGTTAGATCCAA
AAGAAGCCGCCTGCTCCATAGCGATTACATGAATATGACTCCACGCCGCCCTGGC
CCCACAAGGAAACACTACCAGCCTTACGCACCACCTAGAGATTTCGCTGCCTATC
GGAGCAGGGTGAAGTTTTCCAGATCTGCAGATGCACCAGCGTATCAGCAGGGCC
AGAACCAACTGTATAACGAGCTCAACCTGGGACGCAGGGAAGAGTATGACGTTT
TGGACAAGCGCAGAGGACGGGACCCTGAGATGGGTGGCAAACCAAGACGAAAA
AACCCCCAGGAGGGTCTCTATAATGAGCTGCAGAAGGATAAGATGGCTGAAGCC
TATTCTGAAATAGGCATGAAAGGAGAGCGGAGAAGGGGAAAAGGGCACGACGG
TTTGTACCAGGGACTCAGCACTGCTACGAAGGATACTTATGACGCTCTCCACATG
CAAGCCCTGCCAcctagg
Humanized anti-CD19 CAR AA
(SEQ ID NO. 149)
MALPVTALLLPLALLLHAARPDIQMTQSPSSLSASVGDRVTITCRASQDISKYLNWY
QQKPDQAPKLLIKHTSRLHSGVPSRFSGSGSGTDYTLTISSLQPEDFATYYCQQGNTL
PYTFGQGTKLEIKGSTSGSGKPGSGEGSTKGEVQLVESGGGLVQPGRSLRLSCTASG
VSLPDYGVSWIRQPPGKGLEWIGVIWGSETTYYNSALKSRFTISRDNSKNTLYLQMN
SLRAEDTAVYYCAKHYYYGGSYAMDYWGQGTLVTVSSAAALDNEKSNGTIIHVKG
KHLCPSPLFPGPSKPFWVLVVVGGVLACYSLLVTVAFIIFWVRSKRSRLLHSDYMNM
TPRRPGPTRKHYQPYAPPRDFAAYRSRVKFSRSADAPAYQQGQNQLYNELNLGRRE
EYDVLDKRRGRDPEMGGKPRRKNPQEGLYNELQKDKMAEAYSEIGMKGERRRGK
GHDGLYQGLSTATKDTYDALHMQALPPR
Fully human anti-CD19 CAR DNA
(SEQ ID NO. 150)
Atggccctgcctgtgacagctctgctgctgcccctggccctgctgctgcatgccgccagacctgagatcgtgctgacccagtctcccg
gcaccctgtctctcagcccaggagagagagccaccctgagctgcagagccagccagagcgtgtccagcagctacctggcctggtat
cagcagaagcccggacaggcccccagactgctgatctacggcgccagctctagagccaccggcatccccgacagattcagcggca
gcggcagtggcaccgacttcaccctgaccatcagcagactggaacccgaggacttcgccgtgtactactgccagcagtacggcagc
agccggttcaccttcggccctggcaccaaggtggacatcaagggcagcacctccggcagcggcaagcctggctctggcgagggct
ctaccaagggccaggtgcagctggtgcagtctggcgccgaagtgaagaaacccggctctagcgtgaaggtgtcctgcaaggacag
cggcggcaccttcagcagctacgccatcagctgggtgcgccaggccccaggacaggggctggaatggatgggcggcatcatcccc
atcttcggcaccaccaactacgcccagcagttccagggcagagtgaccatcaccgccgacgagagcaccagcaccgcctacatgg
aactgagcagcctgcggagcgaggacacagccgtgtattactgtgcccgcgaggccgtggccgccgactggctggatccttgggg
acagggcaccctggtgacagtgtccagcttcgtgcccgtgttcctgcccgccaagcctaccaccacccctgcccctagacctcccacc
ccagccccaacaatcgccagccagcctctgtccctgcggcccgaagcctgtagacctgctgccggcggagccgtgcacaccagag
gcctggacttcgcctgcgatatctacatctgggcccctctggccggcacctgtggcgtgctgctgctgagcctggtgatcaccctgtact
gcaaccaccggaacagaagcaagcggagccggctgctgcacagcgactacatgaacatgaccccaagacggcctggccccaccc
ggaagcactaccagccttacgcccctcccagagacttcgccgcctaccggtccagagtgaagttcagcagatccgccgacgcccct
gcctaccagcagggacagaaccagctgtacaacgagctgaacctgggcagacgggaagagtacgacgtgctggacaagcggaga
ggccgggaccccgagatgggcggaaagcccagacggaagaacccccaggaaggcctgtataacgaactgcagaaagacaagat
ggccgaggcctacagcgagatcggcatgaagggcgagcggaggcgcggcaagggccacgatggcctgtaccagggcctgagc
accgccaccaaggacacctacgacgccctgcacatgcaggccctgccccccaga
Fully human anti-CD19 CAR AA
(SEQ ID NO. 151)
MALPVTALLLPLALLLHAARPEIVLTQSPGTLSLSPGERATLSCRASQSVSSSYLAWY
QQKPGQAPRLLIYGASSRATGIPDRFSGSGSGTDFTLTISRLEPEDFAVYYCQQYGSSR
FTFGPGTKVDIKGSTSGSGKPGSGEGSTKGQVQLVQSGAEVKKPGSSVKVSCKDSGG
TFSSYAISWVRQAPGQGLEWMGGIIPIFGTTNYAQQFQGRVTITADESTSTAYMELSS
LRSEDTAVYYCAREAVAADWLDPWGQGTLVTVSSFVPVFLPAKPTTTPAPRPPTPAP
TIASQPLSLRPEACRPAAGGAVHTRGLDFACDIYIWAPLAGTCGVLLLSLVITLYCNH
RNRSKRSRLLHSDYMNMTPRRPGPTRKHYQPYAPPRDFAAYRSRVKFSRSADAPAY
QQGQNQLYNELNLGRREEYDVLDKRRGRDPEMGGKPRRKNPQEGLYNELQKDKM
AEAYSEIGMKGERRRGKGHDGLYQGLSTATKDTYDALHMQALPPR
MAGE A3/A6 TCR DNA
(SEQ ID NO. 152)
atggcatgccctggcttcctgtgggcacttgtgatctccacctgtcttgaatttagcatggctcagacagtcactcagtctcaaccagaga
tgtctgtgcaggaggcagagaccgtgaccctgagctgcacatatgacaccagtgagagtgattattatttattctggtacaagcagcctc
ccagcaggcagatgattctcgttattcgccaagaagcttataagcaacagaatgcaacagagaatcgtttctctgtgaacttccagaaag
cagccaaatccttcagtctcaagatctcagactcacagctgggggatgccgcgatgtatttctgtgctctccggagctcaggaacctac
aaatacatctttggaacaggcaccaggctgaaggttttagcaaatatccagaaccctgaacctgctgtgtaccagttaaaagatcctcgg
tctcaggacagcaccctctgcctgttcaccgactttgactcccaaatcaatgtgccgaaaaccatggaatctggaacgttcatcactgac
aaaactgtgctggacatgaaagctatggattccaagagcaatggggccattgcctggagcaaccagacaagcttcacctgccaagat
atcttcaaagagaccaacgccacctaccccagttcagacgttccctgtgatgccacgttgactgagaaaagctttgaaacagatatgaa
cctaaactttcaaaacctgtcagttatgggactccgaatcctcctgctgaaagtagccggatttaacctgctcatgacgctgaggctgtgg
tccagtcgggccaagcggtccggatccggagccaccaacttcagcctgctgaagcaggccggcgacgtggaggagaaccccggc
cccatgggcaccaggctcctcttctgggtggccttctgtctcctgggggcagatcacacaggagctggagtctcccagtcccccagta
acaaggtcacagagaagggaaaggatgtagagctcaggtgtgatccaatttcaggtcatactgccctttactggtaccgacagagcct
ggggcagggcctggagtttttaatttacttccaaggcaacagtgcaccagacaaatcagggctgcccagtgatcgcttctctgcagaga
ggactgggggatccgtctccactctgacgatccagcgcacacagcaggaggactcggccgtgtatctctgtgccagcatccggaca
gggccttttttctctggaaacaccatatattttggagagggaagttggctcactgttgtagaggacctgagaaacgtgaccccacccaag
gtctccttgtttgagccatcaaaagcagagattgcaaacaaacaaaaggctaccctcgtgtgcttggccaggggcttcttccctgaccac
gtggagctgagctggtgggtgaatggcaaggaggtccacagtggggtcagcacggaccctcaggcctacaaggagagcaattata
gctactgcctgagcagccgcctgagggtctctgctaccttctggcacaatcctcgaaaccacttccgctgccaagtgcagttccatggg
ctttcagaggaggacaagtggccagagggctcacccaaacctgtcacacagaacatcagtgcagaggcctggggccgagcagact
gtggaatcacttcagcatcctatcatcagggggttctgtctgcaaccatcctctatgagatcctactggggaaggccaccctatatgctgt
gctggtcagtggcctggtgctgatggctatggtcaaaagaaagaactcatga
MAGE A3/A6 TCR AA
(SEQ ID NO. 153)
M A C P G F L W A L V I S T C L E F S M A Q T V T Q S Q P E M S V Q E A E T V T L
S C T Y D T S E S D Y Y L F W Y K Q P P S R Q M I L V I R Q E A Y K Q Q N A T E N
R F S V N F Q K A A K S F S L K I S D S Q L G D A A M Y F C A L R S S G T Y K Y I F
G T G T R L K V L A N I Q N P E P A V Y Q L K D P R S Q D S T L C L F T D F D S Q I
N V P K T M E S G T F I T D K T V L D M K A M D S K S N G A I A W S N Q T S F T C
Q D I F K E T N A T Y P S S D V P C D A T L T E K S F E T D M N L N F Q N L S V M
G L R I L L L K V A G F N L L M T L R L W S S R A K R S G S G A T N F S L L K Q A
G D V E E N P G P M G T R L L F W V A F C L L G A D H T G A G V S Q S P S N K V T
E K G K D V E L R C D P I S G H T A L Y W Y R Q S L G Q G L E F L I Y F Q G N S A
P D K S G L P S D R F S A E R T G G S V S T L T I Q R T Q Q E D S A V Y L C A SI R T
G P F F S G N T I Y F G E G S W L T V V E D L R N V T P P K V S L F E P S K A E I A
N K Q K A T L V C L A R G F F P D H V E L S W W V N G K E V H S G V S T D P Q A
Y K E S N Y S Y C L S S R L R V S A T F W H N P R N H F R C Q V Q F H G L S E E D
K W P E G S P K P V T Q N I S A E A W G R A D C G I T S A S Y H Q G V L S A T I L
Y E I L L G K A T L Y A V L V S G L V L M A M V K R K N S Stop
Anti-CLL-1 CAR DNA
(SEQ ID NO. 154)
ATGGCACTCCCCGTAACTGCTCTGCTGCTGCCGTTGGCATTGCTCCTGCACGCCG
CACGCCCGCAGGTCCAACTGCAAGAAAGCGGACCCGGACTGGTGAAGCCTTCTG
AGACACTTAGTCTGACGTGCACGGTCAGTGGCGGCTCCATCTCCTCCTATTATTG
GTCATGGATACGACAACCCCCAGGTAAGGGCCTGGAATGGATTGGCTATATCTA
CTATTCAGGAAGCACGAACTACAATCCCAGCCTGAAGTCCCGAGTGACAATTTC
AGTAGATACCAGTAAAAACCAGTTCAGTCTTAAACTGTCAAGCGTGACAGCTGC
CGACACCGCTGTGTATTACTGCGTCTCACTGGTGTATTGTGGAGGGGATTGTTAT
AGCGGGTTCGATTATTGGGGACAGGGAACCCTGGTGACTGTATCTTCCGGCGGC
GGCGGCTCAGGGGGTGGCGGTAGTGGCGGTGGGGGTTCCGATATTCAACTGACA
CAATCCCCCAGCTCACTCAGCGCCAGCGTGGGGGACAGGGTTAGCTTTACCTGTC
AAGCCTCTCAGGATATAAATAACTTTCTGAACTGGTATCAACAGAAGCCTGGGA
AGGCGCCCAAACTCCTGATCTATGATGCGTCCAACCTGGAAACTGGCGTGCCTTC
ACGCTTTAGCGGCTCTGGCAGTGGTACAGACTTCACTTTTACCATCTCTTCACTTC
AGCCGGAGGACATCGCCACATATTACTGTCAACAGTACGGAAACTTGCCCTTTAC
TTTTGGAGGCGGCACCAAAGTTGAAATCAAAAGGGCCGCTGCCCTGGATAACGA
AAAGAGCAATGGGACTATAATACATGTTAAAGGAAAACACCTGTGTCCATCTCC
CCTGTTCCCTGGACCGTCAAAGCCATTTTGGGTGCTCGTGGTTGTCGGTGGCGTT
CTCGCCTGTTATAGCTTGCTGGTGACAGTAGCCTTCATTATCTTTTGGGTGAGATC
CAAAAGAAGCCGCCTGCTCCATAGCGATTACATGAATATGACTCCACGCCGCCCT
GGCCCCACAAGGAAACACTACCAGCCTTACGCACCACCTAGAGATTTCGCTGCCT
ATCGGAGCCGAGTGAAATTTTCTAGATCAGCTGATGCTCCCGCCTATCAGCAGGG
ACAGAATCAACTTTACAATGAGCTGAACCTGGGTCGCAGAGAAGAGTACGACGT
TTTGGACAAACGCCGGGGCCGAGATCCTGAGATGGGGGGGAAGCCGAGAAGGA
AGAATCCTCAAGAAGGCCTGTACAACGAGCTTCAAAAAGACAAAATGGCTGAGG
CGTACTCTGAGATCGGCATGAAGGGCGAGCGGAGACGAGGCAAGGGTCACGAT
GGCTTGTATCAGGGCCTGAGTACAGCCACAAAGGACACCTATGACGCCCTCCAC
ATGCAGGCACTGCCCCCACGC
Anti-CLL-1 CAR AA
(SEQ ID NO. 155)
MALPVTALLLPLALLLHAARPQVQLQESGPGLVKPSETLSLTCTVSGGSISSYYWSWI
RQPPGKGLEWIGYIYYSGSTNYNPSLKSRVTISVDTSKNQFSLKLSSVTAADTAVYYC
VSLVYCGGDCYSGFDYWGQGTLVTVSSGGGGSGGGGSGGGGSDIQLTQSPSSLSAS
VGDRVSFTCQASQDINNFLNWYQQKPGKAPKLLIYDASNLETGVPSRFSGSGSGTDF
TFTISSLQPEDIATYYCQQYGNLPFTFGGGTKVEIKRAAALDNEKSNGTIIHVKGKHL
CPSPLFPGPSKPFWVLVVVGGVLACYSLLVTVAFIIFWVRSKRSRLLHSDYMNMTPR
RPGPTRKHYQPYAPPRDFAAYRSRVKFSRSADAPAYQQGQNQLYNELNLGRREEYD
VLDKRRGRDPEMGGKPRRKNPQEGLYNELQKDKMAEAYSEIGMKGERRRGKGHD
GLYQGLSTATKDTYDALHMQALPPR
Anti-BCMA CAR DNA
(SEQ ID NO. 156)
ATGGCACTCCCCGTAACTGCTCTGCTGCTGCCGTTGGCATTGCTCCTGCACGCCG
CACGCCCGCAGGTGCAGCTGGTGGAGTCTGGGGGAGGCGTGGTCCAGCCTGGGA
GGTCCCTGAGACTCTCCTGTGCAGCGTCTGGATTCACCTTCAGTAGCTATGGCAT
GCACTGGGTCCGCCAGGCTCCAGGCAAGGGGCTGGAGTGGGTGGCAGTTATATC
GTATGATGGAAGTAATAAATACTATGCAGACTCCGTGAAGGGCCGATTCACCAT
CTCCAGAGACAATTCCAAGAACACGCTGTATCTGCAAATGAACAGCCTGAGAGC
CGAGGACACGGCGGTGTACTACTGCGTCAAGGGGCCGTTGCAGGAGCCGCCATA
CGATTATGGAATGGACGTATGGGGCCAGGGAACAACTGTCACCGTCTCCTCAGG
GTCTACATCCGGCTCCGGGAAGCCCGGAAGTGGCGAAGGTAGTACAAAGGGGGA
AATAGTGATGACGCAGTCTCCAGCCACCCTGTCTGTGTCTCCAGGGGAAAGAGC
CACCCTCTCCTGCAGGGCCAGTCAGAGTGTTAGCAGCAACTTAGCCTGGTACCAG
CAGAAACCTGGCCAGGCTCCCAGGCTCCTCATCTATAGCGCATCCACCAGGGCC
ACTGGTATCCCAGCCAGGTTCAGTGGCAGTGGGTCTGGGACAGAGTTCACTCTCA
CCATCAGCAGCCTGCAGTCTGAAGATTTTGCAGTTTATTACTGTCAGCAGCACCA
CGTCTGGCCTCTCACTTTTGGCGGAGGGACCAAGGTTGAGATCAAACGGGCCGCT
GCCCTTGATAATGAAAAGTCAAACGGAACAATCATTCACGTGAAGGGCAAGCAC
CTCTGTCCGTCACCCTTGTTCCCTGGTCCATCCAAGCCATTCTGGGTGTTGGTCGT
AGTGGGTGGAGTCCTCGCTTGTTACTCTCTGCTCGTCACCGTGGCTTTTATAATCT
TCTGGGTTAGATCCAAAAGAAGCCGCCTGCTCCATAGCGATTACATGAATATGAC
TCCACGCCGCCCTGGCCCCACAAGGAAACACTACCAGCCTTACGCACCACCTAG
AGATTTCGCTGCCTATCGGAGCAGGGTGAAGTTTTCCAGATCTGCAGATGCACCA
GCGTATCAGCAGGGCCAGAACCAACTGTATAACGAGCTCAACCTGGGACGCAGG
GAAGAGTATGACGTTTTGGACAAGCGCAGAGGACGGGACCCTGAGATGGGTGGC
AAACCAAGACGAAAAAACCCCCAGGAGGGTCTCTATAATGAGCTGCAGAAGGAT
AAGATGGCTGAAGCCTATTCTGAAATAGGCATGAAAGGAGAGCGGAGAAGGGG
AAAAGGGCACGACGGTTTGTACCAGGGACTCAGCACTGCTACGAAGGATACTTA
TGACGCTCTCCACATGCAAGCCCTGCCACCTAGG
Anti-BCMA CAR AA
(SEQ ID NO. 157)
MALPVTALLLPLALLLHAARPQVQLVESGGGVVQPGRSLRLSCAASGFTFSSYGMH
WVRQAPGKGLEWVAVISYDGSNKYYADSVKGRFTISRDNSKNTLYLQMNSLRAED
TAVYYCVKGPLQEPPYDYGMDVWGQGTTVTVSSGSTSGSGKPGSGEGSTKGEIVMT
QSPATLSVSPGERATLSCRASQSVSSNLAWYQQKPGQAPRLLIYSASTRATGIPARFS
GSGSGTEFTLTISSLQSEDFAVYYCQQHHVWPLTFGGGTKVEIKRAAALDNEKSNGT
IIHVKGKHLCPSPLFPGPSKPFWVLVVVGGVLACYSLLVTVAFIIFWVRSKRSRLLHS
DYMNMTPRRPGPTRKHYQPYAPPRDFAAYRSRVKFSRSADAPAYQQGQNQLYNEL
NLGRREEYDVLDKRRGRDPEMGGKPRRKNPQEGLYNELQKDKMAEAYSEIGMKGE
RRRGKGHDGLYQGLSTATKDTYDALHMQALPPR
C185 E7₁₁₋₁₉ / HLA-A*02:01 Specific TCR DNA
(SEQ ID NO. 158)
atgggcaccagactgttcttttatgtggccctgtgtctgctgtggaccggccacatggatgccggaattacacagagccccagacacaa
agtgaccgagacaggcacccctgtgacactgagatgccaccagaccgagaaccaccgctacatgtactggtacagacaggatccag
gccacggcctgagactgatccactacagctacggcgtgaaggacaccgacaagggcgaagtgtctgacggctacagcgtgtccag
aagcaagaccgaggatttcctgctgaccctggaaagcgccacaagcagccagaccagcgtgtacttttgtgccatcagcggctacaa
gaacaccgaggcctttttcggccaaggcaccaggctgacagtggtggaagatctgaagaacgtgttcccacctgaggtggccgtgttt
gagccttctgaggccgagatcagccacacacagaaagccacactcgtgtgcctggccaccggcttttatcccgatcacgtggaactgt
cttggtgggtcaacggcaaagaggtgcacagcggcgttagcacagaccctcagcctctgaaagagcagcccgctctgaacgacag
cagatactgtctgagcagcagactgagagtgtccgccaccttctggcagaaccccagaaaccacttcagatgccaggtgcagttctac
ggcctgtccgagaatgacgagtggacccaggatagagccaagccagtgacacagattgtgtctgccgaagcctggggcagagccg
attgtggctttacaagcgagagctaccagcagggcgtgctgtctgccacaatcctgtatgagatcctgctgggcaaagccactctgtac
gctgtgctggtgtctgccctggtgctgatggccatggtcaagagaaaggacagcagaggcagagccaagagatctggcagcggcg
ccacaaactttagcctgctgaaacaggccggcgacgtggaagagaaccctggacctatgaagtccctgcgggtgctgctggttattct
gtggctgcagctgagctgggtttggagccagggacagcaagtgatgcagatccctcagtaccagcacgtgcaagaaggcgaggact
tcaccacctactgcaacagcagcacaaccctgagcaacatccagtggtacaagcagaggcctggcgggcaccctgtgtttctgatcc
agctggttaagagcggcgaagtgaagaagcagaagcggctgacctttcagttcggcgaggccaagaagaacagcagcctgcacatt
accgccacacagaccaccgacgtgggcacatatttttgcgctggcagagaaggcggcagcgagaagctggtttttggcaagggcac
caaactgaccgtgaatcccgacattcagaaccccgatccagccgtgtaccagctgagagacagcaagagcagcgacaagagcgtgt
gtctgttcaccgacttcgactcccagaccaatgtgtcccagagcaaggactccgacgtgtacatcaccgataagaccgtgctggacat
gcggagcatggacttcaagagcaatagcgccgtggcttggagcaacaagagcgactttgcctgcgccaacgccttcaacaacagca
tcatccccgaggacacattcttcccaagtcctgagagcagctgcgacgtgaagctggtggaaaagagcttcgagacagacaccaacc
tgaacttccagaacctgagcgtgatcggcttcagaatcctgctgctgaaggtggccggcttcaacctgctgatgactctgagactgtggt
ccagctgaattcggatccaagcttaggcctgctcgctttcttgctgtcccatttctattaaaggttcctttgttc
C185 E7₁₁₋₁₉ / HLA-A*02:01 Specific TCR AA
(SEQ ID NO. 159)
MGTRLFFYVALCLLWTGHMDAGITQSPRHKVTETGTPVTLRCHQTENHRYMYWYR
QDPGHGLRLIHYSYGVKDTDKGEVSDGYSVSRSKTEDFLLTLESATSSQTSVYFCAIS
GYKNTEAFFGQGTRLTVVEDLKNVFPPEVAVFEPSEAEISHTQKATLVCLATGFYPD
HVELSWWVNGKEVHSGVSTDPQPLKEQPALNDSRYCLSSRLRVSATFWQNPRNHFR
CQVQFYGLSENDEWTQDRAKPVTQIVSAEAWGRADCGFTSESYQQGVLSATILYEIL
LGKATLYAVLVSALVLMAMVKRKDSRGRAKRSGSGATNFSLLKQAGDVEENPGPM
KSLRVLLVILWLQLSWVWSQGQQVMQIPQYQHVQEGEDFTTYCNSSTTLSNIQWYK
QRPGGHPVFLIQLVKSGEVKKQKRLTFQFGEAKKNSSLHITATQTTDVGTYFCAGRE
GGSEKLVFGKGTKLTVNPDIQNPDPAVYQLRDSKSSDKSVCLFTDFDSQTNVSQSKD
SDVYITDKTVLDMRSMDFKSNSAVAWSNKSDFACANAFNNSIIPEDTFFPSPESSCDV
KLVEKSFETDTNLNFQNLSVIGFRILLLKVAGFNLLMTLRLWSS
E7₁₁₋₁₉ / HLA-A*02:01 Specific TCR DNA
(SEQ ID NO. 160)
atgggacctggattgctttgttgggccctgctgtgtctgcttggagctggacttgtggatgccggcgtgacacagtctcccacacacctg
atcaagaccagaggccagcaagtgaccctgagatgtagccctaagagcggccacgacaccgtgtcttggtatcagcaggctcttggc
cagggacctcagttcatcttccagtactacgaggaagaggaacggcagcggggcaacttccctgatagattctctggccatcagttcc
ccaactacagcagcgagctgaacgtgaacgctctgctgctgggcgatagcgccctgtatctgtgtgccagttctcttggttggagaggc
ggcagatacaacgagcagttctttggccctggcaccagactgaccgtgctggaagatctgaagaacgtgttcccacctgaggtggcc
gtgtttgagccttctgaggccgagatcagccacacacagaaagccacactcgtgtgcctggccaccggcttttatcccgatcacgtgg
aactgtcttggtgggtcaacggcaaagaggtgcacagcggcgttagcacagaccctcagcctctgaaagagcagcccgctctgaac
gacagcagatactgtctgagcagcagactgagagtgtccgccaccttctggcagaaccccagaaaccacttcagatgccaggtgcag
ttctacggcctgtccgagaatgacgagtggacccaggatagagccaagccagtgacacagattgtgtctgccgaagcctggggcag
agccgattgtggctttacaagcgagagctaccagcagggcgtgctgtctgccacaatcctgtatgagatcctgctgggcaaagccact
ctgtacgctgtgctggtgtctgccctggtgctgatggccatggtcaagagaaaggacagcagaggcagagccaagagatctggcag
cggcgccacaaactttagcctgctgaaacaggccggcgacgtggaagagaaccctggacctatgaagtccctgcgggtgctgctgg
ttattctgtggctgcagctgagctgggtttggagccagggacagaacatcgaccagcctaccgagatgacagccaccgaaggcgcc
atcgtgcagatcaattgcacctaccagaccagcggcttcaacggcctgttctggtatcaacagcatgccggcgaggcccctaccttcct
gagctataatgtgctggacggcctggaagaaaagggcagattcagcagcttcctgtccagaagcaagggctacagctacctgctgct
gaaagaactccagatgaaggacagcgcctcctacctgtgtgcctccgtggatggaaacaacagactggccttcggcaagggcaacc
aggtggtggtcatccccgacattcagaaccccgatccagccgtgtaccagctgagagacagcaagagcagcgacaagagcgtgtgt
ctgttcaccgacttcgactcccagaccaatgtgtcccagagcaaggactccgacgtgtacatcaccgataagaccgtgctggacatgc
ggagcatggacttcaagagcaatagcgccgtggcttggagcaacaagagcgactttgcctgcgccaacgccttcaacaacagcatca
tccccgaggacacattcttcccaagtcctgagagcagctgcgacgtgaagctggtggaaaagagcttcgagacagacaccaacctga
acttccagaacctgagcgtgatcggcttcagaatcctgctgctgaaggtggccggcttcaacctgctgatgactctgagactgtggtcc
agctgaattcggatccaagcttaggcctgctcgctttcttgctgtcccatttctattaaaggttcctttgttc
E7₁₁₋₁₉ / HLA-A*02:01 Specific TCR AA
(SEQ ID NO. 161)
MGPGLLCWALLCLLGAGLVDAGVTQSPTHLIKTRGQQVTLRCSPKSGHDTVSWYQ
QALGQGPQFIFQYYEEEERQRGNFPDRFSGHQFPNYSSELNVNALLLGDSALYLCAS
SLGWRGGRYNEQFFGPGTRLTVLEDLKNVFPPEVAVFEPSEAEISHTQKATLVCLAT
GFYPDHVELSWWVNGKEVHSGVSTDPQPLKEQPALNDSRYCLSSRLRVSATFWQNP
RNHFRCQVQFYGLSENDEWTQDRAKPVTQIVSAEAWGRADCGFTSESYQQGVLSAT
ILYEILLGKATLYAVLVSALVLMAMVKRKDSRGRAKRSGSGATNFSLLKQAGDVEE
NPGPMKSLRVLLVILWLQLSWVWSQGQNIDQPTEMTATEGAIVQINCTYQTSGFNG
LFWYQQHAGEAPTFLSYNVLDGLEEKGRFSSFLSRSKGYSYLLLKELQMKDSASYLC
ASVDGNNRLAFGKGNQVVVIPDIQNPDPAVYQLRDSKSSDKSVCLFTDFDSQTNVSQ
SKDSDVYITDKTVLDMRSMDFKSNSAVAWSNKSDFACANAFNNSIIPEDTFFPSPESS
CDVKLVEKSFETDTNLNFQNLSVIGFRILLLKVAGFNLLMTLRLWSS
Axi Cel (KTE-C19) Leader (CSF2RA) DNA
(SEQ ID NO. 162)
Atgcttctcctggtgacaagccttctgctctgtgagttaccacacccagcattcctcctgatccca
Axi Cel (KTE-C19) Leader (CSF2RA) AA
(SEQ ID NO. 163)
MLLLVTSLLLCELPHPAFLLIP
Axi Cel (KTE-C19) scFv heavy chain DNA
(SEQ ID NO. 164)
gacatccagatgacacagactacatcctccctgtctgcctctctgggagacagagtcaccatcagttgcagggcaagtcaggacatta
gtaaatatttaaattggtatcagcagaaaccagatggaactgttaaactcctgatctaccatacatcaagattacactcaggagtcccatc
aaggttcagtggcagtgggtctggaacagattattctctcaccattagcaacctggagcaagaagatattgccacttacttttgccaacag
ggtaatacgcttccgtacacgttcggaggggggactaagttggaaataaca
Axi Cel (KTE-C19) scFv heavy chain AA
(SEQ ID NO. 165)
DIQMTQTTSSLSASLGDRVTISCRASQDISKYLNWYQQKPDGTVKLLIYHTSRLHSGV
PSRFSGSGSGTDYSLTISNLEQEDIATYFCQQGNTLPYTFGGGTKLEIT
Axi Cel (KTE-C19) Linker (Whitlow) DNA
(SEQ ID NO. 166)
ggctccacctctggatccggcaagcccggatctggcgagggatccaccaagggc
Axi Cel (KTE-C19) Linker (Whitlow) AA
(SEQ ID NO. 167)
GSTSGSGKPGSGEGSTKG
Axi Cel (KTE-C19) scFv light chain DNA
(SEQ ID NO. 168)
gaggtgaaactgcaggagtcaggacctggcctggtggcgccctcacagagcctgtccgtcacatgcactgtctcaggggtctcattac
ccgactatggtgtaagctggattcgccagcctccacgaaagggtctggagtggctgggagtaatatggggtagtgaaaccacatacta
taattcagctctcaaatccagactgaccatcatcaaggacaactccaagagccaagttttcttaaaaatgaacagtctgcaaactgatga
cacagccatttactactgtgccaaacattattactacggtggtagctatgctatggactactggggtcaaggaacctcagtcaccgtctcc
tca
Axi Cel (KTE-C19) scFv light chain AA
(SEQ ID NO. 169)
EVKLQESGPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAMDYWGQ
GTSVTVSS
Axi Cel (KTE-C19) Minispacer DNA
(SEQ ID NO. 170)
gcggccgca
Axi Cel (KTE-C19) Minispacer AA
(SEQ ID NO. 171)
AAA
Axi Cel (KTE-C19) CD28 spacer (extracellular/ TM region of CD28) DNA
(SEQ ID NO. 172)
attgaagttatgtatcctcctccttacctagacaatgagaagagcaatggaaccattatccatgtgaaagggaaacacctttgtccaagtc
ccctatttcccggaccttctaagcccttttgggtgctggtggtggttgggggagtcctggcttgctatagcttgctagtaacagtggccttt
attattttctgggtg
Axi Cel (KTE-C19) CD28 spacer (extracellular / TM region of CD28) AA
(SEQ ID NO. 173)
IEVMYPPPYLDNEKSNGTIIHVKGKHLCPSPLFPGPSKPFWVLVVVGGVLACYSLLVT
VAFIIFWV
Axi Cel (KTE-C19) CD28 costimulatory (intracellular region of CD28) DNA
(SEQ ID NO. 174)
aggagtaagaggagcaggctcctgcacagtgactacatgaacatgactccccgccgccccgggcccacccgcaagcattaccagc
cctatgccccaccacgcgacttcgcagcctatcgctcc
Axi Cel (KTE-C19) CD28 costimulatory (intracellular region of CD28) AA
(SEQ ID NO. 175)
RSKRSRLLHSDYMNMTPRRPGPTRKHYQPYAPPRDFAAYRS
Axi Cel (KTE-C19) CD3 zeta DNA
(SEQ ID NO. 176)
agagtgaagttcagcaggagcgcagacgcccccgcgtaccagcagggccagaaccagctctataacgagctcaatctaggacgaa
gagaggagtacgatgttttggacaagagacgtggccgggaccctgagatggggggaaagccgagaaggaagaaccctcaggaag
gcctgtacaatgaactgcagaaagataagatggcggaggcctacagtgagattgggatgaaaggcgagcgccggaggggcaagg
ggcacgatggcctttaccagggtctcagtacagccaccaaggacacctacgacgcccttcacatgcaggccctgccccctcgc
Axi Cel (KTE-C19) CD3 zeta AA
(SEQ ID NO. 177)
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
Axi Cel (KTE-C19) VL CDR1 (Chothia)
(SEQ ID NO. 178)
RASQDISKYLN
Axi Cel (KTE-C19) VL CDR2 (Chothia)
(SEQ ID NO. 179)
HTSRLHS
Axi Cel (KTE-C19) VL CDR3 (Chothia)
(SEQ ID NO. 180)
QQGNTLPYT
Axi Cel (KTE-C19) VH CDR1 (Chothia)
(SEQ ID NO. 181)
GVSLPDY
Axi Cel (KTE-C19) VH CDR2 (Chothia)
(SEQ ID NO. 182)
WGSET
Axi Cel (KTE-C19) VH CDR3 (Chothia)
(SEQ ID NO. 183)
HYYYGGSYAMDY
Axi Cel (KTE-C19) VL CDR1 (Kabat)
(SEQ ID NO. 184)
RASQDISKYLN
Axi Cel (KTE-C19) VL CDR2 (Kabat)
(SEQ ID NO. 185)
HTSRLHS
Axi Cel (KTE-C19) VL CDR3 (Kabat)
(SEQ ID NO. 186)
QQGNTLPYT
Axi Cel (KTE-C19) VH CDR1 (Kabat)
(SEQ ID NO. 187)
DYGVS
Axi Cel (KTE-C19) VH CDR2 (Kabat)
(SEQ ID NO. 188)
VIWGSETTYYNSALKS
Axi Cel (KTE-C19) VH CDR3 (Kabat)
(SEQ ID NO. 189)
HYYYGGSYAMDY
Humanized anti-CD19 CAR CD8 Leader DNA
(SEQ ID NO. 190)
ATGGCACTCCCCGTAACTGCTCTGCTGCTGCCGTTGGCATTGCTCCTGCACGCCG
CACGCCCG
Humanized anti-CD19 CAR CD8 Leader AA
(SEQ ID NO. 191)
MALPVTALLLPLALLLHAARP
Humanized anti-CD19 CAR scFv heavy chain DNA
(SEQ ID NO. 192)
GATATTCAAATGACCCAGTCCCCGTCCTCCCTGAGTGCCTCCGTCGGTGACCGTG
TTACGATTACCTGCCGTGCGAGCCAAGACATCTCTAAATACCTGAACTGGTATCA
GCAAAAACCGGATCAGGCACCGAAACTGCTGATCAAACATACCTCACGTCTGCA
CTCGGGTGTGCCGAGCCGCTTTAGTGGTTCCGGCTCAGGTACCGATTACACCCTG
ACGATCAGCTCTCTGCAGCCGGAAGACTTTGCCACGTATTACTGCCAGCAAGGTA
ATACCCTGCCGTATACGTTCGGCCAAGGTACCAAACTGGAAATCAAA
Humanized anti-CD19 CAR scFv heavy chain AA
(SEQ ID NO. 193)
DIQMTQSPSSLSASVGDRVTITCRASQDISKYLNWYQQKPDQAPKLLIKHTSRLHSGV
PSRFSGSGSGTDYTLTISSLQPEDFATYYCQQGNTLPYTFGQGTKLEIK
Humanized anti-CD19 CAR Linker (Whitlow) DNA
(SEQ ID NO. 194)
GGCTCGACGAGCGGCTCTGGTAAACCGGGCTCTGGTGAAGGCAGTACCAAAGGT
Humanized anti-CD19 CAR Linker (Whitlow) AA
(SEQ ID NO. 195)
GSTSGSGKPGSGEGSTKG
Humanized anti-CD19 CAR scFv light chain DNA
(SEQ ID NO. 196)
GAAGTGCAGCTGGTTGAAAGCGGTGGTGGTCTGGTTCAACCGGGTCGTTCCCTGC
GTCTGTCATGTACGGCGAGTGGTGTCTCCCTGCCGGACTATGGCGTGTCCTGGAT
TCGTCAGCCGCCGGGTAAAGGCCTGGAATGGATTGGTGTCATCTGGGGCAGTGA
AACCACGTATTACAACTCGGCCCTGAAAAGCCGTTTCACCATCTCTCGCGATAAC
AGTAAAAATACGCTGTACCTGCAGATGAATAGCCTGCGCGCGGAAGACACCGCC
GTTTACTACTGCGCAAAACATTACTACTACGGTGGCAGCTATGCTATGGATTACT
GGGGTCAAGGCACGCTGGTCACCGTTTCGTCA
Humanized anti-CD19 CAR scFv light chain AA
(SEQ ID NO. 197)
EVQLVESGGGLVQPGRSLRLSCTASGVSLPDYGVSWIRQPPGKGLEWIGVIWGSETT
YYNSALKSRFTISRDNSKNTLYLQMNSLRAEDTAVYYCAKHYYYGGSYAMDYWG
QGTLVTVSS
Humanized anti-CD19 CAR Minispacer DNA
(SEQ ID NO. 198)
GCCGCTGCC
Humanized anti-CD19 CAR Minispacer AA
(SEQ ID NO. 199)
AAA
Humanized anti-CD19 CAR CD28T spacer (extracellular / TM region of
CD28) DNA
(SEQ ID NO. 200)
CTTGATAATGAAAAGTCAAACGGAACAATCATTCACGTGAAGGGCAAGCACCTC
TGTCCGTCACCCTTGTTCCCTGGTCCATCCAAGCCATTCTGGGTGTTGGTCGTAGT
GGGTGGAGTCCTCGCTTGTTACTCTCTGCTCGTCACCGTGGCTTTTATAATCTTCT
GGGTT
Humanized anti-CD19 CAR CD28T spacer (extra / TM region of CD28) AA
(SEQ ID NO. 201)
LDNEKSNGTIIHVKGKHLCPSPLFPGPSKPFWVLVVVGGVLACYSLLVTVAFIIFWV
Humanized anti-CD19 CAR CD28 costiminulatory (intracellular region of
CD28) DNA
(SEQ ID NO. 202)
AGATCCAAAAGAAGCCGCCTGCTCCATAGCGATTACATGAATATGACTCCACGC
CGCCCTGGCCCCACAAGGAAACACTACCAGCCTTACGCACCACCTAGAGATTTC
GCTGCCTATCGGAGC
Humanized anti-CD19 CAR costimulatory (intracellular region of CD28) AA
(SEQ ID NO. 203)
RSKRSRLLHSDYMNMTPRRPGPTRKHYQPYAPPRDFAAYRS
Humanized anti-CD19 CAR CD3 zeta DNA
(SEQ ID NO. 204)
AGGGTGAAGTTTTCCAGATCTGCAGATGCACCAGCGTATCAGCAGGGCCAGAAC
CAACTGTATAACGAGCTCAACCTGGGACGCAGGGAAGAGTATGACGTTTTGGAC
AAGCGCAGAGGACGGGACCCTGAGATGGGTGGCAAACCAAGACGAAAAAACCC
CCAGGAGGGTCTCTATAATGAGCTGCAGAAGGATAAGATGGCTGAAGCCTATTC
TGAAATAGGCATGAAAGGAGAGCGGAGAAGGGGAAAAGGGCACGACGGTTTGT
ACCAGGGACTCAGCACTGCTACGAAGGATACTTATGACGCTCTCCACATGCAAG
CCCTGCCAcctagg
Humanized anti-CD19 CAR CD3 zeta AA
(SEQ ID NO. 205)
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
Humanized anti-CD19 CAR VL fully human anti-CD19 CDR1 (Chothia)
(SEQ ID NO. 206)
RASQSVSSSYLA
Humanized anti-CD19 CAR VL fully human anti-CD19 CDR2 (Chothia)
(SEQ ID NO. 207)
GASSRAT
Humanized anti-CD19 CAR VL fully human anti-CD19 CDR3 (Chothia)
(SEQ ID NO. 208)
QQYGSSRFT
Humanized anti-CD19 CAR VH fully human anti-CD19 CDR1 (Chothia)
(SEQ ID NO. 209)
GGTFSSY
Humanized anti-CD19 CAR VH fully human anti-CD19 CDR2 (Chothia)
(SEQ ID NO. 210)
IPIFGT
Humanized anti-CD19 CAR VH fully human anti-CD19 CDR3 (Chothia)
(SEQ ID NO. 211)
EAVAADWLDP
Humanized anti-CD19 CAR VL fully human anti-CD19 CDR1 (Kabat)
(SEQ ID NO. 212)
RASQSVSSSYLA
Humanized anti-CD19 CAR VL fully human anti-CD19 CDR2 (Kabat)
(SEQ ID NO. 213)
GASSRAT
Humanized anti-CD19 CAR VL fully human anti-CD19 CDR3 (Kabat)
(SEQ ID NO. 214)
QQYGSSRFT
Humanized anti-CD19 CAR VH fully human anti-CD19 CDR1 (Kabat)
(SEQ ID NO. 215)
SYAIS
Humanized anti-CD19 CAR VH fully human anti-CD19 CDR2 (Kabat)
(SEQ ID NO. 216)
GIIPIFGTTNYAQQFQG
Humanized anti-CD19 CAR VH fully human anti-CD19 CDR3 (Kabat)
(SEQ ID NO. 217)
EAVAADWLDP
Fully human anti-CD19 CAR Leader (CD8a) DNA
(SEQ ID NO. 218)
Atggccctgcctgtgacagctctgctgctgcccctggccctgctgctgcatgccgccagacct
Fully human anti-CD19 CAR Leader (CD8a) AA
(SEQ ID NO. 219)
MALPVTALLLPLALLLHAARP
Fully human anti-CD19 CAR scFv light chain DNA
(SEQ ID NO. 220)
gagatcgtgctgacccagtctcccggcaccctgtctctcagcccaggagagagagccaccctgagctgcagagccagccagagcgt
gtccagcagctacctggcctggtatcagcagaagcccggacaggcccccagactgctgatctacggcgccagctctagagccaccg
gcatccccgacagattcagcggcagcggcagtggcaccgacttcaccctgaccatcagcagactggaacccgaggacttcgccgtg
tactactgccagcagtacggcagcagccggttcaccttcggccctggcaccaaggtggacatcaag
Fully human anti-CD19 CAR scFv light chain AA
(SEQ ID NO. 221)
EIVLTQSPGTLSLSPGERATLSCRASQSVSSSYLAWYQQKPGQAPRLLIYGASSRATGI
PDRFSGSGSGTDFTLTISRLEPEDFAVYYCQQYGSSRFTFGPGTKVDIK
Fully human anti-CD19 CAR Linker (Whitlow) DNA
(SEQ ID NO. 222)
ggcagcacctccggcagcggcaagcctggctctggcgagggctctaccaagggc
Fully human anti-CD19 CAR Linker (Whitlow) AA
(SEQ ID NO. 223)
GSTSGSGKPGSGEGSTKG
Fully human anti-CD19 CAR scFv heavy chain DNA
(SEQ ID NO. 224)
caggtgcagctggtgcagtctggcgccgaagtgaagaaacccggctctagcgtgaaggtgtcctgcaaggacagcggcggcacctt
cagcagctacgccatcagctgggtgcgccaggccccaggacaggggctggaatggatgggcggcatcatccccatcttcggcacc
accaactacgcccagcagttccagggcagagtgaccatcaccgccgacgagagcaccagcaccgcctacatggaactgagcagcc
tgcggagcgaggacacagccgtgtattactgtgcccgcgaggccgtggccgccgactggctggatccttggggacagggcaccct
ggtgacagtgtccagc
Fully human anti-CD19 CAR scFv heavy chain AA
(SEQ ID NO. 225)
QVQLVQSGAEVKKPGSSVKVSCKDSGGTFSSYAISWVRQAPGQGLEWMGGIIPIFGT
TNYAQQFQGRVTITADESTSTAYMELSSLRSEDTAVYYCAREAVAADWLDPWGQG
TLVTVSS
Fully human anti-CD19 CAR CD8a spacer and TM region DNA
(SEQ ID NO. 226)
ttcgtgcccgtgttcctgcccgccaagcctaccaccacccctgcccctagacctcccaccccagccccaacaatcgccagccagcct
ctgtccctgcggcccgaagcctgtagacctgctgccggcggagccgtgcacaccagaggcctggacttcgcctgcgatatctacatc
tgggcccctctggccggcacctgtggcgtgctgctgctgagcctggtgatcaccctgtactgcaaccaccggaac
Fully human anti-CD19 CAR CD8a spacer and TM region AA
(SEQ ID NO. 227)
FVPVFLPAKPTTTPAPRPPTPAPTIASQPLSLRPEACRPAAGGAVHTRGLDFACDIYIW
APLAGTCGVLLLSLVITLYCNHRN
Fully human anti-CD19 CAR CD28 costim. DNA
(SEQ ID NO. 228)
agaagcaagcggagccggctgctgcacagcgactacatgaacatgaccccaagacggcctggccccacccggaagcactaccag
ccttacgcccctcccagagacttcgccgcctaccggtcc
Fully human anti-CD19 CAR CD28 costim. AA
(SEQ ID NO. 229)
RSKRSRLLHSDYMNMTPRRPGPTRKHYQPYAPPRDFAAYRS
Fully human anti-CD19 CAR CD3 zeta DNA
(SEQ ID NO. 230)
agagtgaagttcagcagatccgccgacgcccctgcctaccagcagggacagaaccagctgtacaacgagctgaacctgggcagac
gggaagagtacgacgtgctggacaagcggagaggccgggaccccgagatgggcggaaagcccagacggaagaacccccagga
aggcctgtataacgaactgcagaaagacaagatggccgaggcctacagcgagatcggcatgaagggcgagcggaggcgcggca
agggccacgatggcctgtaccagggcctgagcaccgccaccaaggacacctacgacgccctgcacatgcaggccctgcccccca
ga
Fully human anti-CD19 CAR CD3 zeta AA
(SEQ ID NO. 231)
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
MAGE A3 / A6 TCR Construct - Variable alpha chain - AV38-2 DNA
(SEQ ID NO. 232)
atggcatgccctggcttcctgtgggcacttgtgatctccacctgtcttgaatttagcatggctcagacagtcactcagtctcaaccagaga
tgtctgtgcaggaggcagagaccgtgaccctgagctgcacatatgacaccagtgagagtgattattatttattctggtacaagcagcctc
ccagcaggcagatgattctcgttattcgccaagaagcttataagcaacagaatgcaacagagaatcgtttctctgtgaacttccagaaag
cagccaaatccttcagtctcaagatctcagactcacagctgggggatgccgcgatgtatttctgtgct
MAGE A3 / A6 TCR Construct - Variable alpha chain - AV38-2 AA
(SEQ ID NO. 233)
M A C P G F L W A L V I S T C L E F S M A Q T V T Q S Q P E M S V Q E A E T V T L
S C T Y D T S E S D Y Y L F W Y K Q P P S R Q M I L V I R Q E A Y K Q Q N A T E N
R F S V N F Q K A A K S F S L K I S D S Q L G D A A M Y F C A L R S S G T Y K Y I F
G T G T R L K V L A N
MAGE A3 / A6 TCR Construct - TRAJ40 DNA
(SEQ ID NO. 234)
ctccggagctcaggaacctacaaatacatctttggaacaggcaccaggctgaaggttttagcaaat
MAGE A3 / A6 TCR Construct - Murine constant alpha - Murine CA DNA
(SEQ ID NO. 235)
atccagaaccctgaacctgctgtgtaccagttaaaagatcctcggtctcaggacagcaccctctgcctgttcaccgactttgactcccaa
atcaatgtgccgaaaaccatggaatctggaacgttcatcactgacaaaactgtgctggacatgaaagctatggattccaagagcaatgg
ggccattgcctggagcaaccagacaagcttcacctgccaagatatcttcaaagagaccaacgccacctaccccagttcagacgttccc
tgtgatgccacgttgactgagaaaagctttgaaacagatatgaacctaaactttcaaaacctgtcagttatgggactccgaatcctcctgc
tgaaagtagccggatttaacctgctcatgacgctgaggctgtggtccagt
MAGE A3 / A6 TCR Construct - Murine constant alpha - Murine CA AA
(SEQ ID NO. 236)
I Q N P E P A V Y Q L K D P R S Q D S T L C L F T D F D S Q I N V P K T M E S G T F
I T D K T V L D M K A M D S K S N G A I A W S N Q T S F T C Q D I F K E T N A T Y
P S S D V P C D A T L T E K S F E T D M N L N F Q N L S V M G L R I L L L K V A G F
N L L M T L R L W S S
MAGE A3 / A6 TCR Construct - Furin-SG SG-P2A DNA
(SEQ ID NO. 237)
cgggccaagcggtccggatccggagccaccaacttcagcctgctgaagcaggccggcgacgtggaggagaaccccggcccc
MAGE A3 / A6 TCR Construct - Furin-SG SG-P2A AA
(SEQ ID NO. 238)
R A K R S G S G A T N F S L L K Q A G D V E E N P G P
MAGE A3 / A6 TCR Construct - Variable beta chain - BV7-2 DNA
(SEQ ID NO. 239)
atgggcaccaggctcctcttctgggtggccttctgtctcctgggggcagatcacacaggagctggagtctcccagtcccccagtaaca
aggtcacagagaagggaaaggatgtagagctcaggtgtgatccaatttcaggtcatactgccctttactggtaccgacagagcctggg
gcagggcctggagtttttaatttacttccaaggcaacagtgcaccagacaaatcagggctgcccagtgatcgcttctctgcagagagga
ctgggggatccgtctccactctgacgatccagcgcacacagcaggaggactcggccgtgtatctctgtgccagca
MAGE A3 / A6 TCR Construct - Variable beta chain - BV7-2 AA
(SEQ ID NO. 240)
M G T R L L F W V A F C L L G A D H T G A G V S Q S P S N K V T E K G K D V E L
R C D P I S G H T A L Y W Y R Q S L G Q G L E F L I Y F Q G N S A P D K S G L P S D
R F S A E R T G G S V S T L T I Q R T Q Q E D S A V Y L C A SI R T G P F F S G N T I
Y F G E G S W L T V V E
MAGE A3 / A6 TCR Construct - TRBJ1-3 DNA
(SEQ ID NO. 241)
tccggacagggccttttttctctggaaacaccatatattttggagagggaagttggctcactgttgtagag
MAGE A3 / A6 TCR Construct - Murine constant beta - Murine CB1 DNA
(SEQ ID NO. 242)
gacctgagaaacgtgaccccacccaaggtctccttgtttgagccatcaaaagcagagattgcaaacaaacaaaaggctaccctcgtgt
gcttggccaggggcttcttccctgaccacgtggagctgagctggtgggtgaatggcaaggaggtccacagtggggtcagcacggac
cctcaggcctacaaggagagcaattatagctactgcctgagcagccgcctgagggtctctgctaccttctggcacaatcctcgaaacc
acttccgctgccaagtgcagttccatgggctttcagaggaggacaagtggccagagggctcacccaaacctgtcacacagaacatca
gtgcagaggcctggggccgagcagactgtggaatcacttcagcatcctatcatcagggggttctgtctgcaaccatcctctatgagatc
ctactggggaaggccaccctatatgctgtgctggtcagtggcctggtgctgatggctatggtcaaaagaaagaactcatga
MAGE A3 / A6 TCR Construct - Murine constant beta - Murine CB1 AA
(SEQ ID NO. 243)
D L R N V T P P K V S L F E P S K A E I A N K Q K A T L V C L A R G F F P D H V E L
S W W V N G K E V H S G V S T D P Q A Y K E S N Y S Y C L S S R L R V S A T F W
H N P R N H F R C Q V Q F H G L S E E D K W P E G S P K P V T Q N I S A E A W G R
A D C G I T S A S Y H Q G V L S A T I L Y E I L L G K A T L Y A V L V S G L V L M A
M V K R K N S Stop
MAGE A3 / A6 TCR Va CDR1
(SEQ ID NO. 244)
TYDTSESDYYLF
MAGE A3 / A6 TCR Va CDR2
(SEQ ID NO. 245)
QEAYKQQ
MAGE A3 / A6 TCR Va CDR3
(SEQ ID NO. 246)
ALRSSGTYKYI
MAGE A3 / A6 TCR Vb CDR1
(SEQ ID NO. 247)
DPISGHTALY
MAGE A3 / A6 TCR Vb CDR2
(SEQ ID NO. 248)
FQGNSAPDKSG
MAGE A3 / A6 TCR Vb CDR3
(SEQ ID NO. 249)
ASIRTGPFFSGNTIY
Anti-CLL-1 CAR CD8 Leader DNA
(SEQ ID NO. 250)
ATGGCACTCCCCGTAACTGCTCTGCTGCTGCCGTTGGCATTGCTCCTGCACGCCG
CACGCCCG
Anti-CLL-1 CAR CD8 Leader AA
(SEQ ID NO. 251)
MALPVTALLLPLALLLHAARP
Anti-CLL-1 CAR scFv heavy chain DNA
(SEQ ID NO. 252)
CAGGTCCAACTGCAAGAAAGCGGACCCGGACTGGTGAAGCCTTCTGAGACACTT
AGTCTGACGTGCACGGTCAGTGGCGGCTCCATCTCCTCCTATTATTGGTCATGGA
TACGACAACCCCCAGGTAAGGGCCTGGAATGGATTGGCTATATCTACTATTCAGG
AAGCACGAACTACAATCCCAGCCTGAAGTCCCGAGTGACAATTTCAGTAGATAC
CAGTAAAAACCAGTTCAGTCTTAAACTGTCAAGCGTGACAGCTGCCGACACCGC
TGTGTATTACTGCGTCTCACTGGTGTATTGTGGAGGGGATTGTTATAGCGGGTTC
GATTATTGGGGACAGGGAACCCTGGTGACTGTATCTTCC
Anti-CLL-1 CAR scFv heavy chain AA
(SEQ ID NO. 253)
QVQLQESGPGLVKPSETLSLTCTVSGGSISSYYWSWIRQPPGKGLEWIGYIYYSGSTN
YNPSLKSRVTISVDTSKNQFSLKLSSVTAADTAVYYCVSLVYCGGDCYSGFDYWGQ
GTLVTVSS
Anti-CLL-1 CAR G4S linker DNA
(SEQ ID NO. 254)
GGCGGCGGCGGCTCAGGGGGTGGCGGTAGTGGCGGTGGGGGTTCC
Anti-CLL-1 CAR G4S linker AA
(SEQ ID NO. 255)
GGGGSGGGGSGGGGS
Anti-CLL-1 CAR scFv light chain DNA
(SEQ ID NO. 256)
GATATTCAACTGACACAATCCCCCAGCTCACTCAGCGCCAGCGTGGGGGACAGG
GTTAGCTTTACCTGTCAAGCCTCTCAGGATATAAATAACTTTCTGAACTGGTATC
AACAGAAGCCTGGGAAGGCGCCCAAACTCCTGATCTATGATGCGTCCAACCTGG
AAACTGGCGTGCCTTCACGCTTTAGCGGCTCTGGCAGTGGTACAGACTTCACTTT
TACCATCTCTTCACTTCAGCCGGAGGACATCGCCACATATTACTGTCAACAGTAC
GGAAACTTGCCCTTTACTTTTGGAGGCGGCACCAAAGTTGAAATCAAAAGG
Anti-CLL-1 CAR scFv light chain AA
(SEQ ID NO. 257)
DIQLTQSPSSLSASVGDRVSFTCQASQDINNFLNWYQQKPGKAPKWYDASNLETGV
PSRFSGSGSGTDFTFTISSLQPEDIATYYCQQYGNLPFTFGGGTKVEIKR
Anti-CLL-1 CAR Minispacer DNA
(SEQ ID NO. 258)
GCCGCTGCC
Anti-CLL-1 CAR Minispacer AA
(SEQ ID NO. 259)
AAA
Anti-CLL-1 CAR CD28T (extracellular / TM region of CD28) DNA
(SEQ ID NO. 260)
CTGGATAACGAAAAGAGCAATGGGACTATAATACATGTTAAAGGAAAACACCTG
TGTCCATCTCCCCTGTTCCCTGGACCGTCAAAGCCATTTTGGGTGCTCGTGGTTGT
CGGTGGCGTTCTCGCCTGTTATAGCTTGCTGGTGACAGTAGCCTTCATTATCTTTT
GGGTG
Anti-CLL-1 CAR CD28T (extracellular / TM region of CD28) AA
(SEQ ID NO. 261)
LDNEKSNGTIIHVKGKHLCPSPLFPGPSKPFWVLVVVGGVLACYSLLVTVAFIIFWV
Anti-CLL-1 CAR CD28 (intracellular costimulatory region of CD28) DNA
(SEQ ID NO. 262)
AGATCCAAAAGAAGCCGCCTGCTCCATAGCGATTACATGAATATGACTCCACGC
CGCCCTGGCCCCACAAGGAAACACTACCAGCCTTACGCACCACCTAGAGATTTC
GCTGCCTATCGGAGC
Anti-CLL-1 CAR CD28 (intracellular costimulatory region of CD28) AA
(SEQ ID NO. 263)
RSKRSRLLHSDYMNMTPRRPGPTRKHYQPYAPPRDFAAYRS
Anti-CLL-1 CAR CD3 zeta DNA
(SEQ ID NO. 264)
CGAGTGAAATTTTCTAGATCAGCTGATGCTCCCGCCTATCAGCAGGGACAGAATC
AACTTTACAATGAGCTGAACCTGGGTCGCAGAGAAGAGTACGACGTTTTGGACA
AACGCCGGGGCCGAGATCCTGAGATGGGGGGGAAGCCGAGAAGGAAGAATCCT
CAAGAAGGCCTGTACAACGAGCTTCAAAAAGACAAAATGGCTGAGGCGTACTCT
GAGATCGGCATGAAGGGCGAGCGGAGACGAGGCAAGGGTCACGATGGCTTGTA
TCAGGGCCTGAGTACAGCCACAAAGGACACCTATGACGCCCTCCACATGCAGGC
ACTGCCCCCACGC
Anti-CLL-1 CAR CD3 zeta AA
(SEQ ID NO. 265)
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
CLL scFv (24C1) VLCDR1
(SEQ ID NO. 266)
QASQDINNFLN
CLL scFv (24C1) VLCDR2
(SEQ ID NO. 267)
DASNLET
CLL scFv (24C1) VLCDR3
(SEQ ID NO. 268)
QQYGNLPFT
CLL scFv (24C1) VHCDR1
(SEQ ID NO. 269)
GGSISSY
CLL scFv (24C1) VHCDR2
(SEQ ID NO. 270)
YYSGS
CLL scFv (24C1) VHCDR3
(SEQ ID NO. 271)
LVYCGGDCYSGFDY
Anti-BCMA CAR Leader (CD8a) DNA
(SEQ ID NO. 272)
ATGGCACTCCCCGTAACTGCTCTGCTGCTGCCGTTGGCATTGCTCCTGCACGCCG
CACGCCCG
Anti-BCMA CAR Leader (CD8a) AA
(SEQ ID NO. 273)
MALPVTALLLPLALLLHAARP
Anti-BCMA CAR scFv heavy chain DNA
(SEQ ID NO. 274)
CAGGTGCAGCTGGTGGAGTCTGGGGGAGGCGTGGTCCAGCCTGGGAGGTCCCTG
AGACTCTCCTGTGCAGCGTCTGGATTCACCTTCAGTAGCTATGGCATGCACTGGG
TCCGCCAGGCTCCAGGCAAGGGGCTGGAGTGGGTGGCAGTTATATCGTATGATG
GAAGTAATAAATACTATGCAGACTCCGTGAAGGGCCGATTCACCATCTCCAGAG
ACAATTCCAAGAACACGCTGTATCTGCAAATGAACAGCCTGAGAGCCGAGGACA
CGGCGGTGTACTACTGCGTCAAGGGGCCGTTGCAGGAGCCGCCATACGATTATG
GAATGGACGTATGGGGCCAGGGAACAACTGTCACCGTCTCCTCA
Anti-BCMA CAR scFv heavy chain AA
(SEQ ID NO. 275)
QVQLVESGGGVVQPGRSLRLSCAASGFTFSSYGMHWVRQAPGKGLEWVAVISYDG
SNKYYADSVKGRFTISRDNSKNTLYLQMNSLRAEDTAVYYCVKGPLQEPPYDYGM
DVWGQGTTVTVSS
Anti-BCMA CAR Linker (Whitlow) DNA
(SEQ ID NO. 276)
GGGTCTACATCCGGCTCCGGGAAGCCCGGAAGTGGCGAAGGTAGTACAAAGGGG
Anti-BCMA CAR Linker (Whitlow) AA
(SEQ ID NO. 277)
GSTSGSGKPGSGEGSTKG
Anti-BCMA CAR scFv light chain DNA
(SEQ ID NO. 278)
GAAATAGTGATGACGCAGTCTCCAGCCACCCTGTCTGTGTCTCCAGGGGAAAGA
GCCACCCTCTCCTGCAGGGCCAGTCAGAGTGTTAGCAGCAACTTAGCCTGGTACC
AGCAGAAACCTGGCCAGGCTCCCAGGCTCCTCATCTATAGCGCATCCACCAGGG
CCACTGGTATCCCAGCCAGGTTCAGTGGCAGTGGGTCTGGGACAGAGTTCACTCT
CACCATCAGCAGCCTGCAGTCTGAAGATTTTGCAGTTTATTACTGTCAGCAGCAC
CACGTCTGGCCTCTCACTTTTGGCGGAGGGACCAAGGTTGAGATCAAACGG
Anti-BCMA CAR scFv light chain AA
(SEQ ID NO. 279)
EIVMTQSPATLSVSPGERATLSCRASQSVSSNLAWYQQKPGQAPRLLIYSASTRATGI
PARFSGSGSGTEFTLTISSLQSEDFAVYYCQQHHVWPLTFGGGTKVEIKR
Anti-BCMA CAR Minispacer DNA
(SEQ ID NO. 280)
GCCGCTGCC
Anti-BCMA CAR Minispacer AA
(SEQ ID NO. 281)
AAA
Anti-BCMA CAR CD28T spacer DNA
(SEQ ID NO. 282)
CTTGATAATGAAAAGTCAAACGGAACAATCATTCACGTGAAGGGCAAGCACCTC
TGTCCGTCACCCTTGTTCCCTGGTCCATCCAAGCCATTCTGGGTGTTGGTCGTAGT
GGGTGGAGTCCTCGCTTGTTACTCTCTGCTCGTCACCGTGGCTTTTATAATCTTCT
GGGTT
Anti-BCMA CAR CD28T spacer AA
(SEQ ID NO. 283)
LDNEKSNGTIIHVKGKHLCPSPLFPGPSKPFWVLVVVGGVLACYSLLVTVAFIIFWV
Anti-BCMA CAR CD28 costimulatory region DNA
(SEQ ID NO. 284)
AGATCCAAAAGAAGCCGCCTGCTCCATAGCGATTACATGAATATGACTCCACGC
CGCCCTGGCCCCACAAGGAAACACTACCAGCCTTACGCACCACCTAGAGATTTC
GCTGCCTATCGGAGC
Anti-BCMA CAR CD28 costimulatory region AA
(SEQ ID NO. 285)
RSKRSRLLHSDYMNMTPRRPGPTRKHYQPYAPPRDFAAYRS
Anti-BCMA CAR CD3 zeta DNA
(SEQ ID NO. 286)
AGGGTGAAGTTTTCCAGATCTGCAGATGCACCAGCGTATCAGCAGGGCCAGAAC
CAACTGTATAACGAGCTCAACCTGGGACGCAGGGAAGAGTATGACGTTTTGGAC
AAGCGCAGAGGACGGGACCCTGAGATGGGTGGCAAACCAAGACGAAAAAACCC
CCAGGAGGGTCTCTATAATGAGCTGCAGAAGGATAAGATGGCTGAAGCCTATTC
TGAAATAGGCATGAAAGGAGAGCGGAGAAGGGGAAAAGGGCACGACGGTTTGT
ACCAGGGACTCAGCACTGCTACGAAGGATACTTATGACGCTCTCCACATGCAAG
CCCTGCCACCTAGG
Anti-BCMA CAR CD3 zeta AA
(SEQ ID NO. 287)
RVKFSRSADAPAYQQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
Anti-BCMA CAR VL CDR1
(SEQ ID NO. 288)
RASQSVSSNLA
Anti-BCMA CAR VL CDR2
(SEQ ID NO. 289)
SASTRAT
Anti-BCMA CAR VL CDR3
(SEQ ID NO. 290)
QQHHVWPLTF
Anti-BCMA CAR VH CDR1
(SEQ ID NO. 291)
GFTFSSY
Anti-BCMA CAR VH CDR2
(SEQ ID NO. 292)
VISYDGSNKYYADSVKG
Anti-BCMA CAR VH CDR3
(SEQ ID NO. 293)
VKGPLQEPPYDYGMDV
C185 E7₁₁₋₁₉/HLA-A*02:01 specific TCR - TCR beta chain variable region
DNA
(SEQ ID NO. 294)
atgggcaccagactgttcttttatgtggccctgtgtctgctgtggaccggccacatggatgccggaattacacagagccccagacacaa
agtgaccgagacaggcacccctgtgacactgagatgccaccagaccgagaaccaccgctacatgtactggtacagacaggatccag
gccacggcctgagactgatccactacagctacggcgtgaaggacaccgacaagggcgaagtgtctgacggctacagcgtgtccag
aagcaagaccgaggatttcctgctgaccctggaaagcgccacaagcagccagaccagcgtgtacttttgtgccatcagcggctacaa
gaacaccgaggcctttttcggccaaggcaccaggctgacagtggtg
C185 E7₁₁₋₁₉/HLA-A*02:01 specific TCR - TCR beta chain variable region
AA
(SEQ ID NO. 295)
MGTRLFFYVALCLLWTGHMDAGITQSPRHKVTETGTPVTLRCHQTENHRYMYWYR
QDPGHGLRLIHYSYGVKDTDKGEVSDGYSVSRSKTEDFLLTLESATSSQTSVYFCAIS
GYKNTEAFFGQGTRLTVV
C185 E7₁₁₋₁₉/HLA-A*02:01 specific TCR - TCR beta chain constant region
DNA
(SEQ ID NO. 296)
gaagatctgaagaacgtgttcccacctgaggtggccgtgtttgagccttctgaggccgagatcagccacacacagaaagccacactc
gtgtgcctggccaccggcttttatcccgatcacgtggaactgtcttggtgggtcaacggcaaagaggtgcacagcggcgttagcacag
accctcagcctctgaaagagcagcccgctctgaacgacagcagatactgtctgagcagcagactgagagtgtccgccaccttctggc
agaaccccagaaaccacttcagatgccaggtgcagttctacggcctgtccgagaatgacgagtggacccaggatagagccaagcca
gtgacacagattgtgtctgccgaagcctggggcagagccgattgtggctttacaagcgagagctaccagcagggcgtgctgtctgcc
acaatcctgtatgagatcctgctgggcaaagccactctgtacgctgtgctggtgtctgccctggtgctgatggccatggtcaagagaaa
ggacagcagaggc
C185 E7₁₁₋₁₉/HLA-A*02:01 specific TCR - TCR beta chain constant region
AA
(SEQ ID NO. 297)
EDLKNVFPPEVAVFEPSEAEISHTQKATLVCLATGFYPDHVELSWWVNGKEVHSGV
STDPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLM
AMVKRKDSRG
C185 E7₁₁₋₁₉/HLA-A*02:01 specific TCR - P2A peptide (with Furin cleavage
site and linker) DNA
(SEQ ID NO. 298)
agagccaagagatctggcagcggcgccacaaactttagcctgctgaaacaggccggcgacgtggaagagaaccctggacct
C185 E7₁₁₋₁₉/HLA-A*02:01 specific TCR - P2A peptide (with Furin cleavage
site and linker) AA
(SEQ ID NO. 299)
RAKRSGSGATNFSLLKQAGDVEENPGP
C185 E7₁₁₋₁₉/HLA-A*02:01 specific TCR- TCR alpha chain variable region
DNA
(SEQ ID NO. 300)
atgaagtccctgcgggtgctgctggttattctgtggctgcagctgagctgggtttggagccagggacagcaagtgatgcagatccctca
gtaccagcacgtgcaagaaggcgaggacttcaccacctactgcaacagcagcacaaccctgagcaacatccagtggtacaagcag
aggcctggcgggcaccctgtgtttctgatccagctggttaagagcggcgaagtgaagaagcagaagcggctgacctttcagttcggc
gaggccaagaagaacagcagcctgcacattaccgccacacagaccaccgacgtgggcacatatttttgcgctggcagagaaggcg
gcagcgagaagctggtttttggcaagggcaccaaactgaccgtgaatccc
C185 E7₁₁₋₁₉/HLA-A*02:01 specific TCR- TCR alpha chain variable region
AA
(SEQ ID NO. 301)
MKSLRVLLVILWLQLSWVWSQGQQVMQIPQYQHVQEGEDFTTYCNSSTTLSNIQW
YKQRPGGHPVFLIQLVKSGEVKKQKRLTFQFGEAKKNSSLHITATQTTDVGTYFCAG
REGGSEKLVFGKGTKLTVNP
C185 E7₁₁₋₁₉/HLA-A*02:01 specific TCR - TCR alpha chain constant region
DNA
(SEQ ID NO. 302)
gacattcagaaccccgatccagccgtgtaccagctgagagacagcaagagcagcgacaagagcgtgtgtctgttcaccgacttcgac
tcccagaccaatgtgtcccagagcaaggactccgacgtgtacatcaccgataagaccgtgctggacatgcggagcatggacttcaag
agcaatagcgccgtggcttggagcaacaagagcgactttgcctgcgccaacgccttcaacaacagcatcatccccgaggacacattc
ttcccaagtcctgagagcagctgcgacgtgaagctggtggaaaagagcttcgagacagacaccaacctgaacttccagaacctgagc
gtgatcggcttcagaatcctgctgctgaaggtggccggcttcaacctgctgatgactctgagactgtggtccagctgaattcggatccaa
gcttaggcctgctcgctttcttgctgtcccatttctattaaaggttcctttgttc
C185 E7₁₁₋₁₉/HLA-A*02:01 specific TCR - TCR alpha chain constant region
AA
(SEQ ID NO. 303)
DIQNPDPAVYQLRDSKSSDKSVCLFTDFDSQTNVSQSKDSDVYITDKTVLDMRSMDF
KSNSAVAWSNKSDFACANAFNNSIIPEDTFFPSPESSCDVKLVEKSFETDTNLNFQNL
SVIGFRILLLKVAGFNLLMTLRLWSS
C185 E7₁₁₋₁₉/HLA-A*02:01 specific TCR- alpha CDR1
(SEQ ID NO. 304)
TTLSN
C185 E7₁₁₋₁₉/HLA-A*02:01 specific TCR- alpha CDR2
(SEQ ID NO. 305)
LVKSGEV
C185 E7₁₁₋₁₉/HLA-A*02:01 specific TCR- alpha CDR3
(SEQ ID NO. 306)
AGREGGSEKLV
C185 E7₁₁₋₁₉/HLA-A*02:01 specific TCR- beta CDR1
(SEQ ID NO. 307)
ENHRY
C185 E7₁₁₋₁₉/HLA-A*02:01 specific TCR- beta CDR2
(SEQ ID NO. 308)
SYGVKD
C185 E7₁₁₋₁₉/HLA-A*02:01 specific TCR- beta CDR3
(SEQ ID NO. 309)
AISGYKNTEAF
E7₁₁₋₁₉/HLA-A*02:01 specific TCR - TCR beta chain variable region DNA
(SEQ ID NO. 310)
atgggacctggattgctttgttgggccctgctgtgtctgcttggagctggacttgtggatgccggcgtgacacagtctcccacacacctg
atcaagaccagaggccagcaagtgaccctgagatgtagccctaagagcggccacgacaccgtgtcttggtatcagcaggctcttggc
cagggacctcagttcatcttccagtactacgaggaagaggaacggcagcggggcaacttccctgatagattctctggccatcagttcc
ccaactacagcagcgagctgaacgtgaacgctctgctgctgggcgatagcgccctgtatctgtgtgccagttctcttggttggagaggc
ggcagatacaacgagcagttctttggccctggcaccagactgaccgtgctg
E7₁₁₋₁₉/HLA-A*02:01 specific TCR - TCR beta chain variable region AA
(SEQ ID NO. 311)
MGPGLLCWALLCLLGAGLVDAGVTQSPTHLIKTRGQQVTLRCSPKSGHDTVSWYQ
QALGQGPQFIFQYYEEEERQRGNFPDRFSGHQFPNYSSELNVNALLLGDSALYLCAS
SLGWRGGRYNEQFFGPGTRLTVL
E7₁₁₋₁₉/HLA-A*02:01 specific TCR - TCR beta chain constant region DNA
(SEQ ID NO. 312)
gaagatctgaagaacgtgttcccacctgaggtggccgtgtttgagccttctgaggccgagatcagccacacacagaaagccacactc
gtgtgcctggccaccggcttttatcccgatcacgtggaactgtcttggtgggtcaacggcaaagaggtgcacagcggcgttagcacag
accctcagcctctgaaagagcagcccgctctgaacgacagcagatactgtctgagcagcagactgagagtgtccgccaccttctggc
agaaccccagaaaccacttcagatgccaggtgcagttctacggcctgtccgagaatgacgagtggacccaggatagagccaagcca
gtgacacagattgtgtctgccgaagcctggggcagagccgattgtggctttacaagcgagagctaccagcagggcgtgctgtctgcc
acaatcctgtatgagatcctgctgggcaaagccactctgtacgctgtgctggtgtctgccctggtgctgatggccatggtcaagagaaa
ggacagcagaggc
E7₁₁₋₁₉/HLA-A*02:01 specific TCR - TCR beta chain constant region AA
(SEQ ID NO. 313)
EDLKNVFPPEVAVFEPSEAEISHTQKATLVCLATGFYPDHVELSWWVNGKEVHSGV
STDPQPLKEQPALNDSRYCLSSRLRVSATFWQNPRNHFRCQVQFYGLSENDEWTQD
RAKPVTQIVSAEAWGRADCGFTSESYQQGVLSATILYEILLGKATLYAVLVSALVLM
AMVKRKDSRG
E7₁₁₋₁₉/HLA-A*02:01 specific TCR - P2A (with Furin cleavage site and
linker) DNA
(SEQ ID NO. 314)
agagccaagagatctggcagcggcgccacaaactttagcctgctgaaacaggccggcgacgtggaagagaaccctggacct
E7₁₁₋₁₉/HLA-A*02:01 specific TCR - P2A (with Furin cleavage site and
linker) AA
(SEQ ID NO. 315)
RAKRSGSGATNFSLLKQAGDVEENPGP
E7₁₁₋₁₉/HLA-A*02:01 specific TCR - TCR alpha chain variable region DNA
(SEQ ID NO. 316)
atgaagtccctgcgggtgctgctggttattctgtggctgcagctgagctgggtttggagccagggacagaacatcgaccagcctaccg
agatgacagccaccgaaggcgccatcgtgcagatcaattgcacctaccagaccagcggcttcaacggcctgttctggtatcaacagc
atgccggcgaggcccctaccttcctgagctataatgtgctggacggcctggaagaaaagggcagattcagcagcttcctgtccagaa
gcaagggctacagctacctgctgctgaaagaactccagatgaaggacagcgcctcctacctgtgtgcctccgtggatggaaacaaca
gactggccttcggcaagggcaaccaggtggtggtcatcccc
E7₁₁₋₁₉/HLA-A*02:01 specific TCR - TCR alpha chain variable region AA
(SEQ ID NO. 317)
MKSLRVLLVILWLQLSWVWSQGQNIDQPTEMTATEGAIVQINCTYQTSGFNGLFWY
QQHAGEAPTFLSYNVLDGLEEKGRFSSFLSRSKGYSYLLLKELQMKDSASYLCASVD
GNNRLAFGKGNQVVVIP
E7₁₁₋₁₉/HLA-A*02:01 specific TCR - TCR alpha chain constant region DNA
(SEQ ID NO. 318)
gacattcagaaccccgatccagccgtgtaccagctgagagacagcaagagcagcgacaagagcgtgtgtctgttcaccgacttcgac
tcccagaccaatgtgtcccagagcaaggactccgacgtgtacatcaccgataagaccgtgctggacatgcggagcatggacttcaag
agcaatagcgccgtggcttggagcaacaagagcgactttgcctgcgccaacgccttcaacaacagcatcatccccgaggacacattc
ttcccaagtcctgagagcagctgcgacgtgaagctggtggaaaagagcttcgagacagacaccaacctgaacttccagaacctgagc
gtgatcggcttcagaatcctgctgctgaaggtggccggcttcaacctgctgatgactctgagactgtggtccagctgaattcggatccaa
gcttaggcctgctcgctttcttgctgtcccatttctattaaaggttcctttgttc
E7₁₁₋₁₉/HLA-A*02:01 specific TCR - TCR alpha chain constant region AA
(SEQ ID NO. 319)
DIQNPDPAVYQLRDSKSSDKSVCLFTDFDSQTNVSQSKDSDVYITDKTVLDMRSMDF
KSNSAVAWSNKSDFACANAFNNSIIPEDTFFPSPESSCDVKLVEKSFETDTNLNFQNL
SVIGFRILLLKVAGFNLLMTLRLWSS
E7₁₁₋₁₉/HLA-A*02:01 specific TCR - alpha CDR1
(SEQ ID NO. 320)
TSGFNG
E7₁₁₋₁₉/HLA-A*02:01 specific TCR - alpha CDR2
(SEQ ID NO. 321)
NVLDGL
E7₁₁₋₁₉/HLA-A*02:01 specific TCR - alpha CDR3
(SEQ ID NO. 322)
ASVDGNNRLA
E7₁₁₋₁₉/HLA-A*02:01 specific TCR - beta CDR1
(SEQ ID NO. 323)
SGHDT
E7₁₁₋₁₉/HLA-A*02:01 specific TCR - beta CDR2
(SEQ ID NO. 324)
YYEEEE
E7₁₁₋₁₉/HLA-A*02:01 specific TCR - beta CDR3
(SEQ ID NO. 325)
ASSLGWRGGRYNEQF

What is claimed is:

1. A method of performing a patient-specific immunotherapy procedure, the method comprising:
receiving, by a computing device, a cell order request to create transfected T cells for a patient;
generating, by the computing device, a patient-specific identifier associated with the cell order request, the patient-specific identifier comprising a patient identity element, a sales order identifier, and a cell order lot number;
initiating, by the computing device, a process to create transfected T cells for infusion into the patient's bloodstream, the process comprising:
performing a leukapheresis procedure on a sample of the patient's blood to collect T cells from the sample,
transferring the collected T cells to a container;
labeling the container with the patient-specific identifier,
transmitting the collected T cells to a manufacturing facility,
creating transfected T cells from the collected T cells using a cell modification technique,
receiving the transfected T cells from the manufacturing facility, and
infusing the transfected T cells into the patient's bloodstream,
wherein the computing device records a tracking event for each step in the process, each tracking event including the patient-specific identifier, and
wherein the tracking events comprise a chain of custody of the patient's T cells during the process.

2. The method of claim 1, wherein the transfected T cells are created by transfecting the collected T cells with a polynucleotide encoding a chimeric antigen receptor (CAR), the CAR comprising an antigen binding molecule that specifically binds to a target molecule.

3. The method of claim 2, wherein the antigen binding molecule is a single chain variable fragment.

4. The method of claim 2, wherein the target molecule is a blood borne cancer-associated antigen.

5. The method of claim 2, wherein the target molecule is a viral infection-associated antigen.

6. The method of claim 2, wherein the chimeric antigen receptor (CAR) further comprises at least one costimulatory domain.

7. The method of claim 2, wherein the chimeric antigen receptor (CAR) further comprises at least one activating domain.

8. The method of claim 2, wherein the polynucleotide is a component of a vector.

9. The method of claim 1, wherein the transfected T cells are created by transfecting the collected T cells with a polynucleotide encoding a T cell receptor (TCR).

10. The method of claim 9, wherein the TCR binds to a tumor-associated antigen.

11. The method of claim 9, wherein the TCR binds to a viral infection-associated antigen.

12. The method of claim 9, wherein the polynucleotide is a component of a vector.

13. The method of claim 1, wherein initiating a process to create transfected T cells further comprises:
receiving, by the computing device, indicia that the transfected T cells have been shipped from a first site after being created; and
receiving indicia that the transfected T cells have been received at a second site before being infused,
wherein the computing device records a tracking event upon receiving the indicia that the transfected T cells have been shipped, and
wherein the computing device records a tracking event upon receiving the indicia that the transfected T cells have been received at a second site.

14. The method of claim 1, wherein the patient identity element comprises a first patient ID associated with the immunotherapy procedure and a second patient ID associated with a facility that administers one or more of: the leukapheresis procedure or the infusion of the transfected T cells.

15. The method of claim 1, wherein the computing device stores the tracking events in an ordered sequence.

16. A method for tracking a cell order during an immunotherapy procedure, the method comprising:
receiving, by a computing device, a cell order request for creating transfected T cells for a patient;
generating, by the computing device, a patient-specific identifier associated with the cell order request, the patient-specific identifier comprising a patient identity element, a sales order identifier, and a cell order lot number;
monitoring, by the computing device, a process to create transfected T cells for infusion into the patient's bloodstream, the process comprising:
receiving indicia that a leukapheresis procedure has been performed on a sample of the patient's blood to collect T cells from the sample,
receiving indicia that the collected T cells have been transferred to a container,
receiving indicia that the container has been labeled with the patient-specific identifier,
receiving indicia that the collected T cells have been transmitted to a manufacturing facility,
receiving indicia that transfected T cells have been created from the collected T cells using a cell modification technique,
receiving indicia that the transfected T cells have been received from the manufacturing facility, and
receiving indicia that the transfected T cells have been infused into the patient's bloodstream,
recording, by the computing device, a tracking event when indicia is received, each tracking event including the patient-specific identifier, and
maintaining, by the computing device, a chain of custody of the patient's T cells by storing the tracking events during the process.

17. The method of claim 16, wherein the transfected T cells are created by transfecting the collected T cells with a polynucleotide encoding a chimeric antigen receptor (CAR), the CAR comprising an antigen binding molecule that specifically binds to a target molecule.

18. The method of claim 17, wherein the antigen binding molecule is a single chain variable fragment.

19. The method of claim 17, wherein the target molecule is a blood borne cancer-associated antigen.

20. The method of claim 17, wherein the target molecule is a viral infection-associated antigen.

21. The method of claim 17, wherein the chimeric antigen receptor (CAR) further comprises at least one costimulatory domain.

22. The method of claim 17, wherein the chimeric antigen receptor (CAR) further comprises at least one activating domain.

23. The method of claim 17, wherein the polynucleotide is a component of a vector.

24. The method of claim 16, wherein the transfected T cells are created by transfecting the collected T cells with a polynucleotide encoding a T cell receptor (TCR).

25. The method of claim 24, wherein the TCR binds to a tumor-associated antigen.

26. The method of claim 24, wherein the TCR binds to a viral infection-associated antigen.

27. The method of claim 24, wherein the polynucleotide is a component of a vector.

28. The method of claim 16, wherein initiating a process to create transfected T cells further comprises:
receiving, by the computing device, indicia that the transfected T cells have been shipped from a first site after being created; and
receiving indicia that the transfected T cells have been received at a second site before being infused,
wherein the computing device records a tracking event upon receiving the indicia that the transfected T cells have been shipped, and
wherein the computing device records a tracking event upon receiving the indicia that the transfected T cells have been received at a second site.

29. The method of claim 16, wherein the patient identity element comprises a first patient ID associated with the immunotherapy procedure and a second patient ID associated with a facility that administers one or more of: the leukapheresis procedure or the infusion of the transfected T cells.

30. The method of claim 16, wherein the computing device stores the tracking events in an ordered sequence.

31. A method of performing a patient-specific immunotherapy procedure, the method comprising:
receiving a cell order request to create transfected T cells for a patient;
generating, by an event tracking module executed on a processor, a patient-specific identifier associated with the cell order request;
initiating a process to create transfected T cells for infusion into the patient's bloodstream, the process comprising:
performing a leukapheresis procedure on a sample of the patient's blood to collect T cells from the sample,
transferring the collected T cells to a container;
labeling the container with the patient-specific identifier,
transmitting the collected T cells to a manufacturing facility,
creating transfected T cells from the collected T cells using a cell modification technique,
receiving the transfected T cells from the manufacturing facility, and
infusing the transfected T cells into the patient's bloodstream,
receiving at the event tracking module, from a first client device located at the point of the leukapheresis procedure, a first tracking event that confirms the leukapheresis procedure and contains the patient-specific identifier;
integrating, by the event tracking module, the first tracking event in a data structure pertaining to the patient-specific identifier, wherein the data structure is stored in a database and the integrating step records a first timestamp with the first tracking event;
receiving, at the event tracking module, from a second client device located at the manufacturing facility, a second tracking event that confirms the receipt of the collected T cells at the manufacturing facility and contains the patient-specific identifier; and
integrating, by the event tracking module, the second tracking event in the data structure pertaining to the patient-specific identifier, wherein the integrating step records a second timestamp with the second tracking event.

32. A method of performing a patient-specific immunotherapy procedure, the method comprising:
receiving, by a tracking module executed on a processor, a cell order request to create transfected T cells for a patient;
generating, by the tracking module, a patient-specific identifier associated with the cell order request, the patient-specific identifier identifying a patient, and a cell order lot;
generating, in a database, a data record for tracking the cell order, the data record identified in the database according to the patient-specific identifier;
receiving, by the tracking module, a first tracking event indicating that the collected T cells are ready for shipment to a manufacturing facility;
updating the data record corresponding to the patient-specific identifier in accordance with the first tracking event;
receiving, by the tracking module based on the container having been received by the manufacturing facility, a second tracking event indicating that the collected T cells have been received by a manufacturing facility;
updating the data record corresponding to the patient-specific identifier in accordance with the second tracking event;
receiving, by the tracking module based on the manufacturing facility having created transfected T cells from the collected T cells using a cell modification technique, a third tracking event indicating that the transfected T cells have been created;
updating the data record corresponding to the patient-specific identifier in accordance with the third tracking event;
receiving, by the tracking module based on the transfected T cells having been received from the manufacturing facility, a fourth tracking event indicating that the transfected T cells have been received;
updating the data record corresponding to the patient-specific identifier in accordance with the fourth tracking event;
receiving, by the tracking module based on the transfected T cells having been infused into the patient's bloodstream, a fifth tracking event indicating that the transfected T cells have been infused into the patient's bloodstream; and
updating the data record corresponding to the patient-specific identifier in accordance with the fifth tracking event,
wherein each of the first, second, third, fourth, and fifth tracking events contains the patient-specific identifier, a timestamp, and an event identifier, and
wherein the data record corresponding to the patient-specific identifier stores, in an ordered sequence, the first, second, third, fourth, and fifth tracking events when the data record is updated in accordance with the respective events.


SEQUENCE LISTING (326 sequences)

SEQ ID NO: 1 - DNA, 294 bp, Artificial Sequence
  source: /note=“Description of Artificial Sequence Synthetic polynucleotide”
cttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagc

SEQ ID NO: 2 - protein, 98 aa, Artificial Sequence
  source: /note=“Description of Artificial Sequence Synthetic polypeptide”
LDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 3 - DNA, 90 bp, Artificial Sequence
  source: /note=“Description of Artificial Sequence Synthetic oligonucleotide”
cttgataatgaaaagtcaaacggaacaatcattcacgtgaagggcaagcacctctgtccgtcacccttgttccctggtccatccaagcca

SEQ ID NO: 4 - protein, 30 aa, Artificial Sequence
  source: /note=“Description of Artificial Sequence Synthetic polypeptide”
LDNEKSNGTIIHVKGKHLCPSPLFPGPSKP

SEQ ID NO: 5 - DNA, 81 bp, Artificial Sequence
  source: /note=“Description of Artificial Sequence Synthetic oligonucleotide”
ttctgggtgttggtcgtagtgggtggagtcctcgcttgttactctctgctcgtcaccgtggcttttataatcttctgggtt

SEQ ID NO: 6 - protein, 27 aa, Artificial Sequence
  source: /note=“Description of Artificial Sequence Synthetic peptide”
FWVLVVVGGVLACYSLLVTVAFIIFWV

SEQ ID NO: 7 - DNA, 123 bp, Artificial Sequence
  source: /note=“Description of Artificial Sequence Synthetic polynucleotide”
agatccaaaagaagccgcctgctccatagcgattacatgaatatgactccacgccgccctggccccacaaggaaacactaccagccttacgcaccacctagagatttcgctgcctatcggagc

SEQ ID NO: 8 - protein, 41 aa, Artificial Sequence
  source: /note=“Description of Artificial Sequence Synthetic polypeptide”
RSKRSRLLHSDYMNMTPRRPGPTRKHYQPYAPPRDFAAYRS

SEQ ID NO: 9 - DNA, 336 bp, Artificial Sequence
  source: /note=“Description of Artificial Sequence Synthetic polynucleotide”
agggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 10 - protein, 112 aa, Artificial Sequence
  source: /note=“Description of Artificial Sequence Synthetic polypeptide”
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 117 bp, Artificial Sequence
  source: /note=“Description of Artificial Sequence Synthetic polynucleotide”
attgaggtgatgtatccaccgccttacctggataacgaaaagagtaacggtaccatcattcacgtgaaaggtaaacacctgtgtccttctcccctcttccccgggccatcaaagccc

SEQ ID NO: 12 - protein, 39 aa, Artificial Sequence
  source: /note=“Description of Artificial Sequence Synthetic polypeptide”
IEVMYPPPYLDNEKSNGTIIHVKGKHLCPSPLFPGPSKP

SEQ ID NO: 13 - DNA, 288 bp, Artificial Sequence
  source: /note=“Description of Artificial Sequence Synthetic polynucleotide”
gctgcagcattgagcaactcaataatgtattttagtcactttgtaccagtgttcttgccggctaagcctactaccacacccgctccacggccacctaccccagctcctaccatcgcttcacagcctctgtccctgcgcccagaggcttgccgaccggccgcagggggcgctgttcataccagaggactggatttcgcctgcgatatctatatctgggcacccctggccggaacctgcggcgtactcctgctgtccctggtcatcacgctctattgtaatcacaggaac

SEQ ID NO: 14 - protein, 96 aa, Artificial Sequence
  source: /note=“Description of Artificial Sequence Synthetic polypeptide”
AAALSNSIMYFSHFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 15 - DNA, 366 bp, Artificial Sequence
  source: /note=“Description of Artificial Sequence Synthetic polynucleotide”
caggtgcagctgcaggaatccggaccggggctggtgaagcccagcgagactctgagtctcacgtgtacagtttctggaggtagcattagctcctactattggtcatggataaggcagccccccgggaagggattggaatggatcggctatatttactacagtgggagcaccaattacaacccctcactgaagtctagagttacaatcagcgttgacacctcaaagaatcagttcagtttgaaattgtctagcgtcacagcagctgatacagccgtctattattgtgtttctctggtctattgcggtggggattgttacagtggctttgactattgggggcagggtactctggttacagtttcttcc

SEQ ID NO: 16 - protein, 122 aa, Artificial Sequence
  source: /note=“Description of Artificial Sequence Synthetic polypeptide”
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSS

SEQ ID NO: 17 - protein, 7 aa, Artificial Sequence
  source: /note=“Description of Artificial Sequence Synthetic peptide”
GGSISSY

SEQ ID NO: 18 - protein, 5 aa, Artificial Sequence
  source: /note=“Description of Artificial Sequence Synthetic peptide”
YYSGS

SEQ ID NO: 19 - protein, 14 aa, Artificial Sequence
  source: /note=“Description of Artificial Sequence Synthetic peptide”
LVYCGGDCYSGFDY

SEQ ID NO: 20 - DNA, 324 bp, Artificial Sequence
  source: /note=“Description of Artificial Sequence Synthetic polynucleotide”
gacatccagttgacacagagcccgagttccttgtccgcctccgtcggggatagagtgtcatttacctgtcaggcctctcaggatattaataactttctgaattggtatcagcaaaagcccggaaaggcacccaagctgttgatttacgacgccagtaacctggagacaggcgtgccctcccggtttagtggtagcggaagcggtacggattttacctttactatcagctctctccaacccgaagacattgcaacctactattgtcaacaatatggaaacctgccttttacatttggcggcggcaccaaggtggagattaagcgg

SEQ ID NO: 21 - protein, 108 aa, Artificial Sequence
  source: /note=“Description of Artificial Sequence Synthetic polypeptide”
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKR

SEQ ID NO: 22 - protein, 11 aa, Artificial Sequence
  source: /note=“Description of Artificial Sequence Synthetic peptide”
QASQDINNFLN

SEQ ID NO: 23 - protein, 7 aa, Artificial Sequence
  source: /note=“Description of Artificial Sequence Synthetic peptide”
DASNLET

SEQ ID NO: 24 - protein, 9 aa, Artificial Sequence
  source: /note=“Description of Artificial Sequence Synthetic peptide”
QQYGNLPFT

SEQ ID NO: 25 - DNA, 1440 bp, Artificial Sequence
  source: /note=“Description of Artificial Sequence Synthetic polynucleotide”
atggcactccccgtaactgctctgctgctgccgttggcattgctcctgcacgccgcacgcccgcaggtccaactgcaagaaagcggacccggactggtgaagccttctgagacacttagtctgacgtgcacggtcagtggcggctccatctcctcctattattggtcatggatacgacaacccccaggtaagggcctggaatggattggctatatctactattcaggaagcacgaactacaatcccagcctgaagtcccgagtgacaatttcagtagataccagtaaaaaccagttcagtcttaaactgtcaagcgtgacagctgccgacaccgctgtgtattactgcgtctcactggtgtattgtggaggggattgttatagcgggttcgattattggggacagggaaccctggtgactgtatcttccggcggcggcggctcagggggtggcggtagtggcggtgggggttccgatattcaactgacacaatcccccagctcactcagcgccagcgtgggggacagggttagctttacctgtcaagcctctcaggatataaataactttctgaactggtatcaacagaagcctgggaaggcgcccaaactcctgatctatgatgcgtccaacctggaaactggcgtgccttcacgctttagcggctctggcagtggtacagacttcacttttaccatctcttcacttcagccggaggacatcgccacatattactgtcaacagtacggaaacttgccctttacttttggaggcggcaccaaagttgaaatcaaaagggccgctgccctggataacgaaaagagcaatgggactataatacatgttaaaggaaaacacctgtgtccatctcccctgttccctggaccgtcaaagccattttgggtgctcgtggttgtcggtggcgttctcgcctgttatagcttgctggtgacagtagccttcattatcttttgggtgagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 26 - protein, 479 aa, Artificial Sequence
  source: /note=“Description of Artificial Sequence Synthetic polypeptide”
MALPVTALLLPLALLLHAARPQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - DNA, 1374 bp, Artificial Sequence
  source: /note=“Description of Artificial Sequence Synthetic polynucleotide”
caggtccaactgcaagaaagcggacccggactggtgaagccttctgagacacttagtctgacgtgcacggtcagtggcggctccatctcctcctattattggtcatggatacgacaacccccaggtaagggcctggaatggattggctatatctactattcaggaagcacgaactacaatcccagcctgaagtcccgagtgacaatttcagtagataccagtaaaaaccagttcagtcttaaactgtcaagcgtgacagctgccgacaccgctgtgtattactgcgtctcactggtgtattgtggaggggattgttatagcgggttcgattattggggacagggaaccctggtgactgtatcttccggcggcggcggctcagggggtggcggtagtggcggtgggggttccgatattcaactgacacaatcccccagctcactcagcgccagcgtgggggacagggttagctttacctgtcaagcctctcaggatataaataactttctgaactggtatcaacagaagcctgggaaggcgcccaaactcctgatctatgatgcgtccaacctggaaactggcgtgccttcacgctttagcggctctggcagtggtacagacttcacttttaccatctcttcacttcagccggaggacatcgccacatattactgtcaacagtacggaaacttgccctttacttttggaggcggcaccaaagttgaaatcaaaagggccgctgccctggataacgaaaagagcaatgggactataatacatgttaaaggaaaacacctgtgtccatctcccctgttccctggaccgtcaaagccattttgggtgctcgtggttgtcggtggcgttctcgcctgttatagcttgctggtgacagtagccttcattatcttttgggtgagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 28 - protein, 458 aa, Artificial Sequence
  source: /note=“Description of Artificial Sequence Synthetic polypeptide”
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - DNA, 1467 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctgcaggaatccggaccggggctggtgaagcccagcgagactctgagtctcacgtgtacagtttctggaggtagcattagctcctactattggtcatggataaggcagccccccgggaagggattggaatggatcggctatatttactacagtgggagcaccaattacaacccctcactgaagtctagagttacaatcagcgttgacacctcaaagaatcagttcagtttgaaattgtctagcgtcacagcagctgatacagccgtctattattgtgtttctctggtctattgcggtggggattgttacagtggctttgactattgggggcagggtactctggttacagtttcttccggggggggaggctctgggggcggaggctcaggtggtggaggcagcgacatccagttgacacagagcccgagttccttgtccgcctccgtcggggatagagtgtcatttacctgtcaggcctctcaggatattaataactttctgaattggtatcagcaaaagcccggaaaggcacccaagctgttgatttacgacgccagtaacctggagacaggcgtgccctcccggtttagtggtagcggaagcggtacggattttacctttactatcagctctctccaacccgaagacattgcaacctactattgtcaacaatatggaaacctgccttttacatttggcggcggcaccaaggtggagattaagcgggcggcagctattgaggtgatgtatccaccgccttacctggataacgaaaagagtaacggtaccatcattcacgtgaaaggtaaacacctgtgtccttctcccctcttccccgggccatcaaagcccttctgggttcttgtggtcgtgggaggcgtgcttgcttgttattctctgctcgttaccgtggcgtttatcattttttgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 30 - protein, 488 aa, Artificial Sequence
  source: /note=“Description of Artificial Sequence Synthetic polypeptide”
MALPVTALLLPLALLLHAARPQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - DNA, 1401 bp, Artificial Sequence
  source: /note=“Description of Artificial Sequence Synthetic polynucleotide”
caggtgcagctgcaggaatccggaccggggctggtgaagcccagcgagactctgagtctcacgtgtacagtttctggaggtagcattagctcctactattggtcatggataaggcagccccccgggaagggattggaatggatcggctatatttactacagtgggagcaccaattacaacccctcactgaagtctagagttacaatcagcgttgacacctcaaagaatcagttcagtttgaaattgtctagcgtcacagcagctgatacagccgtctattattgtgtttctctggtctattgcggtggggattgttacagtggctttgactattgggggcagggtactctggttacagtttcttccggggggggaggctctgggggcggaggctcaggtggtggaggcagcgacatccagttgacacagagcccgagttccttgtccgcctccgtcggggatagagtgtcatttacctgtcaggcctctcaggatattaataactttctgaattggtatcagcaaaagcccggaaaggcacccaagctgttgatttacgacgccagtaacctggagacaggcgtgccctcccggtttagtggtagcggaagcggtacggattttacctttactatcagctctctccaacccgaagacattgcaacctactattgtcaacaatatggaaacctgccttttacatttggcggcggcaccaaggtggagattaagcgggcggcagctattgaggtgatgtatccaccgccttacctggataacgaaaagagtaacggtaccatcattcacgtgaaaggtaaacacctgtgtccttctcccctcttccccgggccatcaaagcccttctgggttcttgtggtcgtgggaggcgtgcttgcttgttattctctgctcgttaccgtggcgtttatcattttttgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 32 - protein, 467 aa, Artificial Sequence
  source: /note=“Description of Artificial Sequence Synthetic polypeptide”
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - DNA, 1548 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcaattgcaagagtccggccccggactcgttaaacccagtgagacgcttagcctgacctgtaccgtctcagggggcagcatctcctcttattactggagctggatcaggcagcctccaggaaaaggccttgaatggattgggtacatctactactctggctcaacaaattataatccatccctgaagtcccgcgtgactatctctgtggacaccagcaagaatcagttttcactgaagttgtctagtgttaccgcggccgacaccgccgtatactactgtgtgtctcttgtgtactgtggcggcgactgctattccgggttcgactactggggccaagggactctggtaaccgtgtcctcaggcggcggcgggtcaggaggaggcggcagtggaggtggcggctccgacatccagctgacacaatcaccatcttccctttcagcttcagtcggggacagagtgtccttcacatgccaggccagccaggatatcaataacttcctgaactggtaccaacagaaacccggaaaggctccaaagctcctgatctatgatgcttccaacctggagaccggcgtgccctccaggttcagtggttcaggatcaggcactgactttacgttcaccatatccagtcttcagcccgaagacattgcaacctattactgccaacaatacgggaaccttccctttacattcggaggcggcaccaaggtggaaatcaaaagggctgcagcattgagcaactcaataatgtattttagtcactttgtaccagtgttcttgccggctaagcctactaccacacccgctccacggccacctaccccagctcctaccatcgcttcacagcctctgtccctgcgcccagaggcttgccgaccggccgcagggggcgctgttcataccagaggactggatttcgcctgcgatatctatatctgggcacccctggccggaacctgcggcgtactcctgctgtccctggtcatcacgctctattgtaatcacaggaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 34 - protein, 515 aa, Artificial Sequence
  source: /note=“Description of Artificial Sequence Synthetic polypeptide”
MALPVTALLLPLALLLHAARPQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - DNA, 1482 bp, Artificial Sequence
  source: /note=“Description of Artificial Sequence Synthetic polynucleotide”
caggtgcaattgcaagagtccggccccggactcgttaaacccagtgagacgcttagcctgacctgtaccgtctcagggggcagcatctcctcttattactggagctggatcaggcagcctccaggaaaaggccttgaatggattgggtacatctactactctggctcaacaaattataatccatccctgaagtcccgcgtgactatctctgtggacaccagcaagaatcagttttcactgaagttgtctagtgttaccgcggccgacaccgccgtatactactgtgtgtctcttgtgtactgtggcggcgactgctattccgggttcgactactggggccaagggactctggtaaccgtgtcctcaggcggcggcgggtcaggaggaggcggcagtggaggtggcggctccgacatccagctgacacaatcaccatcttccctttcagcttcagtcggggacagagtgtccttcacatgccaggccagccaggatatcaataacttcctgaactggtaccaacagaaacccggaaaggctccaaagctcctgatctatgatgcttccaacctggagaccggcgtgccctccaggttcagtggttcaggatcaggcactgactttacgttcaccatatccagtcttcagcccgaagacattgcaacctattactgccaacaatacgggaaccttccctttacattcggaggcggcaccaaggtggaaatcaaaagggctgcagcattgagcaactcaataatgtattttagtcactttgtaccagtgttcttgccggctaagcctactaccacacccgctccacggccacctaccccagctcctaccatcgcttcacagcctctgtccctgcgcccagaggcttgccgaccggccgcagggggcgctgttcataccagaggactggatttcgcctgcgatatctatatctgggcacccctggccggaacctgcggcgtactcctgctgtccctggtcatcacgctctattgtaatcacaggaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 36 - protein, 494 aa, Artificial Sequence
  source: /note=“Description of Artificial Sequence Synthetic polypeptide”
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - DNA, 1440 bp, Artificial Sequence
  source: /note=“Description of Artificial Sequence Synthetic polynucleotide”
atggcactccccgtaactgctctgctgctgccgttggcattgctcctgcacgccgcacgcccggatatccagctcacgcaatccccctcaagcttgagtgcctccgtgggcgaccgggtgtccttcacatgtcaggcaagccaagacataaataatttcctgaattggtaccaacaaaaacccggcaaggctcccaaactcctgatttatgatgcctccaatctggagaccggggtcccttctagattcagcggaagtggcagcggcacagactttacatttactatctcttctctgcaaccagaggacatcgccacatactattgccagcaatacggcaatctgcccttcaccttcggaggcggaaccaaggtagaaattaaaaggggcggtggaggctccggaggggggggctctggcggagggggctcccaagtacaattgcaggagtcagggcctggactcgtgaagccttcagaaactttgtcactgacatgtacagtgtccggcggaagcatttccagttactattggtcctggattagacagccacccggcaaaggactggaatggattggatatatctactactctggatctacaaactataatcccagcctcaaatccagggtcactattagtgtggatacatcaaagaatcagttctccttgaagctgagctcagtcactgctgccgacaccgcagtgtactattgtgtgagcctggtctactgcggcggagattgctacagcggtttcgattactggggccagggcaccctggttaccgttagttccgcggctgctcttgataacgagaagtccaacggtacgattatccacgttaagggtaagcacctttgccctagcccgctgttcccaggccccagtaagcccttttgggtcctcgttgtggtaggtggggtactcgcctgctactccctgctcgtcactgtcgcattcatcatcttctgggtcagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 38 - protein, 470 aa, Artificial Sequence
  source: /note=“Description of Artificial Sequence Synthetic polypeptide”
MALPVTALLLPLALLLHAARPDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDA

SEQ ID NO: 39 - DNA, 1374 bp, Artificial Sequence
  source: /note=“Description of Artificial Sequence Synthetic polynucleotide”
gatatccagctcacgcaatccccctcaagcttgagtgcctccgtgggcgaccgggtgtccttcacatgtcaggcaagccaagacataaataatttcctgaattggtaccaacaaaaacccggcaaggctcccaaactcctgatttatgatgcctccaatctggagaccggggtcccttctagattcagcggaagtggcagcggcacagactttacatttactatctcttctctgcaaccagaggacatcgccacatactattgccagcaatacggcaatctgcccttcaccttcggaggcggaaccaaggtagaaattaaaaggggcggtggaggctccggaggggggggctctggcggagggggctcccaagtacaattgcaggagtcagggcctggactcgtgaagccttcagaaactttgtcactgacatgtacagtgtccggcggaagcatttccagttactattggtcctggattagacagccacccggcaaaggactggaatggattggatatatctactactctggatctacaaactataatcccagcctcaaatccagggtcactattagtgtggatacatcaaagaatcagttctccttgaagctgagctcagtcactgctgccgacaccgcagtgtactattgtgtgagcctggtctactgcggcggagattgctacagcggtttcgattactggggccagggcaccctggttaccgttagttccgcggctgctcttgataacgagaagtccaacggtacgattatccacgttaagggtaagcacctttgccctagcccgctgttcccaggccccagtaagcccttttgggtcctcgttgtggtaggtggggtactcgcctgctactccctgctcgtcactgtcgcattcatcatcttctgggtcagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 40 - protein, 458 aa, Artificial Sequence
  source: /note=“Description of Artificial Sequence Synthetic polypeptide”
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - DNA, 1467 bp, Artificial Sequence
  source: /note=“Description of Artificial Sequence Synthetic polynucleotide”
atggcactccccgtaactgctctgctgctgccgttggcattgctcctgcacgccgcacgcccggatatccagctgacccagtctccatcctctttgagtgcctccgtgggtgaccgcgtctctttcacttgccaagccagccaagacatcaacaactttctgaattggtaccagcagaaaccaggcaaagcaccaaagctcctcatctacgacgcctccaacctggaaaccggggtgcccagcaggtttagcgggagcggttctggcacggattttacgttcaccatctcctctctgcagcccgaggatatagctacttattactgtcagcagtacgggaatctgccatttacttttgggggtggaactaaggtggaaatcaaaaggggcggcgggggaagcgggggcgggggctcaggtggcggagggagccaggtgcaactccaggaaagtggcccaggattggtgaagcccagcgagaccctttcccttacttgtactgttagcggaggcagcataagcagctactattggtcctggatcagacagccaccagggaaagggcttgaatggattggctacatttactattccgggtccaccaactacaacccatccctcaagtcccgcgtgacaatttccgtcgacacaagcaagaaccagttctccctgaaacttagtagcgtcactgctgcagatacagcagtgtactattgtgtcagccttgtctactgtggcggcgactgctacagtggctttgattactggggacagggcacgctcgtgacagtgtccagcgctgcggctatcgaggtaatgtatccgccaccgtatctggacaacgagaagtctaatgggacaatcattcacgtgaaggggaagcacctgtgtccatcccccctgtttccgggtcccagtaaacccttctgggtgcttgttgtcgttggcggggtgctggcctgctattccctgctggtgaccgtcgcgtttattattttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 42 - protein, 488 aa, Artificial Sequence
  source: /note=“Description of Artificial Sequence Synthetic polypeptide”
MALPVTALLLPLALLLHAARPDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - DNA, 1401 bp, Artificial Sequence
  source: /note=“Description of Artificial Sequence Synthetic polynucleotide”
gatatccagctgacccagtctccatcctctttgagtgcctccgtgggtgaccgcgtctctttcacttgccaagccagccaagacatcaacaactttctgaattggtaccagcagaaaccaggcaaagcaccaaagctcctcatctacgacgcctccaacctggaaaccggggtgcccagcaggtttagcgggagcggttctggcacggattttacgttcaccatctcctctctgcagcccgaggatatagctacttattactgtcagcagtacgggaatctgccatttacttttgggggtggaactaaggtggaaatcaaaaggggcggcgggggaagcgggggcgggggctcaggtggcggagggagccaggtgcaactccaggaaagtggcccaggattggtgaagcccagcgagaccctttcccttacttgtactgttagcggaggcagcataagcagctactattggtcctggatcagacagccaccagggaaagggcttgaatggattggctacatttactattccgggtccaccaactacaacccatccctcaagtcccgcgtgacaatttccgtcgacacaagcaagaaccagttctccctgaaacttagtagcgtcactgctgcagatacagcagtgtactattgtgtcagccttgtctactgtggcggcgactgctacagtggctttgattactggggacagggcacgctcgtgacagtgtccagcgctgcggctatcgaggtaatgtatccgccaccgtatctggacaacgagaagtctaatgggacaatcattcacgtgaaggggaagcacctgtgtccatcccccctgtttccgggtcccagtaaacccttctgggtgcttgttgtcgttggcggggtgctggcctgctattccctgctggtgaccgtcgcgtttattattttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 44 - protein, 467 aa, Artificial Sequence
  source: /note=“Description of Artificial Sequence Synthetic polypeptide”
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 1548 bp, Artificial Sequence
  source: /note=“Description of Artificial Sequence Synthetic polynucleotide”
atggcactccccgtaactgctctgctgctgccgttggcattgctcctgcacgccgcacgcccggacattcaattgacccagtcccctagcagtctctcagcaagtgtgggagatagggtgtcattcacctgtcaggcttcacaggacatcaacaacttcctcaattggtatcagcagaagccagggaaggcaccaaagctgctcatatatgacgcttcaaaccttgaaaccggagtacctagccgcttcagcggaagcggatcagggactgacttcacttttaccatctcttcactgcagcccgaagacatcgccacatactactgccagcagtacggaaacttgccttttacatttgggggcggcaccaaagtggagattaagcgagggggaggcggctcaggaggcggtggctccggaggcgggggttcccaggtccagctccaggaatccggcccaggtctggttaagcccagtgaaactttgtccctcacgtgtactgtgagcggtggttcaatctcctcatactattggtcttggatacggcaacctcctggaaagggcctcgagtggatcggctatatctactatagtggctccactaattacaacccttccctcaagtccagagtcaccatttccgtggacacatctaagaaccagttcagtctgaagttgtccagcgttacagccgcagacacagccgtttattactgtgtgtctcttgtttactgcgggggagactgttatagcggcttcgattactggggccagggcaccttggtcacagtctcttccgcggccgccctctctaacagtattatgtacttttctcattttgtacccgtgttccttcccgctaagccaactactaccccggccccacggccgcctacccctgcacccacaatagccagtcagcctttgagcctgagacctgaggcttgtcggccggctgctgggggtgcagtgcacacacgaggtcttgattttgcttgcgacatatacatctgggcccctctggccgggacctgtggggtgctgcttctgagcttggtcatcacgctctattgcaaccatcgcaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 46 - protein, 515 aa, Artificial Sequence
  source: /note=“Description of Artificial Sequence Synthetic polypeptide”
MALPVTALLLPLALLLHAARPDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 1482 bp, Artificial Sequence
  source: /note=“Description of Artificial Sequence Synthetic polynucleotide”
gacattcaattgacccagtcccctagcagtctctcagcaagtgtgggagatagggtgtcattcacctgtcaggcttcacaggacatcaacaacttcctcaattggtatcagcagaagccagggaaggcaccaaagctgctcatatatgacgcttcaaaccttgaaaccggagtacctagccgcttcagcggaagcggatcagggactgacttcacttttaccatctcttcactgcagcccgaagacatcgccacatactactgccagcagtacggaaacttgccttttacatttgggggcggcaccaaagtggagattaagcgagggggaggcggctcaggaggcggtggctccggaggcgggggttcccaggtccagctccaggaatccggcccaggtctggttaagcccagtgaaactttgtccctcacgtgtactgtgagcggtggttcaatctcctcatactattggtcttggatacggcaacctcctggaaagggcctcgagtggatcggctatatctactatagtggctccactaattacaacccttccctcaagtccagagtcaccatttccgtggacacatctaagaaccagttcagtctgaagttgtccagcgttacagccgcagacacagccgtttattactgtgtgtctcttgtttactgcgggggagactgttatagcggcttcgattactggggccagggcaccttggtcacagtctcttccgcggccgccctctctaacagtattatgtacttttctcattttgtacccgtgttccttcccgctaagccaactactaccccggccccacggccgcctacccctgcacccacaatagccagtcagcctttgagcctgagacctgaggcttgtcggccggctgctgggggtgcagtgcacacacgaggtcttgattttgcttgcgacatatacatctgggcccctctggccgggacctgtggggtgctgcttctgagcttggtcatcacgctctattgcaaccatcgcaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 48 - protein, 492 aa, Artificial Sequence
  source: /note=“Description of Artificial Sequence Synthetic polypeptide”
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTADTAVYYCVSLVYCGGDCYSGFDYWGQGTLVTVSSAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGREEYDVLDKRRGRDPEMGGKPRRKNPQEGLYNELQKDKMAEAYSEIGMKGERRRGKGHDGLYQGLSTATKDTYDALHMQALPPR

SEQ ID NO: 49 - DNA, 372 bp, Artificial Sequence
  source: /note=“Description of Artificial Sequence Synthetic polynucleotide”
caggtacagctgcaggaatctgggcccggacttgtcaagccaagtcagacactttctcttacatgtaccgtgagcggcggaagtataagcagtggaggcttttactggtcttggatacggcagcacccaggcaaaggcttggagtggattggatacattcatcattcaggatctacacactataatccatcccttaagtcccgggtcaccattagcattgatacgtctaagaatctgttcagtctcaggctgtcctccgtcactgctgccgacacagccgtgtactactgcgcctccttggtttactgcggaggcgactgttatagcggctttgattattgggggcaggggaccctcgtaaccgtgagctct

SEQ ID NO: 50 - protein, 124 aa, Artificial Sequence
  source: /note=“Description of Artificial Sequence Synthetic polypeptide”
QVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSS

SEQ ID NO: 51 - protein, 9 aa, Artificial Sequence
  source: /note=“Description of Artificial Sequence Synthetic peptide”
GGSISSGGF

SEQ ID NO: 52 - protein, 5 aa, Artificial Sequence
  source: /note=“Description of Artificial Sequence Synthetic peptide”
HHSGS

SEQ ID NO: 53 - protein, 14 aa, Artificial Sequence
  source: /note=“Description of Artificial Sequence Synthetic peptide”
LVYCGGDCYSGFDY

SEQ ID NO: 54 - DNA, 324 bp, Artificial Sequence
  source: /note=“Description of Artificial Sequence Synthetic polynucleotide”
gatatccagctcactcaaagcccctctagtctctctgcctcagtgggggatcgggtcagttttacttgtcaagcttcacaggatatcaacaacttccttaattggtatcagcagaagccaggaaaagcacccaagctgctcatctatgatgcctcaaatttggagacgggtgttcccagtcgattctctgggtcagggtccgggaccgactttacgtttacgatctcctctctgcagcccgaagacatcgccacatactattgtcaacagtacggcaacttgcctttcacatttgggggcgggactaaggttgaaatcaagagg

SEQ ID NO: 55 - protein, 108 aa, Artificial Sequence; identical to SEQ ID NO: 21
  source: /note=“Description of Artificial Sequence Synthetic polypeptide”

SEQ ID NO: 56 - protein, 11 aa, Artificial Sequence
  source: /note=“Description of Artificial Sequence Synthetic peptide”
QASQDINNFLN

SEQ ID NO: 57 - protein, 7 aa, Artificial Sequence
  source: /note=“Description of Artificial Sequence Synthetic peptide”
DASNLET

SEQ ID NO: 58 - protein, 9 aa, Artificial Sequence
  source: /note=“Description of Artificial Sequence Synthetic peptide”
QQYGNLPFT

SEQ ID NO: 59 - DNA, 1446 bp, Artificial Sequence
  source: /note=“Description of Artificial Sequence Synthetic polynucleotide”
atggcactccccgtaactgctctgctgctgccgttggcattgctcctgcacgccgcacgcccgcaggtacagctgcaggaatctgggcccggacttgtcaagccaagtcagacactttctcttacatgtaccgtgagcggcggaagtataagcagtggaggcttttactggtcttggatacggcagcacccaggcaaaggcttggagtggattggatacattcatcattcaggatctacacactataatccatcccttaagtcccgggtcaccattagcattgatacgtctaagaatctgttcagtctcaggctgtcctccgtcactgctgccgacacagccgtgtactactgcgcctccttggtttactgcggaggcgactgttatagcggctttgattattgggggcaggggaccctcgtaaccgtgagctctggagggggtgggagcgggggaggaggttcaggggggggcggctccgatatccagctcactcaaagcccctctagtctctctgcctcagtgggggatcgggtcagttttacttgtcaagcttcacaggatatcaacaacttccttaattggtatcagcagaagccaggaaaagcacccaagctgctcatctatgatgcctcaaatttggagacgggtgttcccagtcgattctctgggtcagggtccgggaccgactttacgtttacgatctcctctctgcagcccgaagacatcgccacatactattgtcaacagtacggcaacttgcctttcacatttgggggcgggactaaggttgaaatcaagagggccgctgcactggacaatgagaagtccaacggcaccatcatccacgtgaagggcaagcacctgtgccctagtcctctgttcccaggcccatccaaacctttttgggttcttgttgtggtcgggggggtgctggcctgctattctctgctggtcacggtggccttcataattttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 60 - protein, 481 aa, Artificial Sequence
  source: /note=“Description of Artificial Sequence Synthetic polypeptide”
MALPVTALLLPLALLLHAARPQVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - DNA, 1380 bp, Artificial Sequence
  source: /note=“Description of Artificial Sequence Synthetic polynucleotide”
caggtacagctgcaggaatctgggcccggacttgtcaagccaagtcagacactttctcttacatgtaccgtgagcggcggaagtataagcagtggaggcttttactggtcttggatacggcagcacccaggcaaaggcttggagtggattggatacattcatcattcaggatctacacactataatccatcccttaagtcccgggtcaccattagcattgatacgtctaagaatctgttcagtctcaggctgtcctccgtcactgctgccgacacagccgtgtactactgcgcctccttggtttactgcggaggcgactgttatagcggctttgattattgggggcaggggaccctcgtaaccgtgagctctggagggggtgggagcgggggaggaggttcaggggggggcggctccgatatccagctcactcaaagcccctctagtctctctgcctcagtgggggatcgggtcagttttacttgtcaagcttcacaggatatcaacaacttccttaattggtatcagcagaagccaggaaaagcacccaagctgctcatctatgatgcctcaaatttggagacgggtgttcccagtcgattctctgggtcagggtccgggaccgactttacgtttacgatctcctctctgcagcccgaagacatcgccacatactattgtcaacagtacggcaacttgcctttcacatttgggggcgggactaaggttgaaatcaagagggccgctgcactggacaatgagaagtccaacggcaccatcatccacgtgaagggcaagcacctgtgccctagtcctctgttcccaggcccatccaaacctttttgggttcttgttgtggtcgggggggtgctggcctgctattctctgctggtcacggtggccttcataattttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 62 - protein, 460 aa, Artificial Sequence
  source: /note=“Description of Artificial Sequence Synthetic polypeptide”
QVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - DNA, 1473 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctgcaggaaagcggtccgggacttgtcaagccgtcccaaacgctgagtctgacgtgtactgtctctggtggctctatttcttccgggggcttttattggtcttggatcagacaacaccctggcaaagggctggagtggatagggtatattcaccactctgggtccactcactacaacccatcattgaaatccagagtgactatctcaatcgacacatccaagaaccttttcagcctgaggttgtcatcagttaccgccgctgacaccgcggtgtattattgcgcctctctcgtgtactgcggtggcgattgttatagtggctttgactactgggggcaggggacattggttaccgtttcaagtggaggcggtgggtctggcgggggcggtagcggaggtggggggagcgacatacagcttacgcagagcccctccagcctttcagcctccgtgggggatagggtgtcctttacctgccaggcttcccaggacataaacaacttcctcaattggtatcagcaaaagcccgggaaagcaccaaagctgctcatctacgatgccagcaacctggaaaccggagtgccgtctcgcttctctggaagtggcagtgggaccgatttcacttttacaatctcaagtttgcagccagaagacattgcaacatactactgtcaacagtacggcaatctcccctttacatttggggggggaactaaagtggagattaagcgcgctgcagccattgaagttatgtatccgcccccgtatctggataacgagaaatctaatggtaccataatacatgtgaaggggaagcacctctgtccatcaccgctgttccccggcccttcaaaacctttctgggtactcgttgtcgtgggtggagttctggcctgctatagtctgctggtgaccgtggcgtttatcatcttctgggtaagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 64 - protein, 490 aa, Artificial Sequence
  source: /note=“Description of Artificial Sequence Synthetic polypeptide”
MALPVTALLLPLALLLHAARPQVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 65 - DNA, 1407 bp, Artificial Sequence
  source: /note=“Description of Artificial Sequence Synthetic polynucleotide”
caggtgcagctgcaggaaagcggtccgggacttgtcaagccgtcccaaacgctgagtctgacgtgtactgtctctggtggctctatttcttccgggggcttttattggtcttggatcagacaacaccctggcaaagggctggagtggatagggtatattcaccactctgggtccactcactacaacccatcattgaaatccagagtgactatctcaatcgacacatccaagaaccttttcagcctgaggttgtcatcagttaccgccgctgacaccgcggtgtattattgcgcctctctcgtgtactgcggtggcgattgttatagtggctttgactactgggggcaggggacattggttaccgtttcaagtggaggcggtgggtctggcgggggcggtagcggaggtggggggagcgacatacagcttacgcagagcccctccagcctttcagcctccgtgggggatagggtgtcctttacctgccaggcttcccaggacataaacaacttcctcaattggtatcagcaaaagcccgggaaagcaccaaagctgctcatctacgatgccagcaacctggaaaccggagtgccgtctcgcttctctggaagtggcagtgggaccgatttcacttttacaatctcaagtttgcagccagaagacattgcaacatactactgtcaacagtacggcaatctcccctttacatttggggggggaactaaagtggagattaagcgcgctgcagccattgaagttatgtatccgcccccgtatctggataacgagaaatctaatggtaccataatacatgtgaaggggaagcacctctgtccatcaccgctgttccccggcccttcaaaacctttctgggtactcgttgtcgtgggtggagttctggcctgctatagtctgctggtgaccgtggcgtttatcatcttctgggtaagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 66 - protein, 469 aa, Artificial Sequence
  source: /note=“Description of Artificial Sequence Synthetic polypeptide”
QVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 1554 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagttgcaggaaagcgggcctggccttgtgaaaccaagccagacactgagcctgacatgcactgtgtccggcgggtccatatcttccgggggtttttattggtcctggatacgccagcatcccgggaaaggacttgaatggattggatatatccaccattccggaagcacccactacaatccaagccttaaatcccgggtgacaatctccatcgacacctcaaagaatcttttttccctgcggttgtcttcagtaactgccgccgataccgctgtgtactactgtgccagcctcgtctattgcggcggagattgttattctgggttcgattattggggtcaaggcacactggtaactgtcagcagcggaggcggcggttccgggggcgggggcagtggagggggcggatctgacattcagcttacgcagtccccatcttcacttagcgccagcgttggcgatcgggtcagcttcacgtgtcaagcaagtcaggatatcaacaactttcttaactggtaccagcagaagccaggcaaggcacccaagttgctgatttacgatgcttctaacctcgagacgggagtgcctagccgcttctccgggagcggcagcggcacagactttacctttacgatttccagtctgcagccagaggatatagcaacttattactgtcagcagtatggcaacctcccttttaccttcggtggtggcacaaaggtcgagattaaaagagccgcagcgttgtccaactccataatgtatttttctcattttgtgcccgtctttctgcctgccaaacctaccaccacccccgccccacgaccacctactccagcccccaccatcgcctcccagcccctcagcctgaggccagaggcttgtcgccctgctgcggggggcgctgtccataccagaggactcgacttcgcctgcgatatttatatatgggcccccctcgccggcacctgcggagtcttgctcctgagccttgtgatcacgctttattgtaaccatcggaatagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 68 - protein, 515 aa, Artificial Sequence
  source: /note=“Description of Artificial Sequence Synthetic polypeptide”
MALPVTALLLPLALLLHAARPQVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPRFSGSGSGTDFTFTISSLQPEDIATYYCQYGNLPFTFGGGTKVEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 69 - DNA, 1488 bp, Artificial Sequence
  source: /note=“Description of Artificial Sequence Synthetic polynucleotide”
caggtgcagttgcaggaaagcgggcctggccttgtgaaaccaagccagacactgagcctgacatgcactgtgtccggcgggtccatatcttccgggggtttttattggtcctggatacgccagcatcccgggaaaggacttgaatggattggatatatccaccattccggaagcacccactacaatccaagccttaaatcccgggtgacaatctccatcgacacctcaaagaatcttttttccctgcggttgtcttcagtaactgccgccgataccgctgtgtactactgtgccagcctcgtctattgcggcggagattgttattctgggttcgattattggggtcaaggcacactggtaactgtcagcagcggaggcggcggttccgggggcgggggcagtggagggggcggatctgacattcagcttacgcagtccccatcttcacttagcgccagcgttggcgatcgggtcagcttcacgtgtcaagcaagtcaggatatcaacaactttcttaactggtaccagcagaagccaggcaaggcacccaagttgctgatttacgatgcttctaacctcgagacgggagtgcctagccgcttctccgggagcggcagcggcacagactttacctttacgatttccagtctgcagccagaggatatagcaacttattactgtcagcagtatggcaacctcccttttaccttcggtggtggcacaaaggtcgagattaaaagagccgcagcgttgtccaactccataatgtatttttctcattttgtgcccgtctttctgcctgccaaacctaccaccacccccgccccacgaccacctactccagcccccaccatcgcctcccagcccctcagcctgaggccagaggcttgtcgccctgctgcggggggcgctgtccataccagaggactcgacttcgcctgcgatatttatatatgggcccccctcgccggcacctgcggagtcttgctcctgagccttgtgatcacgctttattgtaaccatcggaatagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 70 - protein, 496 aa, Artificial Sequence
  source: /note=“Description of Artificial Sequence Synthetic polypeptide”
QVQLQESGPGLVKPSQTLSLTCTVSGGSISSGGFYWSWIRQHPGKGLEWIGYIHHSGSTHYNPSLKSRVTISIDTSKNLFSLRLSSVTAADTAVYYCASLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - DNA, 363 bp, Artificial Sequence
  source: /note=“Description of Artificial Sequence Synthetic polynucleotide”
caggtccaactggtgcagtccggagccgaagtcaagaaaccaggtgcctccgttaaagtgagttgcaaagtctctggatacactctgaccgagctctctatgcactgggtccggcaggcccccggcaagggattggaatggatgggcgggttcgatcctgaggacggagagactatctacgctcaaaaattccagggacgagtgactgtgaccgaagacactagtaccgacactgcctacatggaactttcctctctgcgatcagaagataccgcagtgtactactgtgctactgaatctaggggcattggatggccctacttcgattactggggtcagggaactctggtgactgtctccagc

SEQ ID NO: 72 - protein, 121 aa, Artificial Sequence
  source: /note=“Description of Artificial Sequence Synthetic polypeptide”
QVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSS

SEQ ID NO: 73 - protein, 7 aa, Artificial Sequence
  source: /note=“Description of Artificial Sequence Synthetic peptide”
GYTLTEL

SEQ ID NO: 74 - protein, 6 aa, Artificial Sequence
  source: /note=“Description of Artificial Sequence Synthetic peptide”
DPEDGE

SEQ ID NO: 75 - protein, 12 aa, Artificial Sequence
  source: /note=“Description of Artificial Sequence Synthetic peptide”
ESRGIGWPYFDY

SEQ ID NO: 76 - DNA, 324 bp, Artificial Sequence
  source: /note=“Description of Artificial Sequence Synthetic polynucleotide”
gatattcagatgactcaatctccttcttctctgtccgcttccgtgggcgatagagtgaccattacttgtagggcgtcccagtcaatctccagttatttgaattggtatcagcagaagcccgggaaagcacctaagctgttgatcagcggggcttctagcctgaagagtggggtaccttcacggttcagcggaagcggaagcggaaccgatttcaccctgactatcagcagcctgccacctgaggactttgcaacttactactgccaacagtcatacagcactccgatcactttcggccagggcacccggctcgaaatcaagcgc

SEQ ID NO: 77 - protein, 108 aa, Artificial Sequence
  source: /note=“Description of Artificial Sequence Synthetic polypeptide”
DIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKR

SEQ ID NO: 78 - protein, 11 aa, Artificial Sequence
  source: /note=“Description of Artificial Sequence Synthetic peptide”
RASQSISSYLN

SEQ ID NO: 79 - protein, 7 aa, Artificial Sequence
  source: /note=“Description of Artificial Sequence Synthetic peptide”
GASSLKS

SEQ ID NO: 80 - protein, 9 aa, Artificial Sequence
  source: /note=“Description of Artificial Sequence Synthetic peptide”
QQSYSTPIT

SEQ ID NO: 81 - DNA, 1437 bp, Artificial Sequence
  source: /note=“Description of Artificial Sequence Synthetic polynucleotide”
atggcactccccgtaactgctctgctgctgccgttggcattgctcctgcacgccgcacgcccgcaggtccaactggtgcagtccggagccgaagtcaagaaaccaggtgcctccgttaaagtgagttgcaaagtctctggatacactctgaccgagctctctatgcactgggtccggcaggcccccggcaagggattggaatggatgggcgggttcgatcctgaggacggagagactatctacgctcaaaaattccagggacgagtgactgtgaccgaagacactagtaccgacactgcctacatggaactttcctctctgcgatcagaagataccgcagtgtactactgtgctactgaatctaggggcattggatggccctacttcgattactggggtcagggaactctggtgactgtctccagcggtggaggtggcagcggtggtggcggaagcggggggggcggctctgatattcagatgactcaatctccttcttctctgtccgcttccgtgggcgatagagtgaccattacttgtagggcgtcccagtcaatctccagttatttgaattggtatcagcagaagcccgggaaagcacctaagctgttgatcagcggggcttctagcctgaagagtggggtaccttcacggttcagcggaagcggaagcggaaccgatttcaccctgactatcagcagcctgccacctgaggactttgcaacttactactgccaacagtcatacagcactccgatcactttcggccagggcacccggctcgaaatcaagcgcgctgctgctttggacaatgagaagtcaaacggcaccatcatacatgttaaaggtaaacatctgtgtccctccccgctgttccccggcccttccaaaccgttctgggttctggtggtggtcggaggcgtactcgcttgctatagtctgctggtaactgtcgccttcatcatcttttgggtgagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 82 - protein, 478 aa, Artificial Sequence
  source: /note=“Description of Artificial Sequence Synthetic polypeptide”
MALPVTALLLPLALLLHAARPQVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSSGGGGSGGGGSGGGGSDIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - DNA, 1371 bp, Artificial Sequence
  source: /note=“Description of Artificial Sequence Synthetic polynucleotide”
caggtccaactggtgcagtccggagccgaagtcaagaaaccaggtgcctccgttaaagtgagttgcaaagtctctggatacactctgaccgagctctctatgcactgggtccggcaggcccccggcaagggattggaatggatgggcgggttcgatcctgaggacggagagactatctacgctcaaaaattccagggacgagtgactgtgaccgaagacactagtaccgacactgcctacatggaactttcctctctgcgatcagaagataccgcagtgtactactgtgctactgaatctaggggcattggatggccctacttcgattactggggtcagggaactctggtgactgtctccagcggtggaggtggcagcggtggtggcggaagcggggggggcggctctgatattcagatgactcaatctccttcttctctgtccgcttccgtgggcgatagagtgaccattacttgtagggcgtcccagtcaatctccagttatttgaattggtatcagcagaagcccgggaaagcacctaagctgttgatcagcggggcttctagcctgaagagtggggtaccttcacggttcagcggaagcggaagcggaaccgatttcaccctgactatcagcagcctgccacctgaggactttgcaacttactactgccaacagtcatacagcactccgatcactttcggccagggcacccggctcgaaatcaagcgcgctgctgctttggacaatgagaagtcaaacggcaccatcatacatgttaaaggtaaacatctgtgtccctccccgctgttccccggcccttccaaaccgttctgggttctggtggtggtcggaggcgtactcgcttgctatagtctgctggtaactgtcgccttcatcatcttttgggtgagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 84 - protein, 457 aa, Artificial Sequence
  source: /note=“Description of Artificial Sequence Synthetic polypeptide”
QVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSSGGGGSGGGGSGGGGSDIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - DNA, 1464 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagcttgtgcagagcggggccgaggtgaagaagcccggggccagcgtcaaagtgtcctgtaaggtcagcggttacaccctcaccgagctgagcatgcactgggtacggcaggctcccggcaaaggtcttgagtggatgggtggatttgatccagaagatggagagactatctacgcccagaagttccagggccgggtcaccgtaacagaagacacctcaactgacaccgcttacatggagctgagttcactgcggtccgaggacacggccgtgtattattgtgccaccgagagccgcggaatcggatggccttacttcgactactggggacagggtacacttgttacagtatcatccgggggtggcggctctggtgggggcggctccggagggggtggatcagatatccaaatgactcaaagtccaagttccctgtctgcctcagtcggagatagagtcaccataacctgcagggcaagtcagtccatctcctcctatctgaactggtaccaacagaaacctggaaaggcgcctaagctcctgatctccggagcctcatctttgaaatccggtgtcccatctcgcttcagtggctctggaagcggtacagattttactttgaccattagcagcctcccaccggaagactttgctacatattactgccagcagtcttactcaaccccaatcaccttcgggcaaggcaccagactcgaaataaaaagagcagctgctatcgaggttatgtacccaccgccgtacttggataacgaaaaaagcaatgggaccatcattcatgtgaagggtaagcacctttgccctagcccactgtttcctggcccgagtaaacccttttgggtacttgtggtcgtcggcggcgtgctggcctgctactcactcctggttaccgtcgcattcatcatcttttgggtgagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 86 - protein, 487 aa, Artificial Sequence
  source: /note=“Description of Artificial Sequence Synthetic polypeptide”
MALPVTALLLPLALLLHAARPQVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSSGGGGSGGGGSGGGGSDIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - DNA, 1398 bp, Artificial Sequence
  source: /note=“Description of Artificial Sequence Synthetic polynucleotide”
caggtgcagcttgtgcagagcggggccgaggtgaagaagcccggggccagcgtcaaagtgtcctgtaaggtcagcggttacaccctcaccgagctgagcatgcactgggtacggcaggctcccggcaaaggtcttgagtggatgggtggatttgatccagaagatggagagactatctacgcccagaagttccagggccgggtcaccgtaacagaagacacctcaactgacaccgcttacatggagctgagttcactgcggtccgaggacacggccgtgtattattgtgccaccgagagccgcggaatcggatggccttacttcgactactggggacagggtacacttgttacagtatcatccgggggtggcggctctggtgggggcggctccggagggggtggatcagatatccaaatgactcaaagtccaagttccctgtctgcctcagtcggagatagagtcaccataacctgcagggcaagtcagtccatctcctcctatctgaactggtaccaacagaaacctggaaaggcgcctaagctcctgatctccggagcctcatctttgaaatccggtgtcccatctcgcttcagtggctctggaagcggtacagattttactttgaccattagcagcctcccaccggaagactttgctacatattactgccagcagtcttactcaaccccaatcaccttcgggcaaggcaccagactcgaaataaaaagagcagctgctatcgaggttatgtacccaccgccgtacttggataacgaaaaaagcaatgggaccatcattcatgtgaagggtaagcacctttgccctagcccactgtttcctggcccgagtaaacccttttgggtacttgtggtcgtcggcggcgtgctggcctgctactcactcctggttaccgtcgcattcatcatcttttgggtgagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 88 - protein, 466 aa, Artificial Sequence
  source: /note=“Description of Artificial Sequence Synthetic polypeptide”
QVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSSGGGGSGGGGSGGGGSDIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - DNA, 1545 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagttggtgcaaagcggcgcagaagttaagaaacctggggcgtcagttaaggtgtcttgcaaagtatctggctataccctcactgagctgtccatgcattgggtaaggcaggctcctggaaaggggctcgaatggatgggaggatttgaccctgaagacggagagaccatctacgcccagaaattccagggtagagtaacagtgactgaggacactagcactgacacagcgtacatggagctgagttctctgagaagtgaggacacagccgtttactactgcgctaccgagtccagaggtattggctggccatacttcgactattggggtcagggcaccctggttacagtgagttcaggaggcgggggctctggggggggcggttccggaggggggggctcagatatacagatgacgcagagtccatcaagtctctcagccagcgtgggagatcgcgtgactattacttgccgcgccagccagagtattagctcctatctgaattggtaccagcaaaagcccgggaaggcccctaagcttctgatttctggcgcctcctctttgaagtcaggtgtgccaagcagatttagcgggtctggaagtggcactgactttacacttactatctccagcctgcccccagaggattttgccacatattactgtcagcaaagctactctactccaatcactttcggccagggcacaagattggagattaagagggctgccgcactttcaaattccatcatgtatttcagccattttgtgcctgtttttcttccggccaaacctacaaccactcccgccccacgcccacctactcccgcccctaccattgcctcccagcctctgtctcttagacctgaggcttgtagacctgctgccggcggagccgtgcacactcgcggtctggacttcgcctgcgacatctatatctgggcccctctggccggcacctgcggcgttctccttctctcactcgtaatcacactctattgcaatcacaggaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 90 - protein, 514 aa, Artificial Sequence
  source: /note=“Description of Artificial Sequence Synthetic polypeptide”
MALPVTALLLPLALLLHAARPQVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSSGGGGSGGGGSGGGGSDIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 91 - DNA, 1479 bp, Artificial Sequence
  source: /note=“Description of Artificial Sequence Synthetic polynucleotide”
caggtgcagttggtgcaaagcggcgcagaagttaagaaacctggggcgtcagttaaggtgtcttgcaaagtatctggctataccctcactgagctgtccatgcattgggtaaggcaggctcctggaaaggggctcgaatggatgggaggatttgaccctgaagacggagagaccatctacgcccagaaattccagggtagagtaacagtgactgaggacactagcactgacacagcgtacatggagctgagttctctgagaagtgaggacacagccgtttactactgcgctaccgagtccagaggtattggctggccatacttcgactattggggtcagggcaccctggttacagtgagttcaggaggcgggggctctggggggggcggttccggaggggggggctcagatatacagatgacgcagagtccatcaagtctctcagccagcgtgggagatcgcgtgactattacttgccgcgccagccagagtattagctcctatctgaattggtaccagcaaaagcccgggaaggcccctaagcttctgatttctggcgcctcctctttgaagtcaggtgtgccaagcagatttagcgggtctggaagtggcactgactttacacttactatctccagcctgcccccagaggattttgccacatattactgtcagcaaagctactctactccaatcactttcggccagggcacaagattggagattaagagggctgccgcactttcaaattccatcatgtatttcagccattttgtgcctgtttttcttccggccaaacctacaaccactcccgccccacgcccacctactcccgcccctaccattgcctcccagcctctgtctcttagacctgaggcttgtagacctgctgccggcggagccgtgcacactcgcggtctggacttcgcctgcgacatctatatctgggcccctctggccggcacctgcggcgttctccttctctcactcgtaatcacactctattgcaatcacaggaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 92 - protein, 493 aa, Artificial Sequence
  source: /note=“Description of Artificial Sequence Synthetic polypeptide”
QVQLVQSGAEVKKPGASVKVSCKVSGYTLTELSMHWVRQAPGKGLEWMGGFDPEDGETIYAQKFQGRVTVTEDTSTDTAYMELSSLRSEDTAVYYCATESRGIGWPYFDYWGQGTLVTVSSGGGGSGGGGSGGGGSDIQMTQSPSSLSASVGDRVTITCRASQSISSYLNWYQQKPGKAPKLLISGASSLKSGVPSRFSGSGSGTDFTLTISSLPPEDFATYYCQQSYSTPITFGQGTRLEIKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - DNA, 351 bp, Artificial Sequence
  source: /note=“Description of Artificial Sequence Synthetic polynucleotide”
caggtccagttggtcgaaagtggcggtggtgtagtgcagccgggccgcagtttgaggctttcctgtgcggcttcaggctttactttttccagctatggaatgcactgggtgcggcaggcccccggcaaaggacttgagtgggtggccgtcatttcttatgacggatcagataagtactacgtggacagcgtcaagggcagattcaccatctctagggacaacagtaaaaatagactctacctccagatgaatagcctcagagctgaagacacggccgtctactattgtgctcgggagcggtatagtggcagagactactgggggcagggcacactcgttacagtgagtagc

SEQ ID NO: 94 - protein, 117 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSS

SEQ ID NO: 95 - protein, 7 aa, Artificial Sequence
  source: /note=“Description of Artificial Sequence Synthetic peptide”
GFTFSSY

SEQ ID NO: 96 - protein, 6 aa, Artificial Sequence
  source: /note=“Description of Artificial Sequence Synthetic peptide”
SYDGSD

SEQ ID NO: 97 - protein, 8 aa, Artificial Sequence
  source: /note=“Description of Artificial Sequence Synthetic peptide”
ERYSGRDY

SEQ ID NO: 98 - DNA, 324 bp, Artificial Sequence
  source: /note=“Description of Artificial Sequence Synthetic polynucleotide”
gagattgttatgacccagagtcctgcgaccctctcagtcagccccggggagcgcgcaactttgtcttgcagagctagtcagtccgtgtcctctcttctgacatggtaccagcaaaagcccgggcaggctccgcgccttttgatctttggggcttcaacaagagccactgggattcccgcacgattctctggctccgggagcggtactggtttcaccctgacgattagcagtctccagagcgaggacttcgccgtatactactgccagcagtacgatacgtggccattcacttttggaccagggactaaagtggattttaagcgc

SEQ ID NO: 99 - protein, 108 aa, Artificial Sequence
  source: /note=“Description of Artificial Sequence Synthetic polypeptide”
EIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKR

SEQ ID NO: 100 - protein, 11 aa, Artificial Sequence
  source: /note=“Description of Artificial Sequence Synthetic peptide”
RASQSVSSLLT

SEQ ID NO: 101 - protein, 7 aa, Artificial Sequence
  source: /note=“Description of Artificial Sequence Synthetic peptide”
GASTRAT

SEQ ID NO: 102 - protein, 9 aa, Artificial Sequence
  source: /note=“Description of Artificial Sequence Synthetic peptide”
QQYDTWPFT

SEQ ID NO: 103 - DNA, 1425 bp, Artificial Sequence
  source: /note=“Description of Artificial Sequence Synthetic polynucleotide”
atggcactccccgtaactgctctgctgctgccgttggcattgctcctgcacgccgcacgcccgcaggtccagttggtcgaaagtggcggtggtgtagtgcagccgggccgcagtttgaggctttcctgtgcggcttcaggctttactttttccagctatggaatgcactgggtgcggcaggcccccggcaaaggacttgagtgggtggccgtcatttcttatgacggatcagataagtactacgtggacagcgtcaagggcagattcaccatctctagggacaacagtaaaaatagactctacctccagatgaatagcctcagagctgaagacacggccgtctactattgtgctcgggagcggtatagtggcagagactactgggggcagggcacactcgttacagtgagtagcggcggaggagggagtgggggcggtggctccggtggaggaggttctgagattgttatgacccagagtcctgcgaccctctcagtcagccccggggagcgcgcaactttgtcttgcagagctagtcagtccgtgtcctctcttctgacatggtaccagcaaaagcccgggcaggctccgcgccttttgatctttggggcttcaacaagagccactgggattcccgcacgattctctggctccgggagcggtactggtttcaccctgacgattagcagtctccagagcgaggacttcgccgtatactactgccagcagtacgatacgtggccattcacttttggaccagggactaaagtggattttaagcgcgccgccgctctcgataacgaaaagtcaaatggcaccataatccacgtcaaaggcaagcacctgtgcccttccccgctcttccccggacccagtaaaccattttgggtgctggttgttgtggggggcgtgctggcctgctatagccttttggtcactgtagccttcattattttttgggtcagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 104 - protein, 474 aa, Artificial Sequence
  source: /note=“Description of Artificial Sequence Synthetic polypeptide”
MALPVTALLLPLALLLHAARPQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSGGGGSGGGGSGGGGSEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 105 - DNA, 1359 bp, Artificial Sequence
  source: /note=“Description of Artificial Sequence Synthetic polynucleotide”
caggtccagttggtcgaaagtggcggtggtgtagtgcagccgggccgcagtttgaggctttcctgtgcggcttcaggctttactttttccagctatggaatgcactgggtgcggcaggcccccggcaaaggacttgagtgggtggccgtcatttcttatgacggatcagataagtactacgtggacagcgtcaagggcagattcaccatctctagggacaacagtaaaaatagactctacctccagatgaatagcctcagagctgaagacacggccgtctactattgtgctcgggagcggtatagtggcagagactactgggggcagggcacactcgttacagtgagtagcggcggaggagggagtgggggcggtggctccggtggaggaggttctgagattgttatgacccagagtcctgcgaccctctcagtcagccccggggagcgcgcaactttgtcttgcagagctagtcagtccgtgtcctctcttctgacatggtaccagcaaaagcccgggcaggctccgcgccttttgatctttggggcttcaacaagagccactgggattcccgcacgattctctggctccgggagcggtactggtttcaccctgacgattagcagtctccagagcgaggacttcgccgtatactactgccagcagtacgatacgtggccattcacttttggaccagggactaaagtggattttaagcgcgccgccgctctcgataacgaaaagtcaaatggcaccataatccacgtcaaaggcaagcacctgtgcccttccccgctcttccccggacccagtaaaccattttgggtgctggttgttgtggggggcgtgctggcctgctatagccttttggtcactgtagccttcattattttttgggtcagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 106 - protein, 453 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSGGGGSGGGGSGGGGSEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - DNA, 1452 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctcgtggagtctggcggcggcgtggtccagcccggccggtccctgcgcctgtcctgcgccgccagcgggtttactttttcctcctacggcatgcactgggtgcgccaggctcccggcaagggcctcgagtgggtcgccgtgatctcatacgatgggtcagacaaatactatgtcgattctgttaaagggcggtttaccatttcaagagataactctaagaataggctgtatttgcagatgaacagcctgagggctgaagataccgcagtgtactattgcgctagggagcggtatagtggccgcgattactggggacagggtacactggtgaccgtgagctctgggggtggcggaagcgggggtggcggaagcggcggagggggtagtgaaattgtgatgacccagtctccggctacactttcagtctcccctggggagagagctacactgtcatgcagagcgtcccagtccgtctcttctctccttacctggtatcagcagaagcccggccaggctcctcgactgctgatcttcggtgcctccacaagggcgaccgggattccagcccgcttctcaggttctgggagcggaactggtttcactttgacaatcagttcactgcagtcagaggatttcgccgtgtactactgccagcaatacgacacatggccattcactttcggacccggtaccaaagtcgatttcaagagagccgcggccatcgaggttatgtacccaccaccatatctggacaatgaaaaaagcaatggaaccattatccatgtgaagggtaaacacctctgccctagcccacttttccctggcccatcaaagcccttctgggtcttggtggtcgtggggggtgtgctggcctgttacagccttctggtgacggttgctttcattatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 108 - protein, 483 aa, Artificial Sequence
  source: /note=“Description of Artificial Sequence Synthetic polypeptide”
MALPVTALLLPLALLLHAARPQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSGGGGSGGGGSGGGGSEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 109 - DNA, 1386 bp, Artificial Sequence
  source: /note=“Description of Artificial Sequence Synthetic polynucleotide”
caggtgcagctcgtggagtctggcggcggcgtggtccagcccggccggtccctgcgcctgtcctgcgccgccagcgggtttactttttcctcctacggcatgcactgggtgcgccaggctcccggcaagggcctcgagtgggtcgccgtgatctcatacgatgggtcagacaaatactatgtcgattctgttaaagggcggtttaccatttcaagagataactctaagaataggctgtatttgcagatgaacagcctgagggctgaagataccgcagtgtactattgcgctagggagcggtatagtggccgcgattactggggacagggtacactggtgaccgtgagctctgggggtggcggaagcgggggtggcggaagcggcggagggggtagtgaaattgtgatgacccagtctccggctacactttcagtctcccctggggagagagctacactgtcatgcagagcgtcccagtccgtctcttctctccttacctggtatcagcagaagcccggccaggctcctcgactgctgatcttcggtgcctccacaagggcgaccgggattccagcccgcttctcaggttctgggagcggaactggtttcactttgacaatcagttcactgcagtcagaggatttcgccgtgtactactgccagcaatacgacacatggccattcactttcggacccggtaccaaagtcgatttcaagagagccgcggccatcgaggttatgtacccaccaccatatctggacaatgaaaaaagcaatggaaccattatccatgtgaagggtaaacacctctgccctagcccacttttccctggcccatcaaagcccttctgggtcttggtggtcgtggggggtgtgctggcctgttacagccttctggtgacggttgctttcattatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 110 - protein, 462 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSGGGGSGGGGSGGGGSEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 111 - DNA, 1533 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagttggttgaatcaggagggggtgtggtgcaacccggtcggtcactgcgcctcagttgtgctgcttccgggtttactttcagctcatatgggatgcactgggtacggcaggctccaggtaaaggcttggaatgggtggcggtgatcagctatgacggctctgacaaatattatgtggactccgtgaaaggcagattcaccatcagtcgagacaactcaaagaatagactctacttgcagatgaatagcctccgggccgaagatactgcagtctattattgcgcccgggagcgctacagtggaagagactattgggggcaaggaactcttgtcacagtctcatctggcggcggcggcagcggtgggggcggatctggcgggggcggcagcgaaatcgttatgactcagagtcctgccacactgagcgttagccctggtgagagagcaacacttagctgcagagctagtcagagtgtttccagtcttttgacatggtaccaacagaagcccggtcaagctccacgactgctcatcttcggtgcatccacccgcgcaaccgggatacccgcccggttttccggttctggaagtggcacaggattcacgctcaccatttcttctctgcagtctgaagactttgccgtgtattactgccagcagtacgatacctggccctttacctttggcccaggtactaaagtggattttaaacgagctgctgcactttccaatagtattatgtacttttcacattttgtgcccgtgttcctgcctgcgaagcctacgacaaccccagcccctaggccgcccacaccggccccaactattgcctcccagccattgtctctgagacccgaagcttgcagacctgctgctggaggcgccgttcacacccgaggattggatttcgcatgtgacatttacatctgggcccctttggccggaacctgcggtgtgctgctgctgtcactcgtgattacactttactgcaaccaccgaaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 112 - protein, 509 aa, Artificial Sequence
  source: /note=“Description of Artificial Sequence Synthetic polypeptide”
MALPVTALLLPLALLLHAARPQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGGTLVTVSSGGGGSGGGGSGGGGSEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 113 - DNA, 1467 bp, Artificial Sequence
  source: /note=“Description of Artificial Sequence Synthetic polynucleotide”
caggtgcagttggttgaatcaggagggggtgtggtgcaacccggtcggtcactgcgcctcagttgtgctgcttccgggtttactttcagctcatatgggatgcactgggtacggcaggctccaggtaaaggcttggaatgggtggcggtgatcagctatgacggctctgacaaatattatgtggactccgtgaaaggcagattcaccatcagtcgagacaactcaaagaatagactctacttgcagatgaatagcctccgggccgaagatactgcagtctattattgcgcccgggagcgctacagtggaagagactattgggggcaaggaactcttgtcacagtctcatctggcggcggcggcagcggtgggggcggatctggcgggggcggcagcgaaatcgttatgactcagagtcctgccacactgagcgttagccctggtgagagagcaacacttagctgcagagctagtcagagtgtttccagtcttttgacatggtaccaacagaagcccggtcaagctccacgactgctcatcttcggtgcatccacccgcgcaaccgggatacccgcccggttttccggttctggaagtggcacaggattcacgctcaccatttcttctctgcagtctgaagactttgccgtgtattactgccagcagtacgatacctggccctttacctttggcccaggtactaaagtggattttaaacgagctgctgcactttccaatagtattatgtacttttcacattttgtgcccgtgttcctgcctgcgaagcctacgacaaccccagcccctaggccgcccacaccggccccaactattgcctcccagccattgtctctgagacccgaagcttgcagacctgctgctggaggcgccgttcacacccgaggattggatttcgcatgtgacatttacatctgggcccctttggccggaacctgcggtgtgctgctgctgtcactcgtgattacactttactgcaaccaccgaaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 114 - protein, 489 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSGGGGSGGGGSGGGGSEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 115 - DNA, 1425 bp, Artificial Sequence
  source: /note=“Description of Artificial Sequence Synthetic polynucleotide”
atggcactccccgtaactgctctgctgctgccgttggcattgctcctgcacgccgcacgcccggagattgtgatgacccagtcccctgctaccctgtccgtcagtccgggcgagagagccaccttgtcatgccgggccagccagtccgtcagcagtctcctgacttggtatcagcaaaaaccagggcaggcaccgcggcttttgatttttggtgcaagcacacgcgccactggcattccagctaggttttctggaagtggatctgggacaggcttcactctgacaatcagtagcctgcagagtgaggactttgctgtttactactgtcaacagtacgacacctggccattcacattcgggcccggcaccaaggtcgacttcaagaggggcggtggaggttcaggtggtggcgggtcaggcggcggtgggtctcaggttcaactggtggaatcaggtggcggcgttgtccaaccggggcgatcacttcgactttcctgtgctgcctcaggctttactttttcatcctatgggatgcactgggttcggcaggctcccggaaaaggactcgagtgggttgcagtgatctcttacgatggctcagacaagtattatgtggactcagtcaaggggagattcacaataagccgagacaactccaaaaaccggctttatctccagatgaacagccttagagcggaagataccgcggtatactactgtgcccgcgagaggtattccggcagagactactggggacagggcacactggtcaccgtgagttctgccgcagcgctcgataacgaaaagagcaacggaaccattatccacgttaagggcaagcacctgtgccccagtcccctcttcccaggaccatctaaacccttctgggttctggtagtagttggaggggtccttgcatgttactcccttttggtcaccgtcgccttcattattttctgggtgagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 116 - protein, 474 aa, Artificial Sequence
  source: /note=“Description of Artificial Sequence Synthetic polypeptide”
MALPVTALLLPLALLLHAARPEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRGGGGSGGGGSGGGGSQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - DNA, 1359 bp, Artificial Sequence
  source: /note=“Description of Artificial Sequence Synthetic polynucleotide”
gagattgtgatgacccagtcccctgctaccctgtccgtcagtccgggcgagagagccaccttgtcatgccgggccagccagtccgtcagcagtctcctgacttggtatcagcaaaaaccagggcaggcaccgcggcttttgatttttggtgcaagcacacgcgccactggcattccagctaggttttctggaagtggatctgggacaggcttcactctgacaatcagtagcctgcagagtgaggactttgctgtttactactgtcaacagtacgacacctggccattcacattcgggcccggcaccaaggtcgacttcaagaggggcggtggaggttcaggtggtggcgggtcaggcggcggtgggtctcaggttcaactggtggaatcaggtggcggcgttgtccaaccggggcgatcacttcgactttcctgtgctgcctcaggctttactttttcatcctatgggatgcactgggttcggcaggctcccggaaaaggactcgagtgggttgcagtgatctcttacgatggctcagacaagtattatgtggactcagtcaaggggagattcacaataagccgagacaactccaaaaaccggctttatctccagatgaacagccttagagcggaagataccgcggtatactactgtgcccgcgagaggtattccggcagagactactggggacagggcacactggtcaccgtgagttctgccgcagcgctcgataacgaaaagagcaacggaaccattatccacgttaagggcaagcacctgtgccccagtcccctcttcccaggaccatctaaacccttctgggttctggtagtagttggaggggtccttgcatgttactcccttttggtcaccgtcgccttcattattttctgggtgagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 118 - protein, 453 aa, Artificial Sequence
  source: /note=“Description of Artificial Sequence Synthetic polypeptide”
EIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRGGGGSGGGGSGGGGSQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - DNA, 1452 bp, Artificial Sequence
  source: /note=“Description of Artificial Sequence Synthetic polynucleotide”
atggcactccccgtaactgctctgctgctgccgttggcattgctcctgcacgccgcacgcccggagatcgtcatgacacagagtccagctaccctgagcgtgtcccctggagagagagccaccctgtcctgtagggctagtcagagtgtgtccagcctcctcacctggtatcaacagaagcctggtcaagctccccggctgcttatcttcggggccagcacgcgagccacaggcatcccggccagattctctggctctggcagtggcaccgggttcactctcacgatctcatccctgcagtcagaggatttcgctgtgtattactgtcagcagtacgatacatggcccttcaccttcggcccgggcacaaaagtagatttcaagcgcggcggcgggggtagtgggggcgggggatcaggaggagggggctcccaagtacagctggttgagagcggcggcggggtggttcagcccgggcgcagcctcaggctgagttgcgcagcatcaggattcacattcagttcttatggaatgcattgggtcagacaggctcccgggaagggccttgaatgggtggcagtcattagctacgacggaagcgataagtactatgtggactcagttaaagggagatttactatcagccgcgacaattccaaaaacagattgtatttgcagatgaactccctcagggcggaggacactgctgtatattactgcgcacgagagagatactccggccgagactattggggccaaggaacattggtaactgtgagctccgccgcagctattgaggtcatgtaccccccaccttatctcgataatgagaagagtaatgggactataattcacgtaaagggcaaacacctgtgcccttccccgctgtttccaggtccaagtaagccgttctgggtcctggttgtggtgggaggggtgctggcctgctattctctgttggttaccgtggcctttatcattttctgggtgagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 120 - protein, 483 aa, Artificial Sequence
  source: /note=“Description of Artificial Sequence Synthetic polypeptide”
MALPVTALLLPLALLLHAARPEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRGGGGSGGGGSGGGGSQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - DNA, 1386 bp, Artificial Sequence
  source: /note=“Description of Artificial Sequence Synthetic polynucleotide”
gagatcgtcatgacacagagtccagctaccctgagcgtgtcccctggagagagagccaccctgtcctgtagggctagtcagagtgtgtccagcctcctcacctggtatcaacagaagcctggtcaagctccccggctgcttatcttcggggccagcacgcgagccacaggcatcccggccagattctctggctctggcagtggcaccgggttcactctcacgatctcatccctgcagtcagaggatttcgctgtgtattactgtcagcagtacgatacatggcccttcaccttcggcccgggcacaaaagtagatttcaagcgcggcggcgggggtagtgggggcgggggatcaggaggagggggctcccaagtacagctggttgagagcggcggcggggtggttcagcccgggcgcagcctcaggctgagttgcgcagcatcaggattcacattcagttcttatggaatgcattgggtcagacaggctcccgggaagggccttgaatgggtggcagtcattagctacgacggaagcgataagtactatgtggactcagttaaagggagatttactatcagccgcgacaattccaaaaacagattgtatttgcagatgaactccctcagggcggaggacactgctgtatattactgcgcacgagagagatactccggccgagactattggggccaaggaacattggtaactgtgagctccgccgcagctattgaggtcatgtaccccccaccttatctcgataatgagaagagtaatgggactataattcacgtaaagggcaaacacctgtgcccttccccgctgtttccaggtccaagtaagccgttctgggtcctggttgtggtgggaggggtgctggcctgctattctctgttggttaccgtggcctttatcattttctgggtgagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 122 - protein, 462 aa, Artificial Sequence
  source: /note=“Description of Artificial Sequence Synthetic polypeptide”
EIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRGGGGSGGGGSGGGGSQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 1533 bp, Artificial Sequence
  source: /note=“Description of Artificial Sequence Synthetic polynucleotide”
atggcactccccgtaactgctctgctgctgccgttggcattgctcctgcacgccgcacgcccggaaatagtgatgactcagtccccggccaccctcagcgtgtcccccggggagcgagcgaccctgtcatgcagggcttcccagagtgtcagctccctgctcacttggtatcagcaaaagccggggcaggctccccgcctcctcatcttcggggcatcaactagggccaccggcattcctgcaagattttccgggtctggcagcggcaccggcttcacccttaccattagctctctgcagtctgaggacttcgccgtttactattgtcagcagtatgatacttggccctttaccttcggtcccggaactaaggtggacttcaagcgcggggggggtggatctggaggtggtggctccgggggcggtggaagccaggtccagttggttgagagcggcggcggagtggtgcagcccgggaggtccttgcggctgagctgtgcagcctccggttttactttttctagctatggaatgcattgggtaagacaggctcccggaaaaggcctcgagtgggtggcggtcattagctatgatggatctgataaatactatgtggactcagttaaggggcgcttcacaatctcaagagacaatagcaaaaatagactgtacctgcagatgaatagtctgcgcgccgaggacactgccgtgtactactgcgcccgcgagagatacagcggacgggattactggggccagggtaccctcgtaacggtgtcctccgctgccgcccttagcaacagcattatgtacttttctcatttcgtgccagtctttctcccagcaaagcccaccactaccccggcccccaggccgcctactcctgcccccactatcgcgtctcagcctctctccttgcggcccgaggcctgccggccagccgcagggggcgccgtacatactcggggtttggatttcgcttgcgacatatatatttgggcccccctcgccggcacatgtggagtgctgctcctgagtctcgttataaccctctattgcaaccatagaaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctaggtaa

SEQ ID NO: 124 - protein, 510 aa, Artificial Sequence
  source: /note=“Description of Artificial Sequence Synthetic polypeptide”
MALPVTALLLPLALLLHAARPEIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRGGGGSGGGGSGGGGSQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - DNA, 1467 bp, Artificial Sequence
  source: /note=“Description of Artificial Sequence Synthetic polynucleotide”
gaaatagtgatgactcagtccccggccaccctcagcgtgtcccccggggagcgagcgaccctgtcatgcagggcttcccagagtgtcagctccctgctcacttggtatcagcaaaagccggggcaggctccccgcctcctcatcttcggggcatcaactagggccaccggcattcctgcaagattttccgggtctggcagcggcaccggcttcacccttaccattagctctctgcagtctgaggacttcgccgtttactattgtcagcagtatgatacttggccctttaccttcggtcccggaactaaggtggacttcaagcgcggggggggtggatctggaggtggtggctccgggggcggtggaagccaggtccagttggttgagagcggcggcggagtggtgcagcccgggaggtccttgcggctgagctgtgcagcctccggttttactttttctagctatggaatgcattgggtaagacaggctcccggaaaaggcctcgagtgggtggcggtcattagctatgatggatctgataaatactatgtggactcagttaaggggcgcttcacaatctcaagagacaatagcaaaaatagactgtacctgcagatgaatagtctgcgcgccgaggacactgccgtgtactactgcgcccgcgagagatacagcggacgggattactggggccagggtaccctcgtaacggtgtcctccgctgccgcccttagcaacagcattatgtacttttctcatttcgtgccagtctttctcccagcaaagcccaccactaccccggcccccaggccgcctactcctgcccccactatcgcgtctcagcctctctccttgcggcccgaggcctgccggccagccgcagggggcgccgtacatactcggggtttggatttcgcttgcgacatatatatttgggcccccctcgccggcacatgtggagtgctgctcctgagtctcgttataaccctctattgcaaccatagaaacagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 126 - protein, 489 aa, Artificial Sequence
  source: /note=“Description of Artificial Sequence Synthetic polypeptide”
EIVMTQSPATLSVSPGERATLSCRASQSVSSLLTWYQQKPGQAPRLLIFGASTRATGIPARFSGSGSGTGFTLTISSLQSEDFAVYYCQQYDTWPFTFGPGTKVDFKRGGGGSGGGGSGGGGSQVQLVESGGGVVQPGRSLRLSCAASGFTFSSYGMHWVRQAPGKGLEWVAVISYDGSDKYYVDSVKGRFTISRDNSKNRLYLQMNSLRAEDTAVYYCARERYSGRDYWGQGTLVTVSSAAALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - DNA, 63 bp, Artificial Sequence
  source: /note=“Description of Artificial Sequence Synthetic oligonucleotide”
atggcactccccgtaactgctctgctgctgccgttggcattgctcctgcacgccgcacgcccg

SEQ ID NO: 128 - protein, 21 aa, Artificial Sequence
  source: /note=“Description of Artificial Sequence Synthetic peptide”
MALPVTALLLPLALLLHAARP

SEQ ID NO: 129 - DNA, 45 bp, Artificial Sequence
  source: /note=“Description of Artificial Sequence Synthetic oligonucleotide”
ggcggtggaggctccggaggggggggctctggcggagggggctcc

SEQ ID NO: 130 - protein, 15 aa, Artificial Sequence
  source: /note=“Description of Artificial Sequence Synthetic peptide”
GGGGSGGGGSGGGGS

SEQ ID NO: 131 - DNA, 54 bp, Artificial Sequence
  source: /note=“Description of Artificial Sequence Synthetic oligonucleotide”
gggtctacatccggctccgggaagcccggaagtggcgaaggtagtacaaagggg

SEQ ID NO: 132 - protein, 18 aa, Artificial Sequence
  source: /note=“Description of Artificial Sequence Synthetic peptide”
GSTSGSGKPGSGEGSTKG

SEQ ID NO: 133 - protein, 152 aa, Artificial Sequence
  source: /note=“Description of Artificial Sequence Synthetic polypeptide”
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 134 - protein, 9 aa, Artificial Sequence
  source: /note=“Description of Artificial Sequence Synthetic peptide”
  MOD_RES (2)..(9): Any amino acid
GXXXXXXXX

SEQ ID NO: 135 - protein, 6 aa, Artificial Sequence
  source: /note=“Description of Artificial Sequence Synthetic peptide”
  MOD_RES (1)..(6): Any amino acid
XXXXXX

SEQ ID NO: 136 - protein, 14 aa, Artificial Sequence
  source: /note=“Description of Artificial Sequence Synthetic peptide”
  MOD_RES (1)..(12): Any amino acid
XXXXXXXXXXXXDY

SEQ ID NO: 137 - protein, 11 aa, Artificial Sequence
  source: /note=“Description of Artificial Sequence Synthetic peptide”
  MOD_RES (1)..(1): Any amino acid
  MOD_RES (5)..(9): Any amino acid
  MOD_RES (11)..(11): Any amino acid
XASQXXXXXLX

SEQ ID NO: 138 - protein, 7 aa, Artificial Sequence
  source: /note=“Description of Artificial Sequence Synthetic peptide”
  MOD_RES (1)..(1): Any amino acid
  MOD_RES (4)..(7): Any amino acid
XASXXXX

SEQ ID NO: 139 - protein, 9 aa, Artificial Sequence
  source: /note=“Description of Artificial Sequence Synthetic peptide”
  MOD_RES (3)..(6): Any amino acid
  MOD_RES (8)..(8): Any amino acid
QQXXXXPXT

SEQ ID NO: 140 - DNA, 126 bp, Artificial Sequence
  source: /note=“Description of Artificial Sequence Synthetic polynucleotide”
aagcgcggcaggaagaagctcctctacatttttaagcagccttttatgaggcccgtacagacaacacaggaggaagatggctgtagctgcagatttcccgaggaggaggaaggtgggtgcgagctg

SEQ ID NO: 141 - protein, 42 aa, Artificial Sequence
  source: /note=“Description of Artificial Sequence Synthetic polypeptide”
KRGRKKLLYIFKQPFMRPVQTTQEEDGCSCRFPEEEEGGCEL

SEQ ID NO: 142 - protein, 37 aa, Artificial Sequence
  source: /note=“Description of Artificial Sequence Synthetic polypeptide”
RRDQRLPPDAHKPPGGGSFRTPIQEEQADAHSTLAKI

SEQ ID NO: 143 - protein, 21 aa, Artificial Sequence
  source: /note=“Description of Artificial Sequence Synthetic peptide”
MALPVTALLLPLALLLHAARP

SEQ ID NO: 144 - protein, 20 aa, Artificial Sequence
  source: /note=“Description of Artificial Sequence Synthetic peptide”
GGGGSGGGGSGGGGSGGGGS

SEQ ID NO: 145 - protein, 112 aa, Artificial Sequence
  source: /note=“Description of Artificial Sequence Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - DNA, 1467 bp, Artificial Sequence
  source: /note=“Description of Artificial Sequence Synthetic polynucleotide”
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 147 - protein, 489 aa, Artificial Sequence
  source: /note=“Description of Artificial Sequence Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - DNA, 1437 bp, Artificial Sequence
  source: /note=“Description of Artificial Sequence Synthetic polynucleotide”
atggcactccccgtaactgctctgctgctgccgttggcattgctcctgcacgccgcacgcccggatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaaggctcgacgagcggctctggtaaaccgggctctggtgaaggcagtaccaaaggtgaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtcagccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 149 - protein, 479 aa, Artificial Sequence
  source: /note=“Description of Artificial Sequence Synthetic polypeptide”
MALPVTALLLPLALLLHAARPDIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIKGSTSGSGKPGSGEGSTKGEVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSSAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - DNA, 1506 bp, Artificial Sequence
  source: /note=“Description of Artificial Sequence Synthetic polynucleotide”
atggccctgcctgtgacagctctgctgctgcccctggccctgctgctgcatgccgccagacctgagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaagggcagcacctccggcagcggcaagcctggctctggcgagggctctaccaagggccaggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagcttcgtgcccgtgttcctgcccgccaagcctaccaccacccctgcccctagacctcccaccccagccccaacaatcgccagccagcctctgtccctgcggcccgaagcctgtagacctgctgccggcggagccgtgcacaccagaggcctggacttcgcctgcgatatctacatctgggcccctctggccggcacctgtggcgtgctgctgctgagcctggtgatcaccctgtactgcaaccaccggaacagaagcaagcggagccggctgctgcacagcgactacatgaacatgaccccaagacggcctggccccacccggaagcactaccagccttacgcccctcccagagacttcgccgcctaccggtccagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 151 - protein, 502 aa, Artificial Sequence
  source: /note=“Description of Artificial Sequence Synthetic polypeptide”
MALPVTALLLPLALLLHAARPEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIKGSTSGSGKPGSGEGSTKGQVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - DNA, 1824 bp, Artificial Sequence
  source: /note=“Description of Artificial Sequence Synthetic polynucleotide”
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgctctccggagctcaggaacctacaaatacatctttggaacaggcaccaggctgaaggttttagcaaatatccagaaccctgaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagtcgggccaagcggtccggatccggagccaccaacttcagcctgctgaagcaggccggcgacgtggaggagaaccccggccccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcatccggacagggccttttttctctggaaacaccatatattttggagagggaagttggctcactgttgtagaggacctgagaaacgtgaccccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggctatggtcaaaagaaagaactcatga

SEQ ID NO: 153 - protein, 607 aa, Artificial Sequence
  source: /note=“Description of Artificial Sequence Synthetic polypeptide”
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCALRSSGTYKYIFGTGTRLKVLANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSSRAKRSGSGATNFSLLKQAGDVEENPGPMGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASIRTGPFFSGNTIYFGEGSWLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKNS

SEQ ID NO: 154 - DNA, 1437 bp, Artificial Sequence
  source: /note=“Description of Artificial Sequence Synthetic polynucleotide”
atggcactccccgtaactgctctgctgctgccgttggcattgctcctgcacgccgcacgcccgcaggtccaactgcaagaaagcggacccggactggtgaagccttctgagacacttagtctgacgtgcacggtcagtggcggctccatctcctcctattattggtcatggatacgacaacccccaggtaagggcctggaatggattggctatatctactattcaggaagcacgaactacaatcccagcctgaagtcccgagtgacaatttcagtagataccagtaaaaaccagttcagtcttaaactgtcaagcgtgacagctgccgacaccgctgtgtattactgcgtctcactggtgtattgtggaggggattgttatagcgggttcgattattggggacagggaaccctggtgactgtatcttccggcggcggcggctcagggggtggcggtagtggcggtgggggttccgatattcaactgacacaatcccccagctcactcagcgccagcgtgggggacagggttagctttacctgtcaagcctctcaggatataaataactttctgaactggtatcaacagaagcctgggaaggcgcccaaactcctgatctatgatgcgtccaacctggaaactggcgtgccttcacgctttagcggctctggcagtggtacagacttcacttttaccatctcttcacttcagccggaggacatcgccacatattactgtcaacagtacggaaacttgccctttacttttggaggcggcaccaaagttgaaatcaaaagggccgctgccctggataacgaaaagagcaatgggactataatacatgttaaaggaaaacacctgtgtccatctcccctgttccctggaccgtcaaagccattttgggtgctcgtggttgtcggtggcgttctcgcctgttatagcttgctggtgacagtagccttcattatcttttgggtgagatccaaaagaagccgcctgctccatagcgattacatgaatatgactccacgccgccctggccccacaaggaaacactaccagccttacgcaccacctagagatttcgctgcctatcggagccgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgc

SEQ ID NO: 155 - protein, 479 aa, Artificial Sequence; identical to SEQ ID NO: 26
  source: /note=“Description of Artificial Sequence Synthetic polypeptide”

SEQ ID NO: 156 - DNA, 1449 bp, Artificial Sequence
  source: /note=“Description of Artificial Sequence Synthetic polynucleotide”
atggcactccccgtaactgctctgctgctgccgttggcattgctcctgcacgccgcacgcccgcaggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatcgtatgatggaagtaataaatactatgcagactccgtgaagggccgattcaccatctccagagacaattccaagaacacgctgtatctgcaaatgaacagcctgagagccgaggacacggcggtgtactactgcgtcaaggggccgttgcaggagccgccatacgattatggaatggacgtatggggccagggaacaactgtcaccgtctcctcagggtctacatccggctccgggaagcccggaagtggcgaaggtagtacaaagggggaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatagcgcatccaccagggccactggtatcccagccaggttcagtggcagtgggtctgggacagagttcactctcaccatcagcagcctgcagtctgaagattttgcagtttattactgtcagcagcaccacgtctggcctctcacttttggcggagggaccaaggttgagatcaaacgggccgctgcccttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggttagatccaaaagaagccgcctgctccatagcgattacatgaatatgactccacgccgccctggccccacaaggaaacactaccagccttacgcaccacctagagatttcgctgcctatcggagcagggtgaagttttccagatctgcagatgcaccagcgtatcagcagggccagaaccaactgtataacgagctcaacctgggacgcagggaagagtatgacgttttggacaagcgcagaggacgggaccctgagatgggtggcaaaccaagacgaaaaaacccccaggagggtctctataatgagctgcagaaggataagatggctgaagcctattctgaaataggcatgaaaggagagcggagaaggggaaaagggcacgacggtttgtaccagggactcagcactgctacgaaggatacttatgacgctctccacatgcaagccctgccacctagg

SEQ ID NO: 157 - protein, 483 aa, Artificial Sequence
  source: /note=“Description of Artificial Sequence Synthetic polypeptide”
MALPVTALLLPLALLLHAARPQVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSSGSTSGSGKPGSGEGSTKGEIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKRAAALDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - DNA, 1900 bp, Artificial Sequence
  source: /note=“Description of Artificial Sequence Synthetic polynucleotide”
atgggcaccagactgttcttttatgtggccctgtgtctgctgtggaccggccacatggatgccggaattacacagagccccagacacaaagtgaccgagacaggcacccctgtgacactgagatgccaccagaccgagaaccaccgctacatgtactggtacagacaggatccaggccacggcctgagactgatccactacagctacggcgtgaaggacaccgacaagggcgaagtgtctgacggctacagcgtgtccagaagcaagaccgaggatttcctgctgaccctggaaagcgccacaagcagccagaccagcgtgtacttttgtgccatcagcggctacaagaacaccgaggcctttttcggccaaggcaccaggctgacagtggtggaagatctgaagaacgtgttcccacctgaggtggccgtgtttgagccttctgaggccgagatcagccacacacagaaagccacactcgtgtgcctggccaccggcttttatcccgatcacgtggaactgtcttggtgggtcaacggcaaagaggtgcacagcggcgttagcacagaccctcagcctctgaaagagcagcccgctctgaacgacagcagatactgtctgagcagcagactgagagtgtccgccaccttctggcagaaccccagaaaccacttcagatgccaggtgcagttctacggcctgtccgagaatgacgagtggacccaggatagagccaagccagtgacacagattgtgtctgccgaagcctggggcagagccgattgtggctttacaagcgagagctaccagcagggcgtgctgtctgccacaatcctgtatgagatcctgctgggcaaagccactctgtacgctgtgctggtgtctgccctggtgctgatggccatggtcaagagaaaggacagcagaggcagagccaagagatctggcagcggcgccacaaactttagcctgctgaaacaggccggcgacgtggaagagaaccctggacctatgaagtccctgcgggtgctgctggttattctgtggctgcagctgagctgggtttggagccagggacagcaagtgatgcagatccctcagtaccagcacgtgcaagaaggcgaggacttcaccacctactgcaacagcagcacaaccctgagcaacatccagtggtacaagcagaggcctggcgggcaccctgtgtttctgatccagctggttaagagcggcgaagtgaagaagcagaagcggctgacctttcagttcggcgaggccaagaagaacagcagcctgcacattaccgccacacagaccaccgacgtgggcacatatttttgcgctggcagagaaggcggcagcgagaagctggtttttggcaagggcaccaaactgaccgtgaatcccgacattcagaaccccgatccagccgtgtaccagctgagagacagcaagagcagcgacaagagcgtgtgtctgttcaccgacttcgactcccagaccaatgtgtcccagagcaaggactccgacgtgtacatcaccgataagaccgtgctggacatgcggagcatggacttcaagagcaatagcgccgtggcttggagcaacaagagcgactttgcctgcgccaacgccttcaacaacagcatcatccccgaggacacattcttcccaagtcctgagagcagctgcgacgtgaagctggtggaaaagagcttcgagacagacaccaacctgaacttccagaacctgagcgtgatcggcttcagaatcctgctgctgaaggtggccggcttcaacctgctgatgactctgagactgtggtccagctgaattcggatccaagcttaggcctgctcgctttcttgctgtcccatttctattaaaggttcctttgttc

SEQ ID NO: 159 - protein, 610 aa, Artificial Sequence
  source: /note=“Description of Artificial Sequence Synthetic polypeptide”
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISGYKNTEAFFGQGTRLTVVEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMKSLRVLLVILWLQLSWVWSQGQQVMQIPQYQHVQEGEDFTTYCNSSTTLSNIQWYKQRPGGHPVFLIQLVKSGEVKKQKRLTFQFGEAKKNSSLHITATQTTDVGTYFCAGREGGSEKLVFGKGTKLTVNPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 160 - DNA, 1906 bp, Artificial Sequence
  source: /note=“Description of Artificial Sequence Synthetic polynucleotide”
atgggacctggattgctttgttgggccctgctgtgtctgcttggagctggacttgtggatgccggcgtgacacagtctcccacacacctgatcaagaccagaggccagcaagtgaccctgagatgtagccctaagagcggccacgacaccgtgtcttggtatcagcaggctcttggccagggacctcagttcatcttccagtactacgaggaagaggaacggcagcggggcaacttccctgatagattctctggccatcagttccccaactacagcagcgagctgaacgtgaacgctctgctgctgggcgatagcgccctgtatctgtgtgccagttctcttggttggagaggcggcagatacaacgagcagttctttggccctggcaccagactgaccgtgctggaagatctgaagaacgtgttcccacctgaggtggccgtgtttgagccttctgaggccgagatcagccacacacagaaagccacactcgtgtgcctggccaccggcttttatcccgatcacgtggaactgtcttggtgggtcaacggcaaagaggtgcacagcggcgttagcacagaccctcagcctctgaaagagcagcccgctctgaacgacagcagatactgtctgagcagcagactgagagtgtccgccaccttctggcagaaccccagaaaccacttcagatgccaggtgcagttctacggcctgtccgagaatgacgagtggacccaggatagagccaagccagtgacacagattgtgtctgccgaagcctggggcagagccgattgtggctttacaagcgagagctaccagcagggcgtgctgtctgccacaatcctgtatgagatcctgctgggcaaagccactctgtacgctgtgctggtgtctgccctggtgctgatggccatggtcaagagaaaggacagcagaggcagagccaagagatctggcagcggcgccacaaactttagcctgctgaaacaggccggcgacgtggaagagaaccctggacctatgaagtccctgcgggtgctgctggttattctgtggctgcagctgagctgggtttggagccagggacagaacatcgaccagcctaccgagatgacagccaccgaaggcgccatcgtgcagatcaattgcacctaccagaccagcggcttcaacggcctgttctggtatcaacagcatgccggcgaggcccctaccttcctgagctataatgtgctggacggcctggaagaaaagggcagattcagcagcttcctgtccagaagcaagggctacagctacctgctgctgaaagaactccagatgaaggacagcgcctcctacctgtgtgcctccgtggatggaaacaacagactggccttcggcaagggcaaccaggtggtggtcatccccgacattcagaaccccgatccagccgtgtaccagctgagagacagcaagagcagcgacaagagcgtgtgtctgttcaccgacttcgactcccagaccaatgtgtcccagagcaaggactccgacgtgtacatcaccgataagaccgtgctggacatgcggagcatggacttcaagagcaatagcgccgtggcttggagcaacaagagcgactttgcctgcgccaacgccttcaacaacagcatcatccccgaggacacattcttcccaagtcctgagagcagctgcgacgtgaagctggtggaaaagagcttcgagacagacaccaacctgaacttccagaacctgagcgtgatcggcttcagaatcctgctgctgaaggtggccggcttcaacctgctgatgactctgagactgtggtccagctgaattcggatccaagcttaggcctgctcgctttcttgctgtcccatttctattaaaggttcctttgttc

SEQ ID NO: 161 - protein, 612 aa, Artificial Sequence
  source: /note=“Description of Artificial Sequence Synthetic polypeptide”
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGRAKRSGSGATNFSLLKQAGDVEENPGPMKSLRVLLVILWLQLSWVWSQGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 162 - DNA, 66 bp, Artificial Sequence
  source: /note=“Description of Artificial Sequence Synthetic oligonucleotide”
atgcttctcctggtgacaagccttctgctctgtgagttaccacacccagcattcctcctgatccca

SEQ ID NO: 163 - protein, 22 aa, Artificial Sequence
  source: /note=“Description of Artificial Sequence Synthetic peptide”
MLLLVTSLLLCELPHPAFLLIP

SEQ ID NO: 164 - DNA, 321 bp, Artificial Sequence
  source: /note=“Description of Artificial Sequence Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataaca

SEQ ID NO: 165 - protein, 107 aa, Artificial Sequence
  source: /note=“Description of Artificial Sequence Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 166 - DNA, 54 bp, Artificial Sequence
  source: /note=“Description of Artificial Sequence Synthetic oligonucleotide”
ggctccacctctggatccggcaagcccggatctggcgagggatccaccaagggc

SEQ ID NO: 167 - protein, 18 aa, Artificial Sequence
  source: /note=“Description of Artificial Sequence Synthetic peptide”
GSTSGSGKPGSGEGSTKG

SEQ ID NO: 168 - DNA, 360 bp, Artificial Sequence
  source: /note=“Description of Artificial Sequence Synthetic polynucleotide”
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 169 - protein, 120 aa, Artificial Sequence
  source: /note=“Description of Artificial Sequence Synthetic polypeptide”
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 170 - DNA, 9 bp, Artificial Sequence
  source: /note=“Description of Artificial Sequence Synthetic oligonucleotide”
gcggccgca

SEQ ID NO: 171 - protein, 3 aa, Artificial Sequence
  source: /note=“Description of Artificial Sequence Synthetic peptide”
AAA

SEQ ID NO: 172 - DNA, 198 bp, Artificial Sequence
  source: /note=“Description of Artificial Sequence Synthetic polynucleotide”
attgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtg

SEQ ID NO: 173 - protein, 66 aa, Artificial Sequence
  source: /note=“Description of Artificial Sequence Synthetic polypeptide”
IEVMYPPPYLDNEKSNGTIIHVKGKHLCPSPLFPGPSKPFWVLVVVGGVLACYSLLVTVAFIIFWV

SEQ ID NO: 174 - DNA, 123 bp, Artificial Sequence
  source: /note=“Description of Artificial Sequence Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 175 - protein, 41 aa, Artificial Sequence; identical to SEQ ID NO: 8
  source: /note=“Description of Artificial Sequence Synthetic polypeptide”

SEQ ID NO: 176 - DNA, 336 bp, Artificial Sequence
  source: /note=“Description of Artificial Sequence Synthetic polynucleotide”
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 177 - protein, 112 aa, Artificial Sequence; identical to SEQ ID NO: 10
  source: /note=“Description of Artificial Sequence Synthetic polypeptide”

SEQ ID NO: 178 - protein, 11 aa, Artificial Sequence
  source: /note=“Description of Artificial Sequence Synthetic peptide”
RASQDISKYLN

SEQ ID NO: 179 - protein, 7 aa, Artificial Sequence
  source: /note=“Description of Artificial Sequence Synthetic peptide”
HTSRLHS

SEQ ID NO: 180 - protein, 9 aa, Artificial Sequence
  source: /note=“Description of Artificial Sequence Synthetic peptide”
QQGNTLPYT

SEQ ID NO: 181 - protein, 7 aa, Artificial Sequence
  source: /note=“Description of Artificial Sequence Synthetic peptide”
GVSLPDY

SEQ ID NO: 182 - protein, 5 aa, Artificial Sequence
  source: /note=“Description of Artificial Sequence Synthetic peptide”
WGSET

SEQ ID NO: 183 - protein, 12 aa, Artificial Sequence
  source: /note=“Description of Artificial Sequence Synthetic peptide”
HYYYGGSYAMDY

SEQ ID NO: 184 - protein, 11 aa, Artificial Sequence
  source: /note=“Description of Artificial Sequence Synthetic peptide”
RASQDISKYLN

SEQ ID NO: 185 - protein, 7 aa, Artificial Sequence
  source: /note=“Description of Artificial Sequence Synthetic peptide”
HTSRLHS

SEQ ID NO: 186 - protein, 9 aa, Artificial Sequence
  source: /note=“Description of Artificial Sequence Synthetic peptide”
QQGNTLPYT

SEQ ID NO: 187 - protein, 5 aa, Artificial Sequence
  source: /note=“Description of Artificial Sequence Synthetic peptide”
DYGVS

SEQ ID NO: 188 - protein, 16 aa, Artificial Sequence
  source: /note=“Description of Artificial Sequence Synthetic peptide”
VIWGSETTYYNSALKS

SEQ ID NO: 189 - protein, 12 aa, Artificial Sequence
  source: /note=“Description of Artificial Sequence Synthetic peptide”
HYYYGGSYAMDY

SEQ ID NO: 190 - DNA, 63 bp, Artificial Sequence; identical to SEQ ID NO: 127
  source: /note=“Description of Artificial Sequence Synthetic oligonucleotide”

SEQ ID NO: 191 - protein, 21 aa, Artificial Sequence
  source: /note=“Description of Artificial Sequence Synthetic peptide”
MALPVTALLLPLALLLHAARP

SEQ ID NO: 192 - DNA, 321 bp, Artificial Sequence
  source: /note=“Description of Artificial Sequence Synthetic polynucleotide”
gatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaa

SEQ ID NO: 193 - protein, 107 aa, Artificial Sequence
  source: /note=“Description of Artificial Sequence Synthetic polypeptide”
DIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIK

SEQ ID NO: 194 - DNA, 54 bp, Artificial Sequence
  source: /note=“Description of Artificial Sequence Synthetic oligonucleotide”
ggctcgacgagcggctctggtaaaccgggctctggtgaaggcagtaccaaaggt

SEQ ID NO: 195 - protein, 18 aa, Artificial Sequence
  source: /note=“Description of Artificial Sequence Synthetic peptide”
GSTSGSGKPGSGEGSTKG

SEQ ID NO: 196 - DNA, 360 bp, Artificial Sequence
  source: /note=“Description of Artificial Sequence Synthetic polynucleotide”
gaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtca

SEQ ID NO: 197 - protein, 120 aa, Artificial Sequence
  source: /note=“Description of Artificial Sequence Synthetic polypeptide”
EVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 198 - DNA, 9 bp, Artificial Sequence
  source: /note=“Description of Artificial Sequence Synthetic oligonucleotide”
gccgctgcc

SEQ ID NO: 199 - protein, 3 aa, Artificial Sequence
  source: /note=“Description of Artificial Sequence Synthetic peptide”
AAA

SEQ ID NO: 200 - DNA, 171 bp, Artificial Sequence
  source: /note=“Description of Artificial Sequence Synthetic polynucleotide”
cttgataatgaaaagtcaaacggaacaatcattcacgtgaagggcaagcacctctgtccgtcacccttgttccctggtccatccaagccattctgggtgttggtcgtagtgggtggagtcctcgcttgttactctctgctcgtcaccgtggcttttataatcttctgggtt

SEQ ID NO: 201 - protein, 57 aa, Artificial Sequence
  source: /note=“Description of Artificial Sequence Synthetic polypeptide”
LDNEKSNGTIIHVKGKHLCPSPLFPGPSKPFWVLVVVGGVLACYSLLVTVAFIIFWV

SEQ ID NO: 202 - DNA, 123 bp, Artificial Sequence; identical to SEQ ID NO: 7
  source: /note=“Description of Artificial Sequence Synthetic polynucleotide”

SEQ ID NO: 203 - protein, 41 aa, Artificial Sequence; identical to SEQ ID NO: 8
  source: /note=“Description of Artificial Sequence Synthetic polypeptide”

SEQ ID NO: 204 - DNA, 336 bp, Artificial Sequence; identical to SEQ ID NO: 9
  source: /note=“Description of Artificial Sequence Synthetic polynucleotide”

SEQ ID NO: 205 - protein, 112 aa, Artificial Sequence; identical to SEQ ID NO: 10
  source: /note=“Description of Artificial Sequence Synthetic polypeptide”

SEQ ID NO: 206 - protein, 12 aa, Artificial Sequence
  source: /note=“Description of Artificial Sequence Synthetic peptide”
RASQSVSSSYLA

SEQ ID NO: 207 - protein, 7 aa, Artificial Sequence
  source: /note=“Description of Artificial Sequence Synthetic peptide”
GASSRAT

SEQ ID NO: 208 - protein, 9 aa, Artificial Sequence
  source: /note=“Description of Artificial Sequence Synthetic peptide”
QQYGSSRFT

SEQ ID NO: 209 - protein, 7 aa, Artificial Sequence
  source: /note=“Description of Artificial Sequence Synthetic peptide”
GGTFSSY

SEQ ID NO: 210 - protein, 6 aa, Artificial Sequence
  source: /note=“Description of Artificial Sequence Synthetic peptide”
IPIFGT

SEQ ID NO: 211 - protein, 10 aa, Artificial Sequence
  source: /note=“Description of Artificial Sequence Synthetic peptide”
EAVAADWLDP

SEQ ID NO: 212 - protein, 12 aa, Artificial Sequence
  source: /note=“Description of Artificial Sequence Synthetic peptide”
RASQSVSSSYLA

SEQ ID NO: 213 - protein, 7 aa, Artificial Sequence
  source: /note=“Description of Artificial Sequence Synthetic peptide”
GASSRAT

SEQ ID NO: 214 - protein, 9 aa, Artificial Sequence
  source: /note=“Description of Artificial Sequence Synthetic peptide”
QQYGSSRFT

SEQ ID NO: 215 - protein, 5 aa, Artificial Sequence
  source: /note=“Description of Artificial Sequence Synthetic peptide”
SYAIS

SEQ ID NO: 216 - protein, 17 aa, Artificial Sequence
  source: /note=“Description of Artificial Sequence Synthetic peptide”
GIIPIFGTTNYAQQFQG

SEQ ID NO: 217 - protein, 10 aa, Artificial Sequence
  source: /note=“Description of Artificial Sequence Synthetic peptide”
EAVAADWLDP

SEQ ID NO: 218 - DNA, 63 bp, Artificial Sequence
  source: /note=“Description of Artificial Sequence Synthetic oligonucleotide”
atggccctgcctgtgacagctctgctgctgcccctggccctgctgctgcatgccgccagacct

SEQ ID NO: 219 - protein, 21 aa, Artificial Sequence
  source: /note=“Description of Artificial Sequence Synthetic peptide”
MALPVTALLLPLALLLHAARP

SEQ ID NO: 220 - DNA, 324 bp, Artificial Sequence
  source: /note=“Description of Artificial Sequence Synthetic polynucleotide”
gagatcgtgctgacccagtctcccggcaccctgtctctcagcccaggagagagagccaccctgagctgcagagccagccagagcgtgtccagcagctacctggcctggtatcagcagaagcccggacaggcccccagactgctgatctacggcgccagctctagagccaccggcatccccgacagattcagcggcagcggcagtggcaccgacttcaccctgaccatcagcagactggaacccgaggacttcgccgtgtactactgccagcagtacggcagcagccggttcaccttcggccctggcaccaaggtggacatcaag

SEQ ID NO: 221 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSRFTFGPGTKVDIK

SEQ ID NO: 222 - DNA, 54 bp, Artificial Sequence
  source: /note=“Description of Artificial Sequence Synthetic oligonucleotide”
ggcagcacctccggcagcggcaagcctggctctggcgagggctctaccaagggc

SEQ ID NO: 223 - protein, 18 aa, Artificial Sequence
  source: /note=“Description of Artificial Sequence Synthetic peptide”
GSTSGSGKPGSGEGSTKG

SEQ ID NO: 224 - DNA, 357 bp, Artificial Sequence
  source: /note=“Description of Artificial Sequence Synthetic polynucleotide”
caggtgcagctggtgcagtctggcgccgaagtgaagaaacccggctctagcgtgaaggtgtcctgcaaggacagcggcggcaccttcagcagctacgccatcagctgggtgcgccaggccccaggacaggggctggaatggatgggcggcatcatccccatcttcggcaccaccaactacgcccagcagttccagggcagagtgaccatcaccgccgacgagagcaccagcaccgcctacatggaactgagcagcctgcggagcgaggacacagccgtgtattactgtgcccgcgaggccgtggccgccgactggctggatccttggggacagggcaccctggtgacagtgtccagc

SEQ ID NO: 225 - protein, 119 aa, Artificial Sequence
  source: /note=“Description of Artificial Sequence Synthetic polypeptide”
QVQLVQSGAEVKKPGSSVKVSCKDSGGTFSSYAISWVRQAPGQGLEWMGGIIPIFGTTNYAQQFQGRVTITADESTSTAYMELSSLRSEDTAVYYCAREAVAADWLDPWGQGTLVTVSS

SEQ ID NO: 226 - DNA, 249 bp, Artificial Sequence
  source: /note=“Description of Artificial Sequence Synthetic polynucleotide”
ttcgtgcccgtgttcctgcccgccaagcctaccaccacccctgcccctagacctcccaccccagccccaacaatcgccagccagcctctgtccctgcggcccgaagcctgtagacctgctgccggcggagccgtgcacaccagaggcctggacttcgcctgcgatatctacatctgggcccctctggccggcacctgtggcgtgctgctgctgagcctggtgatcaccctgtactgcaaccaccggaac

SEQ ID NO: 227 - protein, 83 aa, Artificial Sequence
  source: /note=“Description of Artificial Sequence Synthetic polypeptide”
FVPVFLPAKPTTTPAPRPPTPAPTIASQPLSLRPEACRPAAGGAVHTRGLDFACDIYIWAPLAGTCGVLLLSLVITLYCNHRN

SEQ ID NO: 228 - DNA, 123 bp, Artificial Sequence
  source: /note=“Description of Artificial Sequence Synthetic polynucleotide”
agaagcaagcggagccggctgctgcacagcgactacatgaacatgaccccaagacggcctggccccacccggaagcactaccagccttacgcccctcccagagacttcgccgcctaccggtcc

SEQ ID NO: 229 - protein, 41 aa, Artificial Sequence; identical to SEQ ID NO: 8
  source: /note=“Description of Artificial Sequence Synthetic polypeptide”

SEQ ID NO: 230 - DNA, 336 bp, Artificial Sequence
  source: /note=“Description of Artificial Sequence Synthetic polynucleotide”
agagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 231 - protein, 112 aa, Artificial Sequence; identical to SEQ ID NO: 10
  source: /note=“Description of Artificial Sequence Synthetic polypeptide”

SEQ ID NO: 232 - DNA, 339 bp, Artificial Sequence
  source: /note=“Description of Artificial Sequence Synthetic polynucleotide”
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgct

SEQ ID NO: 233 - protein, 135 aa, Artificial Sequence
  source: /note=“Description of Artificial Sequence Synthetic polypeptide”
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCALRSSGTYKYIFGTGTRLKVLAN

SEQ ID NO: 234 - DNA, 66 bp, Artificial Sequence
  source: /note=“Description of Artificial Sequence Synthetic oligonucleotide”
ctccggagctcaggaacctacaaatacatctttggaacaggcaccaggctgaaggttttagcaaat

SEQ ID NO: 235 - DNA, 408 bp, Artificial Sequence
  source: /note=“Description of Artificial Sequence Synthetic polynucleotide”
atccagaaccctgaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagt

SEQ ID NO: 236 - protein, 136 aa, Artificial Sequence
  source: /note=“Description of Artificial Sequence Synthetic polypeptide”
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 237 - DNA, 81 bp, Artificial Sequence
  source: /note=“Description of Artificial Sequence Synthetic oligonucleotide”
cgggccaagcggtccggatccggagccaccaacttcagcctgctgaagcaggccggcgacgtggaggagaaccccggcccc

SEQ ID NO: 238 - protein, 27 aa, Artificial Sequence
  source: /note=“Description of Artificial Sequence Synthetic peptide”
RAKRSGSGATNFSLLKQAGDVEENPGP

SEQ ID NO: 239 - DNA, 340 bp, Artificial Sequence
  source: /note=“Description of Artificial Sequence Synthetic polynucleotide”
atgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagca

SEQ ID NO: 240 - protein, 137 aa, Artificial Sequence
  source: /note=“Description of Artificial Sequence Synthetic polypeptide”
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASIRTGPFFSGNTIYFGEGSWLTVVE

SEQ ID NO: 241 - DNA, 71 bp, Artificial Sequence
  source: /note=“Description of Artificial Sequence Synthetic oligonucleotide”
tccggacagggccttttttctctggaaacaccatatattttggagagggaagttggctcactgttgtagag

SEQ ID NO: 242 - DNA, 519 bp, Artificial Sequence
  source: /note=“Description of Artificial Sequence Synthetic polynucleotide”
gacctgagaaacgtgaccccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggctatggtcaaaagaaagaactcatga

SEQ ID NO: 243 - protein, 172 aa, Artificial Sequence
  source: /note=“Description of Artificial Sequence Synthetic polypeptide”
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKRKNS

SEQ ID NO: 244 - protein, 12 aa, Artificial Sequence
  source: /note=“Description of Artificial Sequence Synthetic peptide”
TYDTSESDYYLF

SEQ ID NO: 245 - protein, 7 aa, Artificial Sequence
  source: /note=“Description of Artificial Sequence Synthetic peptide”
QEAYKQQ

SEQ ID NO: 246 - protein, 11 aa, Artificial Sequence
  source: /note=“Description of Artificial Sequence Synthetic peptide”
ALRSSGTYKYI

SEQ ID NO: 247 - protein, 10 aa, Artificial Sequence
  source: /note=“Description of Artificial Sequence Synthetic peptide”
DPISGHTALY

SEQ ID NO: 248 - protein, 11 aa, Artificial Sequence
  source: /note=“Description of Artificial Sequence Synthetic peptide”
FQGNSAPDKSG

SEQ ID NO: 249 - protein, 15 aa, Artificial Sequence
  source: /note=“Description of Artificial Sequence Synthetic peptide”
ASIRTGPFFSGNTIY

SEQ ID NO: 250 - DNA, 63 bp, Artificial Sequence; identical to SEQ ID NO: 127
  source: /note=“Description of Artificial Sequence Synthetic oligonucleotide”

SEQ ID NO: 251 - protein, 21 aa, Artificial Sequence
  source: /note=“Description of Artificial Sequence Synthetic peptide”
MALPVTALLLPLALLLHAARP

SEQ ID NO: 252 - DNA, 366 bp, Artificial Sequence
  source: /note=“Description of Artificial Sequence Synthetic polynucleotide”
caggtccaactgcaagaaagcggacccggactggtgaagccttctgagacacttagtctgacgtgcacggtcagtggcggctccatctcctcctattattggtcatggatacgacaacccccaggtaagggcctggaatggattggctatatctactattcaggaagcacgaactacaatcccagcctgaagtcccgagtgacaatttcagtagataccagtaaaaaccagttcagtcttaaactgtcaagcgtgacagctgccgacaccgctgtgtattactgcgtctcactggtgtattgtggaggggattgttatagcgggttcgattattggggacagggaaccctggtgactgtatcttcc

SEQ ID NO: 253 - protein, 122 aa, Artificial Sequence; identical to SEQ ID NO: 16
  source: /note=“Description of Artificial Sequence Synthetic polypeptide”

SEQ ID NO: 254 - DNA, 45 bp, Artificial Sequence
  source: /note=“Description of Artificial Sequence Synthetic oligonucleotide”
ggcggcggcggctcagggggtggcggtagtggcggtgggggttcc

SEQ ID NO: 255 - protein, 15 aa, Artificial Sequence
  source: /note=“Description of Artificial Sequence Synthetic peptide”
GGGGSGGGGSGGGGS

SEQ ID NO: 256 - DNA, 324 bp, Artificial Sequence
  source: /note=“Description of Artificial Sequence Synthetic polynucleotide”
gatattcaactgacacaatcccccagctcactcagcgccagcgtgggggacagggttagctttacctgtcaagcctctcaggatataaataactttctgaactggtatcaacagaagcctgggaaggcgcccaaactcctgatctatgatgcgtccaacctggaaactggcgtgccttcacgctttagcggctctggcagtggtacagacttcacttttaccatctcttcacttcagccggaggacatcgccacatattactgtcaacagtacggaaacttgccctttacttttggaggcggcaccaaagttgaaatcaaaagg

SEQ ID NO: 257 - protein, 108 aa, Artificial Sequence; identical to SEQ ID NO: 21
  source: /note=“Description of Artificial Sequence Synthetic polypeptide”

SEQ ID NO: 258 - DNA, 9 bp, Artificial Sequence
  source: /note=“Description of Artificial Sequence Synthetic oligonucleotide”
gccgctgcc

SEQ ID NO: 259 - protein, 3 aa, Artificial Sequence
  source: /note=“Description of Artificial Sequence Synthetic peptide”
AAA

SEQ ID NO: 260 - DNA, 171 bp, Artificial Sequence
  source: /note=“Description of Artificial Sequence Synthetic polynucleotide”
ctggataacgaaaagagcaatgggactataatacatgttaaaggaaaacacctgtgtccatctcccctgttccctggaccgtcaaagccattttgggtgctcgtggttgtcggtggcgttctcgcctgttatagcttgctggtgacagtagccttcattatcttttgggtg

SEQ ID NO: 261 - protein, 57 aa, Artificial Sequence; identical to SEQ ID NO: 201
  source: /note=“Description of Artificial Sequence Synthetic polypeptide”

SEQ ID NO: 262 - DNA, 123 bp, Artificial Sequence; identical to SEQ ID NO: 7
  source: /note=“Description of Artificial Sequence Synthetic polynucleotide”

SEQ ID NO: 263 - protein, 41 aa, Artificial Sequence; identical to SEQ ID NO: 8
  source: /note=“Description of Artificial Sequence Synthetic polypeptide”

SEQ ID NO: 264 - DNA, 336 bp, Artificial Sequence
  source: /note=“Description of Artificial Sequence Synthetic polynucleotide”
cgagtgaaattttctagatcagctgatgctcccgcctatcagcagggacagaatcaactttacaatgagctgaacctgggtcgcagagaagagtacgacgttttggacaaacgccggggccgagatcctgagatgggggggaagccgagaaggaagaatcctcaagaaggcctgtacaacgagcttcaaaaagacaaaatggctgaggcgtactctgagatcggcatgaagggcgagcggagacgaggcaagggtcacgatggcttgtatcagggcctgagtacagccacaaaggacacctatgacgccctccacatgcaggcactgcccccacgc

SEQ ID NO: 265 - protein, 112 aa, Artificial Sequence; identical to SEQ ID NO: 10
  source: /note=“Description of Artificial Sequence Synthetic polypeptide”

SEQ ID NO: 266 - protein, 11 aa, Artificial Sequence
  source: /note=“Description of Artificial Sequence Synthetic peptide”
QASQDINNFLN

SEQ ID NO: 267 - protein, 7 aa, Artificial Sequence
  source: /note=“Description of Artificial Sequence Synthetic peptide”
DASNLET

SEQ ID NO: 268 - protein, 9 aa, Artificial Sequence
  source: /note=“Description of Artificial Sequence Synthetic peptide”
QQYGNLPFT

SEQ ID NO: 269 - protein, 7 aa, Artificial Sequence
  source: /note=“Description of Artificial Sequence Synthetic peptide”
GGSISSY

SEQ ID NO: 270 - protein, 5 aa, Artificial Sequence
  source: /note=“Description of Artificial Sequence Synthetic peptide”
YYSGS

SEQ ID NO: 271 - protein, 14 aa, Artificial Sequence
  source: /note=“Description of Artificial Sequence Synthetic peptide”
LVYCGGDCYSGFDY

SEQ ID NO: 272 - DNA, 63 bp, Artificial Sequence; identical to SEQ ID NO: 127
  source: /note=“Description of Artificial Sequence Synthetic oligonucleotide”

SEQ ID NO: 273 - protein, 21 aa, Artificial Sequence
  source: /note=“Description of Artificial Sequence Synthetic peptide”
MALPVTALLLPLALLLHAARP

SEQ ID NO: 274 - DNA, 369 bp, Artificial Sequence
  source: /note=“Description of Artificial Sequence Synthetic polynucleotide”
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatcgtatgatggaagtaataaatactatgcagactccgtgaagggccgattcaccatctccagagacaattccaagaacacgctgtatctgcaaatgaacagcctgagagccgaggacacggcggtgtactactgcgtcaaggggccgttgcaggagccgccatacgattatggaatggacgtatggggccagggaacaactgtcaccgtctcctca

SEQ ID NO: 275 - protein, 123 aa, Artificial Sequence
  source: /note=“Description of Artificial Sequence Synthetic polypeptide”
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCVKGPLQEPPYDYGMDVWGQGTTVTVSS

SEQ ID NO: 276 - DNA, 54 bp, Artificial Sequence; identical to SEQ ID NO: 131
  source: /note=“Description of Artificial Sequence Synthetic oligonucleotide”

SEQ ID NO: 277 - protein, 18 aa, Artificial Sequence
  source: /note=“Description of Artificial Sequence Synthetic peptide”
GSTSGSGKPGSGEGSTKG

SEQ ID NO: 278 - DNA, 324 bp, Artificial Sequence
  source: /note=“Description of Artificial Sequence Synthetic polynucleotide”
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatagcgcatccaccagggccactggtatcccagccaggttcagtggcagtgggtctgggacagagttcactctcaccatcagcagcctgcagtctgaagattttgcagtttattactgtcagcagcaccacgtctggcctctcacttttggcggagggaccaaggttgagatcaaacgg

SEQ ID NO: 279 - protein, 108 aa, Artificial Sequence
  source: /note=“Description of Artificial Sequence Synthetic polypeptide”
EIVMTQSPATLSVSPGERATLSCRASQSVSSNLAWYQQKPGQAPRLLIYSASTRATGIPARFSGSGSGTEFTLTISSLQSEDFAVYYCQQHHVWPLTFGGGTKVEIKR

SEQ ID NO: 280 - DNA, 9 bp, Artificial Sequence
  source: /note=“Description of Artificial Sequence Synthetic oligonucleotide”
gccgctgcc

SEQ ID NO: 281 - protein, 3 aa, Artificial Sequence
  source: /note=“Description of Artificial Sequence Synthetic peptide”
AAA

SEQ ID NO: 282 - DNA, 171 bp, Artificial Sequence; identical to SEQ ID NO: 200
  source: /note=“Description of Artificial Sequence Synthetic polynucleotide”

SEQ ID NO: 283 - protein, 57 aa, Artificial Sequence; identical to SEQ ID NO: 201
  source: /note=“Description of Artificial Sequence Synthetic polypeptide”

SEQ ID NO: 284 - DNA, 123 bp, Artificial Sequence; identical to SEQ ID NO: 7
  source: /note=“Description of Artificial Sequence Synthetic polynucleotide”

SEQ ID NO: 285 - protein, 41 aa, Artificial Sequence; identical to SEQ ID NO: 8
  source: /note=“Description of Artificial Sequence Synthetic polypeptide”

SEQ ID NO: 286 - DNA, 336 bp, Artificial Sequence; identical to SEQ ID NO: 9
  source: /note=“Description of Artificial Sequence Synthetic polynucleotide”

SEQ ID NO: 287 - protein, 112 aa, Artificial Sequence; identical to SEQ ID NO: 10
  source: /note=“Description of Artificial Sequence Synthetic polypeptide”

SEQ ID NO: 288 - protein, 11 aa, Artificial Sequence
  source: /note=“Description of Artificial Sequence Synthetic peptide”
RASQSVSSNLA

SEQ ID NO: 289 - protein, 7 aa, Artificial Sequence
  source: /note=“Description of Artificial Sequence Synthetic peptide”
SASTRAT

SEQ ID NO: 290 - protein, 10 aa, Artificial Sequence
  source: /note=“Description of Artificial Sequence Synthetic peptide”
QQHHVWPLTF

SEQ ID NO: 291 - protein, 7 aa, Artificial Sequence
  source: /note=“Description of Artificial Sequence Synthetic peptide”
GFTFSSY

SEQ ID NO: 292 - protein, 17 aa, Artificial Sequence
  source: /note=“Description of Artificial Sequence Synthetic peptide”
VISYDGSNKYYADSVKG

SEQ ID NO: 293 - protein, 16 aa, Artificial Sequence
  source: /note=“Description of Artificial Sequence Synthetic peptide”
VKGPLQEPPYDYGMDV

SEQ ID NO: 294 - DNA, 393 bp, Artificial Sequence
  source: /note=“Description of Artificial Sequence Synthetic polynucleotide”
atgggcaccagactgttcttttatgtggccctgtgtctgctgtggaccggccacatggatgccggaattacacagagccccagacacaaagtgaccgagacaggcacccctgtgacactgagatgccaccagaccgagaaccaccgctacatgtactggtacagacaggatccaggccacggcctgagactgatccactacagctacggcgtgaaggacaccgacaagggcgaagtgtctgacggctacagcgtgtccagaagcaagaccgaggatttcctgctgaccctggaaagcgccacaagcagccagaccagcgtgtacttttgtgccatcagcggctacaagaacaccgaggcctttttcggccaaggcaccaggctgacagtggtg

SEQ ID NO: 295 - protein, 131 aa, Artificial Sequence
  source: /note=“Description of Artificial Sequence Synthetic polypeptide”
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISGYKNTEAFFGQGTRLTVV

SEQ ID NO: 296 - DNA, 537 bp, Artificial Sequence
  source: /note=“Description of Artificial Sequence Synthetic polynucleotide”
gaagatctgaagaacgtgttcccacctgaggtggccgtgtttgagccttctgaggccgagatcagccacacacagaaagccacactcgtgtgcctggccaccggcttttatcccgatcacgtggaactgtcttggtgggtcaacggcaaagaggtgcacagcggcgttagcacagaccctcagcctctgaaagagcagcccgctctgaacgacagcagatactgtctgagcagcagactgagagtgtccgccaccttctggcagaaccccagaaaccacttcagatgccaggtgcagttctacggcctgtccgagaatgacgagtggacccaggatagagccaagccagtgacacagattgtgtctgccgaagcctggggcagagccgattgtggctttacaagcgagagctaccagcagggcgtgctgtctgccacaatcctgtatgagatcctgctgggcaaagccactctgtacgctgtgctggtgtctgccctggtgctgatggccatggtcaagagaaaggacagcagaggc

SEQ ID NO: 297 - protein, 179 aa, Artificial Sequence
  source: /note=“Description of Artificial Sequence Synthetic polypeptide”
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 298 - DNA, 81 bp, Artificial Sequence
  source: /note=“Description of Artificial Sequence Synthetic oligonucleotide”
agagccaagagatctggcagcggcgccacaaactttagcctgctgaaacaggccggcgacgtggaagagaaccctggacct

SEQ ID NO: 299 - protein, 27 aa, Artificial Sequence
  source: /note=“Description of Artificial Sequence Synthetic peptide”
RAKRSGSGATNFSLLKQAGDVEENPGP

SEQ ID NO: 300 - DNA, 396 bp, Artificial Sequence
  source: /note=“Description of Artificial Sequence Synthetic polynucleotide”
atgaagtccctgcgggtgctgctggttattctgtggctgcagctgagctgggtttggagccagggacagcaagtgatgcagatccctcagtaccagcacgtgcaagaaggcgaggacttcaccacctactgcaacagcagcacaaccctgagcaacatccagtggtacaagcagaggcctggcgggcaccctgtgtttctgatccagctggttaagagcggcgaagtgaagaagcagaagcggctgacctttcagttcggcgaggccaagaagaacagcagcctgcacattaccgccacacagaccaccgacgtgggcacatatttttgcgctggcagagaaggcggcagcgagaagctggtttttggcaagggcaccaaactgaccgtgaatccc

SEQ ID NO: 301 - protein, 132 aa, Artificial Sequence
  source: /note=“Description of Artificial Sequence Synthetic polypeptide”
MKSLRVLLVILWLQLSWVWSQGQQVMQIPQYQHVQEGEDFTTYCNSSTTLSNIQWYKQRPGGHPVFLIQLVKSGEVKKQKRLTFQFGEAKKNSSLHITATQTTDVGTYFCAGREGGSEKLVFGKGTKLTVNP

SEQ ID NO: 302 - DNA, 493 bp, Artificial Sequence
  source: /note=“Description of Artificial Sequence Synthetic polynucleotide”
gacattcagaaccccgatccagccgtgtaccagctgagagacagcaagagcagcgacaagagcgtgtgtctgttcaccgacttcgactcccagaccaatgtgtcccagagcaaggactccgacgtgtacatcaccgataagaccgtgctggacatgcggagcatggacttcaagagcaatagcgccgtggcttggagcaacaagagcgactttgcctgcgccaacgccttcaacaacagcatcatccccgaggacacattcttcccaagtcctgagagcagctgcgacgtgaagctggtggaaaagagcttcgagacagacaccaacctgaacttccagaacctgagcgtgatcggcttcagaatcctgctgctgaaggtggccggcttcaacctgctgatgactctgagactgtggtccagctgaattcggatccaagcttaggcctgctcgctttcttgctgtcccatttctattaaaggttcctttgttc

SEQ ID NO: 303 - protein, 141 aa, Artificial Sequence
  source: /note=“Description of Artificial Sequence Synthetic polypeptide”
DIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 304 - protein, 5 aa, Artificial Sequence
  source: /note=“Description of Artificial Sequence Synthetic peptide”
TTLSN

SEQ ID NO: 305 - protein, 7 aa, Artificial Sequence
  source: /note=“Description of Artificial Sequence Synthetic peptide”
LVKSGEV

SEQ ID NO: 306 - protein, 11 aa, Artificial Sequence
  source: /note=“Description of Artificial Sequence Synthetic peptide”
AGREGGSEKLV

SEQ ID NO: 307 - protein, 5 aa, Artificial Sequence
  source: /note=“Description of Artificial Sequence Synthetic peptide”
ENHRY

SEQ ID NO: 308 - protein, 6 aa, Artificial Sequence
  source: /note=“Description of Artificial Sequence Synthetic peptide”
SYGVKD

SEQ ID NO: 309 - protein, 11 aa, Artificial Sequence
  source: /note=“Description of Artificial Sequence Synthetic peptide”
AISGYKNTEAF

SEQ ID NO: 310 - DNA, 405 bp, Artificial Sequence
  source: /note=“Description of Artificial Sequence Synthetic polynucleotide”
atgggacctggattgctttgttgggccctgctgtgtctgcttggagctggacttgtggatgccggcgtgacacagtctcccacacacctgatcaagaccagaggccagcaagtgaccctgagatgtagccctaagagcggccacgacaccgtgtcttggtatcagcaggctcttggccagggacctcagttcatcttccagtactacgaggaagaggaacggcagcggggcaacttccctgatagattctctggccatcagttccccaactacagcagcgagctgaacgtgaacgctctgctgctgggcgatagcgccctgtatctgtgtgccagttctcttggttggagaggcggcagatacaacgagcagttctttggccctggcaccagactgaccgtgctg

SEQ ID NO: 311 - protein, 135 aa, Artificial Sequence
  source: /note=“Description of Artificial Sequence Synthetic polypeptide”
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGWRGGRYNEQFFGPGTRLTVL

SEQ ID NO: 312 - DNA, 537 bp, Artificial Sequence; identical to SEQ ID NO: 296
  source: /note=“Description of Artificial Sequence Synthetic polynucleotide”

SEQ ID NO: 313 - protein, 179 aa, Artificial Sequence; identical to SEQ ID NO: 297
  source: /note=“Description of Artificial Sequence Synthetic polypeptide”

SEQ ID NO: 314 - DNA, 81 bp, Artificial Sequence; identical to SEQ ID NO: 298
  source: /note=“Description of Artificial Sequence Synthetic oligonucleotide”

SEQ ID NO: 315 - protein, 27 aa, Artificial Sequence
  source: /note=“Description of Artificial Sequence Synthetic peptide”
RAKRSGSGATNFSLLKQAGDVEENPGP

SEQ ID NO: 316 - DNA, 390 bp, Artificial Sequence
  source: /note=“Description of Artificial Sequence Synthetic polynucleotide”
atgaagtccctgcgggtgctgctggttattctgtggctgcagctgagctgggtttggagccagggacagaacatcgaccagcctaccgagatgacagccaccgaaggcgccatcgtgcagatcaattgcacctaccagaccagcggcttcaacggcctgttctggtatcaacagcatgccggcgaggcccctaccttcctgagctataatgtgctggacggcctggaagaaaagggcagattcagcagcttcctgtccagaagcaagggctacagctacctgctgctgaaagaactccagatgaaggacagcgcctcctacctgtgtgcctccgtggatggaaacaacagactggccttcggcaagggcaaccaggtggtggtcatcccc

SEQ ID NO: 317 - protein, 130 aa, Artificial Sequence
  source: /note=“Description of Artificial Sequence Synthetic polypeptide”
MKSLRVLLVILWLQLSWVWSQGQNIDQPTEMTATEGAIVQINCTYQTSGFNGLFWYQQHAGEAPTFLSYNVLDGLEEKGRFSSFLSRSKGYSYLLLKELQMKDSASYLCASVDGNNRLAFGKGNQVVVIP

SEQ ID NO: 318 - DNA, 493 bp, Artificial Sequence; identical to SEQ ID NO: 302
  source: /note=“Description of Artificial Sequence Synthetic polynucleotide”

SEQ ID NO: 319 - protein, 141 aa, Artificial Sequence; identical to SEQ ID NO: 303
  source: /note=“Description of Artificial Sequence Synthetic polypeptide”

SEQ ID NO: 320 - protein, 6 aa, Artificial Sequence
  source: /note=“Description of Artificial Sequence Synthetic peptide”
TSGFNG

SEQ ID NO: 321 - protein, 6 aa, Artificial Sequence
  source: /note=“Description of Artificial Sequence Synthetic peptide”
NVLDGL

SEQ ID NO: 322 - protein, 10 aa, Artificial Sequence
  source: /note=“Description of Artificial Sequence Synthetic peptide”
ASVDGNNRLA

SEQ ID NO: 323 - protein, 5 aa, Artificial Sequence
  source: /note=“Description of Artificial Sequence Synthetic peptide”
SGHDT

SEQ ID NO: 324 - protein, 6 aa, Artificial Sequence
  source: /note=“Description of Artificial Sequence Synthetic peptide”
YYEEEE

SEQ ID NO: 325 - protein, 15 aa, Artificial Sequence
  source: /note=“Description of Artificial Sequence Synthetic peptide”
ASSLGWRGGRYNEQF

SEQ ID NO: 326 - DNA, 6762 bp, Artificial Sequence
  source: /note=“Description of Artificial Sequence Synthetic polynucleotide”
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgacccggggatggcgcgccagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagccgccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgcgatcgcggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtccttcgaagtagatctttgtcgatcctaccatccactcgacacacccgccagcggccgctgccaagcttccgagctctcgaattaattcacggtacccaccatggcctagggagactagtcgaatcgatatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtccttttcatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggttaattaaagtacctttaagaccaatgacttacaaggcagctgtagatcttagccactttttaaaagaaaaggggggactggaagggcgaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcaggcatgccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttggcgcgccatcgtcgaggttccctttagtgagggttaattgcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac